(19) United States
(12) Patent Application Publication
(10) Pub. No.: US 2022/0016127 A1
(43) Pub. Date: Jan. 20, 2022

(54) COMPOSITIONS AND METHODS FOR INDUCING APPENDAGE AND LIMB REGENERATION

(71) Applicant: California Institute of Technology, Pasadena, CA (US)

(72) Inventors: Lea Goentoro, Pasadena, CA (US); Michael J. Abrams, Berkeley, CA (US); Fayth Tan, Pasadena, CA (US); Martin Heithe, Pasadena, CA (US); Ty Basinger, Bloomsburg, PA (US)

(21) Appl. No.: 17/355,727

(22) Filed: Jun. 23, 2021

Related U.S. Application Data
(60) Provisional application No. 63/043,585, filed on Jun. 24, 2020.

(51) Int. Cl. A61K 31/5377; A61K 31/365; A61K 45/06; A61K 31/198; A61K 38/28

(52) U.S. Cl. CPC A61K 31/5377; A61K 31/365; A61K 38/28; A61K 31/198; A61K 45/06

(57) ABSTRACT

Disclosed herein include methods, compositions, and kits suitable for use in inducing reparative regeneration and/or appendage regeneration. In some embodiments, the method comprises administering to a subject in need thereof a therapeutically effective amount of one or more amino acids and a therapeutically effective amount of one or more sugars, and thereby inducing reparative regeneration or appendage regeneration in the subject. There are provided, in some embodiments, regenerative agents suitable for use in the methods provided herein. Regenerative agents can stimulate mTOR signaling and/or insulin signaling. Regenerative agents can include one or more amino acids, insulin, and/or one or more sugars.

### RELATED APPLICATIONS

This application claims the benefit under 35 U.S.C. § 119(e) of U.S. Provisional Patent Application Ser. No. 63/043,585, filed Jun. 24, 2020, the content of this related application is incorporated herein by reference in its entirety for all purposes.

### REFERENCE TO SEQUENCE LISTING

The present application is being filed along with a Sequence Listing in electronic format. The Sequence Listing is provided as a file entitled 30KJ 302434 US, created Jun. 23, 2021, which is 10 kilobytes in size. The information in the electronic format of the Sequence Listing is incorporated herein by reference in its entirety.

### BACKGROUND

### Field

The present disclosure relates generally to the fields of reparative regeneration and appendage regeneration.

### Description of the Related Art

In contrast to humans' poor ability to regenerate, the animal world is filled with seemingly Homeric tales: a creature that regrows when halved or a whole animal growing from a small body piece. Two views have historically prevailed as to why some animals regenerate better than others. Some biologists, including Charles Darwin and August Weismann, hold that regeneration is an adaptive property of a specific organ. For instance, some lobsters may evolve the ability to regenerate claws because they often lose them in fights and food foraging. Other biologists, including Thomas Morgan, hold that regeneration is not an evolved trait of a particular organ, but inherent in all organisms. Regeneration evolving for a particular organ versus regeneration being organismally inherent is an important distinction, as the latter suggests that the lack of regeneration is not due to the trait never having evolved, but rather due to inactivation—and may therefore be induced. In support of Morgan's view, studies in past decades have converged on one striking insight: many animal phyla have at least one or more species that regenerate body parts. Further, even in poorly regenerative lineages, many embryonic and larval stages can regenerate. In fish, conserved regeneration-responsive enhancers were recently identified, which are also modified in mice. These findings begin to build the case that, rather than many instances of convergence, the ability to regenerate is ancestral. Regeneration being ancestral begs the question: is there a conserved mechanism to activate regenerative state?

There is a need to explore whether limbs can be made to regenerate in animals that do not normally show limb regeneration. In frogs, studies from the early 20th century and few recent ones have induced various degrees of outgrowth in the limb using strategies including repeated trauma, electrical stimulation, local progesterone delivery, progenitor cell implantation, and Wnt activation. Wnt activation restored limb development in chick embryos, but there are no reports of postnatal regeneration induction. In salamanders, a wound site that normally just heals can be induced to grow a limb by supplying nerve connection and skin graft from the contralateral limb, or by delivery of Fgf2, 8, and Bmp2 to the wound site followed by retinoic acid. In mouse digits, a model for exploring limb regeneration in mammals, bone outgrowth or joint-like structure can be induced via local implantation of Bmp2 or 9. Thus far, different strategies gain tractions in different species, and a common denominator appears elusive.

### SUMMARY

Disclosed herein include methods for inducing reparative regeneration. In some embodiments, the method comprises administering to a subject in need thereof: a therapeutically effective amount of a first regenerative agent or a pharmaceutically acceptable salt, ester, solvate, stereoisomer, tautomer, or prodrug thereof, that stimulates mTOR signaling; and a therapeutically effective amount of a second regenerative agent or a pharmaceutically acceptable salt, ester, solvate, stereoisomer, tautomer, or prodrug thereof, that stimulates insulin signaling, and thereby inducing reparative regeneration in the subject. Disclosed herein include methods for inducing reparative regeneration or appendage regeneration, comprising administering to a subject in need thereof a therapeutically effective amount of one or more amino acids and a therapeutically effective amount of one or more sugars, and thereby inducing reparative regeneration or appendage regeneration in the subject.

Disclosed herein include methods for inducing appendage regeneration. In some embodiments, the method comprises administering to a subject in need thereof: a therapeutically effective amount of a first regenerative agent or a pharmaceutically acceptable salt, ester, solvate, stereoisomer, tautomer, or prodrug thereof, that stimulates mTOR signaling; and a therapeutically effective amount of a second regenerative agent or a pharmaceutically acceptable salt, ester, solvate, stereoisomer, tautomer, or prodrug thereof, that stimulates insulin signaling, and thereby inducing appendage regeneration in the subject. In some embodiments, the first regenerative agent comprises MHY1485, 3BDO, CL316,243, or any combination thereof. In some embodiments, the first regenerative agent comprises one or more amino acids.

Disclosed herein include methods for inducing reparative regeneration. In some embodiments, the method comprises administering to a subject in need thereof: a therapeutically effective amount of a first regenerative agent or a pharmaceutically acceptable salt, ester, solvate, stereoisomer, tautomer, or prodrug thereof, wherein the first regenerative agent comprises one or more amino acids; and a therapeutically effective amount of a second regenerative agent or a pharmaceutically acceptable salt, ester, solvate, stereoisomer, tautomer, or prodrug thereof, that stimulates insulin signaling, and thereby inducing reparative regeneration in the subject.

Disclosed herein include methods for inducing appendage regeneration. In some embodiments, the method comprises administering to a subject in need thereof: a therapeutically effective amount of a first regenerative agent or a pharmaceutically acceptable salt, ester, solvate, stereoisomer, tautomer, or prodrug thereof, wherein the first regenerative agent comprises one or more amino acids; and a therapeutically effective amount of a second regenerative agent or a pharmaceutically acceptable salt, ester, solvate, stereoisomer, tautomer, or prodrug thereof, that stimulates insulin signaling, and thereby inducing appendage regeneration in the subject.

In some embodiments, the one or more amino acids comprises alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine, phosphoserine, phosphothreonine, phosphotyrosine, 4-hydroxyproline, hydroxylysine, demosine, isodemosine, gamma-carboxyglutamate, hippuric acid, octahydroindole-2-carboxylic acid, statine, 1,2,3,4-tetrahydroi soquinoline-3-carboxylic acid, penicillamine, ornithine, 3-methylhistidine, norvaline, beta-alanine, gamma-aminobutylic acid, cirtulline, homocysteine, homoserine, methyl-alanine, para-benzoylphenylalanine, phenylglycine, propargylglycine, sarcosine, methionine sulfone, tert-butylglycine, 3,5-dibromotyrosine, 3,5-diiodotyrosine, glycosylated threonine, glyclosylated serine, glycosylated asparagine, or any combination thereof. In some embodiments, the one or more amino acids is in a D- or L-configuration. In some embodiments, the one or more amino acids comprises leucine. In some embodiments, the leucine is in a D- or L-onfiguration. In some embodiments, the one or more amino acids comprises L-leucine. In some embodiments, the one or more amino acids comprises glutamine. In some embodiments, the glutamine is in a D- or L-configuration.

In some embodiments, the second regenerative agent comprises an insulin receptor agonist. In some embodiments, the insulin receptor agonist comprises an insulin analogue, an insulin fragment, an insulin alpha chain, an insulin beta chain, pro-insulin, pre-pro-insulin, porcine insulin, bovine insulin, human insulin, synthetic insulin, Demethylasterriquinone B 1, HNG6A, IGF1, IGF2, or any combination thereof. In some embodiments, the second regenerative agent comprises insulin and/or one or more sugars. In some embodiments, the one or more sugars comprise a monosaccharide, a disaccharide, a polysaccharide, or any combination thereof. In some embodiments, the one or more sugars comprise sucrose, dextrose, maltose, dextrin, xylose, ribose, glucose, mannose, galactose, sucromalt, fructose (levulose), or any combination thereof. In some embodiments, the second regenerative agent increases insulin secretion.

In some embodiments, the subject in need thereof is a subject suffering from or at a risk to develop a disease or disorder, wherein the disease or disorder results in damage, injury, or loss of a limb, organ, tissue, cell, or any combination thereof. In some embodiments, the subject in need is suffering from an acute injury. In some embodiments, the acute injury comprises injury, loss, or amputation of a limb. In some embodiments, the injury, loss, or amputation of the limb is caused by accident, war, cancer, diabetes, congenital disease, or a combination thereof. In some embodiments, the limb comprises an arm, a leg, a hand, a finger, a foot, a toe, a phalange, portions thereof, or any combination thereof. In some embodiments, the limb injury, loss, or amputation is entirely proximal to a visible nail. In some embodiments, the reparative regeneration and/or appendage regeneration comprises regeneration of one or more tissues. In some embodiments, the one or more tissues comprises bone, muscle, epidermis, nervous tissues, connective tissues, epithelial tissues, adipose tissues, or any combination thereof. In some embodiments, the regeneration of the one or more tissues comprises regeneration of a hematopoietic cell, an immune cell, a nerve cell, a neural cell, a glial cell, an astrocyte, a muscle cell, a cardiac cell, a liver cell, a hepatocyte, a pancreatic cell, a fibroblast cell, a connective tissue cell, a skin cell, a melanocyte, an adipose cell, an exocrine cell, a dermal cell, a keratinocyte, a retinal cell, a Muller cell, a mucosal cell, an esophageal cell, an epidermal cell, a bone cell, a chondrocyte, an osteoblast, an osteocyte, a prostate cell, an ovary cell, a testis cell, an adipose tissue cell, or a cancer cell, or any combination thereof. In some embodiments, the reparative regeneration and/or appendage regeneration is patterned. In some embodiments, the reparative regeneration and/or appendage regeneration comprises regeneration of phalange 3 and nail of the lost limb.

In some embodiments, the first and second regenerative agents are administered concurrently. In some embodiments, the first and second regenerative agents are administered as a single composition. In some embodiments, the first and second regenerative agents are administered sequentially. In some embodiments, the administration of the first regenerative agent and the administration of the second regenerative agent overlap in part with each other. In some embodiments, the first regenerative agent is administered before initiating administration of the second regenerative agent. In some embodiments, the second regenerative agent is administered before initiating administration of the first regenerative agent. In some embodiments, the administration of the first regenerative agent continues after cessation of administration of the second regenerative agent. In some embodiments, the administration of the second regenerative agent continues after cessation of administration of the first regenerative agent. In some embodiments, the first regenerative agent and the second regenerative agent are administered in different compositions.

In some embodiments, the administration of one or both of the first and second regenerative agents is initiated within a therapeutically effective time window. In some embodiments, the administration of one or both of the first and second regenerative agents is initiated immediately after, less than one hour after, or more than one hour after the acute injury. In some embodiments, the administration of one or both of the first and second regenerative agents comprises ad libitum administration. In some embodiments, the administration of one or both of the first and second regenerative agents comprises continuous administration. In some embodiments, the administration of one or both of the first and second regenerative agents is repeated one or more times per day. In some embodiments, the administration of one or both of the first and second regenerative agents is repeated hourly, daily, or weekly. In some embodiments, the administration of one or both of the first and second regenerative agents is continued for a period of time comprising 1 week after initiation, 2 weeks after initiation, 3 weeks after initiation, 4 weeks after initiation, 5 weeks after initiation, 6 weeks after initiation, 7 weeks after initiation, 8 weeks after initiation, or more than 8 weeks after initiation.

In some embodiments, one or both of the first and second regenerative agents are administered in an amount of about 1 μg/kg, about 5 μg/kg, about 10 μg/kg, about 20 μg/kg, about 30 μg/kg, about 40 μg/kg, about 50 μg/kg, about 60 μg/kg, about 70 μg/kg, about 80 μg/kg, about 90 μg/kg, about 100 μg/kg, about 200 μg/kg, about 300 μg/kg, about 400 μg/kg, about 500 μg/kg, about 600 μg/kg, about 700 μg/kg, about 800 μg/kg, about 900 μg/kg, about 1 mg/kg, about 5 mg/kg, about 10 mg/kg, about 20 mg/kg, about 30 mg/kg, about 40 mg/kg, about 50 mg/kg, about 60 mg/kg, about 70 mg/kg, about 80 mg/kg, about 90 mg/kg, about 100 mg/kg, about 200 mg/kg, about 300 mg/kg, about 400 mg/kg, about 500 mg/kg, about 600 mg/kg, about 700 mg/kg, about 800 mg/kg, about 900 mg/kg, about 1 g/kg, about 2 g/kg, about 3 g/kg, about 4 g/kg, about 5 g/kg, about 6 g/kg, about 7 g/kg, about 8 g/kg, about 9 g/kg, about 10 g/kg, about 20 g/kg, about 30 g/kg, about 70 g/kg, about 100 g/kg, about 300 g/kg, about 500 g/kg, about 700 g/kg, about 900 g/kg, or about 1000 g/kg.

In some embodiments, one or both of the first and second regenerative agents are in a single unit dosage form. In some embodiments, one or both of the first and second regenerative agents are in two or more unit dosage forms. In some embodiments, the administration of the first regenerative agent, the administration of the second regenerative agent, or both is oral, topical, intravenous, intraperitoneal, intragastric, intravascular, or any combination thereof. In some embodiments, one or both of the first and second regenerative agents are formulated for oral administration. In some embodiments, one or both of the first and second regenerative agents are administered in a foodstuff, a food supplement, or a pharmaceutical composition. In some embodiments, the foodstuff comprises a nutritional complete formula, a dairy product, a chilled or shelf stable beverage, a mineral water, a liquid drink, a shot, a soup, a dietary supplement, a meal replacement bar, a nutritional bar, a confectionery product, a milk, a fermented milk product, a yogurt, a pectin chew, a gummy, a milk based powder, an enteral nutrition product, a cereal product, a fermented cereal based product, an ice cream, a chocolate, coffee, a culinary product, or any combination thereof. In some embodiments, the foodstuff is a beverage. In some embodiments, the food supplement is in the form of capsules, gelatin capsules, soft capsules, tablets, sugar-coated tablets, powders, pills, pastes, pastilles, gums, drinkable solutions, drinkable emulsions, syrups, gels, or any combination thereof. In some embodiments, the pharmaceutical composition is in the form of capsules, gelatin capsules, soft capsules, tablets, chewable tablets, sugar-coated tablets, pills, pastes or pastilles, powders, softgels, chewable softgels, gums, drinkable solutions or emulsions, syrups, gels, or any combination thereof. In some embodiments, the pharmaceutical composition comprises one or more of binding agents, gelling agents, thickeners, colorants, taste masking agents, stabilizers, antioxidants, coatings, sweeteners, taste modifiers, and aroma chemicals. In some embodiments, the pharmaceutical composition comprises one or more pharmaceutically acceptable carriers, diluents, and/or excipients.

The method can comprise administering a third regenerative agent that activates mTOR signaling. In some embodiments, the first regenerative agent and third regenerative agent is selected from the group comprising MHY1485, 3BDO, and CL316,243. In some embodiments, the first and third regenerative agents are different. In some embodiments, the method comprises inducing mTOR expression. In some embodiments, the method does not induce insulin resistance. In some embodiments, the method comprises contacting the subject in need with a scaffold, wherein the scaffold comprises a bandage, beads, a hydrogel, a polymer, or other biomaterial, or any combination thereof; and wherein the scaffold comprises a bone morphogenetic protein (BMP), a hormone, a growth factor, or other agent that induces reparative regeneration and/or appendage regeneration, or any combination thereof. In some embodiments, the contacting results in a synergistic effect on regeneration.

### BRIEF DESCRIPTION OF THE DRAWINGS

The patent or application file contains at least one drawing executed in color. Copies of this patent or patent application publication with drawing(s) will be provided by the Office upon request and payment of the necessary fee.

FIG. 1A-FIG. 1F depict non-limiting exemplary embodiments showing appendage regeneration in Aurelia ephyra can be induced. FIG. 1A depicts non-limiting exemplary embodiments showing the Aurelia life cycle. FIG. 1B depicts non-limiting exemplary embodiments showing amputation was performed across the body, removing three arms. FIG. 1C depicts non-limiting exemplary data showing amputated ephyrae reorganize existing arms, often within hours. FIG. 1D depicts non-limiting exemplary data showing a small bud appears in ˜1 of 50 symmetrizing ephyrae. FIG. 1E depicts non-limiting exemplary data showing a rudimentary arm grows in 2 of 18 ephyrae in the natural habitat. FIG. 1F shows non-limiting exemplary data related to induced arm regeneration (arrows). FIG. 1A is modified from R. Buschbaum et al. Animals without backbones: An introduction to the invertebrates (Chicago University Press, Chicago, Ill., 1987), FIG. 1C is reproduced from M. J. Abrams et al., Proc. Natl. Acad. Sci. U.S.A. 112(26), E3365-73 (2015).

FIG. 2A-FIG. 2C depict non-limiting exemplary embodiments showing arm regeneration was induced using L-leucine and insulin. FIG. 2A shows non-limiting exemplary data related to arm regeneration in high food, 500 nM insulin, hypoxia, or combination thereof. Low and high food amounts differ by two-fold. For frequency measurement, regeneration was quantified as anywhere from rudimentary to complete arms (arrows in FIG. 1F), measured from multiple independent experiments of >3000 ephyrae across clutches; individual experiments are tabulated in Table 2A-Table 2C. FIG. 2B shows non-limiting exemplary data related to amputated ephyrae treated with DMSO (as control), 1 nM sapanisertib, or 1 mM A769662. FIG. 2C shows non-limiting exemplary data related to amputated ephyrae recovering in low-food condition, with or without 100 mM L-leucine. * p-value<0.05, ** p-value<0.01, student's t-test. *** p-value<0.001, student's t-test.

FIG. 3A-FIG. 3G depict non-limiting exemplary embodiments showing feeding with L-leucine and insulin induced leg regeneration in Drosophila. FIG. 3A depicts a non-limiting exemplary drawing of an adult Drosophila melanogaster. FIG. 3B depicts non-limiting exemplary embodiments showing amputation was performed on a hindlimb, across the middle of the tibia. FIG. 3C depicts non-limiting exemplary embodiments showing amputated flies were fed with standard lab food (control) or standard lab food supplemented with 5 mM L-Leucine, 5 mM L-Glutamine, and 0.1 mg/mL insulin (treated). Each fly was examined at multiple time points after amputation. FIG. 3D-FIG. 3E depict non-limiting exemplary data related to close-up images of the tibia 3 days after amputation in control (FIG. 3D) and treated (FIG. 3E) flies. FIG. 3F depicts non-limiting exemplary data showing regrown tibias were observed in the treated population 1-3 weeks after amputation. FIG. 3G shows non-limiting exemplary data related to scanning electron microscopy of a regrown tibia. Top inset: control cut tibia. Bottom inset: close-up of the regenerated joint-like structure. Black asterisk: condyles projecting from the tibial end. Grey asterisk: mid projection from the tibial end. Arrow: opposing tibial/tarsal segments touch along condyle ridge.

FIG. 4A-FIG. 4I depict non-limiting exemplary embodiments showing L-leucine and sucrose induced digit regeneration in adult mice. FIG. 4A-FIG. 4C depict non-limiting exemplary embodiments showing amputation was performed on a hind paw (FIG. 4A), on digit 2 and 4 proximal to the nail (FIG. 4B). It is established in the field that amputation that removes <30% length of the third phalange (P3) regenerates, whereas amputation that removes of >60% length of P3 does not regenerate. Amputation was performed proximal to the 60% boundary, that corresponds to removing the entire visible nail. FIG. 4D-FIG. 4F depict non-limiting exemplary embodiments showing upon amputation, mice were given regular drinking water (control) or drinking water supplemented with 1.5% L-leucine, 1.5% L-Glutamine, and 4% sucrose (% in w/v). Shown are the amputated digits 7 weeks after amputation in control mouse (FIG. 4D) and treated mice (FIG. 4E-FIG. 4F). Arrows indicate regenerating digits. FIG. 4G-FIG. 4I depict non-limiting exemplary data related to whole-mount skeletal staining of the amputated digits. At 7 weeks after amputation, the entire digit (from the base of the first phalange P1) was dissected, and together with the preserved original portions removed, stained with Alizarin red, an anionic dye that binds to calcium. Because 99% of the calcium in the body is localized to the bone, Alizarin red stain strongly localizes to the bone.

FIG. 5A-FIG. 5B depict non-limiting data showing muscular and neuronal networks are regrown in the arm regenerate. Existing (FIG. 5A) and regenerate (FIG. 5B) arm stained with phalloidin and tyrosinated tubulin antibody. Phalloidin stains actin, enriched in the myofibrils of epitheliomuscular cells. Tyrosinated tubulin is enriched in motor nerve net. Arrow indicates distal enrichment of tyrosinated tubulin staining, a marker for the light- and gravity-sensing organ rhopalium.

FIG. 6A-FIG. 6B depict non-limiting exemplary embodiments showing induced regeneration was observed across clonal lines. The animals used in the study originate from a wild population. To develop a genetically clonal line, a polyp was isolated and settled onto a plastic plate. In 1-3 months, with daily feeding of enriched brine shrimps, each plate was re-populated by polyps asexually propagating from the single starter polyp. Within each clonal line, a fraction of the polyps in each plate was regularly passaged to new plates to avoid crowding and expand the clonal population. Regeneration induction was performed in these multiple clonal lines. Results from two clonal lines are shown here from experiments performed side by side. In each line, varying range of regeneration (arrows) was observed suggesting that the variation was not entirely due to genetic variation. Note how variation manifests even within individuals. The data disclosed in the Examples below come from experiments performed in clone 3 (FIG. 6B).

FIG. 7 depicts non-limiting exemplary embodiments showing water current is a permissive requirement for induction of regeneration. Various physical environments for the ephyrae recovering from injury were tested, e.g., shallow vs deep water, seawater with varying salinity, cold vs warm temperature, light versus dark, stagnant versus current, generating the current through various means, including shaking to generate horizontal waves, rotating to generate turbulent mixing, air bubbling to generate vertical current (shown here). A bubbler cone setup pictured here connected to an air pump was used to generate a gentle vertical current of ˜1 bubble/second (FIG. 30). While symmetrization occurred robustly in all conditions, consistent induction of regeneration only occurred in columnar water current.

FIG. 8 depicts non-limiting exemplary embodiments showing ephyrae treated with insulin or grown in reduced oxygen tend to be bigger. In this experiment, amputated ephyrae were fed only (with low amount of food), fed and treated with 500 nM insulin, or fed and grown in hypoxia (by flowing nitrogen). These pictures were taken at 2 weeks after amputation, with the same magnification (scale bar: 2 mm). Black arrows indicate regenerating arms.

FIG. 9A-FIG. 9E depict non-limiting exemplary embodiments showing the mTOR pathway is conserved in Aurelia and across cnidarians. Conservation of the mTOR pathway in Aurelia (FIG. 9A) and four other cnidarian species (FIG. 9B-FIG. 9E). The mTOR pathway was drawn according to the Kyoto Encyclopedia of Genes and Genomes (KEGG) and (R. A. Saxton et al., Cell 168(6), 960-76 (2017)). Gene presence or absence for Aurelia is based on the KEGG annotation of the gene models (file “Aurelia_Trinotate.Report.txt” on GitHub). The other animals are present in the KEGG database, and presence/absence is based on their annotation for the KEGG mTOR pathway (KO04150). Exceptions to this approach include p53, which is not in the KEGG mTOR pathway, and sestrin2, which is not present in the Aurelia genome—the presence of these genes was verified using reciprocal BLAST.

FIG. 10A-FIG. 10D show non-limiting exemplary data related to RNAseq to test whether mTOR and mTOR-related genes are differentially expressed in the regenerating-inducing condition. Ephyrae were sequenced pre-amputation (t =0), 27 hours post-amputation without feeding, and 27 hours post-amputation with abundant food. All ephyrae were treated in the same way before amputation, i.e., fed the same amount of food (see Example 1, Methods). The 27-hour time point was focused on because the first 24 hours post-amputation is dominated by wound closure processes, and this is when the earliest morphological evidence of regeneration was observed. FIG. 10A shows non-limiting exemplary data related to comparing the three conditions to each other, 5,305 differentially expressed genes are recovered (p-value<0.001, log-fold change>4). The heat map was generated using Trinity (see Example 1, Methods). Gene clustering is visualized through the tree on the left side of the figure. Expression levels are normalized using log 2 FPKM (fragments per kilobase per million reads). FIG. 10B depicts non-limiting exemplary data showing the putative Aurelia mTOR (gene XLOC_029150) is recovered as differentially expressed; its expression significantly drops upon amputation, but notably, remains higher in the high-food condition that induces regeneration. FIG. 10C shows non-limiting exemplary data related to clustering analysis to identify genes with expression profiles similar to Aurelia mTOR. The effect of different tree height cutoff values for clustering the gene expression profile was assessed. The dark line in each chart signifies the average expression profile for each cluster. FIG. 10D shows non-limiting exemplary data related to gene ontology analysis of the gene cluster with expression profile similar to Aurelia mTOR. A 10% cutoff value was chosen to balance gene numbers and retaining the general shape of expression profile. The resulting 332 genes in the cluster are enriched in 948 gene ontology (GO) biological process terms, 31% of which include Aurelia mTOR as an associated gene. These mTOR-related GO-terms include growth, regulators of metabolism, responses to nutrients, hypoxia, and leucine, as well as muscle, neuron, and epithelium development. The GO terms were visualized using the REVIGO algorithm, that clusters the terms based on semantic similarity, the degree to which entities are similar in the meaning of their annotations. Circle size indicates significance, with a larger circle denoting a smaller p-value.

FIG. 11A-FIG. 11B depict non-limiting exemplary embodiments showing conservation of the Aurelia mTOR gene. FIG. 11A depicts a non-limiting exemplary mTOR phylogeny constructed using the maximum likelihood inference computed with the IQ-TREE stochastic algorithm, and visualized using ITOL (https://itol.embl.de/upload.cgi). The simple tree is not meant to be comprehensive or limiting, but serves to confirm whether the gene XLOC_029150 in the Aurelia gene models that is annotated as mTOR is indeed an mTOR gene by testing its conservation with known mTOR genes in other organisms. IQ-TREE parameters: consensus tree was constructed from 1000 bootstrap trees; log-likelihood of the consensus tree is −9.96E-4; the Robinson-Foulds distance is 0. FIG. 11B shows non-limiting exemplary data related to alignment of the kinase domain of the human (SEQ ID NO:1) and Aurelia mTOR (SEQ ID NO:2) performed using the NCBI protein blast. Key residues of the ATP binding pocket are: Asn2343, Asp2357, Asp2338, His2340, Trp2239, Ile2163, Pro2169, and Leu2185.

FIG. 12A-FIG. 12G depict non-limiting exemplary embodiments showing flies fed with L-leucine and insulin showed live tip instead of dead clot. FIG. 12A-FIG. 12E depict non-limiting exemplary embodiments showing tibias were dissected, fixed, and mounted in Vectashield mounting medium with DAPI. As reported by others, insect cuticle is variably penetrable by stains; this assay is therefore restricted to assess nuclear staining at the amputated tip. FIG. 12A, FIG. 12C show non-limiting exemplary data showing control tibia (6 of 6), 3 days after amputation, showed clotted tip (FIG. 12A) that does not stain with DAPI (FIG. 12C). FIG. 12B, FIG. 12D show non-limiting exemplary data related to treated tibia (6 of 7), 3 days after amputation, showed live tip (FIG. 12B) that stains positive with DAPI (FIG. 12D). The dashed lines outline the tibia. FIG. 12E shows non-limiting exemplary data related to confocal image of a DAPI-stained treated tibia, 2 weeks after amputation, showing DAPI-positive cells at the tip (inset). FIG. 12F-FIG. 12G show non-limiting exemplary data related to tibia 3 days after amputation, with muscle fibers labeled using tau-GFP driven by muscle-specific myosin heavy chain promoter. The dashed lines in (FIG. 12F) outline the tibia, which does not show any fluorescent signals.

FIG. 13A-FIG. 13C depict non-limiting exemplary embodiments showing Aurelia as a system to identify factors that promote appendage regeneration. FIG. 13A depicts non-limiting exemplary embodiments showing the moon jellyfish Aurelia aurita have a dimorphic life cycle, existing as sessile polyps or free-swimming medusae and ephyrae. Ephyra is the juvenile stage of medusa, a robust stage that can withstand months of starvation. In lab conditions, ephyrae mature into medusae, growing bell tissue and reproductive organs, in 1-2 months. FIG. 13B depicts non-limiting exemplary embodiments showing ephyrae have eight arms, which are swimming appendages that contract synchronously to generate axisymmetric fluid flow, which facilitates propulsion and filter feeding. The eight arms are symmetrically positioned around the stomach and the feeding organ manubrium. Extending into each arm is radial muscle (shown in FIG. 14A-FIG. 14E) and a circulatory canal that transports nutrients. At the end of each arm is the light- and gravity-sensing organ rhopalium. FIG. 13C depicts non-limiting exemplary data showing in response to injury, the majority of ephyrae rapidly reorganize existing body parts and regain radial symmetry. However, performing the experiment in the natural habitat, a few ephyrae (2 of 18) regenerated a small arm (arrow).

FIG. 14A-FIG. 14E depict non-limiting exemplary embodiments showing arm regeneration in Aurelia ephyra can be induced using exogenous factors. FIG. 14A depicts non-limiting exemplary embodiments showing ephyrae were amputated (dashed line) across the body to remove 3 arms, and then let recover in various conditions. Table 5A-Table 5B tabulate the molecular and physical factors tested in the screen. Regeneration was assessed over 1-2 weeks until bell tissues began developing between the arms and obscured scoring. FIG. 14B shows non-limiting exemplary data related to arm regeneration (arrows; from high food condition, see FIG. 15C). FIG. 14C depicts non-limiting exemplary data showing radial circulatory canal in an uncut arm and is reformed in an arm regenerate. FIG. 14D shows non-limiting exemplary data related to muscle, as indicated by phalloidin staining, and neuronal networks, as indicated by antibody against tyrosinated tubulin. The arrows indicate distal enrichment of tyrosinated-tubulin staining, which marks the sensory organ rhopalium (rho). Twenty ephyrae were examined and representative images are shown. FIG. 14E depicts non-limiting exemplary data related to higher magnification of the phalloidin staining shows the striated morphology of the regrown muscle in the arm regenerate (called radial muscle), which extends seamlessly from circular muscle in the body. The specific ephyrae shown came from high-nutrient condition (see FIG. 15A-FIG. 15G), and are representative of the regeneration observed in other conditions. (Also see Table 5A-Table 5B, FIG. 19, FIG. 20A-FIG. 20C)

FIG. 15A-FIG. 15G depict non-limiting exemplary embodiments showing nutrient level, insulin, hypoxia, and leucine increased regeneration frequency in Aurelia ephyra. FIG. 15A depicts non-limiting exemplary embodiments showing an ephyra is regenerating if it has at least one growth from the cut site with a length greater than 0.15 of the uncut arm length. The uncut arm length was determined in each ephyra by measuring 3 uncut arms and taking the average. Lappets, the distal paired flaps, were excluded in the length measurement because their shapes tend to vary across ephyrae. The measurements were performed in ImageJ. FIG. 15B depicts non-limiting exemplary embodiments showing the threshold 0.15 was chosen to balance excluding non-specific growths that show no morphological structures (e.g., as shown, lack of phalloidin-stained structures) and retaining rudimentary arms that show morphological structures, including radial muscle sometime with growing ends (shown, phalloidin stained). FIG. 15C-FIG. 15F depict non-limiting exemplary embodiments showing in each experiment, treated (light grey) and control (dark grey) ephyrae came from the same strobilation. FIG. 15C shows non-limiting exemplary data related to regeneration frequency in lower amount of food (LF) and higher amount of food (HF). The designation “high” and “low” is for simplicity, and does not presume the nutrient level in the wild. Without being bound by any particular theory, the LF amount is likely closer to typical nutrient level in the wild, based on two lines of evidence. First, regeneration frequency in LF is comparable to that observed in the natural habitat experiment. Second, in many of the wild populations studied, ephyrae mature to medusae over 1-3 months, comparable to the growth rate in LF (by contrast, ephyrae in HF mature to medusae over 3-4 weeks). FIG. 15D shows non-limiting exemplary data related to regeneration frequency in 500 nM insulin. FIG. 15E shows non-limiting exemplary data related to regeneration frequency in ASW with reduced oxygen. FIG. 15F shows non-limiting exemplary data related to ephyrae recovering in low food, with or without 100 mM L-leucine. FIG. 15G shows non-limiting exemplary data related to the effect size of a treatment was computed from the ratio between regeneration frequency in treated and control group within an experiment, i.e., the metric Risk Ratio (RR; RR=1 means the treatment has no effect). The statistical significance and reproducibility of a treatment was assessed by analyzing the effect size across experiments using the meta-analysis package, metafor, in R with statistical coefficients based on normal distribution. See Example 2, Methods for more details. A treatment was deemed reproducible if the 95% confidence intervals (95% CI) of RR exclude 1. The p-value evaluates the null hypothesis that the estimate RR is 1. Reproducibility and statistical significance of each treatment were verified using another common size effect metric, Odds Ratio (FIG. 23, Table 6). (Also see FIG. 21, FIG. 22A-FIG. 22B, FIG. 23, FIG. 24A-FIG. 24D, FIG. 25A-FIG. 25B, Table 6-Table 8)

FIG. 16A-FIG. 16E depict non-limiting exemplary embodiments showing experimental design to assess regeneration in Drosophila limb FIG. 16A depicts a non-limiting exemplary drawing of an adult Drosophila. FIG. 16B depicts non-limiting exemplary embodiments showing the Drosophila limb is a jointed limb, with rigid segments connected by flexible joints. Amputation was performed on the fourth segment, the tibia. FIG. 16C depicts non-limiting exemplary embodiments showing a hindlimb before (left) and immediately after (right) amputation. The red-shaded region indicates the amputation site. FIG. 16D depicts non-limiting exemplary embodiments showing after amputation, flies were housed in vials containing standard lab food (control) or standard lab food supplemented L-leucine and insulin (treated). FIG. 16E depicts non-limiting exemplary embodiments showing regeneration was assessed at 7-21 days post amputation (dpa).

FIG. 17A-FIG. 17J depict non-limiting exemplary embodiments showing leucine and insulin induced regeneration in Drosophila limb. In these experiments, upon amputation described in FIG. 16A-FIG. 16E, flies were placed in vials with standard laboratory food (control) or standard lab food added with 5 mM L-Leucine, 5 mM L-Glutamine, and 0.1 mg/mL insulin (treated). Doses were determined through observing the highest order of magnitude dose of amino acid that could be fed to flies over a prolonged period without shortening their lifespan. The flies were then examined at 1, 3, 7, 14, and 21 days post amputation (dpa). Images in FIG. 17A-FIG. 17E were taken from anesthetized live flies, whereas fluorescent images in FIG. 17F-FIG. 17H were from dissected hindlimbs. FIG. 17A depicts non-limiting exemplary data showing a control and a treated fly, imaged at 7 dpa. FIG. 17B depicts non-limiting exemplary data showing an uncut hindlimb, showing distal part of femur, tibia, and proximal part of tarsus. FIG. 17C shows non-limiting exemplary data related to control tibia stumps show melanized clotted ends from 3 dpa onward. FIG. 17D shows non-limiting exemplary data related to at 1-3 dpa, some tibia stumps in the treated population showed no clots. Sometimes a dark bruising appears near the amputation plane. FIG. 17E shows non-limiting exemplary data related to at 7-21 dpa, regrown tibias, which culminate in joints, were observed in the treated population. A dark bruise is present in one of the regrown tibias, suggesting where the amputation was. Also observed at 7-21 dpa in the treated population are some tibias stumps with non-specific growth, which stain positive for DAPI (staining method described next). FIG. 17F-FIG. 17G depict non-limiting exemplary embodiments showing tibia stumps at 3-14 dpa were dissected, fixed, and mounted in Vectashield mounting medium with DAPI. Samples from 14 dpa are shown here. Insect cuticle is not dissected to restrict DAPI penetrance only to the distal tip. Clotted tips of control tibia stumps did not stain with DAPI (FIG. 17F, 10 of 10), whereas unclotted tips of treated tibia stumps stained with DAPI (FIG. 17G, 14 of 16). FIG. 17H shows non-limiting exemplary data related to higher-resolution confocal image of an unclotted tip of a treated tibia stump at 14 dpa showing DAPI-positive cells. FIG. 17I shows non-limiting exemplary data related to fly with a regrown tibia at 21 dpa (an earlier picture of this regrown tibia is the top panel in FIG. 17E) was mounted onto an environmental SEM with a copper stub. Inset shows a clotted tibia stump from a control fly, with the discoloration at the end corresponding to the clot. FIG. 17J shows non-limiting exemplary data related to magnification of the regenerated joint, with the arrows denoting the two condyles and the additional ventral projection.

FIG. 18A-FIG. 18K depict non-limiting exemplary embodiments showing leucine and sucrose induced regeneration in adult mouse digit. FIG. 18A-FIG. 18B depict non-limiting exemplary embodiments showing amputation was performed on hindpaws of adult (3-6 month old) mice, on digits 2 and 4, proximal to the nail. FIG. 18C shows a non-limiting exemplary schematic of the distal phalange (P3) and middle phalange (P2). Amputations that remove <30% of P3 (right dashed line) regenerate, whereas amputations that remove >60% of P3 (left dashed line) do not regenerate. Amputations in the intermediate region can occasionally show partial regenerative response. FIG. 18D depicts non-limiting exemplary embodiments showing amputations in this study were performed within the triangle. FIG. 18E depicts non-limiting exemplary embodiments showing amputated mice were given regular drinking water (control) or drinking water supplemented with 1.5% L-leucine, 1.5% L-glutamine, and 4-10 w/v % sucrose (2 exps with 4%, 6 exps with 10%). Drinking water, control and treated, was refreshed weekly. FIG. 18F shows non-limiting exemplary data related to a representative paw from the control group. The amputated digits 2 and 4 simply healed the wound and did not regrow the distal phalange. FIG. 18G depicts non-limiting exemplary data showing in this treated mouse, digit 2 (arrow) regrew the distal phalange and nail. Insets on the right show the digit at earlier time points. At week 1, the amputation site still appeared inflamed. At week 3, the beginning of the nail appears (arrow). At week 3, a clear nail plate was observed. FIG. 18H depicts non-limiting exemplary data showing in this treated mouse, digit 4 (arrow) regrew and began to show nail reformation by week 4 (top inset, see arrow), that turns into a clear nail plate by week 7 (middle inset), as can be seen more clearly from the side-view darkfield image (bottom inset). FIG. 18I-FIG. 18K show non-limiting exemplary data related to whole-mount skeletal staining. Dissected digits were stained with Alizarin red, an anionic dye that highly localizes to the bone. Top panels show illustration of the amputation plane, bottom left panels show skeletal staining of the portions removed, and bottom right panels show skeletal staining of the digit stumps 7 weeks after amputation. (Also see FIG. 26A-FIG. 26B, FIG. 27A-FIG. 27F, Table 10-Table 12)

FIG. 19 depicts non-limiting exemplary embodiments showing bell growth limited the time window for assessing arm regeneration. Ephyrae in the lab mature into full-belled medusae within ˜4 weeks. The transition to medusa commences at 1-2 weeks after strobilation, with the onset of bell growth. Over 2-3 weeks, body tissues gradually grow and fill between the discrete arms to form a continuous bell characteristic of a medusa. Arm regeneration can be unambiguously scored in ephyrae before the bell has significantly grown. Bell growth also limited testable doses in some factors, e.g., testing higher food amounts than reported here led to accelerated bell growth at a rate that did not allow enough time window to quantify regeneration.

FIG. 20A-FIG. 20C depict non-limiting exemplary embodiments showing variable extent of regeneration was observed in clonal lines. FIG. 20A depicts non-limiting exemplary embodiments showing to develop genetically clonal lines, single polyps were isolated and settled onto tissue culture dishes. Within 1-3 months, with daily feeding of enriched brine shrimps, each dish was re-populated with polyps asexually budding from the single parental polyp. FIG. 20B shows non-limiting exemplary data related to regeneration induction with high food performed in two clonal lines. Arrows indicate arm regenerates. FIG. 20C shows non-limiting exemplary data related to regeneration frequency in the clonal and original mixed populations measured in the same experiment. The data described in the Examples below come from experiments performed in clone 3.

FIG. 21 depicts non-limiting exemplary embodiments showing water current is a permissive requirement for arm regeneration induction. Various physical environments for the ephyrae recovering from injury were tested, e.g., shallow vs deep water, seawater with varying salinity, cold vs warm temperature, light versus dark, stagnant water vs current, generating water current through various means, including shaking or rotating to generate turbulent mixing and as shown here air bubbling a conical tube to generate vertical current (shown here). While symmetrization occurred robustly in all conditions, consistent induction of regeneration only occurred in the presence of columnar water current. The experiments presented in this study were performed in the bubbler cone setup, where a 1L sand settling cone was repurposed into an aquarium and connected to an air pump to generate a gentle current of ˜1 bubble/second (FIG. 30). Each cone housed 30 ephyrae in 500 mL ASW or treated ASW, refreshed weekly, to avoid crowding and fouling. In the bubbler cone, ephyrae continually move along water current, either the upward bubble-generated current or the downward gravity-generated current. The conical geometry helps minimize stagnant spots, where the ephyrae could get stuck.

FIG. 22A-FIG. 22B depict non-limiting exemplary embodiments showing conservation of insulin receptor and HIFα in Aurelia. Phylogenies of insulin receptor (FIG. 22A) and HIFα (FIG. 22B) genes were constructed using the maximum likelihood inference computed with the IQ-TREE stochastic algorithm, and visualized using ITOL (https://itol.embl.de/upload.cgi). These trees verify the simple trees are not meant to be comprehensive or limiting, but a verification of the genes annotated as insulin-like protein receptor (ILPR) and HIFα in the Aurelia gene models by testing conservation with their known counterparts in other organisms. IQ-TREE parameters: Insulin receptor consensus tree is constructed from 1000 bootstrap trees; log-likelihood of consensus tree is -45374.0; the Robinson-Foulds distance between ML and consensus tree is 0. HIFα consensus tree is constructed from 1000 bootstrap trees; log-likelihood of consensus tree is −24414.4; the Robinson-Foulds distance between ML and consensus tree is 0.

FIG. 23 shows non-limiting exemplary data related to statistical significance of regeneration induction in Aurelia assessed using Odds Ratio. In addition to RR analysis presented in FIG. 15G, another common measure of effect size is the Odds Ratio (OR). OR compares the odds of outcome in the presence vs. absence of treatment (see Example 2, Methods). Analysis of OR across experiments was performed using the metafor package in R with statistical coefficients based on normal distribution (see Example 2, Methods). A treatment is reproducible if the 95% confidence intervals (95% CI) exclude 1. The p-value evaluates the null hypothesis that the estimate OR is 1. Also see Table 6.

FIG. 24A-FIG. 24D depict non-limiting exemplary embodiments showing regeneration phenotypes in (FIG. 24A) high amount of nutrients, (FIG. 24B) insulin, (FIG. 24C) hypoxia, and (FIG. 24D) L-leucine. For each treatment, Left: The percentage of ephyrae that regenerate (from left to right) 0, 1, 2, or 3 arms. Middle: The length(s) of arm regenerate(s) in ephyrae that regenerate (from left to right) 1 arm, 2 arms, and 3 arms—normalized to the average length of uncut arms in the same ephyra. For ephyrae with multiple arm regenerates, lengths of all arms were measured and plotted individually. Boxplot: median (line), average (cross), 1st and 3rd quartiles (the box), 5th and 95th percentile (whiskers), and individual data points (black circles). Right: The percentage of ephyrae that reform rhopalia in control (bottom) and treated (top) groups.

FIG. 25A-FIG. 25B depict non-limiting exemplary embodiments showing ephyrae in high food, insulin, or hypoxia, and L-leucine tend to be bigger in size. FIG. 25A depicts non-limiting exemplary data showing representative images of ephyrae growing in low food, 500 nM insulin, and hypoxia. Black arrows indicate regenerating arms. FIG. 25B shows non-limiting exemplary data related to effect size analysis of the body size increase was performed using the metafor package in R (see Example 2, Methods). A treatment effect is reproducible if the 95% CI exclude1. The p-value evaluates the hypothesis that there is no effect. Also see Table 8.

FIG. 26A-FIG. 26B depict non-limiting exemplary embodiments showing mouse digit phenotypes. Whole-mount skeletal staining was performed with Alizarin red. wpa: week post amputation, P1: phalange 1, P2: phalange 2, P3: phalange 3, s: sesamoid bone. FIG. 26A shows non-limiting exemplary data related to skeletal staining of unamputated digits (digit 3) from control and treated groups show no obvious differences in uncut digits due to the treatment. FIG. 26B shows non-limiting exemplary data related to skeletal staining of digits stumps at 7 wpa and the original portion removed from the digits. Some digit stumps show no change or appear to have undergone histolysis resulting in reduced bone mass (Phenotype 1 and 2). Some digit stumps show regenerative response, either recovery of some morphological characteristics (Phenotype 3, detailed more in FIG. 18A-18K) or excess, ectopic bone mass (Phenotype 4). It was erred on the conservative side in scoring phenotype 3 and 4; when in doubt, digits were classified into phenotype 1 or 2. Also see Table 10-Table 12.

FIG. 27A-FIG. 27F depict non-limiting exemplary embodiments showing regenerative response observed in mouse digit. Six digit stumps (of total 48 examined) show regenerative response. The most dramatic two are presented in FIG. 18A-FIG. 18K. The remaining four are presented here. wpa: week post amputation, P1: phalange 1, P2: phalange 2, P3: phalange 3, s: sesamoid bone FIG. 27A shows non-limiting exemplary data related to an uncut digit, shown for a comparison. Magnified is the P2/P3 joint area to highlight key morphological markers: the knobby epiphyseal cap of P2 and the sesamoid bone embedded in the tendon on the flexor side of P2. FIG. 27B shows non-limiting exemplary data related to digit stumps from control mice show either bone stump histolysis (top and middle, phenotypel) and no visible changes in bone stump (bottom, phenotype 2). FIG. 27C-FIG. 27F show nonlimiting exemplary data related to digit stumps from treated mice that show regenerative response. FIG. 27C depicts non-limiting exemplary data showing in this digit, the amputation removed all P3 by a cut through the joint. At 7 wpa, the P2 stump is reduced, but recovered the epiphyseal-like end (dashed line)—marked by solid curved shape, as opposed to irregularly shaped histolyzing bone. FIG. 27D depicts non-limiting exemplary data showing in this digit, the amputation removed a significant portion of P2 and the sesamoid bone. The P2 stump does not regain an epiphyseal end (the end is concave and irregular). However, the sesamoid bone is reformed, as identified by its location on the flexor side of P2 and wingnut shape under the microscope. The recovery of sesamoid bone is non-trivial, as digit sesamoids form in juxtaposition to the condensing phalange, detaching from the phalange by formation of a cartilaginous joint. FIG. 27E depicts non-limiting exemplary data showing in this digit, the amputation removed a significant portion of P2 and the sesamoid bone. At 7 wpa, the P2 stump appears to be reforming an epiphyseal, rounded end (thick dashed line). There is a small bone distal to P2, whose curvature articulates with the P2 end, but there are not enough morphological characters to identify the bone. FIG. 27F depicts non-limiting exemplary data showing in this digit, the amputation removed the epiphyseal cap of P2 and the sesamoid bone. The P2 stump appears to have lost some mass, but reforms an epiphyseal-like end (thick dashed line). There is an additional small bone located where the sesamoid bone should be, but lacks sufficient morphological characters to identify.

FIG. 28A-FIG. 28B show non-limiting exemplary data related to control digits amputated at proximal P3 do not regenerate. wpa: weeks post amputation.

FIG. 29 depicts non-limiting exemplary embodiments showing a time-lapse series of images of regeneration pulsing in Aurelia. The black arrow above the images denotes time moving from left to right. Dashed boxes outline a pulse.

FIG. 30 depicts non-limiting exemplary embodiments showing the Aurelia experimental setup. The black arrow above the images denotes time moving from left to right. The white arrow points to an individual bubble moving up the cone over time.

FIG. 31A-FIG. 31B shows non-limiting exemplary data related to treatments using bovine serum albumin and argon. FIG. 31A depicts non-limiting exemplary data showing that treatment with 500 nM bovine serum albumin (BSA) did not produce significant effect in regeneration frequency (95% CI [0.9, 1.9-fold change], p-value=0.20). FIG. 31B depicts non-limiting exemplary data showing that reducing oxygen using argon flow increased regeneration frequency (95% CI [1.99,3.3-foldchange],p-value<10⁻⁴). Effect size was computed using the metric Risk Ratio (see Methods below). LF is low food, HF is high food, Exp: Experiment, % is percentage of regenerating ephyra in control (black) and treated (light grey), N: number of ephyrae in the control (black) and treated (light grey) group.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

Disclosed herein include methods for inducing reparative regeneration. In some embodiments, the method comprises administering to a subject in need thereof: a therapeutically effective amount of a first regenerative agent or a pharmaceutically acceptable salt, ester, solvate, stereoisomer, tautomer, or prodrug thereof, that stimulates mTOR signaling; and a therapeutically effective amount of a second regenerative agent or a pharmaceutically acceptable salt, ester, solvate, stereoisomer, tautomer, or prodrug thereof, that stimulates insulin signaling, and thereby inducing reparative regeneration in the subject.

Disclosed herein include methods for inducing appendage regeneration. In some embodiments, the method comprises administering to a subject in need thereof: a therapeutically effective amount of a first regenerative agent or a pharmaceutically acceptable salt, ester, solvate, stereoisomer, tautomer, or prodrug thereof, that stimulates mTOR signaling; and a therapeutically effective amount of a second regenerative agent or a pharmaceutically acceptable salt, ester, solvate, stereoisomer, tautomer, or prodrug thereof, that stimulates insulin signaling, and thereby inducing appendage regeneration in the subject.

Disclosed herein include methods for inducing reparative regeneration. In some embodiments, the method comprises administering to a subject in need thereof: a therapeutically effective amount of a first regenerative agent or a pharmaceutically acceptable salt, ester, solvate, stereoisomer, tautomer, or prodrug thereof, wherein the first regenerative agent comprises one or more amino acids; and a therapeutically effective amount of a second regenerative agent or a pharmaceutically acceptable salt, ester, solvate, stereoisomer, tautomer, or prodrug thereof, that stimulates insulin signaling, and thereby inducing reparative regeneration in the subject.

Disclosed herein include methods for inducing appendage regeneration. In some embodiments, the method comprises administering to a subject in need thereof: a therapeutically effective amount of a first regenerative agent or a pharmaceutically acceptable salt, ester, solvate, stereoisomer, tautomer, or prodrug thereof, wherein the first regenerative agent comprises one or more amino acids; and a therapeutically effective amount of a second regenerative agent or a pharmaceutically acceptable salt, ester, solvate, stereoisomer, tautomer, or prodrug thereof, that stimulates insulin signaling, and thereby inducing appendage regeneration in the subject.

Disclosed herein include methods for treating a disease or disorder, wherein the disease or disorder results in damage, injury, or loss of a limb, organ, tissue, cell, or any combination thereof. In some embodiments, the method comprises administering to a subject in need a therapeutically effective amount of a first regenerative agent or a pharmaceutically acceptable salt, ester, solvate, stereoisomer, tautomer, or prodrug thereof, wherein the first regenerative agent comprises one or more amino acids; and, a therapeutically effective amount of a second regenerative agent or a pharmaceutically acceptable salt, ester, solvate, stereoisomer, tautomer, or prodrug thereof, wherein the first regenerative agent stimulates insulin signaling, and thereby treating the disease or disorder, wherein the disease or disorder results in damage, injury, or loss of a limb, organ, tissue, cell, or any combination thereof.

Disclosed herein include methods for treating an acute injury in a subject, wherein the acute injury comprises injury, loss, or amputation of a limb. In some embodiments, the method comprises administering to a subject in need a therapeutically effective amount of a first regenerative agent or a pharmaceutically acceptable salt, ester, solvate, stereoisomer, tautomer, or prodrug thereof, wherein the first regenerative agent comprises one or more amino acids; and, a therapeutically effective amount of a second regenerative agent or a pharmaceutically acceptable salt, ester, solvate, stereoisomer, tautomer, or prodrug thereof, wherein the second regenerative agent stimulates insulin signaling, and thereby treating the acute injury.

### Definitions

Unless defined otherwise, technical and scientific terms used herein have the same meaning as commonly understood by one of ordinary skill in the art to which the present disclosure belongs. See, e.g. Singleton et al., Dictionary of Microbiology and Molecular Biology 2nd ed., J. Wiley & Sons (New York, N.Y. 1994); Sambrook et al., Molecular Cloning, A Laboratory Manual, Cold Spring Harbor Press (Cold Spring Harbor, N.Y. 1989). For purposes of the present disclosure, the following terms are defined below.

As used herein, a “subject” refers to an animal that is the object of treatment, observation or experiment. “Animals” include cold- and warm-blooded vertebrates and invertebrates such as fish, shellfish, reptiles and, in particular, mammals. “Mammal” includes, without limitation, mice; rats; rabbits; guinea pigs; dogs; cats; sheep; goats; cows; horses; primates, such as monkeys, chimpanzees, and apes, and, in particular, humans.

As used herein, “administration” or “administering” refers to a method of giving a dosage of a pharmaceutically active ingredient or agent to a vertebrate.

As used herein, “therapeutically effective amount” or “pharmaceutically effective amount” is meant an amount of therapeutic agent, which has a therapeutic effect. The dosages of a pharmaceutically active ingredient or agent which are useful in treatment are therapeutically effective amounts. Thus, as used herein, a therapeutically effective amount means those amounts of therapeutic agent which produce the desired therapeutic effect as judged by clinical trial results and/or model animal studies.

As used herein, a “dosage” refers to the combined amount of the active ingredients (e.g., first and second regenerative agents).

As used herein, a “unit dosage” refers to an amount of therapeutic agent administered to a patient in a single dose.

As used herein, a “daily dosage” refers to the total amount of therapeutic agent administered to a patient in a day.

As used herein, a “therapeutic effect” relieves, to some extent, one or more of the symptoms of a disease or disorder. For example, a therapeutic effect may be detected by observation of the subject (e.g., regeneration of a lost limb).

“Treat,” “treatment,” or “treating,” as used herein refers to administering a regenerative agent or a composition (e.g., a nutritional composition or a pharmaceutical composition) to a subject for prophylactic and/or therapeutic purposes. The term “prophylactic treatment” refers to treating a subject who does not yet exhibit symptoms of a disease or disorder, or acute injury but who is susceptible to, or otherwise at risk of, a particular disease or disorder, or acute injury whereby the treatment reduces the likelihood that the patient will develop the disease or disorder, or acute injury. The term “therapeutic treatment” refers to administering treatment to a subject already suffering from a disease or disorder, or acute injury.

The terms “prevent”, “preventing” and “prevention” as used herein refer to a method of preventing the onset of a disease and/or its attendant symptoms or barring a subject from acquiring a disease. As used herein, “prevent”, “preventing” and “prevention” also include delaying the onset of a disease and/or its attendant symptoms and reducing a subject's risk of acquiring a disease.

As used herein, a “synergistic” or “synergizing” effect can be such that the one or more effects of the single compositions are greater than the one or more effects of each component alone, or they can be greater than the sum of the one or more effects of each component alone. The synergistic effect can be about, or greater than about 5, 10, 20, 30, 50, 75, 100, 110, 120, 150, 200, 250, 350, or 500% or even more than the effect on a subject with one of the components alone, or the additive effects of each of the components when administered individually. The effect can be any of the measurable effects described herein.

The term “agent,” as used herein, refers to any molecule, entity, or moiety. For example, an agent may be a protein, an amino acid, a peptide, a polynucleotide, a carbohydrate or sugar, a lipid, a metal atom, a non-polypeptide polymer, a synthetic polymer, or chemical compound, such as a small molecule. In some embodiments, the agent is a regenerative agent. Additional agents suitable for use in embodiments of the present invention will be apparent to the skilled artisan. Embodiments provided herein are not limited in this respect

“Amino acid,” as used herein refers broadly to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified (e.g., hydroxyproline, γ-carboxyglutamate, and O-phosphoserine). Amino acid analogs refers to compounds that have the same basic chemical structure as a naturally occurring amino acid (i.e., an a carbon that is bound to a hydrogen, a carboxyl group, an amino group), and an R group (e.g., homoserine, norleucine, methionine sulfoxide, methionine methyl sulfonium.) Analogs may have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

The term “sugar” as used herein refers to substantially all sugars and sugar substitutes, including any monosaccharide such as glucose or fructose, disaccharides such as lactose, sucrose or maltose, polysaccharides such as starch, oligosaccharide, sugar alcohols, or other carbohydrate forms such as gums that are starch based, vegetable based or seaweed based.

As used herein, “insulin” refers to any and all substances having an insulin action, and exemplified by, for example, animal insulin extracted from bovine or porcine pancreas, semi-synthesized human insulin that is enzymatically synthesized from insulin extracted from porcine pancreas, and human insulin synthesized by genetic engineering techniques typically using E. coli or yeasts, etc. Insulin may be in the form of its fragments or derivatives. Insulin may also include insulin-like substances and insulin agonists. While insulin is available in a variety of types such as super immediate-acting, immediate-acting, bimodal-acting, intermediate-acting, long-acting, etc., these types can be appropriately selected according to the subject's need.

### Overview

At least 1 in 190 Americans live with limb loss, a number that is projected to double by the year 2050. Worldwide, one million amputations take place every year—that is, 1 every 30 seconds. Causes of limb loss include diabetes, accident, war, cancer, and congenital disease. Limb loss is deeply devastating to the patient—being incapacitated, coping with pain, change in self-image, loss of job, and increased risk of depression and anxiety. The family's life is turned upside down, organized around surgery, rehabilitation, and adapting to limitations. The collective cost, to private and public insurance, totals more than 12 billion dollars a year. A breakthrough in the technology to induce limb regeneration could render much suffering and burden to taxpayers unnecessary.

As noted above, different regenerative strategies gain tractions in different species, and a common denominator appears elusive. Across animal phylogeny, some physiological features show interesting correlation with regenerative ability. First, regeneration tends to decrease with age, with juveniles and larvae more likely to regenerate than adults. For instance, the mammalian heart rapidly loses the ability to regenerate after birth and anurans cease to regenerate limbs upon metamorphosis. Second, animals that continue to grow throughout life tend to also regenerate. For instance, most annelids continue adding body segments and regenerate well, a striking exception of which is leeches that make exactly 32 segments and one of the few annelids that do not regenerate body segments. Consistent with the notion of regeneration as ancestral, indeterminate growth is thought of as the ancestral state. Finally, a broad correlate of regenerative ability across animal phylogeny is thermal regulation. Poikilotherms, which include most invertebrates, fish, reptiles and amphibians, tend to have greater regenerative abilities than homeotherms—birds and mammals are animal lineages with poorest regeneration. These physiological correlates, taken together, are united by the notion of energy expenditure. The transition from juvenile to adult is a period of intense energy usage, continued growth is generally underlined by sustained anabolic processes, and regulating body temperature is energetically expensive compared to allowing for fluctuation. Regeneration itself entails activation of anabolic processes to rebuild lost tissues. These physiological correlates thus raise the notion of a key role of energetics in the evolution of regeneration in animals. Specifically, it was wondered whether energy inputs can promote regenerative state.

There are provided, in some embodiments, methods, compositions, and kits suitable for use in inducing reparative regeneration and/or appendage regeneration. The method can comprise administering to a subject in need thereof a therapeutically effective amount of one or more regenerative agents (e.g., first regenerative agent, second regenerative agent, third regenerative agent). Regenerative agents can stimulate mTOR signaling and/or insulin signaling. Regenerative agents (e.g., first regenerative agent, second regenerative agent, third regenerative agent) can include one or more amino acids, insulin, and/or one or more sugars. In some embodiments, a regenerative agent disclosed herein (e.g., first regenerative agent, second regenerative agent, third regenerative agent) comprises hypoxia (e.g., exposure to hypoxic conditions). In some embodiments, a regenerative agent disclosed herein (e.g., first regenerative agent, second regenerative agent, third regenerative agent) activates and/or stimulates HIFa signaling.

### Improvements Over Existing Methods

Prostheses is currently the primary approach to regain some functionalities upon limb loss. However, prosthesis use is challenging even for daily functions, lacks sensory feedback, and costly as custom attachments are needed for different daily tasks. On the surgery front, progress has been made in transplanting fingers, hands, and even in a few cases, whole arms. However, success relies on preserving the lost limb, unavailable in crush injuries or disease, while grafting from a donor means immunosuppression, with motor and sensory recovery a slow, year-scale process of unguaranteed success. On the bioengineering front, there is ongoing effort to grow limbs in vitro with stem cell engineering. In the most recent progress, a rat limb was successfully grown in vitro: by taking a cadaver rat limb, stripping away all the cells, and re-seeding the limb scaffold with stem cells. The rat limb tissues regrew, and showed some sensory response.

The dream in the field is to induce the body's own capacity to regenerate limb. This would solve the whole gamut of problems with limited motor-sensory recovery, immunocompatibility, the need for limb donors, the use of cadavers, and associated ethical problems.

Few studies have looked into inducing limb regeneration. In amphibians, three methods have been presented that can induce substantial limb regrowth: (i) Continuous delivery of the hormone progesterone to the amputation site; (ii) Surgical implantation of stem cells combined multiple growth hormones into a genetic mutant; (iii) Surgical procedure to reroute neuronal connection and cell transplantation. In chick embryos, injection of a mutant protein can induce regeneration of a developing limb bud. However, none of these methods has been shown to work in mammals.

In mammals specifically, where the mouse digit is the model for exploring limb regeneration, two methods have been presented to induce regenerative response in the digit. First, implantation at the amputation site of beads coated with developmental proteins was shown to induce specific tissue regrowth, i.e., muscle elongation with Bmp2/4 protein, or joint-like structure with Bmp9 protein. However inducing muscle elongation or joint formation is not the same as inducing a limb with all its multiple tissues to regrow. The second method involves generating a mutant mouse strain to reactivate an embryonic gene 1in28. Newborns with the mutation can excitingly regenerate the distal digit phalange, but the effect does not translate beyond early neonates.

Disclosed herein is a novel method to induce appendage or limb regeneration across species: Administration of amino acids and sugar/insulin induces appendage regeneration in the moon jellyfish Aurelia aurita, limb regeneration in the fruit fly Drosophila melanogaster, and digit regrowth in adult mice. The compositions and methods disclosed herein improve the existing methods in several ways: (i) Rather than species-specific, the method works across multiple species. A method that works across species suggests a more fundamental regulation. (ii) The method works in mammals, i.e., induces digit regeneration in mice. (iii) The method works in adult mice. (iv) The method induces regenerative response from the most dramatic digit amputation tested so far (i.e., mid-phalangeal injury). (v) The method does not require special device for local delivery, surgical transplantation, or engineering genetic mutation. The method also does not require producing special types of proteins or cells. Instead, the method utilizes readily available molecules, amino acid, sugar, and insulin, which can be delivered via dietary supplementation (for amino acid and sugar) or intravenously (for insulin).

### Alternative Embodiments, Variations, Possible Applications of the Disclosed Compositions and Methods

Delivery schedule: In some embodiments, continuous or pulsed delivery of amino acids and sugar/insulin through food or drinking water can induce regeneration.

Systemic vs. local delivery: In some embodiments, ad libitum, systemic delivery of amino acids and sugar/insulin through food or drinking water is sufficient to induce regeneration.

Combination of molecules: In some embodiments, administration of amino acids and sugar/insulin produces substantive regenerative response.

Regeneration of limb vs. other body parts: Amino acid and insulin functions are conserved across all body parts. In some embodiments, the method can work to induce regeneration of other body parts. For example, in cardiovascular disease where metabolic parameters have been found to be important, the method may be used as part of treatment to promote heart regeneration or preventative strategy through dietary modulation.

Disclosed herein include methods for inducing reparative regeneration. In some embodiments, the method comprises administering to a subject in need thereof: a therapeutically effective amount of a first regenerative agent or a pharmaceutically acceptable salt, ester, solvate, stereoisomer, tautomer, or prodrug thereof, that stimulates mTOR signaling; and a therapeutically effective amount of a second regenerative agent or a pharmaceutically acceptable salt, ester, solvate, stereoisomer, tautomer, or prodrug thereof, that stimulates insulin signaling, and thereby inducing reparative regeneration in the subject.

Disclosed herein include methods for inducing appendage regeneration. In some embodiments, the method comprises administering to a subject in need thereof: a therapeutically effective amount of a first regenerative agent or a pharmaceutically acceptable salt, ester, solvate, stereoisomer, tautomer, or prodrug thereof, that stimulates mTOR signaling; and a therapeutically effective amount of a second regenerative agent or a pharmaceutically acceptable salt, ester, solvate, stereoisomer, tautomer, or prodrug thereof, that stimulates insulin signaling, and thereby inducing appendage regeneration in the subject.

As used herein, the term “reparative regeneration” refers to regeneration of: a limb (e.g., finger, toe), an organ (e.g., heart, liver), a tissue (e.g., muscle tissue, nervous tissue), a cell (e.g., muscle cell, epidermal cell), or any combination thereof. As used herein, the term “appendage regeneration” refers to regeneration of an appendage. As used herein, “appendage” means any part that projects from an animal or human body, such as a limb, head or other extremity. The appendage regeneration can comprise regeneration of a limb (e.g., leg, hand), a tissue (e.g., bone tissue, connective tissue), a cell (e.g., epidermal cell, muscle cell), or any combination thereof.

Disclosed herein include methods for inducing reparative regeneration. In some embodiments, the method comprises administering to a subject in need thereof: a therapeutically effective amount of a first regenerative agent or a pharmaceutically acceptable salt, ester, solvate, stereoisomer, tautomer, or prodrug thereof, wherein the first regenerative agent comprises one or more amino acids; and a therapeutically effective amount of a second regenerative agent or a pharmaceutically acceptable salt, ester, solvate, stereoisomer, tautomer, or prodrug thereof, that stimulates insulin signaling, and thereby inducing reparative regeneration in the subject.

Disclosed herein include methods for inducing appendage regeneration. In some embodiments, the method comprises administering to a subject in need thereof: a therapeutically effective amount of a first regenerative agent or a pharmaceutically acceptable salt, ester, solvate, stereoisomer, tautomer, or prodrug thereof, wherein the first regenerative agent comprises one or more amino acids; and a therapeutically effective amount of a second regenerative agent or a pharmaceutically acceptable salt, ester, solvate, stereoisomer, tautomer, or prodrug thereof, that stimulates insulin signaling, and thereby inducing appendage regeneration in the subject.

### Regenerative Agents

First Regenerative Agents

Disclosed herein are first regenerative agents. In some embodiments, the first regenerative agent stimulates mTOR signaling. mTOR (mammalian target of rapamycin) is a major regulator of cell growth and proliferation in response to both growth factors and cellular nutrients. It is a key regulator of the rate limiting step for translation of mRNA into protein, the binding of the ribosome to mRNA. mTOR exists in at least 2 distinct multiprotein complexes described as raptor-mTOR complex (mTORC1) and rictor-mTOR complex (mTORC2) in mammalian cells (sometimes referred to as just TORC1 and TORC2). The term “mTOR1” or “mTOR Complex 1 (mTORC1),” as used herein, means a complex composed of mTOR, regulatory-associated protein of mTOR (Raptor), mammalian LST8/G-protein (3-subunit like protein (mLST8/GβL), and, optionally, the recently identified partners PRAS40 and DEPTOR. mTORC1 is a rapamycin-sensitive complex as its kinase activity is inhibited by FKB12-rapamycin in vitro. The drug rapamycin does not displace GβL or raptor from mTOR but does strongly destabilize the raptor-mTOR interaction. Extensive work with rapamycin indicates that mTORC1 complex positively regulates cell growth. The raptor branch of the mTOR pathway modulates number of processes, including mRNA translation, ribosome biogenesis, nutrient metabolism and autophagy. The two mammalian proteins, S6 Kinase 1 (S6K1) and 4E-BP1, which are linked to protein synthesis, are downstream targets of mTORC1. mTORC1 has been shown to phosphorylates S6K1 at T389 and is inhibited by FKBP12-rapamycin in vitro and by rapamycin in vivo. mTORC1 can also phosphorylate 4E-BP1 at T37/46 in vitro and in vivo.

The first regenerative agent can comprise MHY1485, 3BDO, CL316,243, or any combination thereof. The first regenerative agent can comprise one or more amino acids.

In some embodiments, the one or more amino acids comprise naturally occurring amino acids. In some embodiments, the one or more amino acids comprise synthetic amino acids. In some embodiments, the one or more amino acids comprise amino acid analogs and amino acid mimetics that function in a manner similar to the naturally occurring amino acids. Naturally occurring amino acids are those encoded by the genetic code, as well as those amino acids that are later modified (e.g., hydroxyproline, γ-carboxyglutamate, and O-phosphoserine.) Amino acid analogs refers to compounds that have the same basic chemical structure as a naturally occurring amino acid (i.e., an a carbon that is bound to a hydrogen, a carboxyl group, an amino group), and an R group (e.g., homoserine, norleucine, methionine sulfoxide, methionine methyl sulfonium.) Analogs may have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

The one or more amino acids can comprise alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine, phosphoserine, phosphothreonine, phosphotyrosine, 4-hydroxyproline, hydroxylysine, demosine, isodemosine, gamma-carboxyglutamate, hippuric acid, octahydroindole-2-carboxylic acid, statine, 1,2,3,4-tetrahydroisoquinoline-3-carboxylic acid, penicillamine, ornithine, 3-methylhistidine, norvaline, beta-alanine, gamma-aminobutylic acid, cirtulline, homocysteine, homoserine, methyl-alanine, para-benzoylphenylalanine, phenylglycine, propargylglycine, sarcosine, methionine sulfone, tert-butylglycine, 3,5-dibromotyrosine, 3,5-diiodotyrosine, glycosylated threonine, glyclosylated serine, glycosylated asparagine, or any combination thereof. The one or more amino acids can be in a D- or L-configuration. The one or more amino acids can comprise leucine. The leucine can be in a D- or L-configuration. The one or more amino acids can comprise L-leucine. The one or more amino acids can comprise glutamine. The glutamine can be in a D- or L-configuration.

Leucine is an essential amino acid, being part of a diverse number of proteins and, together with valine and isoleucine, belongs to the group of branched-chain amino acids. Leucine may be used as a free amino acid, or in a bound form, such as a dipeptide, an oligopeptide, a polypeptide or a protein. Common protein sources of leucine are dairy proteins such as whey, casein, micellar casein, caseinate, and glycomacroprotein (GMP), and vegetable proteins such as wheat, rice, pea, lupine and soy proteins. Said sources of protein may provide intact proteins, hydrolysates or mixtures thereof. Leucine is known as a potent stimulator of mTOR signaling. In some embodiments, glutamine enhances leucine uptake.

In some embodiments, administration of the first regenerative agent stimulates mTOR signaling by at least about 2% (e.g., 2%, 3%, 4%, 5%, 6%, 7%, 8%, 9%, 10%, 11%, 12%, 13%, 14%, 15%, 16%, 17%, 18%, 19%, 20%, 25%, 30%, 40%, 50%, 75%, 100%, or higher and overlapping ranges therein) as compared to no administration of the first regenerative agent.

Second Regenerative Agents

Disclosed herein are second regenerative agents. In some embodiments, the second regenerative agent stimulates insulin signaling. Insulin is a potent metabolic and growth promoting hormone that acts on cells to stimulate glucose, protein, and lipid metabolism, as well as RNA and DNA synthesis. A well-known effect of insulin is the regulation of glucose levels in the body. This effect occurs predominantly in liver, fat, and muscle tissue. In the liver, insulin stimulates glucose incorporation into glycogen and inhibits the production of glucose. In muscle and fat tissue, insulin stimulates glucose uptake, storage, and metabolism. Defects in glucose utilization are very common in the population, giving rise to diabetes.

Insulin action is mediated by signal transduction by the insulin receptor. The insulin receptor belongs to the super-family of receptor tyrosine kinases and consists of 2 extracellular alpha subunits and 2 intracellular beta subunits. Insulin binding to the alpha subunit results in a conformational change, which leads to activation of the tyrosine kinase in the intracellular domain, adenosine triphosphate binding and finally receptor autophosphorylation.

Insulin receptor autophosphorylation is followed by phosphorylation of the insulin-receptor substrates (IRS). IRS are related by functional properties and not sequence similarity. Four substrates belong to the family of IRS, IRS-1, IRS-2, IRS-3, and IRS-4. Other substrates include growth factor receptor-bound protein 2 (GRB2)-associated binding protein 1 (Gab-1), p60dok, the c-Cbl proto-oncogene (Cbl), adaptor protein with pleckstrin homology (PH) and Src homology 2 domains (APS) and 3 isoforms of Src homology 2 (SH2) domain-containing alpha-2 collagen-related protein (Shc). IRS contain an NH2-terminal PH domain and/or a phosphotyrosine-binding domain, COOH-terminal tyrosine residues that create SH2 protein-binding sites, proline-rich regions that engage Src homology 3 (SH3) domains or WW domains (protein modules that bind proline-rich lig-ands) and serine-threonine-rich regions that bind other proteins. All substrates, except Shc, contain a SH2 domain that targets the substrate to the insulin receptor. There are 3 main pathways that propagate the signal generated through the insulin receptor: the IRS/phosphatidylinositol 3 (PI3)-kinase pathway; the retrovirus-associated DNA sequences (RAS)/mitogen-activated protein kinase (MAPK) pathway; and the Cbl-associated protein (CAP)/Cbl pathway.

The second regenerative agent can comprise an insulin receptor agonist. The insulin receptor agonist can comprise an insulin analogue, an insulin fragment, an insulin alpha chain, an insulin beta chain, pro-insulin, pre-pro-insulin, porcine insulin, bovine insulin, human insulin, synthetic insulin, Demethylasterriquinone B 1, HNG6A, IGF1, IGF2, or any combination thereof.

As used herein, “insulin” refers to any and all substances having an insulin action. In some embodiments, the insulin is animal insulin extracted from bovine or porcine pancreas. In some embodiments, the insulin is semi-synthesized human insulin that is enzymatically synthesized from insulin extracted from porcine pancreas. In some embodiment, the insulin is human insulin synthesized by genetic engineering techniques typically using E. coli or yeasts, etc. Insulin may be in the form of its fragments or derivatives. The insulin may also include insulin-like substances and insulin agonists. While insulin is available in a variety of types such as super immediate-acting, immediate-acting, bimodal-acting, intermediate-acting, long-acting, etc., these types can be appropriately selected according to the subject's need.

The second regenerative agent can comprise insulin and/or one or more sugars. The one or more sugars can comprise a monosaccharide, a disaccharide, a polysaccharide, or any combination thereof. The one or more sugars can comprise sucrose, dextrose, maltose, dextrin, xylose, ribose, glucose, mannose, galactose, sucromalt, fructose (levulose), or any combination thereof.

In some embodiments, the second regenerative agent increases insulin secretion.

In some embodiments, administration of the second regenerative agent stimulates insulin signaling by at least about 2% (e.g., 2%, 3%, 4%, 5%, 6%, 7%, 8%, 9%, 10%, 11%, 12%, 13%, 14%, 15%, 16%, 17%, 18%, 19%, 20%, 25%, 30%, 40%, 50%, 75%, 100%, or higher and overlapping ranges therein) as compared to no administration of the second regenerative agent.

Synergism

As disclosed herein, co-administration of particular ratios and/or amounts of the first regenerative agent and the second regenerative agent can result in synergistic effects in inducing reparative and/or appendage regeneration. These synergistic effects can be such that the one or more effects of the co-administration are greater than the one or more effects of each component alone at a comparable dosing level, or they can be greater than the predicted sum of the effects of all of the components at a comparable dosing level, assuming that each component acts independently. The synergistic effect can be about, or greater than about, 5, 10, 20, 30, 50, 75, 100, 110, 120, 150, 200, 250, 350, or 500% better than the effect of treating a subject with one of the components alone, or the additive effects of each of the components when administered individually. The effect can be any of the measurable effects described herein. The composition comprising a plurality of components can be such that the synergistic effect is an enhancement in reparative and/or appendage regeneration and that reparative and/or appendage regeneration is increased to a greater degree as compared to the sum of the effects of administering each component, determined as if each component exerted its effect independently, also referred to as the predicted additive effect herein. For example, if a composition comprising component (a) yields an effect of a 20% improvement in reparative and/or appendage regeneration and a composition comprising component (b) yields an effect of 50% improvement in reparative and/or appendage regeneration, then a composition comprising both component (a) and component (b) would have a synergistic effect if the single composition's effect on reparative and/or appendage regeneration was greater than 70%.

A synergistic single composition can have an effect that is greater than the predicted additive effect of administering each component of the single composition alone as if each component exerted its effect independently. For example, if the predicted additive effect is 70%, an actual effect of 140% is 70% greater than the predicted additive effect or is 1 fold greater than the predicted additive effect. The synergistic effect can be at least about 20, 50, 75, 90, 100, 150, 200 or 300% greater than the predicted additive effect. In some embodiments, the synergistic effect can be at least about 0.2, 0.5, 0.9, 1.1, 1.5, 1.7, 2, or 3 fold greater than the predicted additive effect.

In some embodiments, the synergistic effect of the single compositions can also allow for reduced dosing amounts, leading to reduced side effects to the subject and reduced cost of treatment. Furthermore, the synergistic effect can allow for results that are not achievable through any other treatments. Therefore, proper identification, specification, and use of single compositions can allow for significant improvements in inducing reparative and/or appendage regeneration.

### Subjects

The subject in need thereof can be a subject suffering from or at a risk to develop a disease or disorder, wherein the disease or disorder results in damage, injury, or loss of a limb, organ, tissue, cell, or any combination thereof.

In some embodiments, the disease or disorder is a neurodegenerative disease of the central or peripheral nervous system, the result of retinal neuronal cell death, the result of cell death of cardiac muscle, the result of cell death of cells of the immune system; stroke, liver disease, pancreatic disease, the result of cell death associated with renal failure; heart, mesenteric, retinal, hepatic or brain ischemic injury, ischemic injury during organ storage, head trauma, septic shock, coronary heart disease, cardiomyopathy, myocardial infarction, bone avascular necrosis, sickle cell disease, muscle wasting, gastrointestinal disease, tuberculosis, diabetes, alteration of blood vessels, muscular dystrophy, graft-versus-host disease, viral infection, Crohn's disease, ulcerative colitis, asthma, atherosclerosis, a chronic or acute inflammatory condition, pain, or any disease or disorder, wherein the disease or disorder results in damage, injury, or loss of a limb, organ, tissue, cell, or any combination thereof.

In some embodiments, the disease or disorder is hepatic or brain ischemic injury, or ischemic injury during organ storage, head trauma, septic shock, or coronary heart disease. In some embodiments, the disease or disorder is stroke. In other embodiments, the disease or disorder is myocardial infarction. In some embodiments, the disease or disorder is pain (e.g., inflammatory pain, diabetic pain, pain associated with a burn, or pain associated with trauma). In other embodiments, the disease or disorder is atherosclerosis. In some embodiments, the disease or disorder is a chronic or acute inflammatory condition (e.g., rheumatoid arthritis, psoriasis, or Stevens-Johnson syndrome). In some embodiments, the disease or disorder is diabetes.

The subject in need can be suffering from an acute injury. As used herein, the term “acute injury” includes injuries that have occurred suddenly or recently occurred. For example, an acute injury may have occurred suddenly, e.g., due to a traumatic event (external or internal) (e.g., accident or amputation), infections (e.g., caused by bacterial viruses, fungi and parasites), stroke (cerebral circulatory disturbance and intracerebral or subarachnoid hemorrhage), myocardial infarction, and traumatic lesions.

The acute injury can comprise injury, loss, or amputation of a limb. The injury, loss, or amputation of the limb can be caused by accident, war, cancer, diabetes, congenital disease, or a combination thereof. The limb can comprise an arm, a leg, a hand, a finger, a foot, a toe, a phalange, portions thereof, or any combination thereof. The limb injury, loss, or amputation can be entirely proximal to a visible nail. The age for the subject in need can vary. For example, the subject can be an adult, for example a middle-aged adult, or an elderly adult. In some embodiments, the subject is of the age of 20, 25, 30, 35, 40, 45, 50, 55, 60, 65, 70, 75, or more. The gender of the subject in need thereof can vary. In some embodiments, the subject is a female. In some embodiments, the subject is a male.

### Regeneration

Disclosed herein include methods for inducing reparative regeneration. Disclosed herein include methods for inducing appendage regeneration. In some embodiments, the reparative regeneration comprises regeneration of an organ (e.g., bladder, brain, nervous tissue, esophagus, fallopian tube, heart, pancreas, intestines, gallbladder, kidney, liver, lung, ovaries, prostate, spinal cord, spleen, stomach, testes, thymus, thyroid, trachea, ureter, urethra, and uterus.). The reparative regeneration and/or appendage regeneration can comprise regeneration of one or more tissues. The one or more tissues can comprise bone, muscle, epidermis, nervous tissues, connective tissues, epithelial tissues, adipose tissues, or any combination thereof.

The regeneration of the one or more tissues can comprise regeneration of a hematopoietic cell, an immune cell, a nerve cell, a neural cell, a glial cell, an astrocyte, a muscle cell, a cardiac cell, a liver cell, a hepatocyte, a pancreatic cell, a fibroblast cell, a connective tissue cell, a skin cell, a melanocyte, an adipose cell, an exocrine cell, a dermal cell, a keratinocyte, a retinal cell, a Muller cell, a mucosal cell, an esophageal cell, an epidermal cell, a bone cell, a chondrocyte, an osteoblast, an osteocyte, a prostate cell, an ovary cell, a testis cell, an adipose tissue cell, or a cancer cell, or any combination thereof.

The regeneration of the one or more tissues can comprise regeneration of gland cells (e.g., exocrine secretory epithelial cells, salivary gland mucous cells, salivary gland serous cells, Von Ebner's gland cells, mammary gland cells, lacrimal gland cells, ceruminous gland cells, eccrine sweat gland dark cells, eccrine sweat gland clear cells, apocrine sweat gland cells, gland of Moll cells, aebaceous gland cells, Bowman's gland cells, Brunner's gland cells, seminal vesicle cells, prostate gland cells, bulbourethral gland cells, bartholin's gland cells, gland of littre cells, uterine endometrial cells, isolated goblet cells, stomach lining mucous cells, gastric gland zymogenic cells, gastric gland oxyntic cells, pancreatic acinar cells, paneth cells, type II pneumocyte cells, and Clara cells), hormone secreting cells (e.g., anterior pituitary cells, intermediate pituitary cells, magnocellular neurosecretory cells, gut and respiratory tract cells, thyroid gland cells, parathyroid gland cells, adrenal gland cells, chromaffin cells, Leydig theca interna cells, corpus luteum cells, granulosa lutein cells, theca lutein cells, juxtaglomerular cells, racula densa cells, peripolar cells, and mesangial cells), epithelial cells lining closed internal body cavities (e.g., blood vessel and lymphatic vascular endothelial fenestrated cells, blood vessel and lymphatic vascular endothelial continuous cells, blood vessel and lymphatic vascular endothelial splenic cells, synovial cells, serosal cells, squamous cells, columnar cells, dark cells, vestibular membrane cells, stria vascularis basal cells, stria vascularis marginal cells, Claudius cells, Boettcher cells, choroid plexus cells, pia-arachnoid squamous cells, pigmented and non-pigmented ciliary epithelial cells, corneal endothelial cells, and peg cells), ciliated cells of the respiratory tract cells, oviduct cells, uterine endometrium cells, rete testis cells, and ductulus efferens cells, ciliated ependymal cells of central nervous system, keratinizing epithelial cells (e.g., epidermal keratinocyte, epidermal basal cells, keratinocytes, nail bed basal cells, medullary hair shaft cells, cortical hair shaft cells, cuticular hair shaft cells, cuticular hair root sheath cells, hair root sheath cell of Huxley's layer, hair root sheath cell of Henle's layer, external hair root sheath cells, and hair matrix cells), wet stratified barrier epithelial cells (e.g., surface epithelial cell of stratified squamous epithelium of cornea, tongue, oral cavity, esophagus, anal canal, distal urethra and vagina; basal cell of epithelia of the cornea, tongue, oral cavity, esophagus, anal canal, distal urethra, and vagina; and urinary epithelium cells), cells of the nervous system (e.g., sensory transducer cells, auditory inner hair cell of organ of corti, auditory outer hair cell of organ of corti, basal cell of olfactory epithelium, cold-sensitive primary sensory neurons, heat-sensitive primary sensory neurons, Merkel cell of epidermis, olfactory receptor neurons, pain-sensitive primary sensory neurons, photoreceptor cells of the retina, proprioceptive primary sensory neurons, touch-sensitive primary sensory neurons, cholinergic neurons, adrenergic neurons, peptidergic neural cells, inner and outer pillar cells, inner and outer phalangeal cells, border cells, hensen cells, taste bud supporting cells, olfactory epithelium supporting cells, Schwann cells, satellite cells, enteric glial cells, central nervous system neural and glial cells, and lens cells), hepatocyte, adipocytes, liver lipocytes, kidney cells (e.g., glomerulus parietal cells, glomerulus podocyte cells, proximal tubule brush border loop of Henle thin segment cells, distal tubule cells, and collecting duct cells), lung cells, Type I pneumocytes, pancreatic duct cells, nonstriated duct cells, principal cells, intercalated cells, duct cells, intestinal brush border cells, exocrine gland striated duct cells, gall bladder epithelial cells, ductulus efferens nonciliated cells, epididymal principal cells, epididymal basal cells, extracellular matrix cells, ameloblast epithelial cells, planum semilunatum epithelial cells, loose connective tissue fibroblasts, corneal fibroblasts, tendon fibroblasts, bone marrow reticular tissue fibroblasts, nucleus pulpous cells, cementoblast/cementocytes, odontoblast/odontocytes, hyaline cartilage chondrocytes, fibrocartilage chondrocytes, fibroblast cartilage chondrocytes, osteoblast/osteocytes, osteoprogenitor cells, hyalocytes of vitreous body of eye, stellate cells of perilymphatic space of ear, hepatic stellate cells, pancreatic stele cells, contractile cells, skeletal muscle cells, heart muscle cells, smooth muscle cells, blood and immune cells (e.g., erythrocyte, megakaryocyte, monocyte, connective tissue macrophage, epidermal langerhans, osteoclast, dendritic cell, microglial cell, neutrophil granulocyte, eosinophil granulocyte, basophil granulocyte, mast cell, T cell, suppressor T cell, cytotoxic T cell, natural killer T cell, B cell, and reticulocyte), Stem cells and committed progenitors for the blood and immune system (e.g., pigment cells, melanocytes, and retinal pigmented epithelial cells), germ cells (e.g., oocyte, spermatid, spermatocyte, spermatogonium cell, and spermatozoon, nurse cells (e.g., ovarian follicle cell, and sertoli cells, and thymus epithelial cells), interstitial cells, or any combination thereof.

The reparative regeneration and/or appendage regeneration can be patterned. The reparative regeneration and/or appendage regeneration can comprise regeneration of phalange 3 and nail of the lost limb.

The percentage of the damage, injury, or loss of a limb, organ, tissue, cell, that is regenerated can be, or be about, 0.000000001%, 0.00000001%, 0.0000001%, 0.000001%, 0.00001%, 0.0001%, 0.001%, 0.01%, 0.1%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values.

### Administration

Methods of inducing reparative regeneration and/or appendage regeneration. In some embodiments, the method comprises administering a first regenerative agent and a second regenerative agent as disclosed herein. In general, an amount of one or both of the first and second regenerative agent in the levels sufficient to induce reparative regeneration and/or appendage regeneration is administered for a therapeutically effective period of time. Also disclosed herein are one or more compositions. The one or more compositions can comprise one or both of the first and second regenerative agents. The one or more compositions can comprise additional regenerative agents (e.g., a third regenerative agent). The one or more compositions can comprise additional agents (e.g., an anti-bacterial agent). The one or more compositions can comprise additional ingredients (e.g., stabilizers).

The first and second regenerative agents can be administered concurrently. The first and second regenerative agents can be administered as a single composition. The first and second regenerative agents can be administered sequentially. In some embodiments, administration of the first regenerative agent and the administration of the second regenerative agent overlap in part with each other.

The first regenerative agent can be administered before initiating administration of the second regenerative agent. The second regenerative agent can be administered before initiating administration of the first regenerative agent. In some embodiments, the administration of the first regenerative agent continues after cessation of administration of the second regenerative agent. In some embodiments, the administration of the second regenerative agent continues after cessation of administration of the first regenerative agent. The first regenerative agent and the second regenerative agent can be administered in different compositions.

The administration of one or both of the first and second regenerative agents can be initiated within a therapeutically effective time window. The administration of one or both of the first and second regenerative agents can be initiated immediately after, less than one hour after, or more than one hour after the acute injury. The administration of one or both of the first and second regenerative agents can be initiated 1 hour, 2, hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day after the acute injury.

The administration of one or both of the first and second regenerative agents can comprise ad libitum administration. The administration of one or both of the first and second regenerative agents can comprise continuous administration. The administration of one or both of the first and second regenerative agents can be repeated one or more times per day. The administration of one or both of the first and second regenerative agents can be repeated hourly, daily, or weekly.

One or both of the first and second regenerative agents can be administered periodically. For example, one or both of the first and second regenerative agents can be administered one, two, three, four times a day, or even more frequent. One or both of the first and second regenerative agents can be administered every 1, 2, 3, 4, 5, 6, or 7 days. The administration can be concurrent with meal time of a subject. The period of administration can be for about 1, 2, 3, 4, 5, 6, 7, 8, or 9 days, 2 weeks, 1-11 months, or 1 year, 2 years, 5 years, or even longer. The period of administration can be a period of 1 hour, 2, hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 1 week, 8 days, 9 days, 10 days, 11 days, 12 days, 13 days, 2 weeks, 15 days, 16 days, 17 days, 18 days, 19 days, 20 days, 3 weeks, 22 days, 23 days, 24 days, 25 days, 26 days, 27 days, 28 days, 29 days, 30 days, 31 days, 1 month, 2 months, 3 months, 4 months, 5 months, 6 months, 7 months, 8 months, 9 months, 10 months, 11 months, 1 year, 13 months, 14 months, 15 months, 16 months, 17 months, 18 months, 19 months, 20 months, 21 months, 22 months, 23 months, 2 years, 3 years, 4 years, 5 years, 6 years, 7 years, 8 years, 9 years, 10 years, 11 years, 12 years, 13 years, 14 years, 15 years, 16 years, 17 years, 18 years, 19 years, 20 years, 21 years, 22 years, 23 years, 24 years, 25 years, 26 years, 27 years, 28 years, 29 years, 30 years, 31 years, 32 years, 33 years, 34 years, 35 years, 36 years, 37 years, 38 years, 39 years, 40 years, 41 years, 42 years, 43 years, 44 years, 45 years, 46 years, 47 years, 48 years, 49 years, 50 years, 55 years, 60 years, 65 years, or more than 65 years.

In some embodiments disclosed herein, the dosages that are administered to a subject can change or remain constant over the period of treatment. For example, the daily dosing amounts can increase or decrease over the period of administration.

In some embodiments, the dosing regimen of the compositions disclosed herein is administered for a period of time, which time period can be, for example, from at least about 1 week to at least about 4 weeks, from at least about 4 weeks to at least about 8 weeks, from at least about 4 weeks to at least about 12 weeks, from at least about 4 weeks to at least about 16 weeks, or longer. The dosing regimen of the compositions disclosed herein can be administered three times a day, twice a day, daily, every other day, three times a week, every other week, three times per month, once monthly, substantially continuously or continuously.

The administration of one or both of the first and second regenerative agents can be continued for a period of time comprising 1 week after initiation, 2 weeks after initiation, 3 weeks after initiation, 4 weeks after initiation, 5 weeks after initiation, 6 weeks after initiation, 7 weeks after initiation, 8 weeks after initiation, or more than 8 weeks after initiation.

The length of the period of administration and/or the dosing amounts can be determined by a physician, a nutritionist, or any other type of clinician. The period of time can be one, two, three, four or more weeks. In some embodiments, the period of time can be one, two, three, four, five, six or more months.

One or both of the first and second regenerative agents can be administered in an amount of about 1 μg/kg, about 5 μg/kg, about 10 μg/kg, about 20 μg/kg, about 30 μg/kg, about 40 μg/kg, about 50 μg/kg, about 60 μg/kg, about 70 μg/kg, about 80 μg/kg, about 90 μg/kg, about 100 μg/kg, about 200 μg/kg, about 300 μg/kg, about 400 μg/kg, about 500 μg/kg, about 600 μg/kg, about 700 μg/kg, about 800 μg/kg, about 900 μg/kg, about 1 mg/kg, about 5 mg/kg, about 10 mg/kg, about 20 mg/kg, about 30 mg/kg, about 40 mg/kg, about 50 mg/kg, about 60 mg/kg, about 70 mg/kg, about 80 mg/kg, about 90 mg/kg, about 100 mg/kg, about 200 mg/kg, about 300 mg/kg, about 400 mg/kg, about 500 mg/kg, about 600 mg/kg, about 700 mg/kg, about 800 mg/kg, about 900 mg/kg, about 1 g/kg, about 2 g/kg, about 3 g/kg, about 4 g/kg, about 5 g/kg, about 6 g/kg, about 7 g/kg, about 8 g/kg, about 9 g/kg, about 10 g/kg, about 20 g/kg, about 30 g/kg, about 70 g/kg, about 100 g/kg, about 300 g/kg, about 500 g/kg, about 700 g/kg, about 900 g/kg, or about 1000 g/kg.

One or both of the first and second regenerative agents can be in a single unit dosage form. One or both of the first and second regenerative agents can be in two or more unit dosage forms.

A unit dose can be chosen such that the subject is administered about or greater than about 1 μg of one or both of the first and second regenerative agents (e.g. about or more than about 1 μg, 2 μg, 3 μg, 4 μg, 5 μg, 6 μg, 7 μg, 8 μg, 9 μg, 10 μg, 11 μg, 12 μg, 13 μg, 14 μg, 15 μg, 16 μg, 17 μg, 18 μg, 19 μg, 20 μg, 25 μg, 30 μg, 35 μg, 40 μg, 45 μg, 50 μg, 60 μg, 70 μg, 80 μg, 90 μg, 100 μg, 110 μg, 120 μg, 128 μg, 130 μg, 140 μg, 150 μg, 160 μg, 170 μg, 180 μg, 190 μg, 200 μg, 210 μg, 220 μg, 230 μg, 240 μg, 250 μg, 260 μg, 270 μg, 280 μg, 290 μg, 300 μg, 310 μg, 320 μg, 330 μg, 340 μg, 350 μg, 360 μg, 370 μg, 380 μg, 390 μg, 400 μg, 410 μg, 420 μg, 430 μg, 440 μg, 450 μg, 460 μg, 470 μg, 480 μg, 490 μg, 500 μg, 510 μg, 520 μg, 530 μg, 540 μg, 550 μg, 560 μg, 570 μg, 580 μg, 590 μg, 600 μg, 610 μg, 620 μg, 630 μg, 640 μg, 650 μg, 660 μg, 670 μg, 680 μg, 690 μg, 700 μg, 710 μg, 720 μg, 730 μg, 740 μg, 750 μg, 760 μg, 770 μg, 780 μg, 790 μg, 800 μg, 810 μg, 820 μg, 830 μg, 840 μg, 850 μg, 860 μg, 870 μg, 880 μg, 890 μg, 900 μg, 910 μg, 920 μg, 930 μg, 940 μg, 950 μg, 960 μg, 970 μg, 980 μg, 990 μg, 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 25 mg, 30 mg, 35 mg, 40 mg, 45 mg, 50 mg, 60 mg, 70 mg, 80 mg, 90 mg, 100 mg, 110 mg, 120 mg, 128 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg, 400 mg, 410 mg, 420 mg, 430 mg, 440 mg, 450 mg, 460 mg, 470 mg, 480 mg, 490 mg, 500 mg, 510 mg, 520 mg, 530 mg, 540 mg, 550 mg, 560 mg, 570 mg, 580 mg, 590 mg, 600 mg, 610 mg, 620 mg, 630 mg, 640 mg, 650 mg, 660 mg, 670 mg, 680 mg, 690 mg, 700 mg, 710 mg, 720 mg, 730 mg, 740 mg, 750 mg, 760 mg, 770 mg, 780 mg, 790 mg, 800 mg, 810 mg, 820 mg, 830 mg, 840 mg, 850 mg, 860 mg, 870 mg, 880 mg, 890 mg, 900 mg, 910 mg, 920 mg, 930 mg, 940 mg, 950 mg, 960 mg, 970 mg, 980 mg, 990 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300 mg, 2400 mg, 2500 mg, 2600 mg, 2700 mg, 2800 mg, 2900 mg, 3000 mg, 3250 mg, 3500 mg, 3750 mg, 4000 mg, 4250 mg, 4500 mg, 4750 mg, 5000 mg, 5500 mg, 6000 mg, 6500 mg, 7000 mg, 7500 mg, 8000 mg, 8500 mg, 9000 mg, 9500 mg, 10000 mg, or more).

A unit dose can be chosen such that the subject is administered about or greater than about 1 μg of one or both of the first and second regenerative agents (e.g. about or more than about 1 μg, 2 μg, 3 μg, 4 μg, 5 μg, 6 μg, 7 μg, 8 μg, 9 μg, 10 μg, 11 μg, 12 μg, 13 μg, 14 μg, 15 μg, 16 μg, 17 μg, 18 μg, 19 μg, 20 μg, 25 μg, 30 μg, 35 μg, 40 μg, 45 μg, 50 μg, 60 μg, 70 μg, 80 μg, 90 μg, 100 μg, 110 μg, 120 μg, 128 μg, 130 μg, 140 μg, 150 μg, 160 μg, 170 μg, 180 μg, 190 μg, 200 μg, 210 μg, 220 μg, 230 μg, 240 μg, 250 μg, 260 μg, 270 μg, 280 μg, 290 μg, 300 μg, 310 μg, 320 μg, 330 μg, 340 μg, 350 μg, 360 μg, 370 μg, 380 μg, 390 μg, 400 μg, 410 μg, 420 μg, 430 μg, 440 μg, 450 μg, 460 μg, 470 μg, 480 μg, 490 μg, 500 μg, 510 μg, 520 μg, 530 μg, 540 μg, 550 μg, 560 μg, 570 μg, 580 μg, 590 μg, 600 μg, 610 μg, 620 μg, 630 μg, 640 μg, 650 μg, 660 μg, 670 μg, 680 μg, 690 μg, 700 μg, 710 μg, 720 μg, 730 μg, 740 μg, 750 μg, 760 μg, 770 μg, 780 μg, 790 μg, 800 μg, 810 μg, 820 μg, 830 μg, 840 μg, 850 μg, 860 μg, 870 μg, 880 μg, 890 μg, 900 μg, 910 μg, 920 μg, 930 μg, 940 μg, 950 μg, 960 μg, 970 μg, 980 μg, 990 μg, 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 25 mg, 30 mg, 35 mg, 40 mg, 45 mg, 50 mg, 60 mg, 70 mg, 80 mg, 90 mg, 100 mg, 110 mg, 120 mg, 128 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg, 400 mg, 410 mg, 420 mg, 430 mg, 440 mg, 450 mg, 460 mg, 470 mg, 480 mg, 490 mg, 500 mg, 510 mg, 520 mg, 530 mg, 540 mg, 550 mg, 560 mg, 570 mg, 580 mg, 590 mg, 600 mg, 610 mg, 620 mg, 630 mg, 640 mg, 650 mg, 660 mg, 670 mg, 680 mg, 690 mg, 700 mg, 710 mg, 720 mg, 730 mg, 740 mg, 750 mg, 760 mg, 770 mg, 780 mg, 790 mg, 800 mg, 810 mg, 820 mg, 830 mg, 840 mg, 850 mg, 860 mg, 870 mg, 880 mg, 890 mg, 900 mg, 910 mg, 920 mg, 930 mg, 940 mg, 950 mg, 960 mg, 970 mg, 980 mg, 990 mg, 1 g, 2 g, 3 g, 4 g, 5 g, 6 g, 7 g, 8 g, 9 g, 10 g, 11 g, 12 g, 13 g, 14 g, 15 g, 16 g, 17 g, 18 g, 19 g, 20 g, 25 g, 30 g, 35 g, 40 g, 45 g, 50 g, 60 g, 70 g, 80 g, 90 g, 100 g, 110 g, 120 g, 128 g, 130 g, 140 g, 150 g, 160 g, 170 g, 180 g, 190 g, 200 g, 210 g, 220 g, 230 g, 240 g, 250 g, 260 g, 270 g, 280 g, 290 g, 300 g, 310 g, 320 g, 330 g, 340 g, 350 g, 360 g, 370 g, 380 g, 390 g, 400 g, 410 g, 420 g, 430 g, 440 g, 450 g, 460 g, 470 g, 480 g, 490 g, 500 g, 510 g, 520 g, 530 g, 540 g, 550 g, 560 g, 570 g, 580 g, 590 g, 600 g, 610 g, 620 g, 630 g, 640 g, 650 g, 660 g, 670 g, 680 g, 690 g, 700 g, 710 g, 720 g, 730 g, 740 g, 750 g, 760 g, 770 g, 780 g, 790 g, 800 g, 810 g, 820 g, 830 g, 840 g, 850 g, 860 g, 870 g, 880 g, 890 g, 900 g, 910 g, 920 g, 930 g, 940 g, 950 g, 960 g, 970 g, 980 g, 990 g, 1000 g, or more).

A unit dose can be a fraction of the daily dose, such as the daily dose divided by the number of unit doses to be administered per day. A unit dose can be a fraction of the daily dose that is the daily dose divided by the number of unit doses to be administered per day and further divided by the number of unit doses (e.g., tablets) per administration. The number of unit doses per administration may be about, less than about, or more than about 1, 2, 3, 4, 5, 6, 7, 8, 9, 10, or more. The number of doses per day may be about, less than about, or more than about 1, 2, 3, 4, 5, 6, 7, 8, 9, 10, or more. The number of unit doses per day may be determined by dividing the daily dose by the unit dose, and may be about, less than about, or more than about 1, 2, 3, 4, 5, 6, 7, 8, 9, 10, 11, 12, 13, 14, 15, 6, 17, 18, 19, 20, or more unit doses per day. For example, a unit dose can be about ½, ½, ¼, Vs, ½, 1/7, ¼, 1/9, or 1/10. A unit dose can be about one-third of the daily amount and administered to the subject three times daily. A unit dose can be about one-half of the daily amount and administered to the subject twice daily. A unit dose can be about one-fourth of the daily amount with two unit doses administered to the subject twice daily.

In some embodiments, the administration of the first and second regenerative agents, either as separate compositions or a single composition, can have a specified ratio of the first regenerative agent to the second regenerative agent. The specified ratio can provide for effective reparative and/or appendage regeneration. For example, the specified ratios can cause regeneration of a phalange (e.g., phalange 3 and nail of the lost limb). Such beneficial effects can result from, in part, a stimulation of mTOR signaling and/or insulin signaling, or a variety of other changes in cellular metabolism or the energy metabolism pathway. The ratio of the first regenerative agent to the second regenerative agent can be a mass ratio, a molar ratio, or a volume ratio. In some embodiments, the mass ratio of the first regenerative agent to the second regenerative agent is about, greater than about, or less than about 45, 50, 55, 60, 65, 70, 75, 80, 85, 90, 100, 110, 120, 130, 140, 150, 175, 200, 250, 500, 750, 1000, or more. In some embodiments, the molar ratio of the first regenerative agent to the second regenerative agent co-administered is about, greater than about, or less than about 90, 95, 90, 95, 100, 105, 110, 120, 130, 140, 150, 160, 170, 180, 190, 200, or more.

In some embodiments, administration of the first agent and second regenerative agent, either as separate compositions or a single composition, is effective for inducing reparative and/or appendage regeneration in subject in need thereof. In some embodiments, the percentage of the damage, injury, or loss of a limb, organ, tissue, cell, that is regenerated can be, or be about, 0.000000001%, 0.00000001%, 0.0000001%, 0.000001%, 0.00001%, 0.0001%, 0.001%, 0.01%, 0.1%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values.

In some embodiments, the dosing level can be adjusted based on the subject's characteristics, such as weight, height, ethnicity, genetics, or baseline energy metabolism level.

The physician, nutritionist, or clinician can observe the subject's response to the administered regenerative agents and adjust the dosing based on the observed regeneration. For example, dosing levels can be increased for subjects that show no reparative and/or appendage regeneration.

In some embodiments, the compositions administered to a subject can be optimized for a given subject. For example, the ratio of the first regenerative agent to the second regenerative agent or the particular components in a single composition can be adjusted. The ratio and/or particular components can be selected after evaluation of the subject after being administered one or more compositions with varying ratios of the first regenerative agent to the second regenerative agent or varying single composition components.

As disclosed herein, the first and second regenerative agents do not have to be administered in the same composition to perform the claimed methods. For example, separate capsules, pills, mixtures, etc. of the first and second regenerative agents may be administered to a subject to carry out the claimed methods. The administration of the first and second regenerative agents may be at the same time or at different times. In some embodiments, administration of the first and second regenerative agents is at the same time. The first and second regenerative agents can be administered in a single composition, in order to facilitate the compliance of the subject to adhere to a schedule of administration.

It is contemplated that there will be some variation in effectiveness due to differences among individuals in physiological and biochemical parameters (e.g., body weight and basal metabolism), exercise, and other aspects (e.g., diet).

The formulation, route of administration and dosage for the compositions disclosed herein can be chosen by the individual physician in view of the patient's condition. Typically, the dose range of one or both of the first and second regenerative agents administered to the patient can be from about 0.1 mg/kg to about 1000 g/kg of the patient's body weight. The dosage may be a single one or a series of two or more given in the course of one or more days, as is needed by the patient. In instances where human dosages for the compositions have been established for at least some disease or disorder, or acute injury, the present disclosure will use those same dosages, or dosages that are between about 0.1% and about 5000%, more preferably between about 25% and about 1000% of the established human dosage. Where no human dosage is established, as will be the case for newly-discovered pharmaceutical compounds, a suitable human dosage can be inferred from ED₅₀ or ID₅₀ values, or other appropriate values derived from in vitro or in vivo studies, as qualified by toxicity studies and efficacy studies in animals.

It should be noted that the attending physician would know how to and when to terminate, interrupt, or adjust administration due to toxicity or organ dysfunctions. Conversely, the attending physician would also know to adjust treatment to higher levels if the clinical response were not adequate (precluding toxicity). The magnitude of an administrated dose in the management of the disease or disorder, or acute injury of interest will vary with the severity of the disease or disorder, or acute injury to be treated and to the route of administration. The severity of the disease or disorder, or acute injury may, for example, be evaluated, in part, by standard prognostic evaluation methods. Further, the dose and perhaps dose frequency, will also vary according to the age, body weight, and response of the individual patient. A program comparable to that discussed above may be used in veterinary medicine

Routes of Administration

The administration of the first regenerative agent, the administration of the second regenerative agent, or both can be oral, topical, intravenous, intraperitoneal, intragastric, intravascular, or any combination thereof.

One or both of the first and second regenerative agents can be formulated for oral administration. One or both of the first and second regenerative agents can be formulated for oral administration in the form of a tablet, a capsule, or any other form described herein. One or both of the first and second regenerative agents can be administered to a subject orally or by any other methods. Methods of oral administration include, in some embodiments, administering one or both of the first and second regenerative agents as a liquid, a solid, or a semisolid that can be taken in the form of a dietary supplement or a foodstuff

The first and second regenerative agents may be administered separately or together, provided that the total amount of the first and second regenerative agents is an effective amount in combination per day to have a substantial impact on reparative and/or appendage regeneration.

According to a further aspect, one or both of the first and second regenerative agents can be administered in a foodstuff, a food supplement, or a pharmaceutical composition. The foodstuff can comprise a nutritional complete formula, a dairy product, a chilled or shelf stable beverage, a mineral water, a liquid drink, a shot, a soup, a dietary supplement, a meal replacement bar, a nutritional bar, a confectionery product, a milk, a fermented milk product, a yogurt, a pectin chew, a gummy, a milk based powder, an enteral nutrition product, a cereal product, a fermented cereal based product, an ice cream, a chocolate, coffee, a culinary product, or any combination thereof.

In some embodiments, a food composition for human consumption is supplemented by the above composition. For example, the food composition can be, or comprise, a nutritional complete formula, a dairy product, a chilled or shelf stable beverage, a powdered beverage, a mineral or purified water, a liquid drink, a soup, a dietary supplement, a meal replacement, a nutritional bar, a confectionery, a milk, a fermented milk product, a yoghurt, a milk based powder, an enteral nutrition product, an infant formula, an infant nutritional product, a cereal product or a fermented cereal-based product, an ice cream, a chocolate, coffee, a culinary product such as mayonnaise, tomato puree, salad dressings, a pet food, or any combination thereof. The foodstuff can be a beverage.

For ingestion, many embodiments of oral administration and in particular of food supplements are possible. They are formulated by means of the usual methods for producing sugar-coated tablets, pills, pastes, gums, gelatin capsules, gels, emulsions, tablets, capsules or drinkable solutions or emulsions, which can then be taken directly with water or by any other known means.

The food supplement can be in the form of capsules, gelatin capsules, soft capsules, tablets, sugar-coated tablets, powders, pills, pastes, pastilles, gums, drinkable solutions, drinkable emulsions, syrups, gels, or any combination thereof.

The food supplement for oral administration may be in capsules, gelatin capsules, soft capsules, tablets, sugar-coated tablets, pills, pastes or pastilles, gums, or drinkable solutions or emulsions, syrups or gels, with a dose of about 0.001 to 100% of the primary composition, which can then be taken directly with water or by any other known means. This supplement may also include, a stabilizer, an additive, a flavoring or a colorant. A supplement for cosmetic purpose can additionally comprises a compound active with respect to the skin. Methods for preparing them are common knowledge.

Also, the formulation as described above may be incorporated into any other forms of food supplements or of enriched foods, for example food bars, or compacted or non-compacted powders. Methods for preparing them are common knowledge.

The food composition or food supplement may also include, a stabilizer, an antioxidant, an additive, a flavoring or a colorant. The composition may also contain synthetic or natural bioactive ingredients such as amino acids, fatty acids, vitamins, minerals, carotenoids, polyphenols, etc. that can be added either by dry or by wet mixing to said composition before pasteurization and/or drying. According to some embodiments, the composition disclosed herein can be used cosmetically. By “cosmetic use” is meant a non-therapeutic use which may improve the aesthetic aspect or comfort of the skin, coat and/or hair of humans or pets.

The pharmaceutical composition can be in the form of capsules, gelatin capsules, soft capsules, tablets, chewable tablets, sugar-coated tablets, pills, pastes or pastilles, powders, softgels, chewable softgels, gums, drinkable solutions or emulsions, syrups, gels, or any combination thereof. The pharmaceutical composition can comprise one or more of binding agents, gelling agents, thickeners, colorants, taste masking agents, stabilizers, antioxidants, coatings, sweeteners, taste modifiers, and aroma chemicals. The pharmaceutical composition can comprise one or more pharmaceutically acceptable carriers, diluents, and/or excipients.

In some embodiments, a pharmaceutical composition can be administered for prophylactic and/or therapeutic treatments. In therapeutic applications, one or both regenerative agents are administered to a patient already suffering from a disease, as described herein, in an amount sufficient to cure or at least partially arrest the symptoms of the disease and its complications (e.g., limb loss). An amount adequate to accomplish this is defined as “a therapeutically effective dose” or a “therapeutically effective amount”. Amounts effective for this will depend on the severity of the disease or disorder, or acute injury. In prophylactic applications, compositions disclosed herein are administered to a patient susceptible to or otherwise at risk of a particular disease. Such an amount is defined to be “a prophylactic effective dose”. In this use, the precise amounts again depend on the patient's state of health and weight. The compositions disclosed herein are, in some embodiments, administered with a pharmaceutically acceptable carrier, the nature of the carrier differing with the mode of administration, for example, enteral, oral and topical (including ophthalmic) routes. The desired formulation can be made using a variety of excipients including, for example, pharmaceutical grades of magnesium stearate, sodium saccharin, cellulose, magnesium carbonate. This composition may be a tablet, a capsule, a pill, a solution, a suspension, a syrup, a dried oral supplement, a wet oral supplement.

Furthermore, in some embodiments one or both of the first and second regenerative agents can be intravenously administered in any suitable manner. For administration via intravenous infusion, one or both of the first and second regenerative agents are preferably in a water-soluble non-toxic form. Intravenous administration is particularly suitable for hospitalized patients that are undergoing intravenous (IV) therapy. For example, one or both of the first and second regenerative agents can be dissolved in an IV solution (e.g., a saline solution) being administered to the patient. The amounts of one or both of the first and second regenerative agents to be administered intravenously can be similar to levels used in oral administration. Intravenous infusion may be more controlled and accurate than oral administration.

As disclosed herein, one or both of the first and second regenerative agents can be formulated for administration in a pharmaceutical composition comprising a physiologically acceptable surface active agents, carriers, diluents, excipients, smoothing agents, suspension agents, film forming substances, coating assistants, or a combination thereof. In some embodiments, one or both of the first and second regenerative agents are formulated for administration with a pharmaceutically acceptable carrier or diluent. One or both of the first and second regenerative agents can be formulated as a medicament with a standard pharmaceutically acceptable carrier(s) and/or excipient(s) as is routine in the pharmaceutical art. The exact nature of the formulation will depend upon several factors including the desired route of administration. Typically, one or both of the first and second regenerative agents are formulated for oral, intravenous, intragastric, intravascular or intraperitoneal administration. Standard pharmaceutical formulation techniques may be used, such as those disclosed in Remington's The Science and Practice of Pharmacy, 21st Ed., Lippincott Williams & Wilkins (2005), incorporated herein by reference in its entirety. The term “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the pharmaceutical compositions is contemplated. In addition, various adjuvants such as are commonly used in the art may be included. Considerations for the inclusion of various components in pharmaceutical compositions are described, e.g., in Gilman et al. (Eds.) (1990); Goodman and Gilman' s: The Pharmacological Basis of Therapeutics, 8th Ed., Pergamon Press, which is incorporated herein by reference in its entirety.

Some examples of substances, which can serve as pharmaceutically-acceptable carriers or components thereof, are solid lubricants, such as stearic acid and magnesium stearate; calcium sulfate; vegetable oils, such as peanut oil, cottonseed oil, sesame oil, olive oil, corn oil and oil of theobroma; polyols such as propylene glycol, glycerin, sorbitol, mannitol, and polyethylene glycol; alginic acid; emulsifiers, such as the TWEENS; wetting agents, such sodium lauryl sulfate; coloring agents; flavoring agents; tableting agents, stabilizers; antioxidants; preservatives; pyrogen-free water; isotonic saline; and phosphate buffer solutions.

The choice of a pharmaceutically-acceptable carrier to be used in conjunction with a composition can be determined by the way the composition is to be administered.

One or both of the first and second regenerative agents can be in a single unit dosage form. One or both of the first and second regenerative agents can be in two or more unit dosage forms. As used herein, a “unit dosage form” is a composition that is suitable for administration to an animal, preferably mammal subject, in a single dose, according to good medical practice. The preparation of a single or unit dosage form however, does not imply that the dosage form is administered once per day or once per course of therapy. Such dosage forms are contemplated to be administered once, twice, thrice or more per day and may be administered as infusion over a period of time (e.g., from about 30 minutes to about 2-6 hours), or administered as a continuous infusion, and may be given more than once during a course of therapy, though a single administration is not specifically excluded. The skilled artisan will recognize that the formulation does not specifically contemplate the entire course of therapy and such decisions are left for those skilled in the art of treatment rather than formulation.

The compositions useful as described above may be in any of a variety of suitable forms for a variety of routes for administration, for example, for oral, nasal, rectal, topical (including transdermal), ocular, intracerebral, intracranial, intrathecal, intra-arterial, intravenous, intramuscular, or other parental routes of administration. The skilled artisan will appreciate that oral and nasal compositions include compositions that are administered by inhalation, and made using available methodologies. Depending upon the particular route of administration desired, a variety of pharmaceutically-acceptable carriers well-known in the art may be used. Pharmaceutically-acceptable carriers include, for example, solid or liquid fillers, diluents, hydrotropies, surface-active agents, and encapsulating substances. Optional pharmaceutically-active materials may be included, which do not substantially interfere with the activity of the composition. The amount of carrier employed in conjunction with the composition is sufficient to provide a practical quantity of material for administration per unit dose of the composition. Techniques and compositions for making dosage forms useful in the methods described herein are described in the following references, all incorporated by reference herein: Modern Pharmaceutics, 4th Ed., Chapters 9 and 10 (Banker & Rhodes, editors, 2002); Lieberman et ah, Pharmaceutical Dosage Forms: Tablets (1989); and Ansel, Introduction to Pharmaceutical Dosage Forms 8th Edition (2004).

Various oral dosage forms can be used, including such solid forms as tablets, capsules, and granules. Tablets can be compressed, tablet triturates, enteric-coated, sugar-coated, film-coated, or multiple-compressed, containing suitable binders, lubricants, diluents, disintegrating agents, coloring agents, flavoring agents, flow-inducing agents, and melting agents. Liquid oral dosage forms include aqueous solutions, emulsions, suspensions, solutions and/or suspensions reconstituted from non-effervescent granules, and effervescent preparations reconstituted from effervescent granules, containing suitable solvents, preservatives, emulsifying agents, suspending agents, diluents, melting agents, coloring agents and flavoring agents. The pharmaceutically-acceptable carriers suitable for the preparation of unit dosage forms for peroral administration is well-known in the art. Tablets typically comprise conventional pharmaceutically-compatible adjuvants as inert diluents, such as calcium carbonate, sodium carbonate; disintegrants such as starch, alginic acid and croscarmelose; lubricants such as magnesium stearate, stearic acid and talc. Glidants such as silicon dioxide can be used to improve flow characteristics of the powder mixture. Coloring agents, such as the FD&C dyes, can be added for appearance. Flavoring agents, such as aspartame, saccharin, menthol, peppermint, and fruit flavors, are useful adjuvants for chewable tablets. Capsules typically comprise one or more solid diluents disclosed above. The selection of carrier components depends on secondary considerations like taste, cost, and shelf stability, which are not critical, and can be readily made by a person skilled in the art.

Peroral compositions also include liquid solutions, emulsions, suspensions, and the like. The pharmaceutically-acceptable carriers suitable for preparation of such compositions are well known in the art. Typical components of carriers for syrups, elixirs, emulsions and suspensions include ethanol, glycerol, propylene glycol, polyethylene glycol, sorbitol and water. For a suspension, typical suspending agents include sodium carboxymethyl cellulose, AVICEL RC-591, tragacanth and sodium alginate; typical wetting agents include lecithin and polysorbate 80; and typical preservatives include methyl paraben and sodium benzoate. Peroral liquid compositions may also contain one or more components such as sweeteners, flavoring agents and colorants disclosed above.

Other compositions useful for attaining systemic delivery can be in, for example, sublingual, buccal and/or nasal dosage forms. Such compositions typically comprise one or more of soluble filler substances such as sorbitol and mannitol; and binders such as acacia, microcrystalline cellulose, carboxymethyl cellulose and hydroxypropyl methyl cellulose. Glidants, lubricants, colorants, antioxidants and flavoring agents disclosed above may also be included.

For topical use, creams, ointments, gels, solutions or suspensions, etc., containing the compositions disclosed herein are employed. Topical formulations may generally be comprised of a pharmaceutical carrier, co-solvent, emulsifier, penetration enhancer, preservative system, and emollient. For intravenous administration, the compositions described herein may be dissolved or dispersed in a pharmaceutically acceptable diluent, such as a saline solution. Suitable excipients may be included to achieve the desired pH, including but not limited to NaOH, sodium carbonate, sodium acetate, HCl, and citric acid. In various embodiments, the pH of the final composition ranges from 2 to 8, or preferably from 4 to 7. Antioxidant excipients may include sodium bisulfite, acetone sodium bisulfite, sodium formaldehyde, sulfoxylate, thiourea, and EDTA. Other non-limiting examples of suitable excipients found in the final intravenous composition may include sodium or potassium phosphates, citric acid, and tartaric acid. Further acceptable excipients are described in Powell, et al, Compendium of Excipients for Parenteral Formulations, PDA J Pharm Sci and Tech 1998, 52 238-311 and Nema et al., Excipients and Their Role in Approved Injectable Products: Current Usage and Future Directions, PDA J Pharm Sci and Tech 2011, 65 287-332, both of which are incorporated herein by reference in their entirety. Antimicrobial agents may also be included to achieve a bacteriostatic or fungistatic solution, including but not limited to phenylmercuric nitrate, thimerosal, benzethonium chloride, benzalkonium chloride, phenol, cresol, and chlorobutanol.

The compositions for intravenous administration may be provided to caregivers in the form of one more solids that are reconstituted with a suitable diluent such as sterile water or saline in water shortly prior to administration. In some embodiments, the compositions are provided in solution ready to administer parenterally. In some embodiments, the compositions are provided in a solution that is further diluted prior to administration. In embodiments that include administering the compositions described herein and another agent(s) (e.g., a third regenerative agent), the combination may be provided to caregivers as a mixture, or the caregivers may mix the two agents prior to administration, or the two agents may be administered separately.

In some embodiments, a single composition comprises one or both of the first and second regeneration agents and one or more additional ingredients. An additional ingredient may serve one or more functions. In some embodiments, an additional ingredient accounts for about, less than about, or more than about 0.1%, 0.5%, 1%, 2%, 3%, 4%, 5%, 6%, 7%, 8%, 9%, 10%, 15%, 20%, 25%, 30%, 40%, 50%, 60%, 70%, 80%, 90%, 95%, or more of the mass or volume of the single composition. Non-limiting examples of additional ingredients include sweeteners, bulking agents, stabilizers, acidulants, preservatives, binders, lubricants, disintegrants, fillers, solubilizers, coloring agents (such as fruit juice and vegetable juice), and other additives and excipients known in the art. In some embodiments, a single composition comprises one or more (e.g. 1, 2, 3, 4, 5, 6, 7, 8, 9, 10, 15, 20, or more) bulking agents. Non-limiting examples of bulking agents include guar gum, locust bean gum, cassia gum, pectin from botanical sources, high molecular weight carboxymethylcellulose, carrageenan, alginate, and xanthane. In some embodiments, one or more bulking agents may be added to enhance the viscosity of a liquid formulation.

In some embodiments, a single composition comprises one or more (e.g. 1, 2, 3, 4, 5, 6, 7, 8, 9, 10, 15, 20, or more) stabilizers. Non-limiting examples of stabilizers include pectin, polysaccharide hydrolysates comprising dextrin, agar, can-ageenan, tamarind seed polysaccharides, angelica gum, karaya gum, xanthan gum, sodium alginate, tragacanth gum, guar gum, locust bean gum, pullulan, gellan gum, gum arabic, carboxymethylcellulose, and propylene glycol alginate ester. In some embodiments, one or more stabilizers are added to the single composition to enhance the shelf-life of the single composition. In general, shelf-life refers to the amount of time the container and composition therein can be held at ambient conditions (approximately room temperature, e.g. about 18-28° C.) or less, without degradation of the composition and/or container occurring to the extent that the composition cannot be used in the manner and for the purpose for which it was intended. In some embodiments, the single composition has a shelf life of about, less than about, or more than about 1, 2, 3, 4, 5, 6, 7, 8, 9, 10, 1 1, 12, 14, 30, 60, 90, or more days; or about, less than about, or more than about 1, 2, 3, 4, 5, 6, 7, 8, 9, 10, 11, 12, or more months or years. In some embodiments, the single composition remains non-perishable for a period of time after opening a container containing the composition. In general, perishability refers to degradation to an extent that the composition cannot be used in the manner and purpose for which it was designed. In some embodiments, the single composition remains non-perishable for about, less than about, or more than about 1, 2, 3, 4, 5, 6, 7, 8, 9, 10, 11, 12, 18, 24, 30, 36, 48, 60, 72, 90, or more hours or days after opening; or about, less than about, or more than about 1, 2, 3, 4, 5, 6, 8, 10, 11, 12, or more months or years after opening. In some embodiments, the single composition remains nonperishable for a period of time at room temperature (e.g. about 18-28° C.). In some embodiments, the single composition remains non-perishable for a period of time upon refrigeration, such as storage below about 20° C., 15° C., 10° C., 5° C., 4° C., 3° C., 2° C., 1° C., 0° C., −1° C., −2° C., −3° C., −4° C., −5° C., −10° C., −20° C., or lower. In some embodiments, a single composition comprises one or more (e.g. 1, 2, 3, 4, 5, 6, 7, 8, 9, 10, 15, 20, or more) acidulants. Non-limiting examples of acidulants include C2-C30 carboxylic acids, substituted hydroxyl C1-C30 carboxylic acids, benzoic acid, substituted benzoic acids (e.g. 2,4-dihydroxybenzoic acid), substituted cinnamic acids, hydroxyacids, substituted hydroxybenzoic acids, substituted cyclohexyl carboxylic acids, tannic acid, lactic acid, tartaric acid, citric acid, gluconic acid, glucoheptonic acids, adipic acid, hydroxycitric acid, malic acid, fruitaric acid (a blend of malic, fumaric, and tartaric acids), fimaric acid, maleic acid, succinic acid, chlorogenic acid, salicylic acid, creatine, glucosamine hydrochloride, glucono delta lactone, caffeic acid, bile acids, acetic acid, ascorbic acid, alginic acid, erythorbic acid, polyglutamic acid, and their alkali or alkaline earth metal salt derivatives thereof.

In some embodiments, a single composition comprises one or more (e.g. 1, 2, 3, 4, 5, 6, 7, 8, 9, 10, 15, 20, or more) preservatives. Non-limiting examples of preservatives include sorbic acid, benzoic acid, and salts thereof, including (but not limited to) calcium sorbate, sodium sorbate, potassium sorbate, calcium benzoate, sodium benzoate, potassium benzoate, and mixtures thereof.

In some embodiments, the compositions can be a food product, for example a snack bar, or a beverage, comprising one or both of the first and second regenerative agents. For example, the snack bar can be a chocolate bar, a granola bar, or a trail mix bar. In some embodiments, the present dietary supplement or food compositions are formulated to have suitable and desirable taste, texture, and viscosity for consumption. Any suitable food carrier can be used in the present food compositions. Food carriers of the compositions described herein include practically any food product. Examples of such food carriers include, but are not limited to food bars (granola bars, protein bars, candy bars, etc.), cereal products (oatmeal, breakfast cereals, granola, etc.), bakery products (bread, donuts, crackers, bagels, pastries, cakes, etc.), beverages (milk-based beverage, sports drinks, fruit juices, alcoholic beverages, bottled waters), pastas, grains (rice, corn, oats, rye, wheat, flour, etc.), egg products, snacks (candy, chips, gum, chocolate, etc.), meats, fruits, and vegetables. In some embodiments, food carriers employed herein can mask the undesirable taste (e.g., bitterness). Where desired, the food composition presented herein exhibit more desirable textures and aromas than that of any of the components described herein. For example, liquid food carriers can be used to obtain the present food compositions in the form of beverages, such as supplemented juices, coffees, teas, shakes (e.g., milk shakes), smoothies, and the like. In some embodiments, solid food carriers can be used to obtain the present food compositions in the form of meal replacements, such as supplemented snack bars, pasta, breads, and the like. In some embodiments, semi-solid food carriers can be used to obtain the present food compositions in the form of gums, chewy candies or snacks, and the like.

Salts

As disclosed herein, the first and second regenerative agents can be administered separately or simultaneously (e.g., in a single unit dosage form). In some embodiments, the first regenerative agent, the second regeneration, and/or the first and second regenerative agents are administered as pharmaceutically acceptable salts. The term “pharmaceutically acceptable salt” refers to salts that retain the biological effectiveness and properties of a compound and, which are not biologically or otherwise undesirable for use in a pharmaceutical. In many cases, the compounds disclosed herein are capable of forming acid and/or base salts by virtue of the presence of amino and/or carboxyl groups or groups similar thereto. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable salts can also be formed using inorganic and organic bases. Inorganic bases from which salts can be derived include, for example, bases that contain sodium, potassium, lithium, ammonium, calcium, magnesium, iron, zinc, copper, manganese, aluminum, and the like; particularly preferred are the ammonium, potassium, sodium, calcium and magnesium salts. In some embodiments, treatment of the compounds disclosed herein with an inorganic base results in loss of a labile hydrogen from the compound to afford the salt form including an inorganic cation such as Li, Na, K, Mg and Ca and the like. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. Many such salts are known in the art, as described in WO 87/05297 published Sep. 11, 1987 (incorporated by reference herein in its entirety).

Kits

Also provided herein are kits comprising one or more compositions described herein, in suitable packaging, and may further comprise written material that can include instructions for use, discussion of clinical studies, listing of side effects, and the like. Such kits may also include information, such as scientific literature references, package insert materials, clinical trial results, and/or summaries of these and the like, which indicate or establish the activities and/or advantages of the composition, and/or which describe dosing, administration, side effects, drug interactions, or other information useful to the health care provider. Such information may be based on the results of various studies, for example, studies using experimental animals involving in vivo models and studies based on human clinical trials. A kit may comprise one or more unit doses described herein. In some embodiments, a kit comprises about, less than about, or more than about 1, 2, 3, 4, 5, 6, 7, 8, 9, 10, 11, 12, 13, 14, 15, 16, 17, 18, 19, 20, 30, 31, 60, 90, 120, 150, 180, 210, or more unit doses. Instructions for use can comprise dosing instructions, such as instructions to take 1, 2, 3, 4, 5, 6, 7, 8, 9, 10, or more unit doses 1, 2, 3, 4, 5, 6, 7, 8, 9, 10, or more times per day. For example, a kit may comprise a unit dose supplied as a tablet, with each tablet package separately, multiples of tablets packaged separately according to the number of unit doses per administration (e.g. pairs of tablets), or all tablets packaged together (e.g. in a bottle). As a further example, a kit may comprise a unit dose supplied as a bottled drink, the kit comprising 1, 2, 3, 4, 5, 6, 7, 8, 9, 10, 11, 12, 13, 14, 24, 28, 36, 48, 72, or more bottles. The kit can further contain another agent. In some embodiments, the first and second regenerative agents are provided as separate compositions in separate containers within the kit. In some embodiments, the first and second regenerative agents are provided as a single composition within a container in the kit. Suitable packaging and additional articles for use (e.g., measuring cup for liquid preparations, foil wrapping to minimize exposure to air, and the like) are known in the art and may be included in the kit. Kits described herein can be provided, marketed and/or promoted to health providers, including but not limited to, physicians, nurses, pharmacists, formulary officials, and the like. Kits can also, in some embodiments, be marketed directly to the consumer.

In some embodiments, a kit can comprise a multi-day supply of unit dosages. The unit dosages can be any unit dosage described herein. The kit can comprise instructions directing the administration of the multi-day supply of unit dosages over a period of multiple days. The multi-day supply can be a one-month supply, a 30-day supply, or a multi-week supply. The multi-day supply can be a 90-day, 180-day, 3-month or 6-month supply. The kit can include packaged daily unit dosages, such as packages of 1, 2, 3, 4, or 5 unit dosages. The kit can be packaged with, for example, other dietary supplements, vitamins, and meal replacement bars, mixes, and beverages.

### Additional Therapeutic Agents

In some embodiments, the method comprises administering to the subject in need thereof one or more additional therapeutic agents (e.g., regenerative agents). The additional therapeutic agents (e.g., regenerative agents) can be co-administered to the subject with the composition (e.g., the single composition comprising one or both of the first and second regenerative agents). The additional therapeutic agents (e.g., regenerative agents) can be administered to the subject before the administration of the composition, after the administration of the composition, concurrently with the administration of the composition or any combination thereof. The composition (e.g., the single composition comprising one or both of the first and second regenerative agents). can comprise one or more additional therapeutic agents (e.g., regenerative agents).

The method can comprise administering a third regenerative agent that activates mTOR signaling. The first regenerative agent and third regenerative agent can be selected from the group comprising MHY1485, 3BDO, and CL316,243. The first and third regenerative agents can be different. The method can comprise inducing mTOR expression. In some embodiments, the method does not induce insulin resistance.

The method can comprise contacting the subject in need with a scaffold. The scaffold can comprise a bandage, beads, a hydrogel, a polymer, or other biomaterial, or any combination thereof. Components of the scaffolds are organized in a variety of geometric shapes (e.g., beads, pellets), niches, planar layers (e.g., sheets). For example, sheetlike are used in bandages or wound dressings. The scaffold can be placed on or administered into a target tissue. Scaffolds can be introduced into or onto a bodily tissue using a variety of known methods and tools, e.g., spoon, tweezers or graspers, hypodermic needle, endoscopic manipulator, endo- or trans-vascular-catheter, stereotaxic needle, snake device, organ-surface-crawling robot (United States Patent Application 20050154376; Ota et al., 2006, Innovations 1:227-231), minimally invasive surgical devices, surgical implantation tools, and transdermal patches.

A scaffold or scaffold device is the physical structure upon which or into which cells associate or attach, and a scaffold composition is the material from which the structure is made. For example, scaffold compositions include biodegradable or permanent materials such as those listed below. The mechanical characteristics of the scaffold may vary according to the application or tissue type for which regeneration is sought. The scaffold can be biodegradable (e.g., collagen, alginates, polysaccharides, polyethylene glycol (PEG), poly(glycolide) (PGA), poly(L-lactide) (PLA), or poly(lactide-co-glycolide) (PLGA) or permanent (e.g., silk). In the case of biodegradable structures, the composition is degraded by physical or chemical action, e.g., level of hydration, heat or ion exchange or by cellular action, e.g., elaboration of enzyme, peptides, or other compounds by nearby or resident cells. The consistency varies from a soft/pliable (e.g., a gel) to glassy, rubbery, brittle, tough, elastic, stiff. The structures contain pores, which are nanoporous, microporous, or macroporous, and the pattern of the pores is optionally homogeneous, heterogenous, aligned, repeating, or random.

Differences in scaffold formulation control the kinetics of scaffold degradation. Release rates of regenerative agents (e.g., BMP) or other bioactive substances from scaffolds is controlled by scaffold formulation to present regenerative agents in a spatially and temporally controlled manner. This controlled release can be used to create a microenvironment that activates host cells at the implant site. The scaffold can comprise a biocompatible polymer matrix that is optionally biodegradable in whole or in part. A hydrogel is one example of a suitable polymer matrix material. Examples of materials which can form hydrogels include polylactic acid, polyglycolic acid, PLGA polymers, alginates and alginate derivatives, gelatin, collagen, agarose, natural and synthetic polysaccharides, polyamino acids such as polypeptides particularly poly(lysine), polyesters such as polyhydroxybutyrate and poly-epsilon.-caprolactone, polyanhydrides; polyphosphazines, poly(vinyl alcohols), poly(alkylene oxides) particularly poly(ethylene oxides), poly(allylamines)(PAM), poly(acrylates), modified styrene polymers such as poly(4-aminomethylstyrene), pluronic polyols, polyoxamers, poly(uronic acids), poly(vinylpyrrolidone) and copolymers of the above, including graft copolymers. The scaffolds can be fabricated from a variety of synthetic polymers and naturally-occurring polymers such as, but not limited to, collagen, fibrin, hyaluronic acid, agarose, and laminin-rich gels.

The scaffold can comprise a bone morphogenetic protein (BMP), a hormone, a growth factor, or other agent that induces reparative regeneration and/or appendage regeneration, or any combination thereof. In some embodiments, the contacting results in a synergistic effect on regeneration.

As disclosed herein, co-administration of particular ratios and/or amounts of the first regenerative agent and the second and/or, e.g., the third regenerative agent can result in synergistic effects in inducing reparative and/or appendage regeneration. These synergistic effects can be such that the one or more effects of the single compositions are greater than the one or more effects of each component alone at a comparable dosing level, or they can be greater than the predicted sum of the effects of all of the components at a comparable dosing level, assuming that each component acts independently. The synergistic effect can be about, or greater than about, 5, 10, 20, 30, 50, 75, 100, 110, 120, 150, 200, 250, 350, or 500% better than the effect of treating a subject with one of the components alone, or the additive effects of each of the components when administered individually. The effect can be any of the measurable effects described herein. The composition comprising a plurality of components can be such that the synergistic effect is an enhancement in inducing reparative and/or appendage regeneration and that inducing reparative and/or appendage regeneration is increased to a greater degree as compared to the sum of the effects of administering each component, determined as if each component exerted its effect independently, also referred to as the predicted additive effect herein. For example, if a composition comprising component (a) yields an effect of a 20% improvement in inducing reparative and/or appendage regeneration and a composition comprising component (b) yields an effect of 50% improvement in inducing reparative and/or appendage regeneration, then a composition comprising both component (a) and component (b) would have a synergistic effect if the single composition's effect on inducing reparative and/or appendage regeneration was greater than 70%.

A synergistic single composition can have an effect that is greater than the predicted additive effect of administering each component of the single composition alone as if each component exerted its effect independently. For example, if the predicted additive effect is 70%, an actual effect of 140% is 70% greater than the predicted additive effect or is 1 fold greater than the predicted additive effect. The synergistic effect can be at least about 20, 50, 75, 90, 100, 150, 200 or 300% greater than the predicted additive effect. In some embodiments, the synergistic effect can be at least about 0.2, 0.5, 0.9, 1.1, 1.5, 1.7, 2, or 3 fold greater than the predicted additive effect.

In some embodiments, the synergistic effect of the single compositions can also allow for reduced dosing amounts, leading to reduced side effects to the subject and reduced cost of treatment. Furthermore, the synergistic effect can allow for results that are not achievable through any other treatments. Therefore, proper identification, specification, and use of single compositions can allow for significant improvements in inducing reparative and/or appendage regeneration

### Additional Regenerative Agents

In some embodiments, the method comprises administering additional regenerative agents (e.g., BMP) to a subject in need. In some embodiments, a regenerative agent can be a hormone. The term “hormone” refers to polypeptide hormones, which are generally secreted by glandular organs with ducts. Hormones include proteins from natural sources or from recombinant cell culture and biologically active equivalents of the native sequence hormone, including synthetically produced small-molecule entities and pharmaceutically acceptable derivatives and salts thereof. Included among the hormones are, for example, growth hormone such as human growth hormone, N-methionyl human growth hormone, and bovine growth hormone; parathyroid hormone; thyroxine; relaxin; prorelaxin; glycoprotein hormones such as follicle stimulating hormone (FSH), thyroid stimulating hormone (TSH), and luteinizing hormone (LH); prolactin, placental lactogen, mouse gonadotropin-associated peptide, inhibin; activin; mullerian-inhibiting substance; and thrombopoietin, growth hormone (GH), adrenocorticotropic hormone (ACTH), dehydroepiandrosterone (DHEA), cortisol, epinephrine, thyroid hormone, estrogen, progesterone, placental lactogens (somatomammotropins, e.g. CSH1, CHS2), testosterone and neuroendocrine hormones. In certain examples, the hormone is secreted from pancreas, e.g., glucagon, somatostatin, pancreatic polypeptide and ghrelin.

In some embodiments, the regenerative agent can comprise growth factors, e.g., fibroblast growth factor (FGF) family, bone morphogenic protein (BMP) family, platelet derived growth factor (PDGF) family, transforming growth factor beta (TGFbeta) family, nerve growth factor (NGF) family, epidermal growth factor (EGF) family, insulin related growth factor (IGF) family, hepatocyte growth factor (HGF) family, hematopoietic growth factors (HeGFs), platelet-derived endothelial cell growth factor (PD-ECGF), angiopoietin, vascular endothelial growth factor (VEGF) family, and glucocorticoids.

In some embodiments, the regenerative agent is a neurohormone, a hormone produced and released by neuroendocrine cells. Neurohormones include Thyrotropin-releasing hormone, Corticotropin-releasing hormone, Histamine, Growth hormone-releasing hormone, Somatostatin, Gonadotropin-releasing hormone, Serotonin, Dopamine, Neurotensin, Oxytocin, Vasopressin, Epinephrine, and Norepinephrine.

In some embodiments, the method comprises co-administration with an analgesic, anti-infective, antibiotic, antifungal, or antiviral agents.

Analgesics

Analgesics include aspirin, phenybutazone, idomethacin, sulindac, tolmetic, ibuprofen, piroxicam, fenamates, acetaminophen, phenacetin, morphine sulfate, codeine sulfate, meperidine, nalorphine, opioids (e.g., codeine sulfate, fentanyl citrate, hydrocodone bitartrate, loperamide, morphine sulfate, noscapine, norcodeine, normorphine, thebaine, nor-binaltorphimine, buprenorphine, chlomaltrexamine, funaltrexamione, nalbuphine, nalorphine, naloxone, naloxonazine, naltrexone, and naltrindole), procaine, lidocain, tetracaine and dibucaine.

Anti-Infective Agents

Anti-Infective Agents. The agent may be an anti-infective agent including without limitation an anti-bacterial agent, an anti-viral agent, an anti-parasitic agent, an anti-fungal agent, and an anti-mycobacterial agent.

Anti-Bacterial Agents

Anti-bacterial agents may be without limitation β-lactam antibiotics, penicillins (such as natural penicillins, aminopenicillins, penicillinase-resistant penicillins, carboxy penicillins, ureido penicillins), cephalosporins (first generation, second generation, and third generation cephalosporins), other β-lactams (such as imipenem, monobactams), β-lactamase inhibitors, vancomycin, aminoglycosides and spectinomycin, tetracyclines, chloramphenicol, erythromycin, lincomycin, clindamycin, rifampin, metronidazole, polymyxins, sulfonamides and trimethoprim, or quinolines.

Other anti-bacterials may be without limitation Acedapsone; Acetosulfone Sodium; Alamecin; Alexidine; Amdinocillin; Amdinocillin Pivoxil; Amicycline; Amifloxacin; Amifloxacin Mesylate; Amikacin; Amikacin Sulfate; Aminosalicylic acid; Aminosalicylate sodium; Amoxicillin; Amphomycin; Ampicillin; Ampicillin Sodium; Apalcillin Sodium; Apramycin; Aspartocin; Astromicin Sulfate; Avilamycin; Avoparcin; Azithromycin; Azlocillin; Azlocillin Sodium; Bacampicillin Hydrochloride; Bacitracin; Bacitracin Methylene Disalicylate; Bacitracin Zinc; Bambermycins; Benzoylpas Calcium; Berythromycin; Betamicin Sulfate; Biapenem; Biniramycin; Biphenamine Hydrochloride; Bispyrithione Magsulfex; Butikacin; Butirosin Sulfate; Capreomycin Sulfate; Carbadox; Carbenicillin Disodium; Carbenicillin Indanyl Sodium; Carbenicillin Phenyl Sodium; Carbenicillin Potassium; Carumonam Sodium; Cefaclor; Cefadroxil; Cefamandole; Cefamandole Nafate; Cefamandole Sodium; Cefaparole; Cefatrizine; Cefazaflur Sodium; Cefazolin; Cefazolin Sodium; Cefbuperazone; Cefdinir; Cefepime; Cefepime Hydrochloride; Cefetecol; Cefixime; Cefmenoxime Hydrochloride; Cefmetazole; Cefmetazole Sodium; Cefonicid Monosodium; Cefonicid Sodium; Cefoperazone Sodium; Ceforanide; Cefotaxime Sodium; Cefotetan; Cefotetan Disodium; Cefotiam Hydrochloride; Cefoxitin; Cefoxitin Sodium; Cefpimizole; Cefpimizole Sodium; Cefpiramide; Cefpiramide Sodium; Cefpirome Sulfate; Cefpodoxime Proxetil; Cefprozil; Cefroxadine; Cefsulodin Sodium; Ceftazidime; Ceftibuten; Ceftizoxime Sodium; Ceftriaxone Sodium; Cefuroxime; Cefuroxime Axetil; Cefuroxime Pivoxetil; Cefuroxime Sodium; Cephacetrile Sodium; Cephalexin; Cephalexin Hydrochloride; Cephaloglycin; Cephaloridine; Cephalothin Sodium; Cephapirin Sodium; Cephradine; Cetocycline Hydrochloride; Cetophenicol; Chloramphenicol; Chloramphenicol Palmitate; Chloramphenicol Pantothenate Complex; Chloramphenicol Sodium Succinate; Chlorhexidine Phosphanilate; Chloroxylenol; Chlortetracycline Bisulfate; Chlortetracycline Hydrochloride; Cinoxacin; Ciprofloxacin; Ciprofloxacin Hydrochloride; Cirolemycin; Clarithromycin; Clinafloxacin Hydrochloride; Clindamycin; Clindamycin Hydrochloride; Clindamycin Palmitate Hydrochloride; Clindamycin Phosphate; Clofazimine; Cloxacillin Benzathine; Cloxacillin Sodium; Cloxyquin; Colistimethate Sodium; Colistin Sulfate; Coumermycin; Coumermycin Sodium; Cyclacillin; Cycloserine; Dalfopristin; Dapsone; Daptomycin; Demeclocycline; Demeclocycline Hydrochloride; Demecycline; Denofungin; Diaveridine; Dicloxacillin; Dicloxacillin Sodium; Dihydrostreptomycin Sulfate; Dipyrithione; Dirithromycin; Doxycycline; Doxycycline Calcium; Doxycycline Fosfatex; Doxycycline Hyclate; Droxacin Sodium; Enoxacin; Epicillin; Epitetracycline Hydrochloride; Erythromycin; Erythromycin Acistrate; Erythromycin Estolate; Erythromycin Ethylsuccinate; Erythromycin Gluceptate; Erythromycin Lactobionate; Erythromycin Propionate; Erythromycin Stearate; Ethambutol Hydrochloride; Ethionamide; Fleroxacin; Floxacillin; Fludalanine; Flumequine; Fosfomycin; Fosfomycin Tromethamine; Fumoxicillin; Furazolium Chloride; Furazolium Tartrate; Fusidate Sodium; Fusidic Acid; Gentamicin Sulfate; Gloximonam; Gramicidin; Haloprogin; Hetacillin; Hetacillin Potassium; Hexedine; Ibafloxacin; Imipenem; Isoconazole; Isepamicin; Isoniazid; Josamycin; Kanamycin Sulfate; Kitasamycin; Levofuraltadone; Levopropylcillin Potassium; Lexithromycin; Lincomycin; Lincomycin Hydrochloride; Lomefloxacin; Lomefloxacin Hydrochloride; Lomefloxacin Mesylate; Loracarbef; Mafenide; Meclocycline; Meclocycline Sulfosalicylate; Megalomicin Potassium Phosphate; Mequidox; Meropenem; Methacycline; Methacycline Hydrochloride; Methenamine; Methenamine Hippurate; Methenamine Mandelate; Methicillin Sodium; Metioprim; Metronidazole Hydrochloride; Metronidazole Phosphate; Mezlocillin; Mezlocillin Sodium; Minocycline; Minocycline Hydrochloride; Mirincamycin Hydrochloride; Monensin; Monensin Sodium; Nafcillin Sodium; Nalidixate Sodium; Nalidixic Acid; Natamycin; Nebramycin; Neomycin Palmitate; Neomycin Sulfate; Neomycin Undecylenate; Netilmicin Sulfate; Neutramycin; Nifuradene; Nifuraldezone; Nifuratel; Nifuratrone; Nifurdazil; Nifurimide; Nifurpirinol; Nifurquinazol; Nifurthiazole; Nitrocycline; Nitrofurantoin; Nitromide; Norfloxacin; Novobiocin Sodium; Ofloxacin; Ormetoprim; Oxacillin Sodium; Oximonam; Oximonam Sodium; Oxolinic Acid; Oxytetracycline; Oxytetracycline Calcium; Oxytetracycline Hydrochloride; Paldimycin; Parachlorophenol; Paulomycin; Pefloxacin; Pefloxacin Mesylate; Penamecillin; Penicillin G Benzathine; Penicillin G Potassium; Penicillin G Procaine; Penicillin G Sodium; Penicillin V; Penicillin V Benzathine; Penicillin V Hydrabamine; Penicillin V Potassium; Pentizidone Sodium; Phenyl Aminosalicylate; Piperacillin Sodium; Pirbenicillin Sodium; Piridicillin Sodium; Pirlimycin Hydrochloride; Pivampicillin Hydrochloride; Pivampicillin Pamoate; Pivampicillin Probenate; Polymyxin B Sulfate; Porfiromycin; Propikacin; Pyrazinamide; Pyrithione Zinc; Quindecamine Acetate; Quinupristin; Racephenicol; Ramoplanin; Ranimycin; Relomycin; Repromicin; Rifabutin; Rifametane; Rifamexil; Rifamide; Rifampin; Rifapentine; Rifaximin; Rolitetracycline; Rolitetracycline Nitrate; Rosaramicin; Rosaramicin Butyrate; Rosaramicin Propionate; Rosaramicin Sodium Phosphate; Rosaramicin Stearate; Rosoxacin; Roxarsone; Roxithromycin; Sancycline; Sanfetrinem Sodium; Sarmoxicillin; Sarpicillin; Scopafungin; Sisomicin; Sisomicin Sulfate; Sparfloxacin; Spectinomycin Hydrochloride; Spiramycin; Stallimycin Hydrochloride; Steffimycin; Streptomycin Sulfate; Streptonicozid; Sulfabenz; Sulfabenzamide; Sulfacetamide; Sulfacetamide Sodium; Sulfacytine; Sulfadiazine; Sulfadiazine Sodium; Sulfadoxine; Sulfalene; Sulfamerazine; Sulfameter; Sulfamethazine; Sulfamethizole; Sulfamethoxazole; Sulfamonomethoxine; Sulfamoxole; Sulfanilate Zinc; Sulfanitran; Sulfas alazine; Sulfasomizole; Sulfathiazole; Sulfazamet; Sulfisoxazole; Sulfisoxazole Acetyl; Sulfisoxazole Diolamine; Sulfomyxin; Sulopenem; Sultamicillin; Suncillin Sodium; Talampicillin Hydrochloride; Teicoplanin; Temafloxacin Hydrochloride; Temocillin; Tetracycline; Tetracycline Hydrochloride; Tetracycline Phosphate Complex; Tetroxoprim; Thiamphenicol; Thiphencillin Potassium; Ticarcillin Cresyl Sodium; Ticarcillin Disodium; Ticarcillin Monosodium; Ticlatone; Tiodonium Chloride; Tobramycin; Tobramycin Sulfate; Tosufloxacin; Trimethoprim; Trimethoprim Sulfate; Trisulfapyrimidines; Troleandomycin; Trospectomycin Sulfate; Tyrothricin; Vancomycin; Vancomycin Hydrochloride; Virginiamycin; or Zorbamycin. Anti-mycobacterial agents may be without limitation Myambutol (Ethambutol Hydrochloride), Dapsone (4,4′-diaminodiphenylsulfone), Paser Granules (aminosalicylic acid granules), Priftin (rifapentine), Pyrazinamide, Isoniazid, Rifadin (Rifampin), Rifadin IV, Rifamate (Rifampin and Isoniazid), Rifater (Rifampin, Isoniazid, and Pyrazinamide), Streptomycin Sulfate or Trecator-SC (Ethionamide).

Antifungal Agents

Anti-fungal agents may be without limitation imidazoles and triazoles, polyene macrolide antibiotics, griseofulvin, amphotericin B, and flucytosine. Antiparasites include heavy metals, antimalarial quinolines, folate antagonists, nitroimidazoles, benzimidazoles, avermectins, praxiquantel, ornithine decarboxylase inhibitors, phenols (e.g., bithionol, niclosamide); synthetic alkaloid (e.g., dehydroemetine); piperazines (e.g., diethylcarbamazine); acetanilide (e.g., diloxanide furonate); halogenated quinolines (e.g., iodoquinol (diiodohydroxyquin)); nitrofurans (e.g., nifurtimox); diamidines (e.g., pentamidine); tetrahydropyrimidine (e.g., pyrantel pamoate); or sulfated naphthylamine (e.g., suramin). Other anti-infective agents may be without limitation Difloxacin Hydrochloride; Lauryl Isoquinolinium Bromide; Moxalactam Disodium; Ornidazole; Pentisomicin; Sarafloxacin Hydrochloride; Protease inhibitors of HIV and other retroviruses; Integrase Inhibitors of HIV and other retroviruses; Cefaclor (Ceclor); Acyclovir (Zovirax); Norfloxacin (Noroxin); Cefoxitin (Mefoxin); Cefuroxime axetil (Ceftin); Ciprofloxacin (Cipro); Aminacrine Hydrochloride; Benzethonium Chloride: Bithionolate Sodium; Bromchlorenone; Carbamide Peroxide; Cetalkonium Chloride; Cetylpyridinium Chloride: Chlorhexidine Hydrochloride; Clioquinol; Domiphen Bromide; Fenticlor; Fludazonium Chloride; Fuchsin, Basic; Furazolidone; Gentian Violet; Halquinols; Hexachlorophene: Hydrogen Peroxide; Ichthammol; Imidecyl Iodine; Iodine; Isopropyl Alcohol; Mafenide Acetate; Meralein Sodium; Mercufenol Chloride; Mercury, Ammoniated; Methylbenzethonium Chloride; Nitrofurazone; Nitromersol; Octenidine Hydrochloride; Oxychlorosene; Oxychlorosene Sodium; Parachlorophenol, Camphorated; Potassium Permanganate; Povidone-Iodine; Sepazonium Chloride; Silver Nitrate; Sulfadiazine, Silver; Symclosene; Thimerfonate Sodium; Thimerosal; or Troclosene Potassium.

Antiviral Agents

Anti-viral agents may be without limitation amantidine and rimantadine, ribivarin, acyclovir, vidarabine, trifluorothymidine, ganciclovir, zidovudine, retinovir, and interferons. Anti-viral agents may be without limitation further include Acemannan; Acyclovir; Acyclovir Sodium; Adefovir; Alovudine; Alvircept Sudotox; Amantadine Hydrochloride; Aranotin; Arildone; Atevirdine Mesylate; Avridine; Cidofovir; Cipamfylline; Cytarabine Hydrochloride; Delavirdine Mesylate; Desciclovir; Didanosine; Disoxaril; Edoxudine; Enviradene; Enviroxime; Famciclovir; Famotine Hydrochloride; Fiacitabine; Fialuridine; Fosarilate; Foscarnet Sodium; Fosfonet Sodium; Ganciclovir; Ganciclovir Sodium; Idoxuridine; Kethoxal; Lamivudine; Lobucavir; Memotine Hydrochloride; Methisazone; Nevirapine; Penciclovir; Pirodavir; Ribavirin; Rimantadine Hydrochloride; Saquinavir Mesylate; Somantadine Hydrochloride; Sorivudine; Statolon; Stavudine; Tilorone Hydrochloride; Trifluridine; Valacyclovir Hydrochloride; Vidarabine; Vidarabine Phosphate; Vidarabine Sodium Phosphate; Viroxime; Zalcitabine; Zidovudine; Zinviroxime or integrase inhibitors.

### EXAMPLES

Some aspects of the embodiments discussed above are disclosed in further detail in the following examples, which are not in any way intended to limit the scope of the present disclosure.

### Example 1

### Inducing Regeneration

Can appendage regeneration be induced? Few have pursued this question, and a unifying framework has yet to arise to suggest a conserved mechanism across animals. Disclosed herein is a conserved strategy for inducing appendage regeneration across three species. Beginning in the moon jelly Aurelia aurita ephyra, it was found that appendage regeneration can be induced with few exogenous stimuli, including the amino acid L-leucine and the growth hormone insulin. In the fruit fly Drosophila melanogaster, L-leucine and insulin administration induced tibia regeneration. In adult mouse Mus muculus, L-leucine and sucrose administration induced digit regeneration, including one from mid-phalangeal amputation—the most dramatic so far reported. The efficacy of L-leucine and insulin/sugar across species >500 million years diverged suggests a conserved role of energetic parameters in unlocking regeneration—the present disclosure suggests the role of the mTOR pathway. The ease by which ad libitum administration of amino acid and sugar effects the outcome can help accelerate progress in inducing appendage and limb regeneration across animals.

In contrast to humans' dismal ability to regenerate, the animal world is filled with seemingly Homeric tales: a creature that regrows when halved or a whole animal growing from a small body piece. Two views have historically prevailed as to why some animals regenerate better than others. Some biologists, including Charles Darwin and August Weismann, hold that regeneration is an adaptive property of a specific organ. For instance, some lobsters may evolve the ability to regenerate their claws because they often lose them in fights and food foraging. Other biologists, including Thomas Morgan, hold that regeneration is not an evolved trait of a particular organ, but inherent in organisms. Regeneration evolving for a particular organ versus regeneration being organismally inherent is an important distinction, as the latter suggests that the lack of regeneration is not due to the trait never having evolved, but rather inactivation—and could therefore possibly be induced. In support of Morgan's view, studies in the past two decades have converged on one striking insight: many animal phyla have at least one or more species that regenerate body parts. Further, even in poorly regenerative lineages, many embryonic and larval stages can regenerate. This raises the possibility that, rather than many instances of convergence, the ability to regenerate is more likely to be ancestral. Regeneration being ancestral begs the question: is there a conserved mechanism to activate regenerative state?

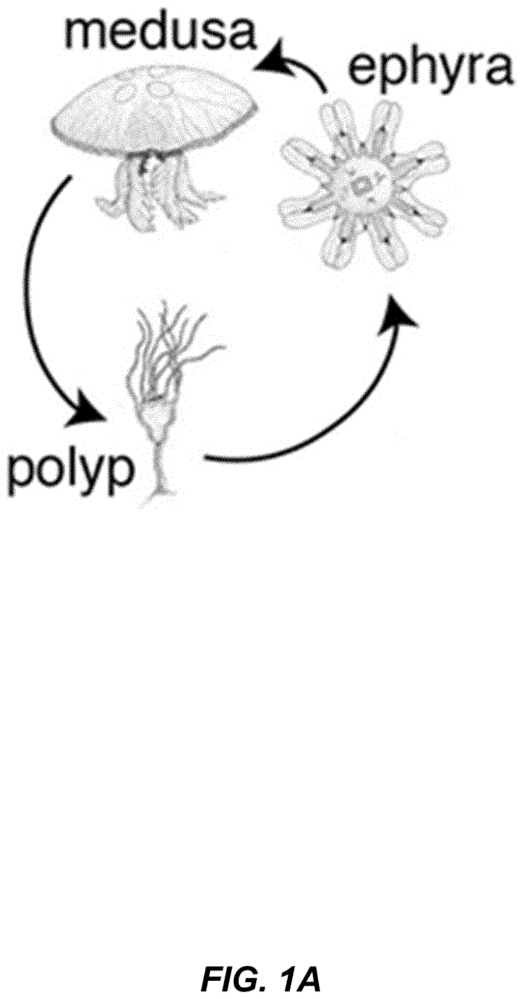

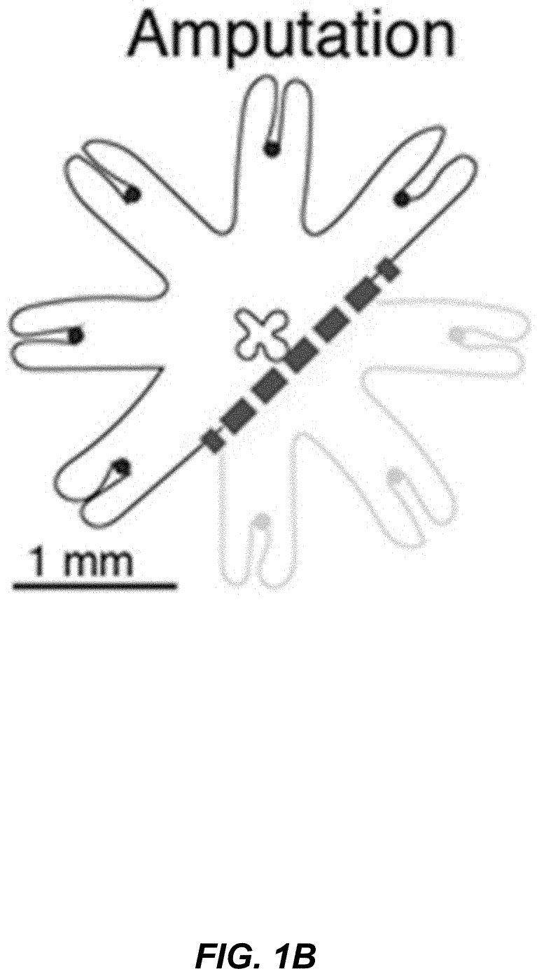

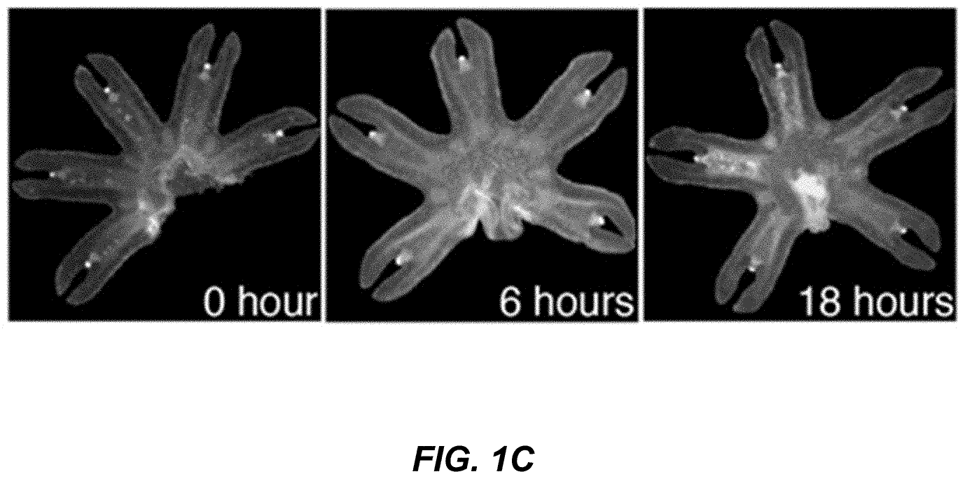

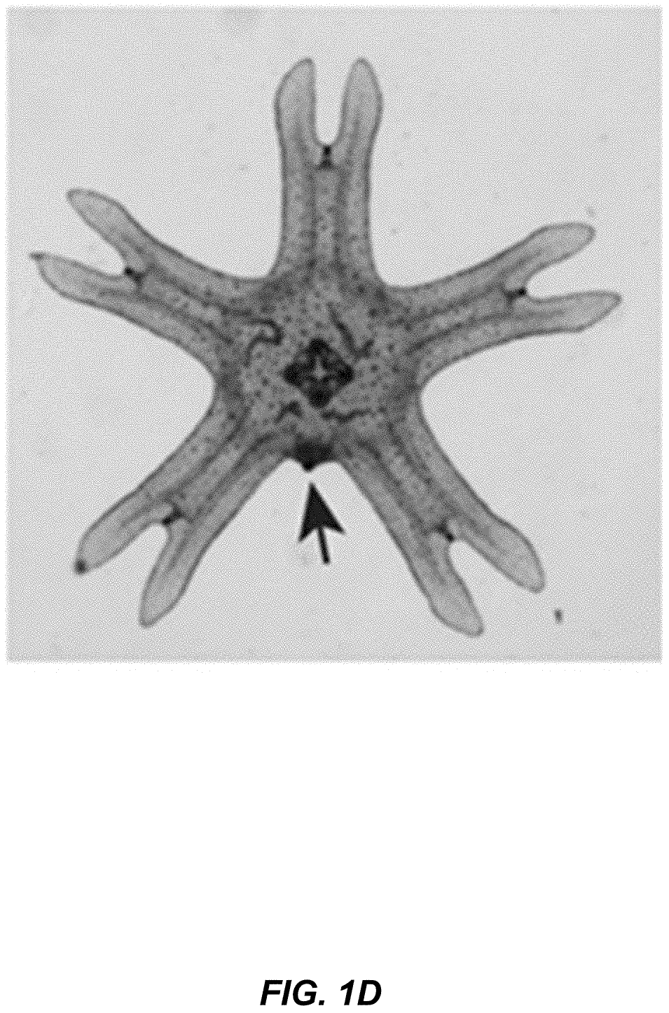

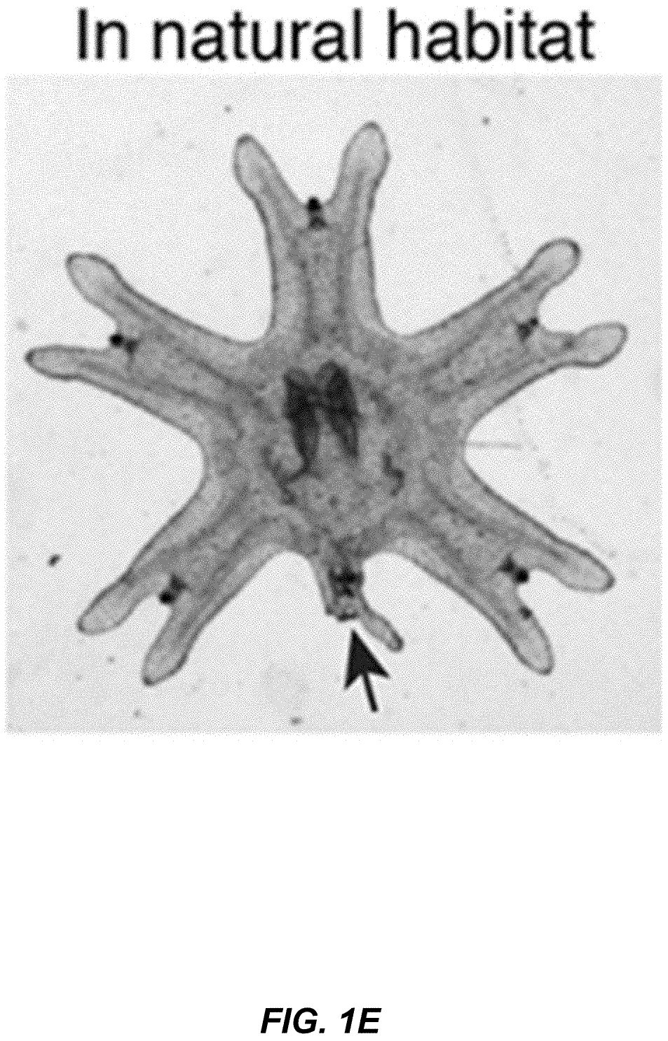

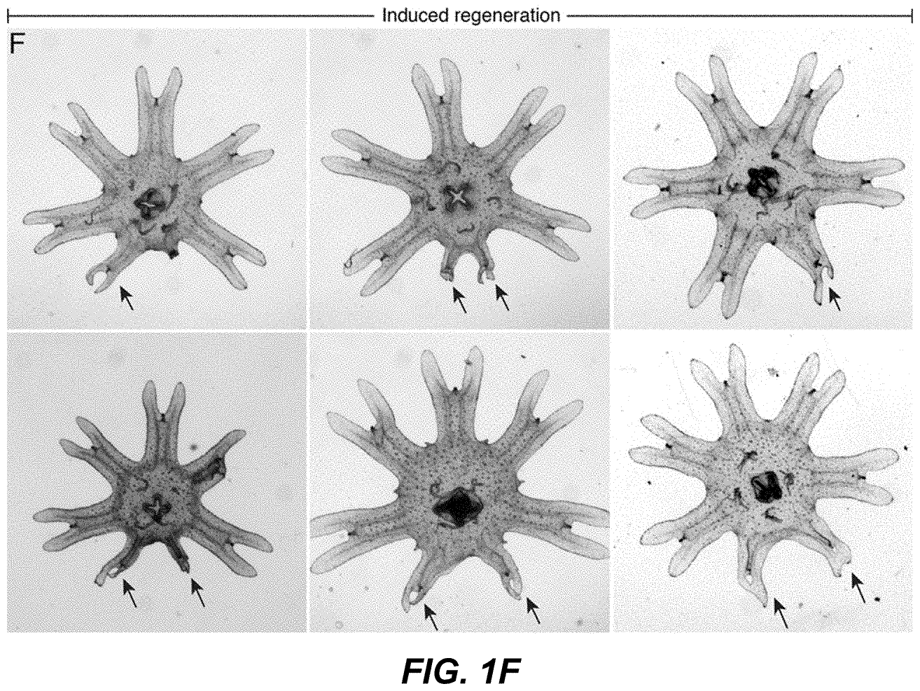

It was reasoned that if there was a conserved mechanism to induce regeneration, it is more likely to be intact in early-branching lineages. In the basal phylum Cnidaria, the ability to regenerate is well known in polyps, e.g., hydras, sea anemones, corals. Some cnidarians, notably the medusozoans (jellyfish), do not only exist as sessile polyps, but also as free-swimming ephyra and medusa (FIG. 1A-FIG. 1B). In contrast to the ability of polyps to regenerate, regeneration in ephyrae and medusae appears more restricted. The moon jellyfish Aurelia aurita sp. 1 was focused on, specifically on the ephyra stage, whose eight discrete arms (FIG. 1B) facilitate morphological tracking. The arms are swimming appendages that synchronously contract to generate axisymmetric fluid flow facilitating propulsion and filter feeding. Ephyrae can regenerate tips of arms, but upon more dramatic amputations, such as removing a whole arm or halving the body, do not regenerate and instead reorganize existing arms and regain radial symmetry (FIG. 1C).

Intriguingly, in ˜1 of 50 symmetrizing ephyrae, a small bud appears at the amputation site (FIG. 1D). The experiment was repeated in the habitat where the founding polyps of the present population were first collected, off the coast of Long Beach, CA (see Methods below). Two weeks after amputation, a rudimentary arm grew in 2 out of 18 animals (FIG. 1E). Growing a rudimentary arm over 2 weeks at a low frequency is far from what is typically observed as regeneration, e.g., hydras perfectly regrow half their bodies in 4 days, planarians perfectly regrow their heads or tails within a week. And yet, this attempt at regeneration suggests an inherent ability to regenerate, and presents an opportunity: Can arm regeneration be induced in the lab, as a way to understand how to switch on the regenerative state?

Various molecular and physical factors were screened (Table 1A-Table 1B).

TABLE 1A
FACTORS TESTED FOR INDUCING ARM REGENERATION
IN THE MOON JELLYFISH EPHYRA.

| Factor | Highest dose | | Source |
|---|---|---|---|
| Modulators of | | | |
| signaling pathways | | | |
| Erbstatin | 5 | μM | Sigma D2667 |
| hEGF recombinant | 20 | ng/mL | Sigma E9644 |
| UO126 | 1 | μM | Millipore 6625 |
| Dorsomorphin | 1 | μM | Sigma P5499 |
| LiCl | 250 | mM | Sigma L4408 |
| CHIR99021 | 12.5 | μM | Sigma SML1046 |
| IWR-1 | 10 | μM | Sigma I0161 |
| XAV939 | 2 | μM | Sigma X3004 |
| Purmorphamine | 2 | μM | Sigma SML0868 |
| hTGF-β1 | 1.2 | ng/mL | Peprotech 100-21 |
| Modulations of | | | |
| metabolism, immune | | | |
| system, stress response | | | |
| Diosmetin | 10 | μM | Sigma D7321 |
| 17-DMAG | 1 | μM | TSZ Chemicals R1028 |
| Geranylgeranylacetone | 1 | μM | Sigma G5408 |
| KNK437 | 4 | nM | Sigma SML0964 |
| MKT-077 | 2.5 | μM | Sigma M5449 |
| Bromopyruvic acid | 125 | nM | Sigma 16490 |
| 6-Phosphogluconic acid | 20 | μM | Sigma P7877 |
| Antamycin A | 650 | nM | Sigma A8674 |
| 3PO | 10 | μM | Millipore 525330 |
| ATP | 5 | μM | Sigma A3377 |
| 3BDO | 3 | μM | Sigma SML1687 |
| D-Fructose 1.6-bisphosphate | 20 | μM | Sigma F6803 |
| DMOG | 50 | μM | Millipore 400091 |
| Rapamycin | 1 | μM | Sigma R8781 |
| L-Leucine methyl | 100 | μM | Sigma L 1002 |
| esther hydrochloride | | | |
| (cell permeable form) | | | |
| Resveratrol | 5 | μM | Sigma R5010 |
| Sapanisertib | 2 | nM | Selleck Chemicals |
| | | | S2811 |
| MHY1485 | 2 | μM | Sigma SML0810 |
| Insulin, human | 500 | nM | Sigma I0908 |
| AICAR | 25 | μM | Santa Cruz SC-200659A |
| A769662 | 5 | μM | Santa Cruz sc-203790 |
| D-Eryhtrose 4-phosphate | 20 | μM | Sigma E0377 |
| CoCl | 450 | nM | Sigma 60818 |
| Miscellaneous | | | |
| BSA | 500 | nM | Sigma A7906 |
| Ethanol | 20 | μL/L | VWR 89125-170 |
| CsCl | 5 | μL/L | Sigma C4036 |

For each molecular factor, the typical doses used in animal models and cell culture systems were first researched, and used those doses as an order-of-magnitude start.
Around this order-of-magnitude estimate, various doses in the ephyrae were tested, aiming to determine the highest dose that could be implemented (listed in the table), which is limited by either solubility in salt water or the beginning of non-specific adverse effects, such as degrowth, lethargy, and lethality.
The highest dose used for each treatment is reported here.
All factors were added to the artificial seawater upon amputation.
Likewise, physical parameters tested were implemented right upon amputation.

TABLE 1B
FACTORS TESTED FOR INDUCING ARM REGENERATION
IN THE MOON JELLYFISH EPHYRA.

| Factor | What was tested | Implementation |
|---|---|---|
| Heat shock | 30 sec at 42° C. and/or 30 | Animals were placed in a |
| | min at 37° C., right after | tube then submersed in a |
| | or 1 d after amputation | heat bath |
| Nutrient | 1-50 rotifers/animal | Various combinations of |
| | 0-5 brine shrimps/animal | feeding amount and |
| | Combination of both | frequency were tested. |
| Water current | 0-60 bubbles per minute | Ambient air was bubbled |
| | | to the cone with Tetra |
| | | Whisper pump |
| Temperature | 18-25° C. | Cooler or heater as |
| | | appropriate. |
| Aquarium | Beaker, plate, tube, cone | Amputated ephyrae were |
| | | let to recover in different |
| | | settings |
| Water volume | 100 mL-1 L | |
| Animal density | 10-100 ephyrae/L | |

For each molecular factor, the typical doses used in animal models and cell culture systems were first researched, and used those doses as an order-of-magnitude start.
Around this order-of-magnitude estimate, various doses in the ephyrae were tested, aiming to determine the highest dose that could be implemented (listed in the table), which is limited by either solubility in salt water or the beginning of non-specific adverse effects, such as degrowth, lethargy, and lethality.
The highest dose used for each treatment is reported here.
All factors were added to the artificial seawater upon amputation.
Likewise, physical parameters tested were implemented right upon amputation.

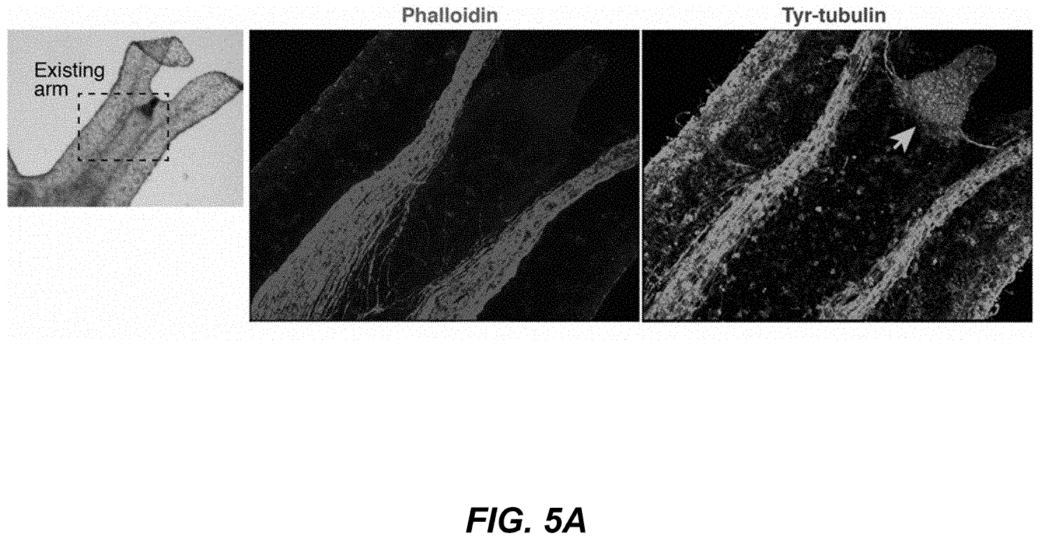

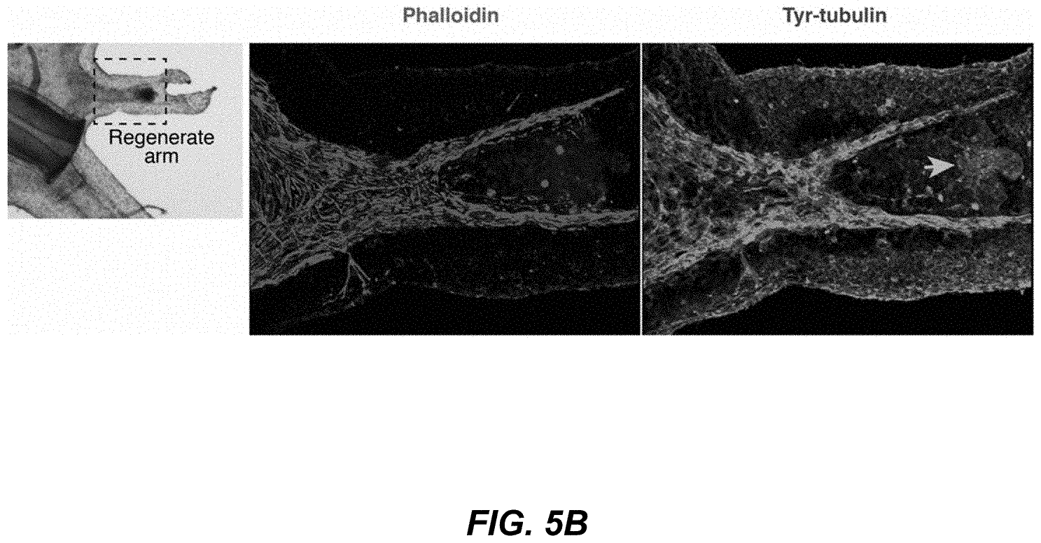

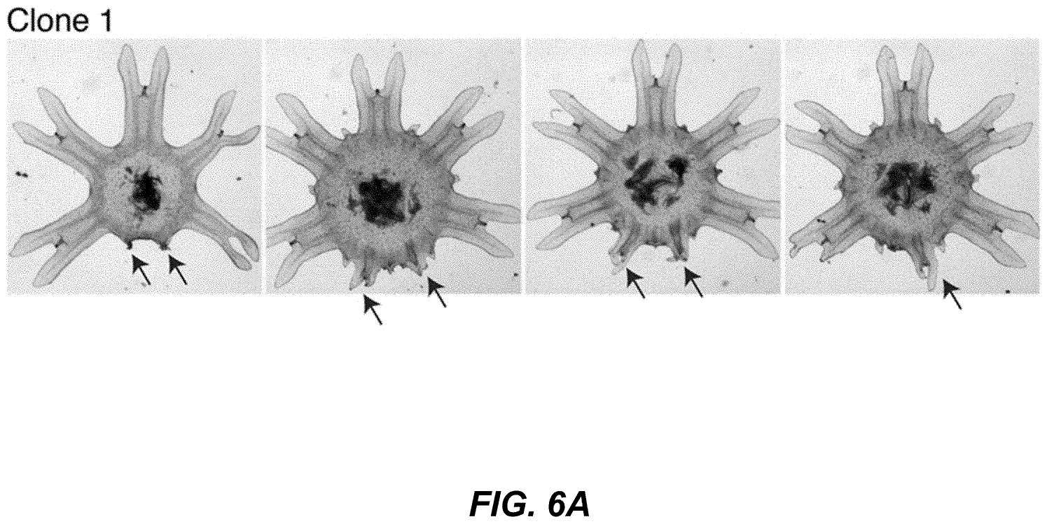

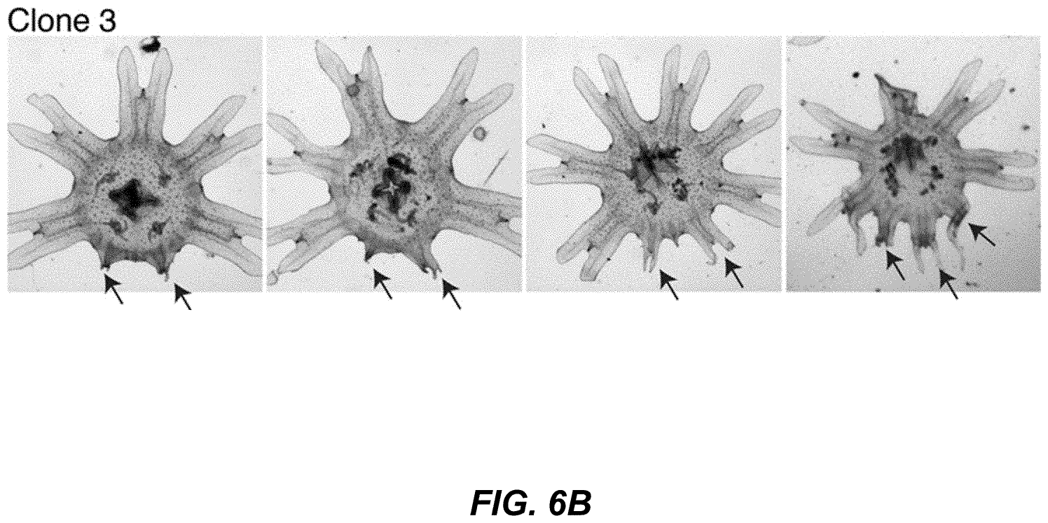

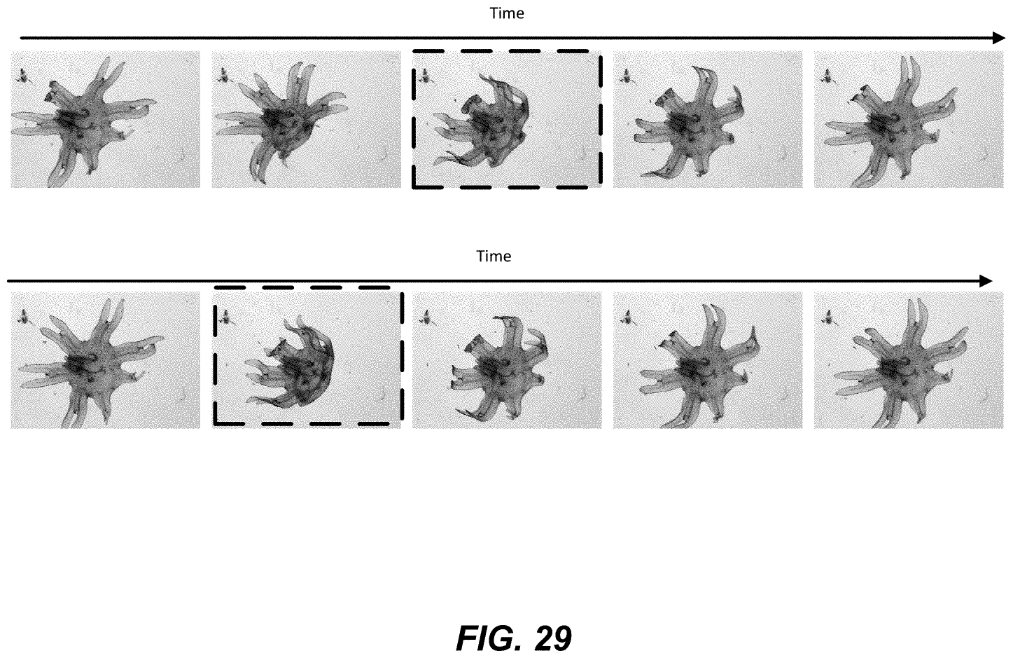

Molecularly, developmental signaling pathways often implicated in regeneration literature were focused on as well as physiological pathways such as metabolism, stress response, immune/inflammatory response. Physically, environmental parameters were explored, e.g., temperature, oxygen level, water current, nutrient level. Parameter changes were introduced or molecular modulators (e.g., peptides, small molecules) were administered upon amputation. After 3 years of screen, 3 stimuli emerged that strongly induce regeneration. Small buds began appearing from the amputation site within 3-4 days, and arm regrowth was tracked for 1-2 weeks, after which bell tissue began to grow and complicated assessment. Of the three arms removed, generally 1 arm regenerates, occasionally 2 arms, and only in rare instances 3 arms. Multiple tissues were regenerated: muscle, neurons, circulatory canals, and the sensory organ rhopalium (FIG. 5A-FIG. 5B). The regenerated arms contract synchronously with the existing arms (FIG. 29), demonstrating a functional neuromuscular network. The frequency and extent of induced regeneration varied. Frequency of regeneration varied across clutches, i.e., strobilation cohorts, even when the experiments were performed side by side. Extent of regeneration varied anywhere from small buds to rudimentary arms to almost complete arms—even within individuals (FIG. 1F). The variation persists even across genetically clonal populations (FIG. 6A-FIG. 6B). Thus, unlike the robust regeneration in e.g., axolotl, planaria, or hydra, arm regeneration in Aurelia requires exogenous factors, is sensitive to environmental parameters, and manifests variably.

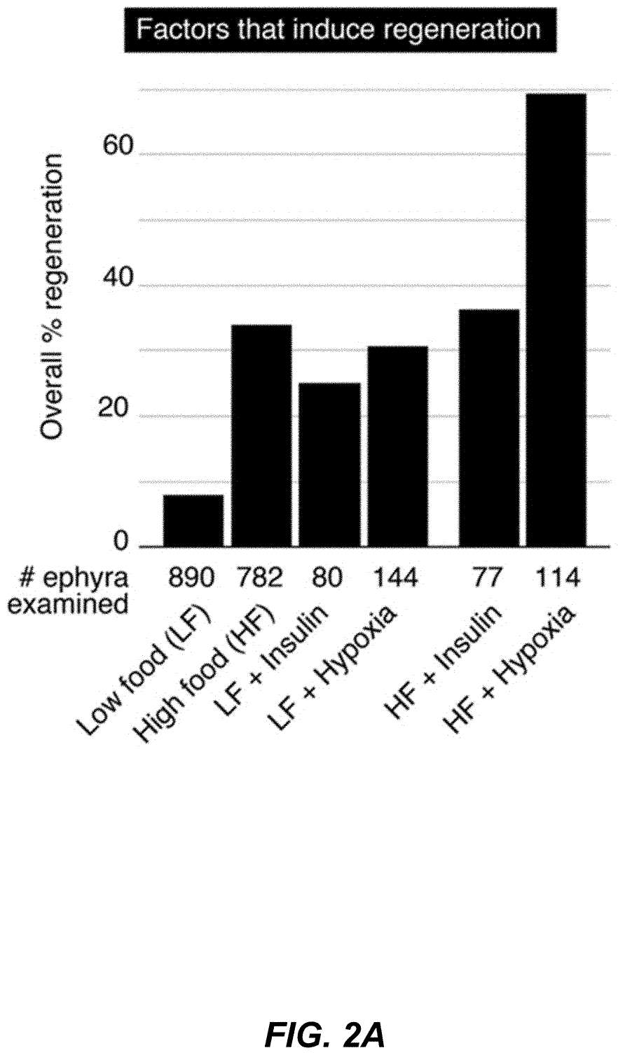

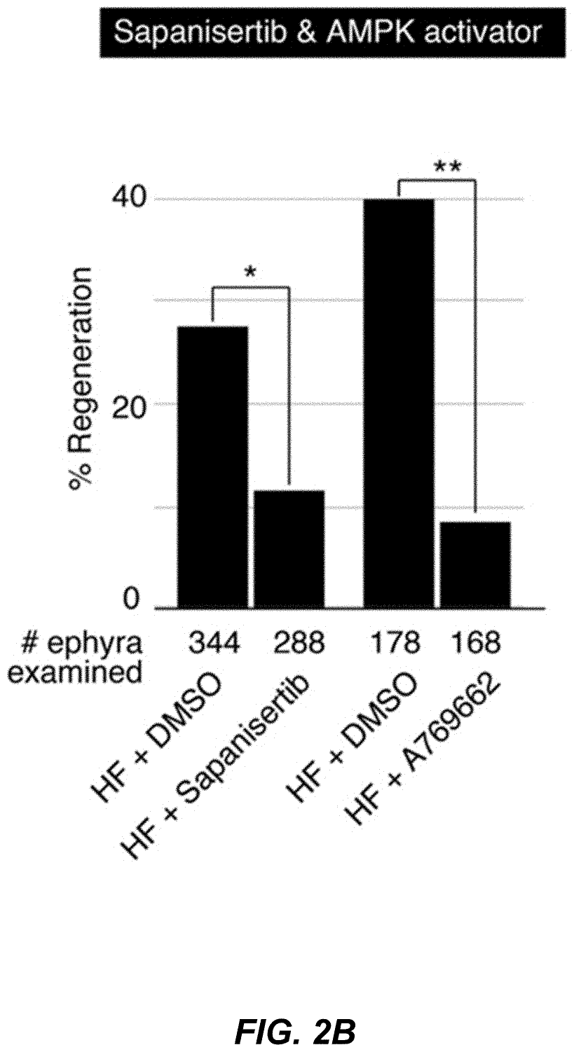

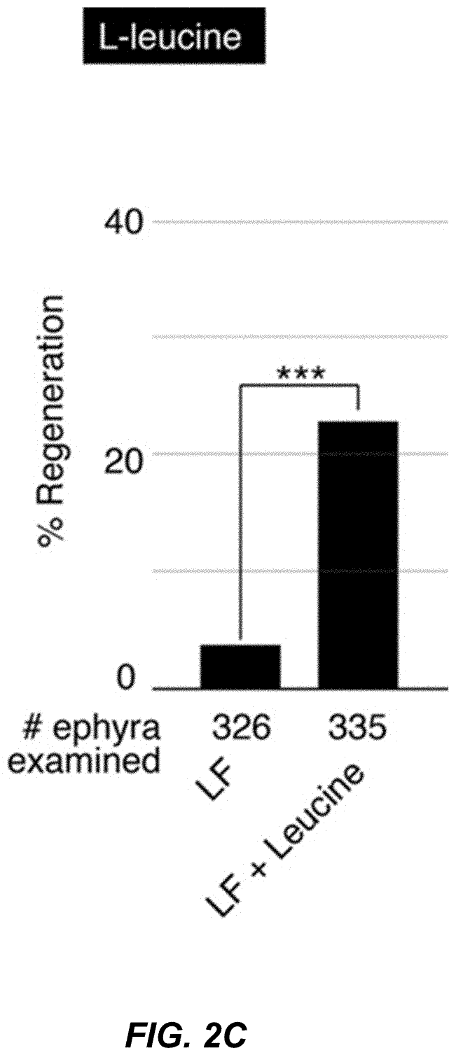

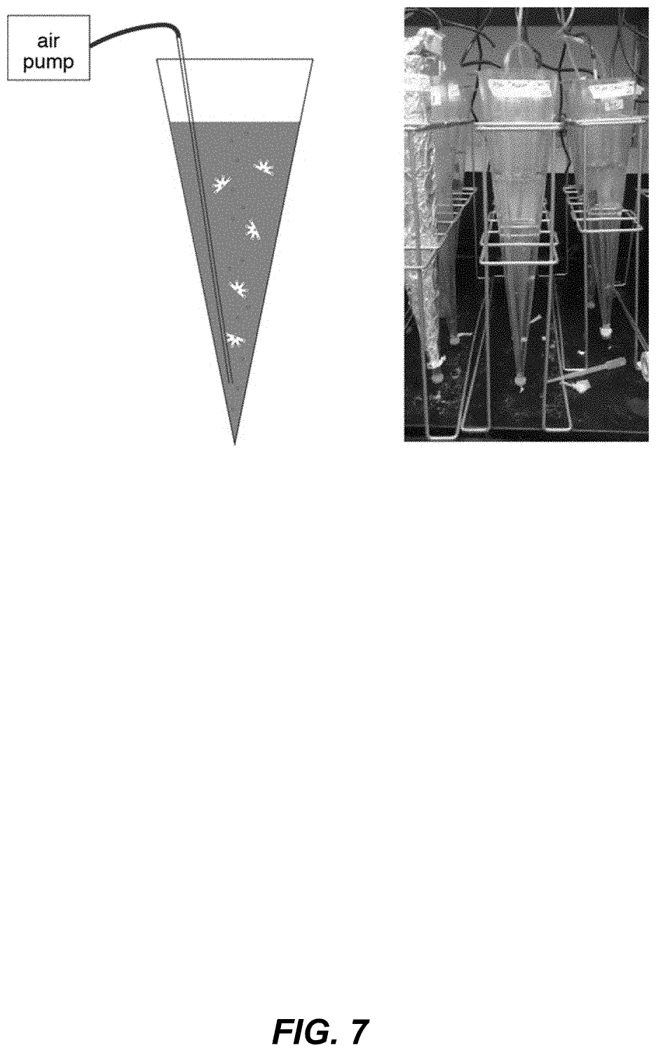

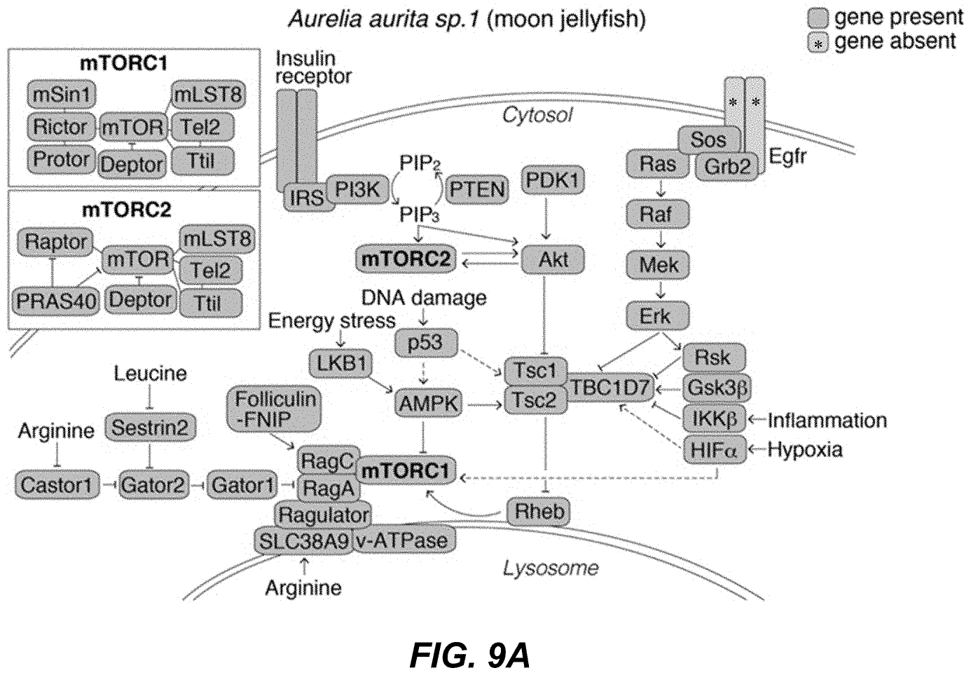

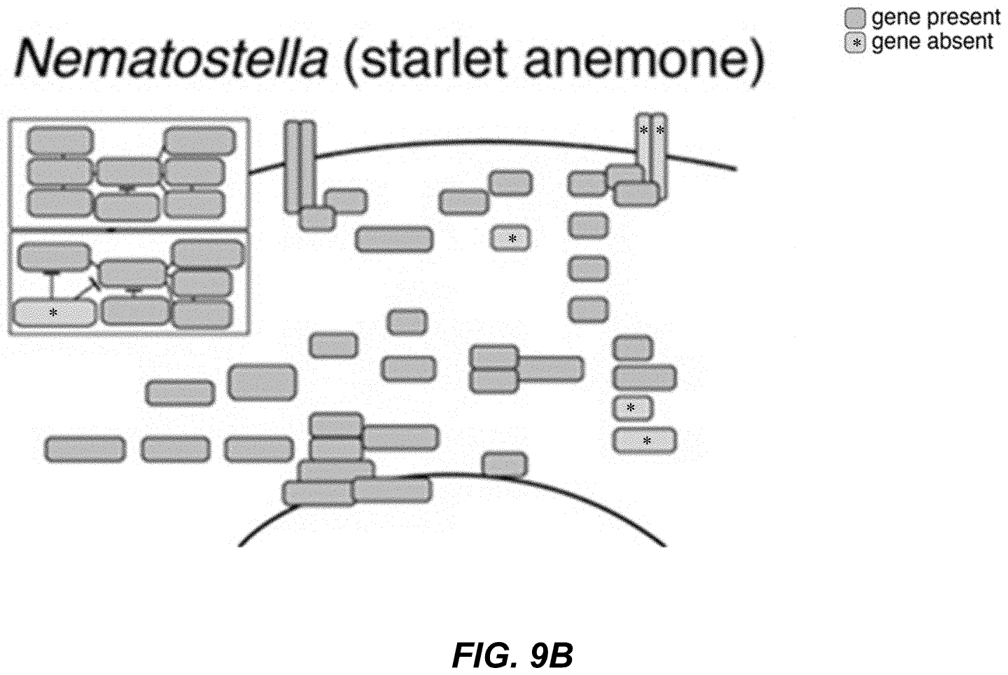

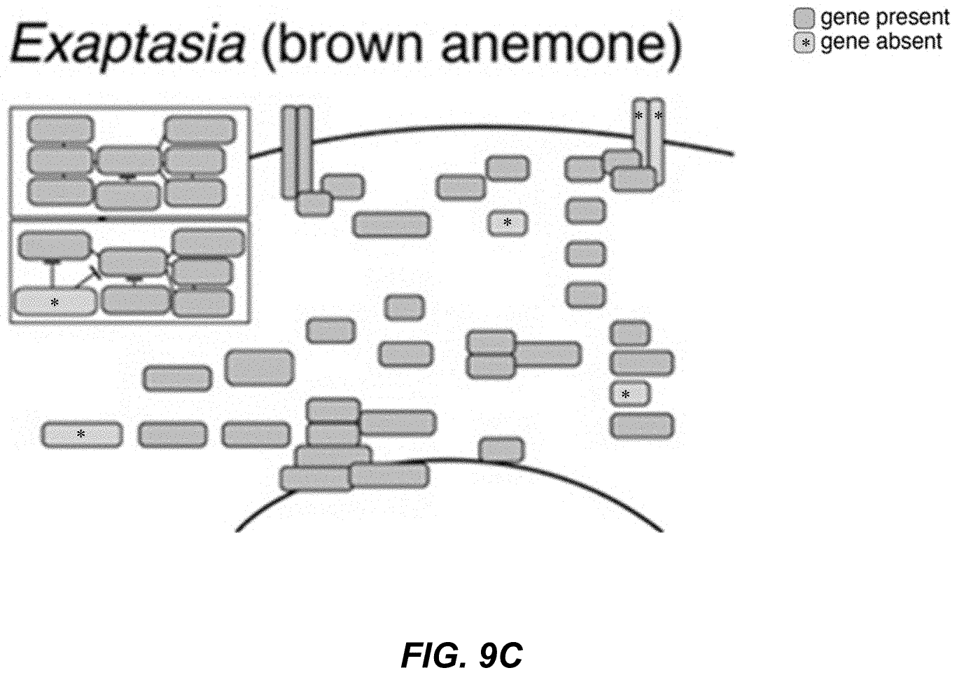

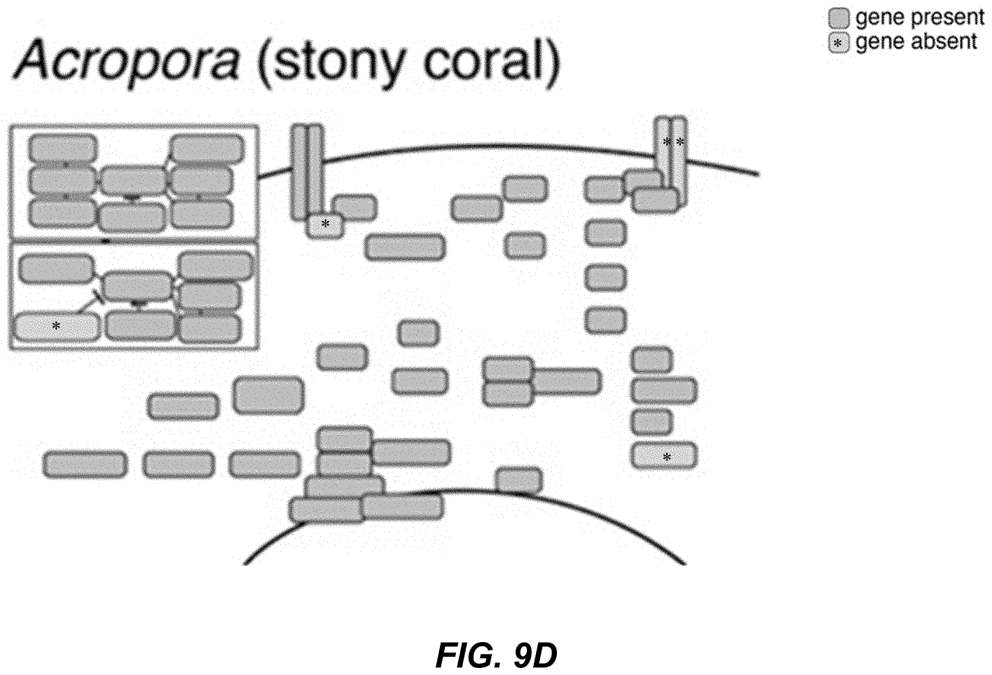

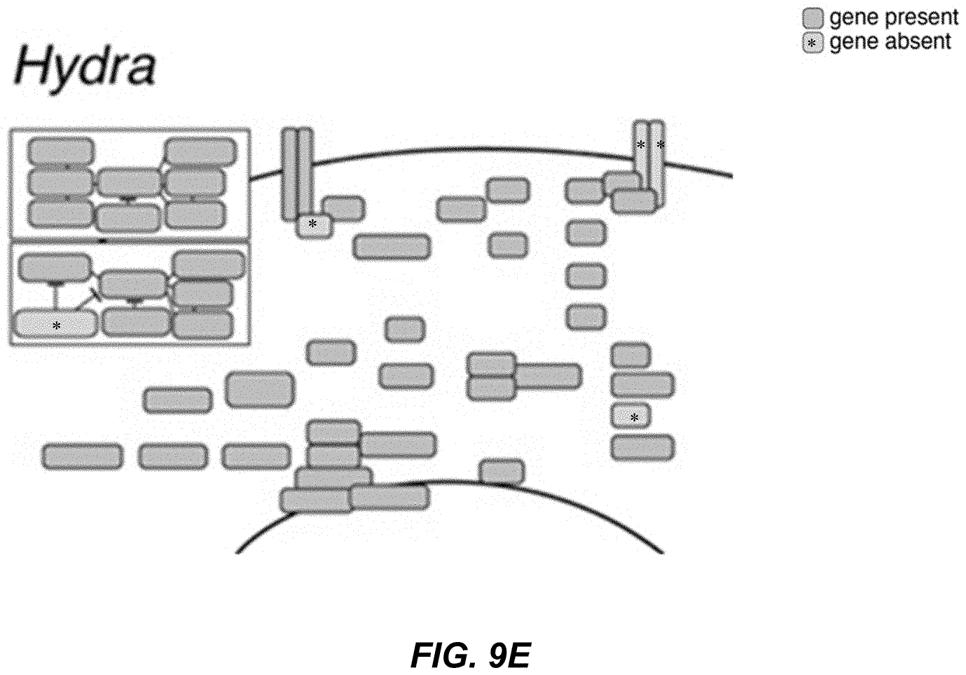

What are the stimuli that induce regeneration? Notably, modulation of developmental signaling pathways did not induce regeneration (e.g., Wnt, Bmp, Tgfb). First, water current was identified as necessary (FIG. 7). Behaviorally, this condition promotes activity. In stagnant water, ephyrae rest at bottom and pulse stationarily. In the presence of water current, ephyrae actively swim and ride the current. In this permissive condition, the first stimulus that induces regeneration is nutrient level: increasing food amount increases frequency and extent of arm regeneration (FIG. 2A). As control, low amount of food was supplied that recapitulates the regeneration frequency in the natural habitat. The second inducer is insulin (FIG. 2A). It was verified that the insulin receptor is conserved in Aurelia (FIG. 9A-FIG. 9E). The insulin effect was not likely due to non-specific addition of proteins, because other proteins such as Egf, Tgfb, and BSA showed no effect. Finally, the third inducer is hypoxia (FIG. 2A). It was verified that the ancient oxygen sensor HIFα is present in Aurelia (FIG. 9A-FIG. 9E). To reduce oxygen, nitrogen was flown into the seawater, achieving ˜50% reduction in dissolved oxygen level. The effect was not due to increased nitrogen, since reducing oxygen using argon flow also dramatically induced regeneration (Table 2A-Table 2C). There appears to be synergy between some stimuli, especially between high food and hypoxia (FIG. 2A).

TABLE 2A
REGENERATION INDUCTION EXPERIMENTS

| Factor | | # ephyrae | Percent |
|---|---|---|---|
| Nutrients | Exp ID | examined | regeneration |
| Low Food (LF) | 78 | 81 | 0 |
| ~20 rotifers | 78* | 48 | 0 |
| ephyra | | | |
| | 79 | 88 | 0 |
| | 81 | 83 | 13.3 |
| ~10 rotifers | 82 | 89 | 4.5 |
| ephyra | | | |
| | 83 | 85 | 32.9 |
| | 84 | 90 | 16.7 |
| | 91 | 85 | 0 |
| | 92 | 84 | 4.8 |
| | 93 | 82 | 4.9 |
| | 94 | 75 | 5.3 |
| | | Total | Average |
| | | 890 | 7.9 |
| High Food (HF) | 78 | 81 | 0 |
| ~40 rotifers | 79 | 82 | 12.2 |
| ephyra | | | |
| | 81 | 87 | 62.1 |
| | 82 | 89 | 7.9 |
| | 83 | 58 | 34.5 |
| | 84 | 89 | 37.1 |
| | 85 | 90 | 24.4 |
| | 86 | 57 | 15.8 |
| | 87 | 89 | 68.5 |
| | 88 | 60 | 83.3 |
| | | Total | Average |
| | | 782 | 34.0 |

Experiments were performed on a clonal line (clone 3 in FIG. 6A-FIG. 6B).
In each experiment, a plate of polyps was strobilated (a plate may contain 50-100 polyps), and tests were performed on the ephyrae.
Food amount, at the indicated density, was administered daily.
Insulin, mTOR activators or inhibitors were administered weekly.
To generate hypoxic condition, nitrogen or argon, instead of ambient air, was flown into the bubbler cones to achieve ~50% reduction in oxygen level.

TABLE 2B
REGENERATION INDUCTION EXPERIMENTS

| | | # ephyrae | |
|---|---|---|---|
| Factor | Exp ID | examined | Percent regeneration |
| Insulin | | | |
| LF | 79 | 88 | 0 |
| LF + Insulin | | 80 | 25 |
| HF | | 82 | 12.2 |
| HF + Insulin | | 77 | 36.4 |
| Hypoxia | | | |
| LF | 79 | 88 | 0 |
| LF + nitrogen | | 77 | 32.5 |
| LF* | | 48 | 0 |
| LF* + nitrogen | | 67 | 28.4 |
| | | Total | Average |
| LF + nitrogen | | 144 | 30.6 |
| HF | 79 | 82 | 12.2 |
| HF + nitrogen | | 56 | 82.1 |
| HF | 81 | 87 | 62.1 |
| HF + nitrogen | | 58 | 56.9 |
| | | Total | Average |
| HF + nitrogen | | 114 | 69.3 |
| HF | 84 | 89 | 37.1 |
| HF + argon | | 69 | 72.5 |

Experiments were performed on a clonal line (clone 3 in FIG. 6A-FIG. 6B).
In each experiment, a plate of polyps was strobilated (a plate may contain 50-100 polyps), and tests were performed on the ephyrae.
Food amount, at the indicated density, was administered daily.
Insulin, mTOR activators or inhibitors were administered weekly.
To generate hypoxic condition, nitrogen or argon, instead of ambient air, was flown into the bubbler cones to achieve ~50% reduction in oxygen level.

TABLE 2C
REGENERATION INDUCTION EXPERIMENTS

| | | # ephyrae | Percent |
|---|---|---|---|
| Factor | Exp ID | examined | regeneration |
| Sapanisertib | | | |
| HF + DMSO | 94 | 86 | 57.0 |
| HF + Sap | | 43 | 11.6 |
| HF + DMSO | 93 | 89 | 22.5 |
| HF + Sap | | 82 | 15.9 |
| HF + DMSO | 92 | 89 | 19.1 |
| HF + Sap | | 84 | 16.7 |
| HF + DMSO | 91 | 80 | 10.0 |
| HF + Sap | | 79 | 1.3 |
| | | Total | Average |
| HF + DMSO | | 344 | 27.3 |
| HF + Sap | | 288 | 11.5 |
| TTEST | | | 0.029 |
| AMPK activator | | | |
| HF + DMSO | 73 | 88 | 34.1 |
| HF + A769662 | | 82 | 9.8 |
| HF + DMSO | 75 | 90 | 45.6 |
| HF + A769662 | | 86 | 7.0 |
| | | Total | Average |
| HF + DMSO | | 178 | 39.9 |
| HF + A769662 | | 168 | 8.3 |
| TTEST | | | 0.007 |
| L-leucine | | | |
| LF | 91 | 85 | 0 |
| LF + leucine | | 89 | 23.6 |
| LF | 93 | 82 | 4.9 |
| LF + leucine | | 83 | 16.9 |
| LF | 92 | 84 | 4.8 |
| LF + leucine | | 82 | 13.4 |
| LF | 94 | 75 | 5.3 |
| LF + leucine | | 81 | 38.3 |
| | | Total | Average |
| LF | | 326 | 3.7 |
| LF + leucine | | 335 | 23.0 |
| TTEST | | | 5E−5 |

Experiments were performed on a clonal line (clone 3 in FIG. 6A-FIG. 6B).
In each experiment, a plate of polyps was strobilated (a plate may contain 50-100 polyps), and tests were performed on the ephyrae.
Food amount, at the indicated density, was administered daily.
Insulin, mTOR activators or inhibitors were administered weekly.
To generate hypoxic condition, nitrogen or argon, instead of ambient air, was flown into the bubbler cones to achieve ~50% reduction in oxygen level.

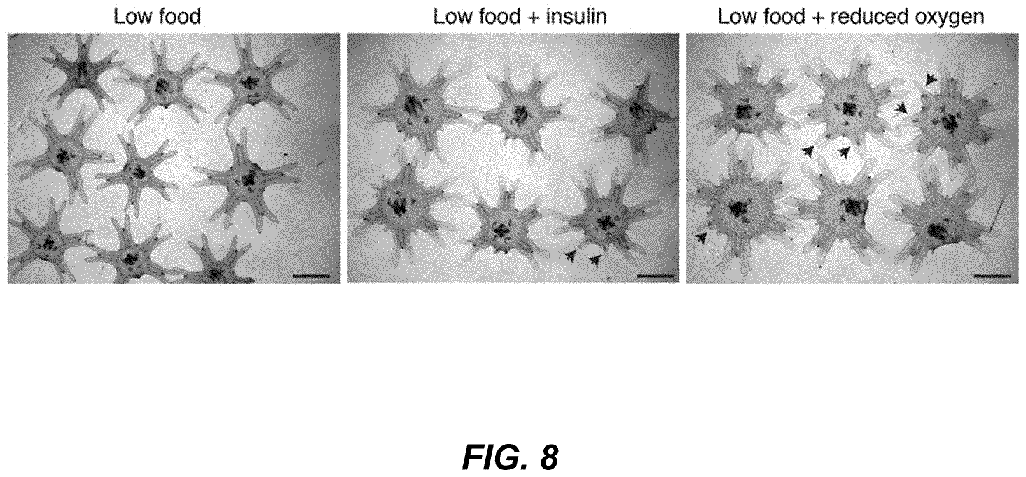

There could be more inducers to be found, but given the strong induction already observed, the identified factors likely hint a key strategy. Notably, the stimuli induced growth: treated ephyrae were larger than control ones (FIG. 8). This is tantalizing because growth regulation is deeply conserved in eukaryotes: From yeasts to mammals, the master controller of growth is the mechanistic target of rapamycin (mTOR) pathway. The mTOR pathway coordinates growth with multiple inputs, including nutrients (sensing specific amino acids), growth factors (e.g., insulin, IGF), forms of stress (e.g., hypoxia), as well as physical activity—the very factors identified in the screen.

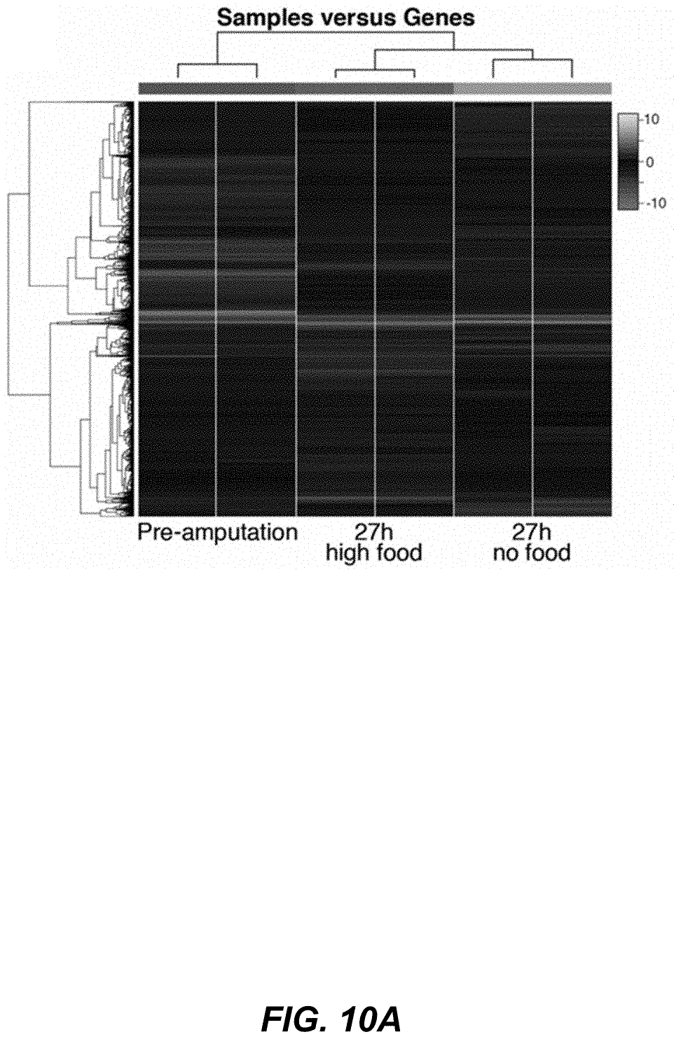

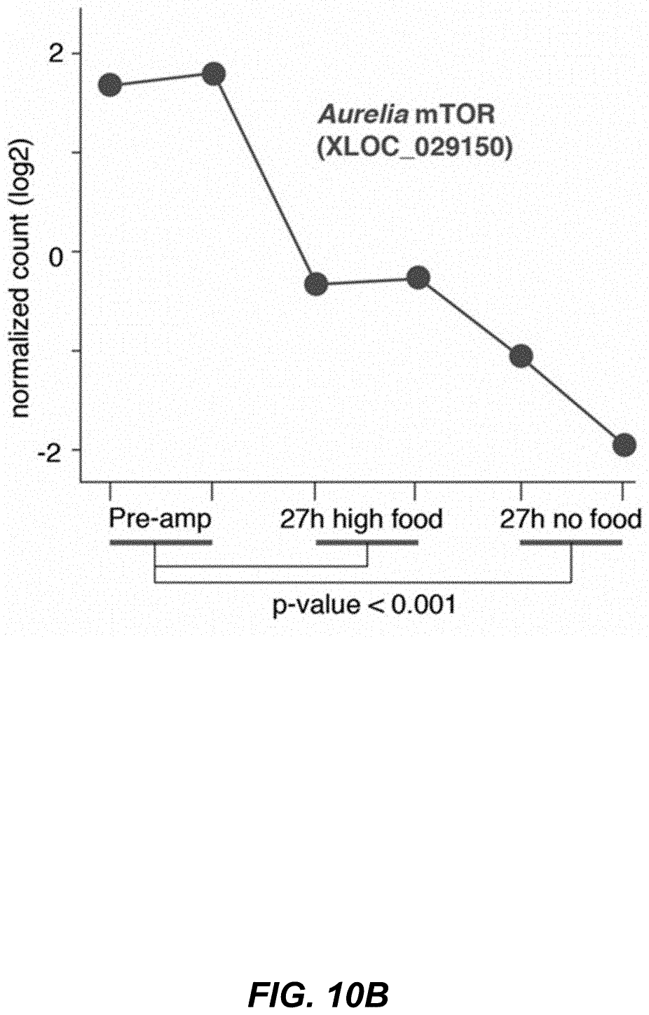

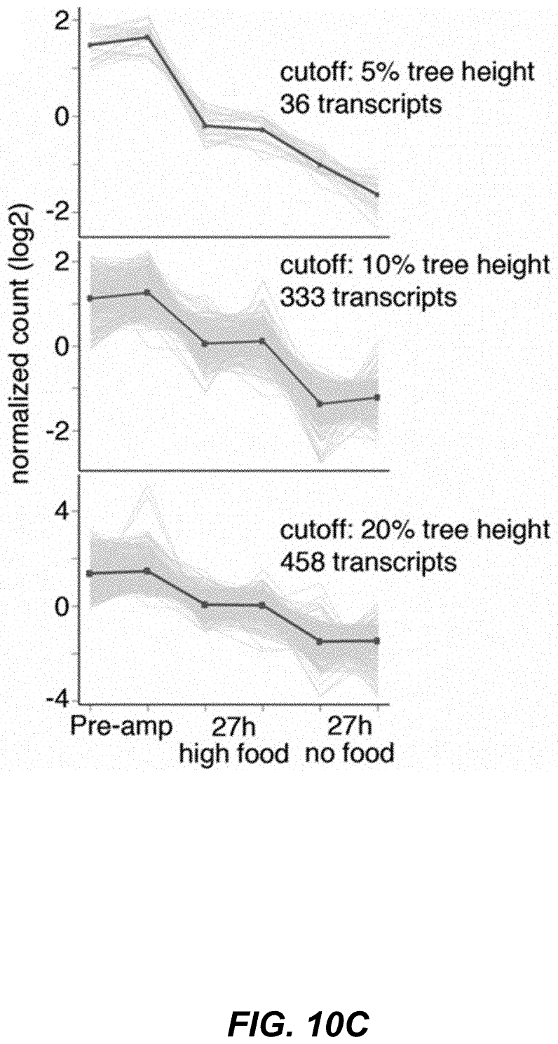

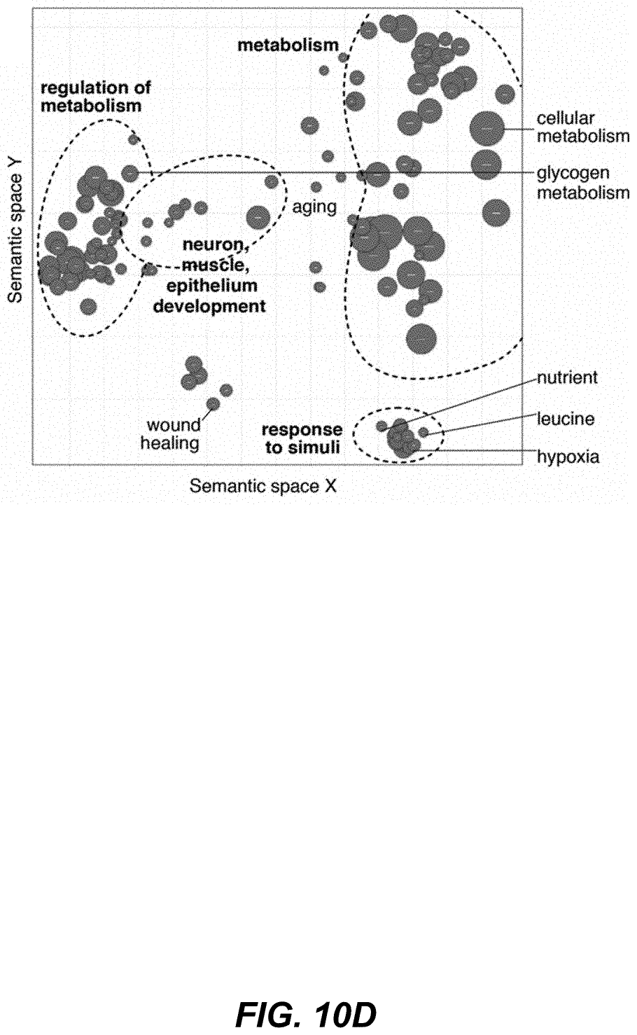

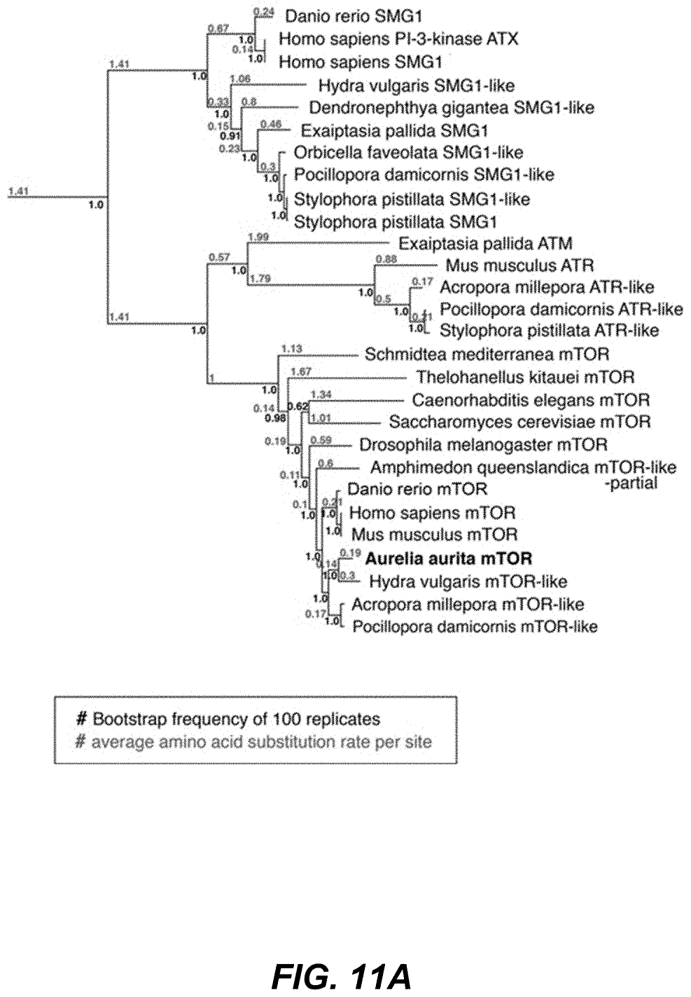

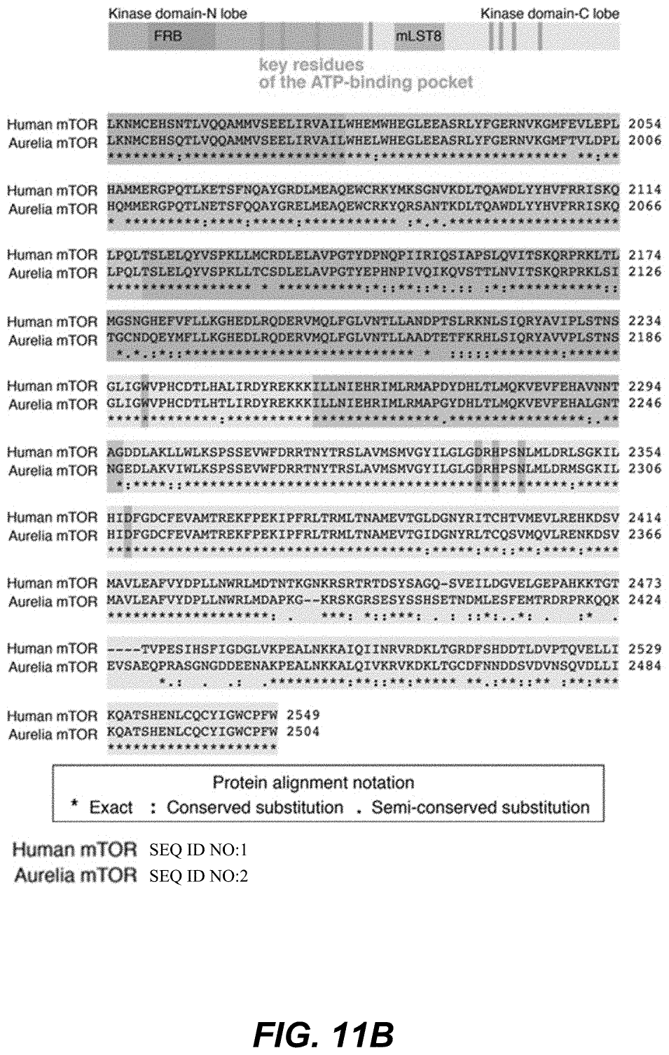

It was verified that the mTOR pathway is preserved in Aurelia, and across cnidarians (FIG. 9A-FIG. 9E). Performing RNA sequencing, expression of mTOR and mTOR-related genes are downregulated upon injury correlating with the poor regenerative response, but activated in high-food condition that correlates with regeneration induction (FIG. 10A-FIG. 10D). Beyond correlation, it was tested if mTOR inhibitors would repress regeneration. Sapanisertib is a potent pan-mTORC1/2 inhibitor that competes for the ATP binding pocket (conserved in the Aurelia mTOR, FIG. 11A-FIG. 11B). Administration of sapanisertib dramatically inhibited arm regeneration (FIG. 2B). A different strategy to inhibit mTOR is through the energy sensor AMP-activated Protein Kinase (AMPK), which antagonizes mTOR, ensuring growth only proceeds in plentiful condition. Administration of A769662, a thienopyridone that mimics the allosteric effect of AMP in activating AMPK, inhibited arm regeneration (FIG. 2B). Finally, it was tested if a known mTOR activator can induce regeneration. A direct input to mTOR is the amino acid L-leucine. In addition to its role as a proteogenic amino acid, leucine acts as a signaling molecule, in large part through activating mTOR. Leucine binds to Sestrin2 (conserved in Aurelia, FIG. 9A-FIG. 9E), relieving the inhibition of Rag GTPases and activating mTOR. Because animals typically have poor ability to metabolize leucine, extracellular concentration of leucine fluctuates with consumption, and dietary leucine directly controls mTOR activity. Indeed, feeding amputated ephyrae with L-leucine induced arm regeneration (FIG. 2C).

These findings suggest that the regeneration inducers identified act at least partially through the mTOR pathway. Next, it was explored whether the same strategy for inducing appendage regeneration might apply to other animals. For the next poorly regenerating system, the fruit fly Drosophila melanogaster was turned to. Along with beetles and butterflies, Drosophila belong to the holometabolans, a vast group of insects with complete metamorphosis that as adults do not regenerate limbs or other appendages. Motivated by the findings in Aurelia, it was tested if administration of L-leucine and insulin could induce limb regeneration in Drosophila. L-glutamine was added to enhance leucine uptake. Of the inducers found, L-leucine and insulin were tested because they were the most straightforward to administer. Additionally, while hypoxia promotes growth in Aurelia, Drosophila is extremely resistant to hypoxia.

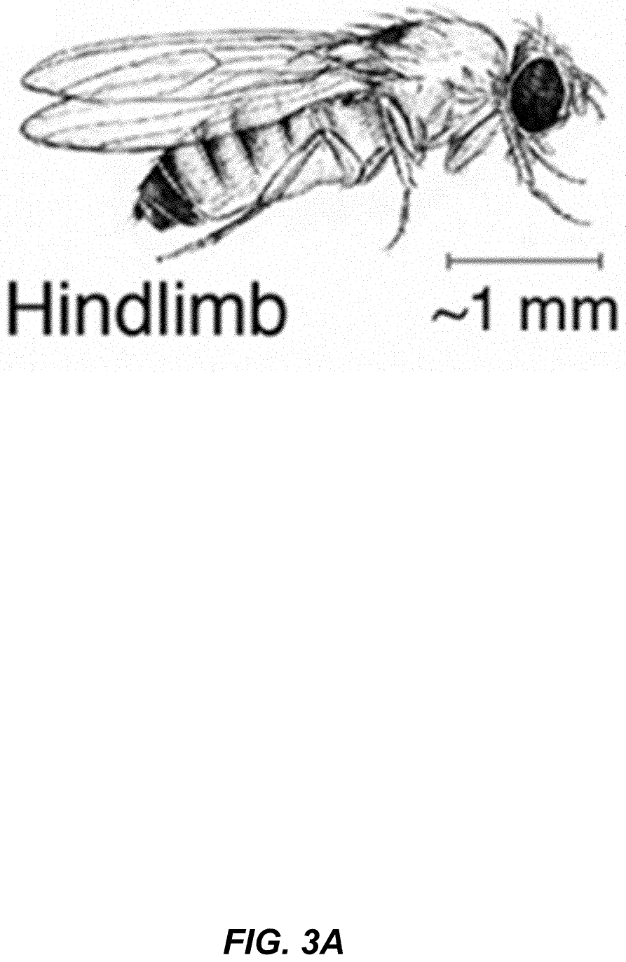

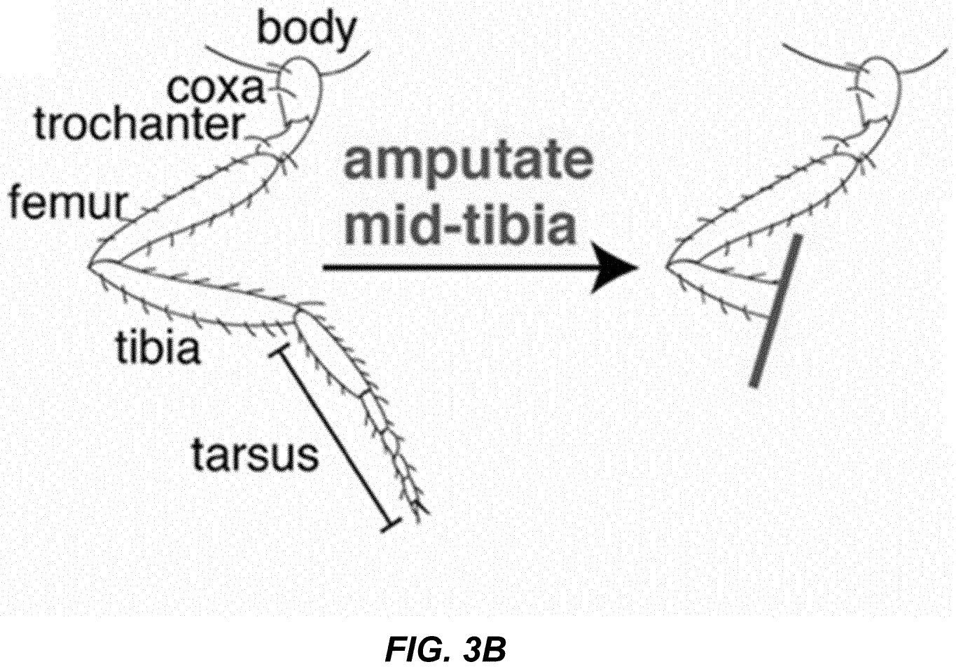

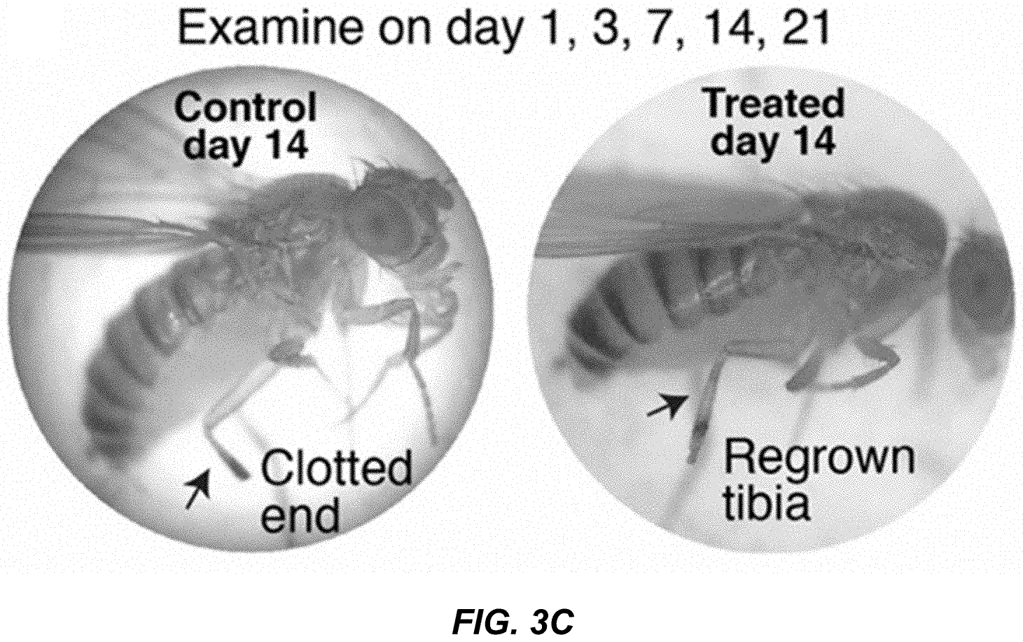

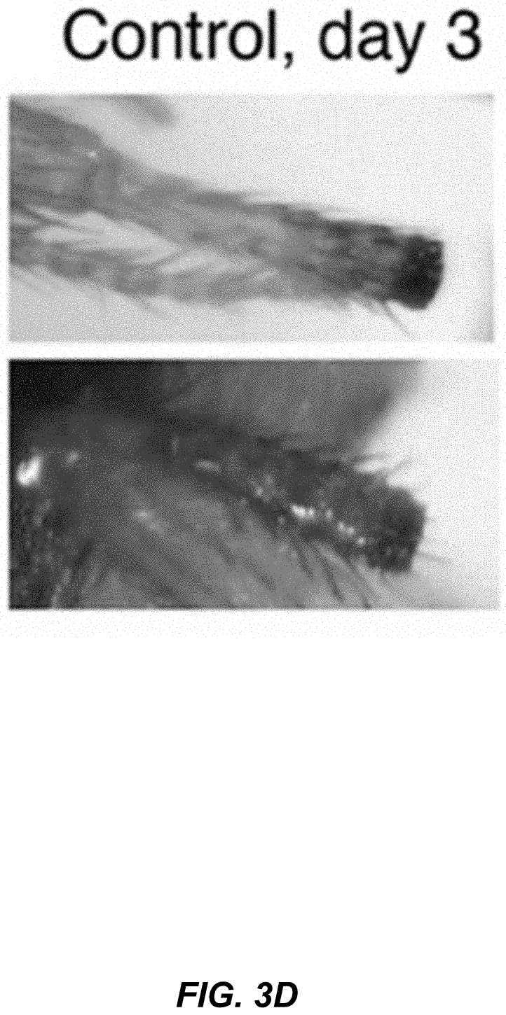

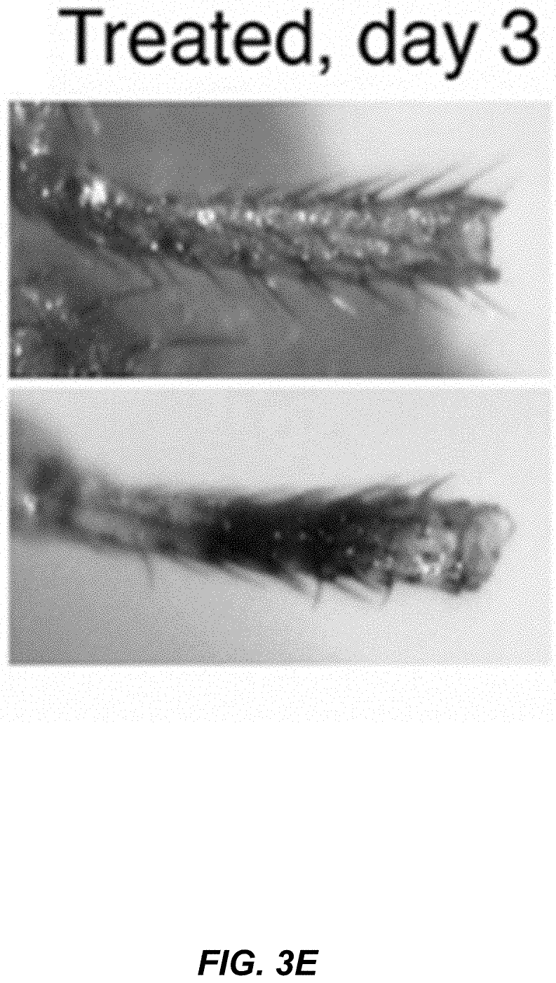

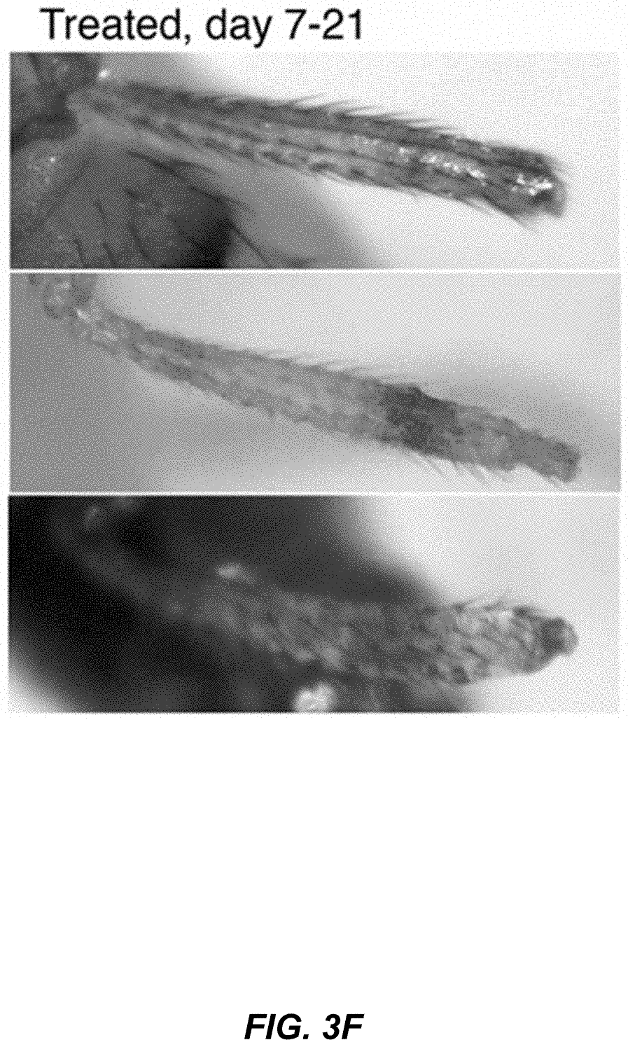

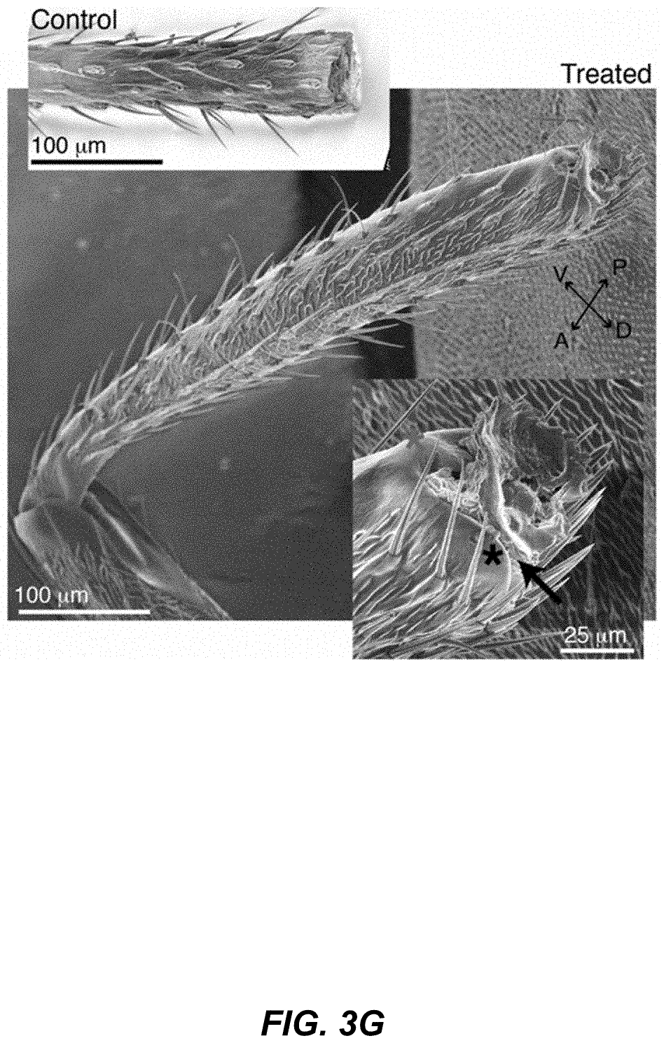

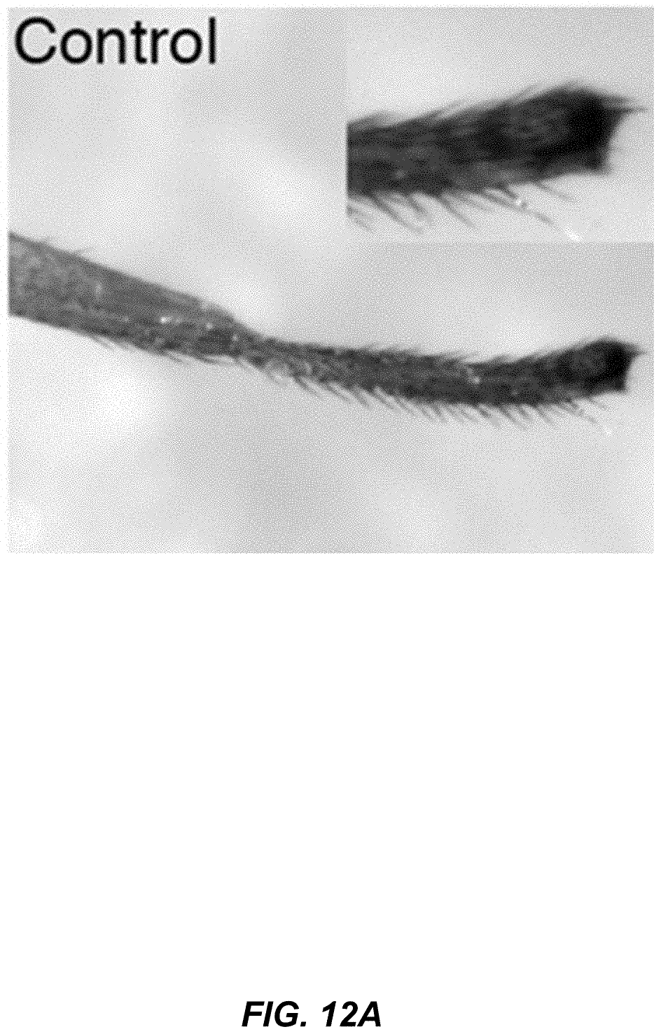

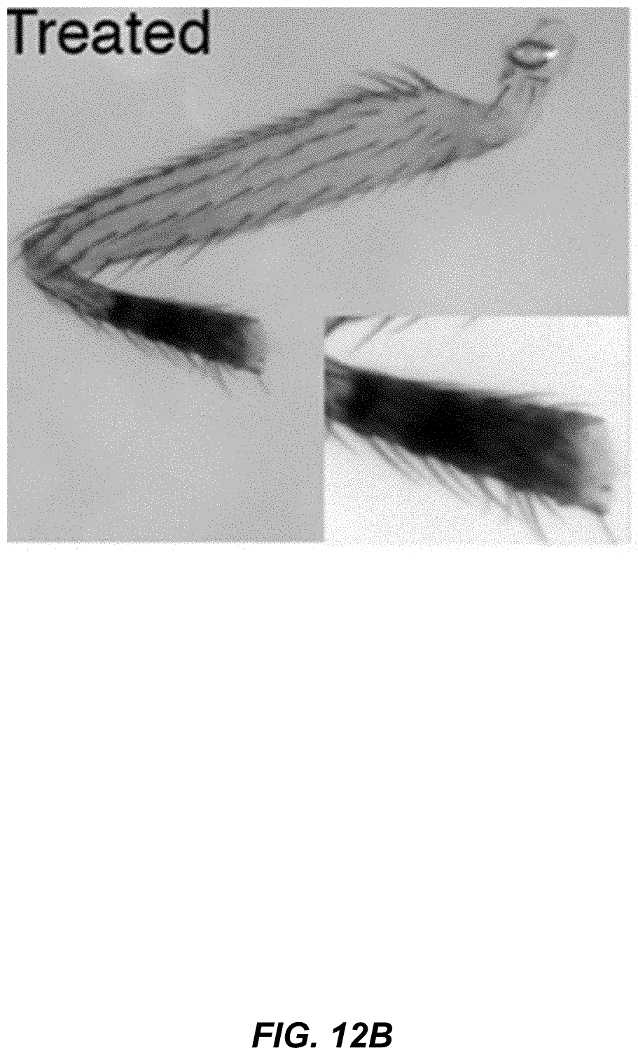

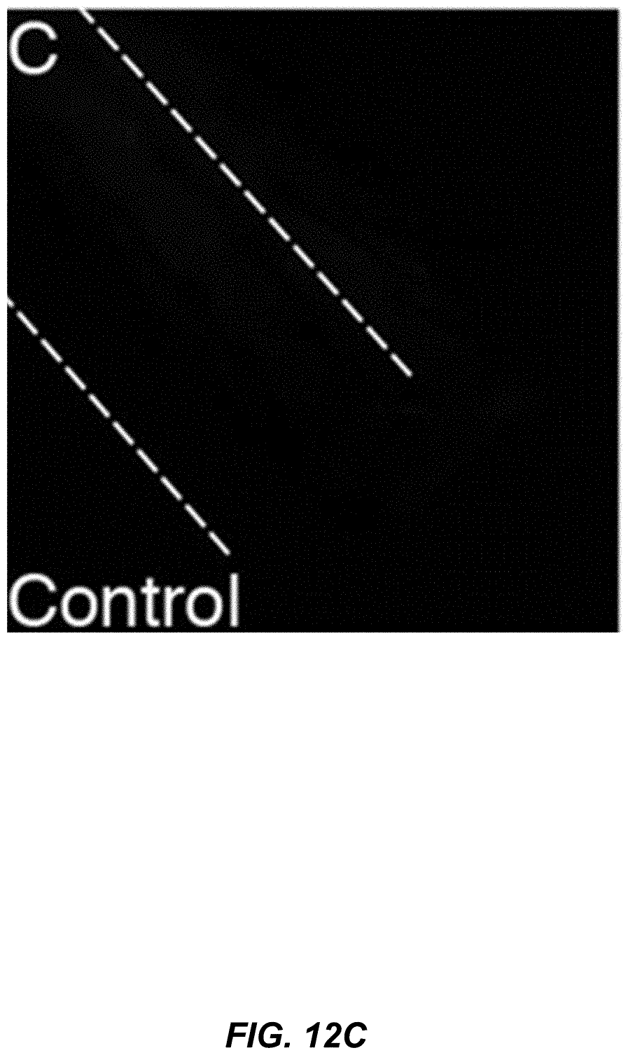

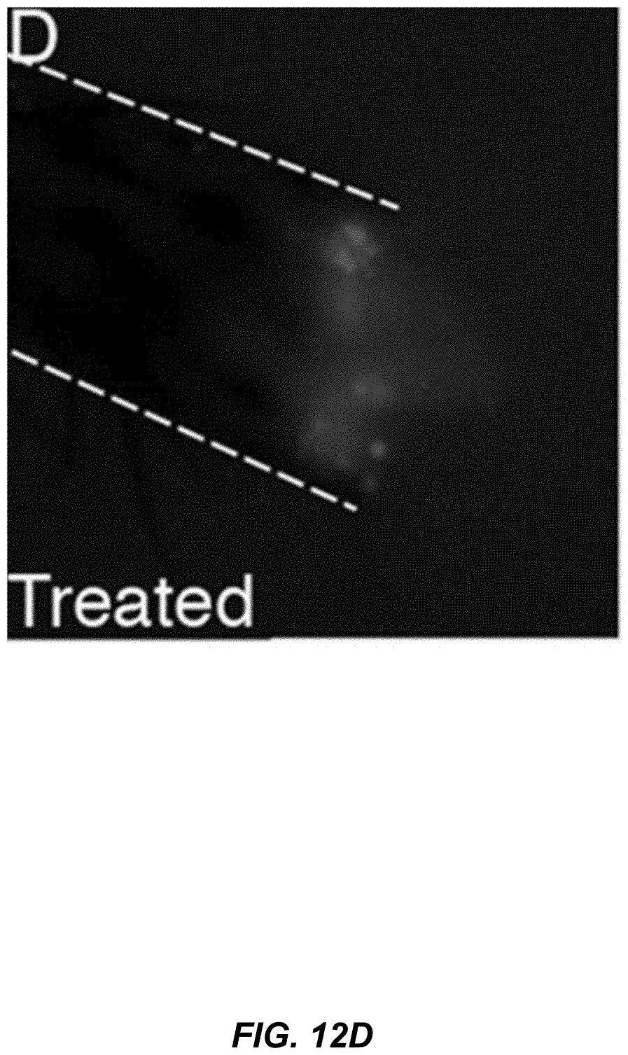

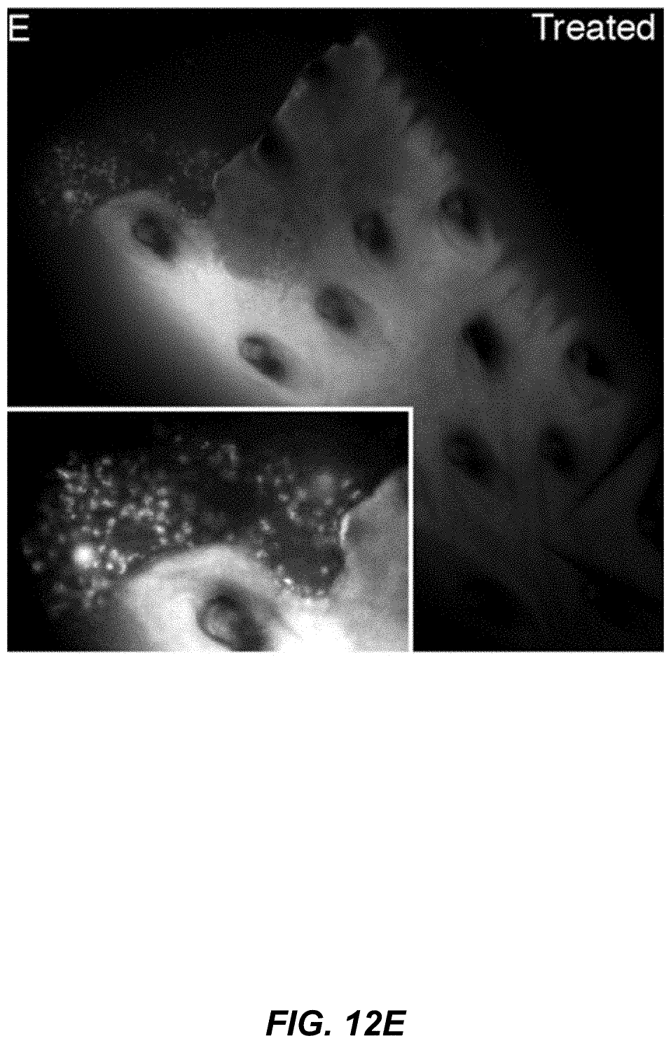

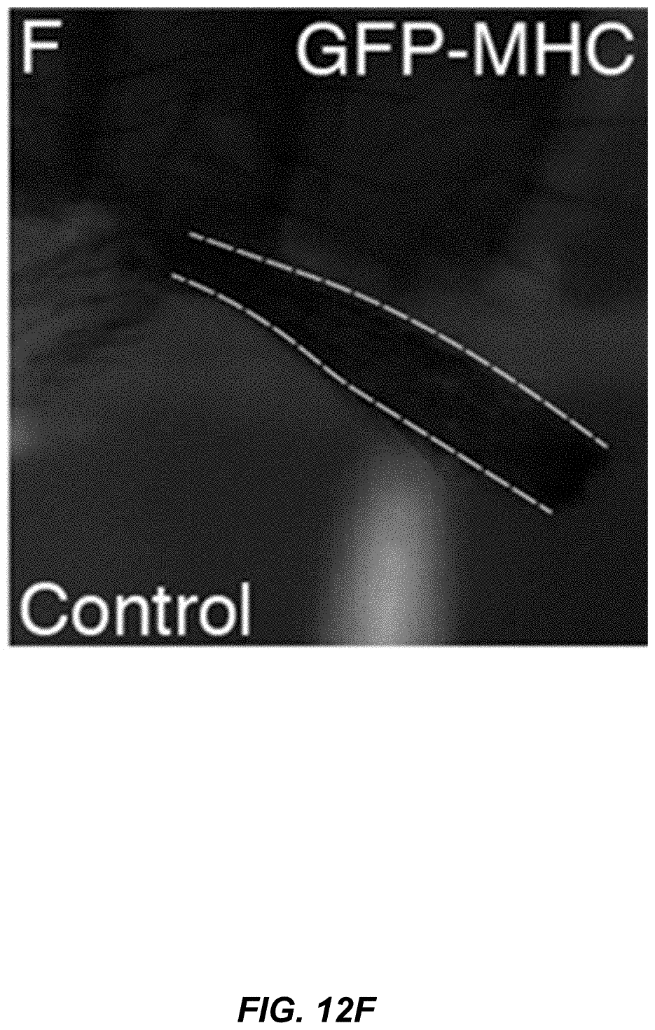

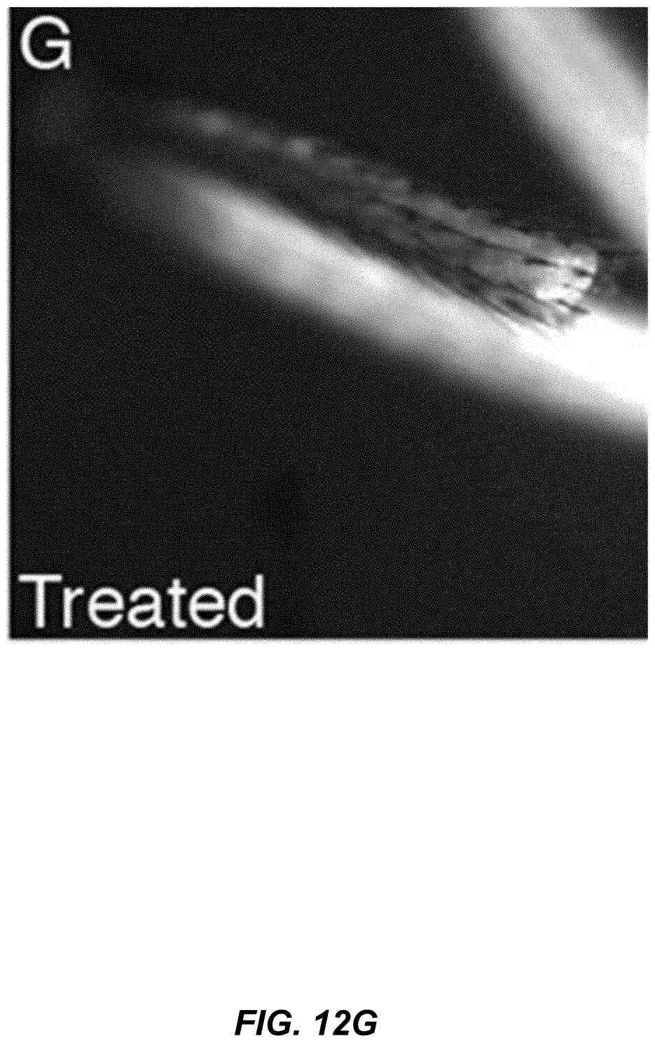

Drosophila has 6 limbs extending from the thorax, each a jointed limb with rigid leg segments connected by flexible joints. Amputation was performed on a hindlimb (FIG. 3A), across the fourth segment of the limb, the tibia (FIG. 3B). Each fly was examined at multiple times after amputation to assess signs of regeneration (FIG. 3C)—and on day 1 and 3, any possible contamination (e.g., of flies with uncut tibia), if any, was promptly removed. The control amputated legs quickly sealed and, within 1-3 days, form a dark melanized clot (FIG. 3B, FIG. 3D)-consistent with normal wound healing process. By contrast, within 3 days after amputation, at least 12.1% of the treated flies (N=387) showed no clot, but instead white-colored live cellular material at the wound site (FIG. 3E, FIG. 12A-FIG. 12G), as confirmed by cell nuclear staining and muscle signal (FIG. 12A-FIG. 12G).

The switch from dead clot to live tissue suggests induction of regenerative state. Indeed, 1-3 weeks after amputation, 1.3% of the flies (N=387) showed a fully regrown tibial segment (FIG. 3C, FIG. 3F), compared to 0% in 860 control flies. This reported frequency could be a lower estimate because to be fully certain only full tibial regrowth was scored (i.e., one that culminates in a joint-like structure), and therefore might be missing any partial regrowth. To further verify that the regrown tibia was indeed new regrowth, all the flies with live-tip tibia were separated on day 3, and it was confirmed that regrown tibia was only observed in this population (Table 3).

TABLE 3
INDUCED TIBIA REGENERATION IN DROSOPHILA OREGONR STRAIN.

| | Control | Control | Control | Treated | Treated | Treated |
|---|---|---|---|---|---|---|
| Experiment | # flies | Live tip | tibia | # flies | Live tip | tibia |
| Leucine† | 80 | 0 | 0 | 240 | * | 3 |
| Leucine + Insulin‡ | 20 | 0 | 0 | 71 | 22 | 1 |
| Leucine + Insulin + | 40 | 0 | 0 | 76 | 25 | 1 |
| Fructose | | | | | | |
| 1,6-bisphosphate‡, ∥ | | | | | | |
| Control (combined | 720 | 0 | 0 | | | |
| from all other exps) | | | | | | |
| Total | 860 | 0 | 0 | 387 | 47 | 5 |
| % Phenotype | | 0 | 0 | | >12.1 | 1.3 |

Adult flies, within 1 week after eclosion, were amputated across the hind tibia.
Afterward, the flies were examined at day 1, 3, 7, 14, and 21.
Various doses L-leucine (2-100 mM), L-Glutamine (2-10 mM), and insulin (0.1-0.5 mg/mL) were tested. 5 mM L-Leucine, 5 mM L-Glutamine, and 0.1 mg/mL insulin were found to be the most effective.
In other doses, either no phenotypes or lower frequency of phenotype were seen.
The experiments listed here were performed using the optimal doses, and used to compute the % phenotype reported in the present Example.
* The live-tip phenotype was also observed in the leucine-only experiment, but was not counted. The 12.1% total live-tip count reported is therefore a lower estimate.
†Treatment for the first week only.
No significant differences were observed between treatment for the first week only or for the entire duration of experiment.
‡Treatment for the entire exp.
Flies with live-tip were separated on day 3, tibia regrowth was observed only from this population.
∥ Fructose 1,6-bisphosphate was an effort to see if adding a metabolic modulator would enhance the effect of leucine and insulin, but the result simply reproduced the leucin/insulin treatment, hence included here.

Scanning electron microscopy (FIG. 3G) shows a regenerated tibia enclosed in sclerotized cuticle lined with bristles in the usual longitudinal rows. The distal tibial end tapers, articulating from which appears to be the beginning of a next segment. A close-up of the joint-like structure shows the expected bilateral symmetry of a tibial/tarsal joint (as opposed to the radially symmetrical tarsal/tarsal joint). The tibial/tarsal joint is a dicondylic joint, consisting of a peg and socket (called condyle), which serves as rigid points of articulation between opposing leg segments, enabling bending in one plane. Indeed, the regenerate tibial end shows at the anterior/posterior side the expected condylic protrusions (black asterisk), whose ridges serve as points of contact with the opposing segment (arrow). Moreover, a unique feature of the tibial/tarsal joint of the hindlimb (but not of fore or midlimb) is an additional ventral projection that restrict too much bending in that direction—the ventral projection is also observed in the regenerated tibia (grey asterisk). In contrast to the patterned regenerating tibia, the amputated control tibia (top inset) shows a blunt end from the amputation and discoloration corresponding to the clot.

Despite the low frequency, this is the first observation that limb regeneration can be induced in insects. The short lifespan of Drosophila and the accelerated aging, expected from administration of known mTOR activators, prevented observation much beyond 3 weeks. L-leucine appears to be the stronger stimulus: L-leucine can by itself effect tibia regrowth, but co-treatment with insulin enhances the frequency of live-tip phenotype (Table 3). In addition to leucine and insulin, multiple other factors were screened; only leucine and insulin co-treatment resulted in tibia regrowth so far. Finally, the induced tibia regeneration was reproducible in a different Drosophila strain. Notably, as in Aurelia, not all induced regeneration was perfect; occasionally unpatterned response was observed.

The conserved effect of amino acid and insulin supplementation in inducing appendage/limb regeneration in Aurelia and Drosophila motivated us to test a vertebrate system. The mouse digit is the mammalian model for exploring limb regeneration. One hope that limb regeneration may someday be feasible in humans is that fingertips regenerate. Like humans—and reminiscent to ephyrae, mice regenerate digit tips, but not anything more proximal. There is evidence that the digit retains regenerative response. Implanting beads coated with developmental signals leads to regrowth of specific tissues from a proximal stump (e.g., bone elongation with Bmp2, or joint-like structure with Bmp9). Beyond inducing specific tissues, reactivation the embryonic gene 1in28 excitingly leads to two-day old neonates regrowing distal phalange—but the effect does not extend beyond early neonates.

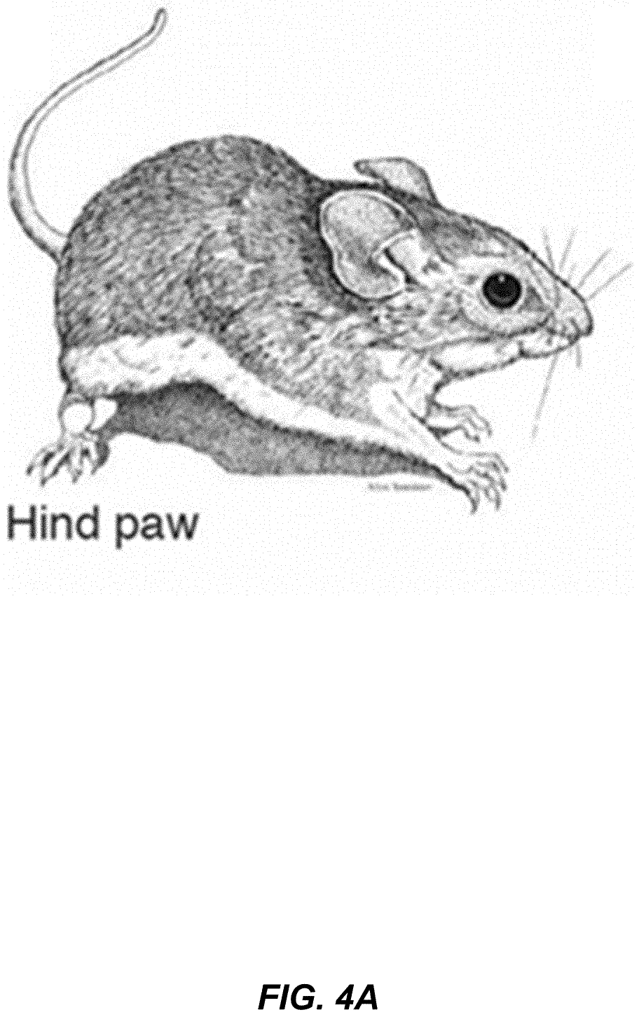

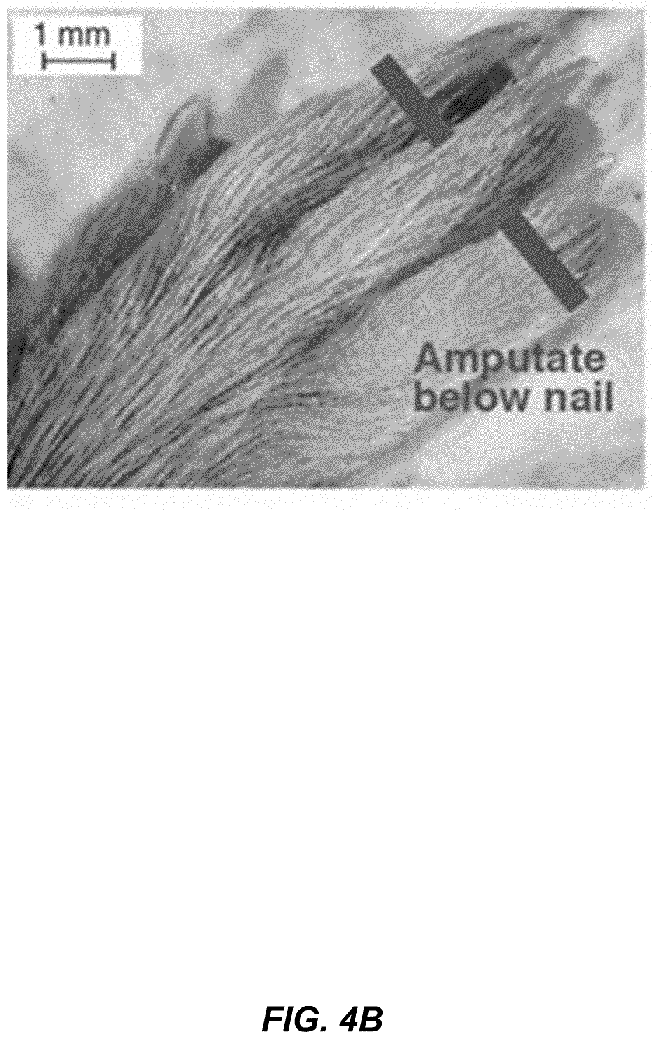

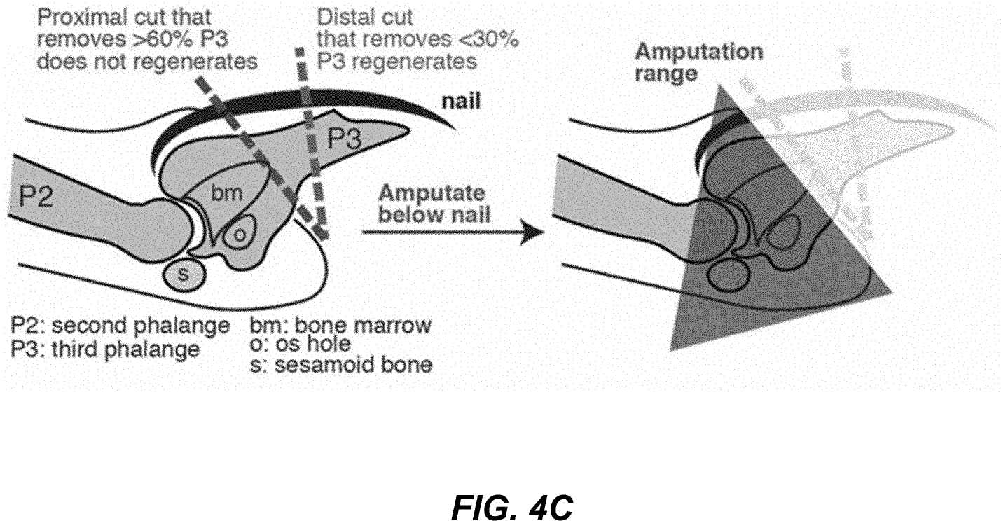

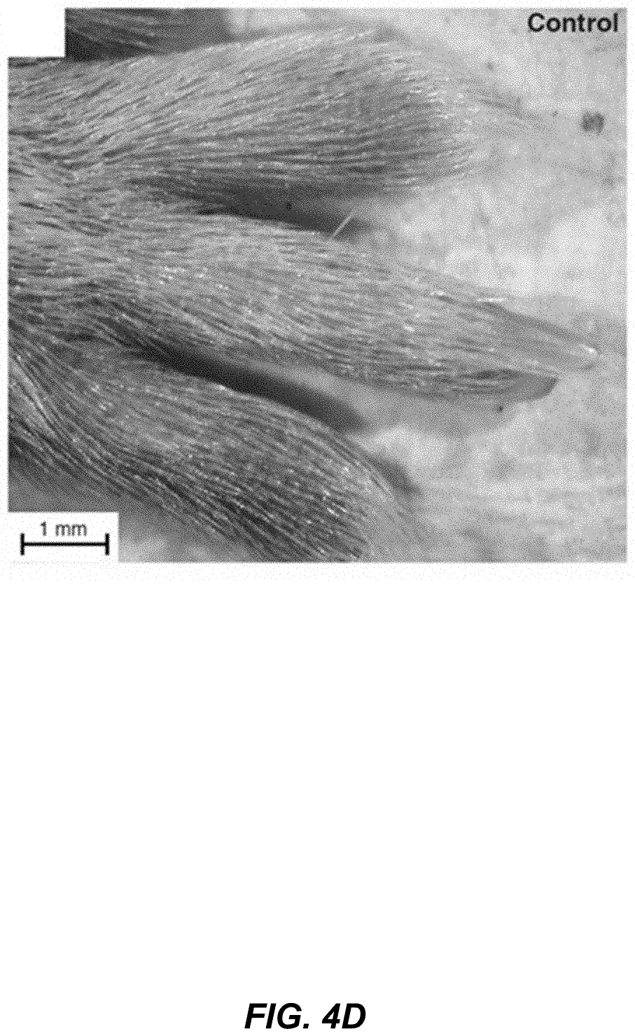

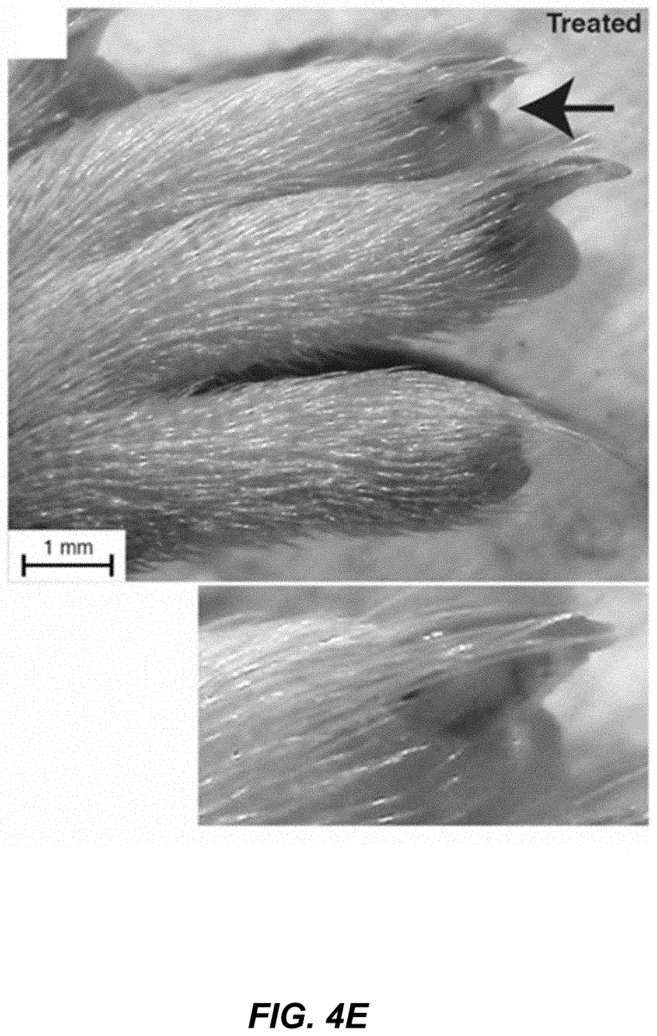

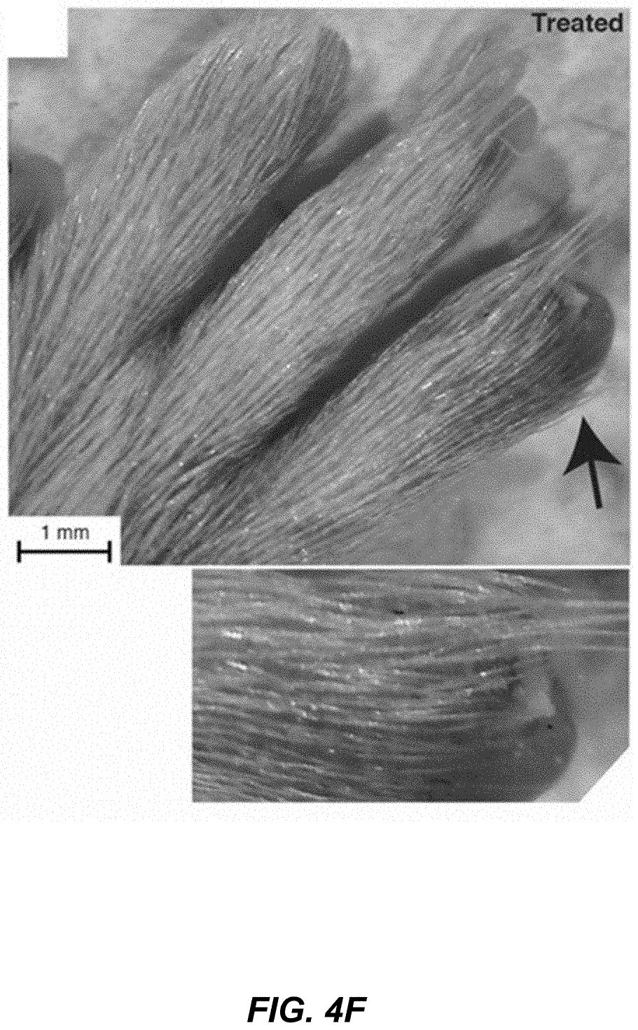

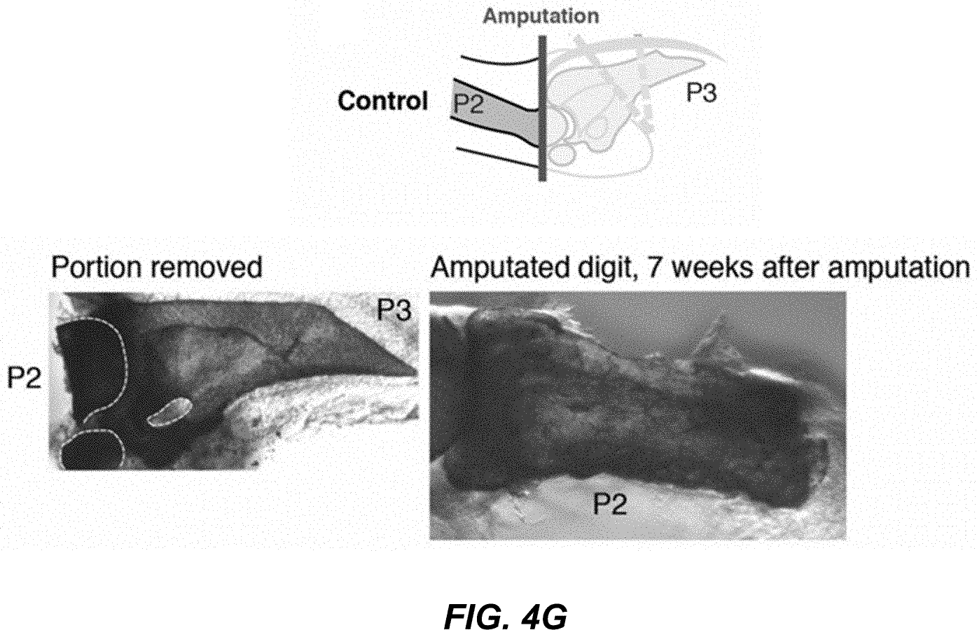

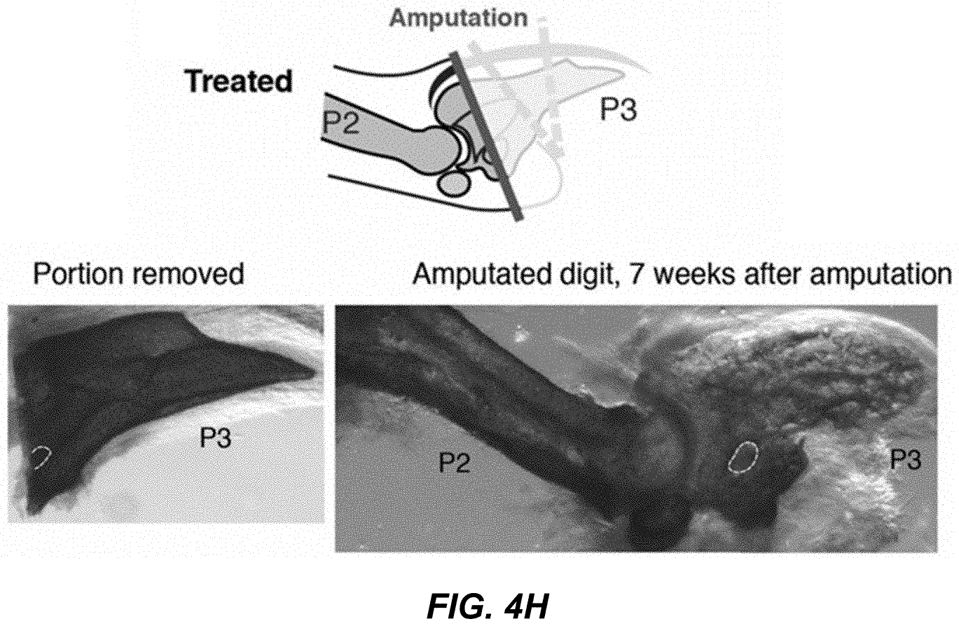

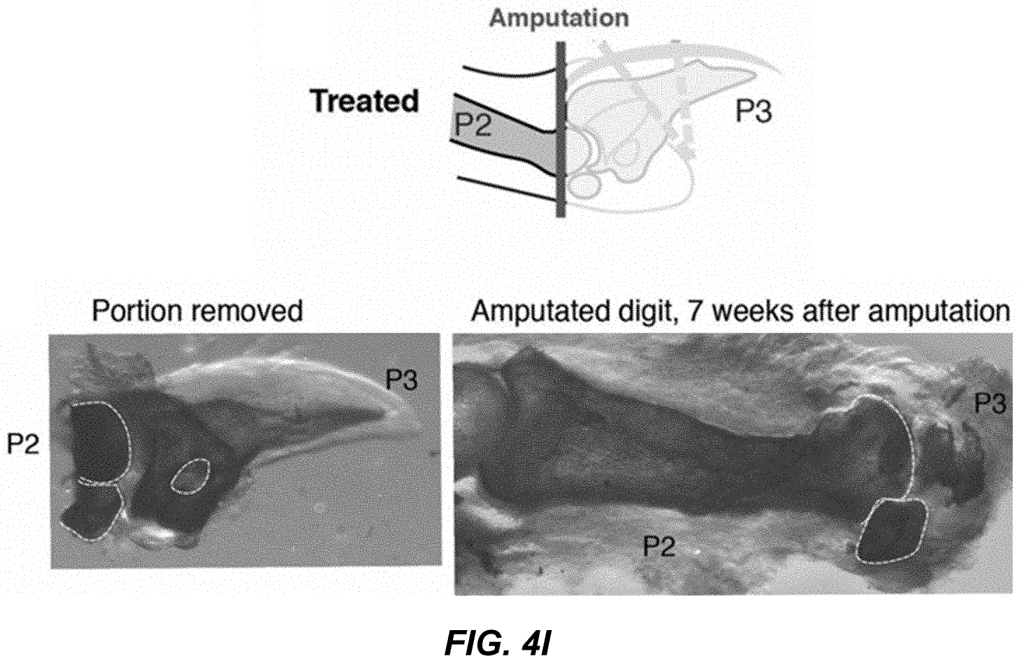

Digit amputation was performed on adult mice (3 and 6 months old). Amputation was performed on a hindlimb (FIG. 4A), on digit 2 and 4, with digit 3 as the internal control (FIG. 4B). To perform non-regenerating amputation, a clear morphological marker is the nail (FIG. 4B). The mouse digits, except for the thumbs, have three digital bones, or phalanges. Amputation that removes <30% length of the distal phalange (P3) regenerates, whereas amputation that removes >60% length of P3, i.e., almost the entire nail bed, does not regenerate (FIG. 4C). Accordingly, to ensure non-regenerative cut, amputation was performed entirely proximal to the visible nail (FIG. 4B-FIG. 4C)—giving, within the range of amputation precision, a cut across anywhere between the proximal end of P3 and the distal end of mid-phalange (P2) (triangle, FIG. 4C). Motivated by the findings in Aurelia and Drosophila, L-leucine and insulin were tested. Since insulin is proteolytically digested in mammalian gut, condition was used instead. L-leucine and sucrose were administered upon amputation, by mixing into the drinking water, refreshed weekly. The portion of the digit removed was immediately fixed for control. The amputated digits were monitored for 7-8 weeks to assess for regeneration, and subsequently dissected for skeletal staining.

As expected for amputation below the nail, no regeneration was observed (N=10 mice, 20 digits). Amputated digits simply heal and re-epithelialize the wound (FIG. 4D). Skeletal staining of the control digit stumps (FIG. 4G) shows blunted end as well as the expected distal bone erosion (in FIG. 4G, the remaining P2 is shorter than expected), with no overlap with original section removed. Bone erosion upon amputation has been well observed. By contrast, treated mice (N=20 mice, 40 digits) showed pervasive regenerative response with varying extent of bone regrowth. The most dramatic response was observed in 2 digits. In one digit from a 3-month old mouse (FIG. 4E), one digit shows almost complete regrowth and nail reformation (arrow in FIG. 4E, and inset); notice that the other digit in the same paw also shows some regrowth. Skeletal staining of the digit (FIG. 4H) shows that the digit was amputated at proximal P3 transecting the os hole, and regrew by 7 weeks an almost complete P3. The regrown P3 shows a woven, trabecular appearance that is similar in general structure but not identical to the original P3—reminiscent to the trabecular bone regenerated in digit tips. Another dramatic regeneration was observed in a digit from a 6-month old mouse (arrow in FIG. 4F), which shows the beginning of what appears to be a nail bed (inset in FIG. 4F). Skeletal staining (FIG. 4I) shows that the digit was amputated across the mid-phalange P2 removing the entire epiphyseal end, the ventral sesamoid process, and the P2/P3 joint. The digit regrew the sesamoid bone and reformed the knobby epiphyseal end of P2 articulating from which appears to be the beginning of the next segment (FIG. 4I). Self-organized regrowth in adult mice from as far back as mid-phalangeal injury is the most dramatic regenerative response reported so far in the mouse digit.

Altogether, these findings present L-leucine and insulin/sugar administration as a conserved strategy for inducing appendage regeneration across three wildly diverged species. Notably, despite the common inducers, morphogenesis proceeds in a species-specific mode: While regrowth of Drosophila limb and mouse digit proceed progressively, growing from the distal end, regrowth of Aurelia arm proceeds in a global manner; partial regeneration means a small, but recognizably whole, arm. Without being bound by any particular theory, the present strategy of inducing limb regeneration might be common with those that have been found to promote organ or tissue regeneration: hypoxia was shown to promote cardiomyocyte regeneration, whereas mTOR activation induces retinal axon regrowth. This study began with the presumption that inducing regeneration would require reconstituting detailed developmental mechanisms, or modulation of specific genes in a lineage-specific way—and predicted, if it is possible at all, having to administer complex combination of molecules at different times to effect regeneration. The surprise was the simplicity by which appendage regeneration, or at least a significant step of it, can be induced without having to reconstruct detailed patterning and differentiation processes, but by dietary supplementation of specific amino acids and sugar.

### Methods

Aurelia aurita

Aurelia culture. The Aurelia aurita sp. 1 strain, also alternatively named Aurelia coerulea based on recent molecular classification, come from polyps originally collected off the coast of Long Beach, Calif. (33°46′04.2″N 118°07′44.2″W, GPS: 33.7678376,-118.1289559). Polyps were reared at 68° F., in 32 ppt artificial seawater (Instant Ocean), and fed daily with brine shrimps (Artremia nauplii) enriched with Nannochloropsis algae. To induce strobilation, polyps were incubated in 25 μM 5-methoxy-2-methyl-indole (Sigma M15451) at 68° F. for an hour. Ephyrae typically began to strobilate within a week.

Amputation. Strobilated ephyrae were fed daily with rotifers (Brachionus plicatilis) until amputation time. 2-3 days old ephyrae were anesthetized in 400 μM menthol and amputated using a razor blade mounted on an x-acto knife handle. After amputation, ephyrae were let to recover in bubbler cones (FIG. 7). Regeneration was assessed at various times until 1-2 weeks after amputation, before onset of maturation to medusa.

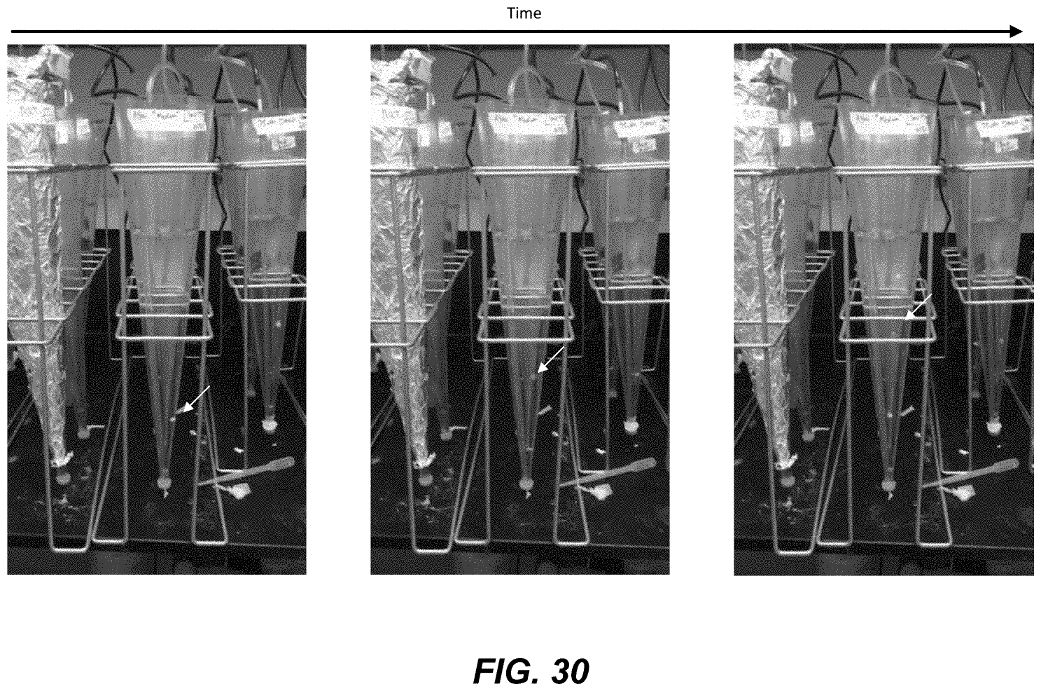

Inducing regeneration. Experimental setup. 0.5-1 L sand settling cones were repurposed as jellyfish aquaria (Nalgene Imhoff; FIG. 7). Each cone was equipped with an airline from a Tetra Whisper air pump 100 to create a gentle vertical current (˜1 bubble/sec, see FIG. 30). The conical geometry eliminates stagnant spots, where the ephyrae could get stuck. In this “bubbler cone” setup, the ephyrae were continually in water current either in the upward air bubble-generated current or the downward self-generated current. 500 mL artificial seawater and 30 ephyrae were placed in each cone to avoid crowding and fouling. Artificial seawater was changed every week. Nutrients. In some embodiments, the food amount given here only serves as an initial estimate; percentage of regeneration can easily vary with lab culture condition (e.g., polyp and rotifer size, age, health), as well as subject to variation across polyps and strobilation batches (see Table 2A-Table 2C for a sense of the variation). Amputated ephyrae were fed daily with rotifers. The number of rotifers administered daily was estimated using a 6-well plate fitted with STEMgrid™ (the same principle as using a hemocytometer). In some embodiments, low food is ˜100-200 rotifers/ephyra, whereas high food is ˜400 rotifers/ephyra. Insulin. Human recombinant insulin was administered to the amputated ephyrae at 500 nM immediately after amputation (by mixing in the artificial seawater). If the experiment proceeds longer than a week, fresh insulin was added at the one-week time point. Hypoxia. Nitrogen or argon flow, instead of ambient air flow, was introduced via the airline into the bubbler cone immediately after amputation, and maintained throughout the duration of the experiment. The nitrogen flow was adjusted to achieve 50% reduction in the dissolved oxygen level.

Experiment in the original habitat. Amputated ephyrae were let to recover in the waters off the coast of Long Beach, Calif. (33°46′04.2″N 118°07′44.2″W, GPS: 33.7678376,−118.1289559). For submersing the ephyrae in the ocean, a two-layered aquarium was built. Ephyrae were placed in plastic canisters with a 7 cm diameter hole cut in the lid, which was covered with a 250 μm plastic screen. The canisters were then placed in a thick plastic tank outfitted with a 500 μm plastic screen on top. This design offers protection to the ephyrae against predators and strong waves, while at the same time allowing exchange of water, zooplanktons, and particulates. Ephyrae were collected after two weeks.

Staining. All steps were performed at room temperature, unless indicated otherwise. Ephyrae were first anesthetized in 400 μM menthol, which minimizes curling during fixing. Next, ephyrae were fixed in 3.7% (v/v) formaldehyde (in PBS) for 15 minutes, permeabilized in 0.5% Triton X-100 (in PBS) for 5 minutes, and blocked in 3% (w/v) BSA for 2 minutes. For neuron staining, ephyrae were incubated in 1:200 mouse anti-tyrosinated alpha tubulin antibody (Sigma MAB18644) overnight at 4° C., and then in 1:200 goat-anti-mouse Alexa Flour 488 (Sigma A11029) overnight in the dark at 4° C. Primary or secondary antibodies were diluted in 3% BSA. For actin staining, ephyrae were incubated in 1:20 Alexa Fluor 488 Phalloidin (Life Technologies A12379) overnight or for 2 hours in the dark at 4° C. For nuclei staining, ephyrae were incubated in 1:10 Hoechst 33342 (Sigma B2261) for 30 minutes in the dark.

Edu assays. Proliferating cells were stained using Click EdU Alexa Fluor 594 (Life Technologies C10339) according to the supplier's protocol with modifications: Ephyrae were incubated in 1:1000 EdU (in artificial seawater) for 24 hours in the dark, rotating in an HAG rotisserie rotator (FinePCR) at 7-10 rpm. Prior to fixing, ephyrae were extensively washed in artificial seawater for 1 hour, fixed in 3.7% (v/v) formaldehyde (in PBS) for 15 minutes, and blocked with 3% (w/v) BSA diluted in 0.5% Triton X-100 (in PBS) for 20 minutes. Ephyrae were then incubated in the Click-iT reaction mixture for 30 minutes in the dark.

Microscopy. Ephyrae were imaged anesthetized in menthol. Brightfield images, fluorescent images, and movies were taken with the Zeiss AxioZoom.V16 stereo zoom microscope and AxioCam HR 13-megapixel camera. Optical sectioning was performed with ApoTome.2.

RNA sequencing. All code and relevant input/intermediary files are available on GitHub (https://github.com/DavidGoldLab/2020_Regeneration_Induction).

RNA extraction was performed using a modified TRIzol (Invitrogen) protocol. Animals were sampled prior to amputation (“uninjured control”) and 27 hours post-amputation with daily feeding (“fed”) or no feeding (“non-fed”). Each condition was collected in duplicate, and three ephyrae were collected for each RNA extraction (six ephyra total per condition). The animals were lysed (in 200 μl/tube of 100 mM Tris-HCL pH 5.5, 10 mM EDTA pH 8, 0.1 M NaCl, 1% SDS, 1% β-mercaptoethanol), homogenized with a motorized mortar and pestle, and treated for 10 minutes at 55° C. with Proteinase K (2.5 μl/tube of 20 mg/mL stock). 1mL of TRIzol was added to each sample, and RNA isolation was performed using the Invitrogen protocol. Extracted RNA was sent to the Millard and Muriel Jacobs Genetics and Genomics Laboratory at Caltech. The quality of total RNA was checked on an Agilent 2100 Bioanalyzer, and cDNA libraries were constructed using the Illumina TruSeq RNA Library Prep Kit v2. 100 basepair single-end RNA-Seq reads were produced on an Illumina Genome Analyzer lix sequencer.

Analysis of differentially expressed genes. RNA-seq reads were mapped to the recently published Aurelia genome using HISAT2. The mapped reads were inputted to the Cufflinks package to assemble an updated set of gene models. This pipeline resulted in a gene count table for all samples (the “genes.count_table” file in GitHub). This table was inputted to EdgeR to analyze for differential gene expression (the R scripts used in EdgeR are provided on GitHub). A total of 5,305 genes were differentially expressed based on stringent cutoffs (p-value <0.001; log-fold change >4).

Gene and protein model annotation were performed using the Trinity software package. Gene assignments were performed using BLAST 2.9.0+: BLASTp for Aurelia protein queries and BLASTx for Aurelia nucleotide queries against the Uniprot SwissProt database of reference proteins. Conserved protein domains were identified using HMMER v.3.3 and Pfam-A database. The results from these analyses were loaded into an SQL database using Trinotate; gene ontology (GO) and Kyoto Encyclopedia of Genes and Genomes (KEGG) terms were populated into the database based on BLASTp results. An additional round of KEGG annotation was performed by submitting the protein models to the KAAS-KEGG Automatic Annotation Server using bi-directional best hit method and standard settings. The KEGG IDs produced by Trinotate and the KAAS-KEGG server were used to analyze the gene presence/absence in FIG. 9A-FIG. 9E. Results of the annotation are provided on GitHub as “Trinotate.Report.txt”.

Aurelia mTOR clustering analysis. The BLAST and PFAM annotation identified the gene “XLOC_029150” as the Aurelia mTOR ortholog. Clustering for gene with similar expression profile was performed using the “analyze_diff_expr.pl” Perl script in Trinity. The gene counts were normalized into log2 fpkm values (Fragments Per Kilobase of transcript per Million mapped reads) with the row mean subtracted from each data point (i.e., centered). Using these centered counts, the genes were clustered into a hierarchical tree using Euclidean distance (FIG. 10A). The tree was divided into subclusters using the “define_clusters_by_cutting_tree.pl” Perl script in Trinity. As there is no “correct” cutoff value for defining genes with similar expression profiles, a range of cutoff values were tested (FIG. 10C). Raising the cutoff value increased the number of genes recovered but decreased the similarity between their averaged expression profile and the expression profile of Aurelia mTOR. A 10% cutoff value was chosen for further analysis as a balance between gene numbers and similarity of expression profiles. The list of genes at 10% cutoff is provided on GitHub (“GoSeq_List.P10.subcluster_7.txt”). To probe the processes enriched in this cluster, the “run_Goseq.pl” Perl script in Trinity was used, using all differentially expressed genes as background dataset. To visualize the enriched GO terms (FIG. 10D), the REVIGO algorithm was used.

Inhibiting or activating mTOR. 1 nM Sapanisertib (Selleck Chemicals S2811), 1 μM A769662 (Santa Cruz sc-203790), or 100 μM L-leucine (Sigma L1002, the cell-permeable methyl ester hydrochloride form) was administered into the artificial seawater right after amputation, and refreshed weekly. Statistical analysis. Percent regeneration was first normalized to the control (for sapanisertib and A769662, the HF+DMSO control; for leucine data the LF control), and then the student's t-test (two tailed, equal variances) in Excel was used to test the null hypothesis that the fold change in % regeneration is zero. The experimental replicates and p-values are tabulated in Table 2A-Table 2C. Drosophila melanogaster

Drosophila culture. Oregon R and Canton S Drosophila strains were reared under standard conditions at 23° C. MHC-tauGFP was obtained from the Bloomington Drosophila Stock Centre (BDSC_38462).

Amputation. Adult flies 2-7 days after eclosion were anesthetized with CO2. Amputations were performed under a dissection microscope using a spring scissors (Fine Science Tools, 91500-09) and superfine dissecting forceps (VWR, 82027-402). Recovering Drosophila were fed with standard fly food (control) or standard fly food supplemented with 2 mM L-leucine (Sigma L8000), 2 mM L-Glutamine (Sigma G3126), and 0.1 mg/mL insulin (MP Biomedicals 0219390080) or other molecular factors. To introduce molecular factors into fly food, standard fly food was microwaved in short pulses, such that the topmost layer of the food was liquified. Molecular factors in aqueous medium were then pipetted into this liquified layer. Food was allowed to re-set at 4° C. for at least 20 minutes. New food was prepared fresh every 2 days throughout the course of the 2-week experiment. Regeneration was assessed at 3, 6, 9 and 14 days post amputation.

Staining. Fly tibias were dissected and washed in 70% ethanol to decrease the hydrophobicity of the cuticle (<1min) and washed in PBS with 0.3% Triton-X for 10 minutes. The legs were fixed in 4% paraformaldehyde (in PBS) overnight at 4° C. and washed five times for 20 minutes each in PBS with 0.3% Triton-X. The legs were equilibrated in Vectashield mounting medium with DAPI (Vector H-1200) overnight at 4° C., and imaged using Zeiss AxioZoom.V16 stereo zoom microscope and AxioCam HR 13-megapixel camera.

Live fly imaging. Flies anesthetized on a CO2 bed were imaged under dissection scope equipped with the Zeiss AxioCam 503 color camera.

Electron microscopy. Environmental scanning electron microscopy (ESEM) was performed on a FEI Quanta 200F (FEI, Hillsboro, Oregon). Whole live flies were mounted onto the SEM stub with copper tape. ESEM images were attained at a pressure of 0.1 mbar and 5 kV at a working distance of 9-12 mm, with water as the ionizing gas.

Mus musculus

Mouse strains. Adult female (12 weeks or older) WT CD1 mice (Charles River Laboratories strain 022) were used for all regeneration studies. Digits were harvested 8 weeks post amputation.

Mouse digit amputation. Digit amputations were carried out on 3-6-month-old mice. Mice were anesthetized with 1-5% isoflurane in oxygen in an induction chamber, followed by maintenance on a nosecone. The mouse was positioned on its belly with its hind paws outstretched and the ventral side of the paw facing upwards. Sustained-Release Buprenorphine was administered (Buprenorphine SR LAB®) at 0.5 mg/kg subcutaneously as an analgesic. Blood flow to the hindlimb was stemmed by tying a rubber band around the ankle and clamping it with a hemostat. All surgical procedures were carried out under a Zeiss Stemi 305 dissection microscope. An initial incision, parallel to the position of foot, was made through the ventral fat pad using Vannas spring scissors (World Precision Instruments, 14003). The length of this incision was determined by the amount of ventral skin needed to seal the digit amputation wound completely. The ventral skin freed in the initial incision was peeled back using surgical forceps, and a no. 10 scalpel (Sklar, 06-3110) was used to amputate and bisect the digit completely through the second or third phalange. Digits 2 and 4 on the right hind paw were operated on in this fashion, while digit 3 remained unamputated as a control. The amputation wound was immediately closed with the ventral skin flap and sealed with GLUture (Zoetis, Kalamazoo, Mich.). All studies comply with relevant ethical regulations for animal testing and research, and received ethical approval by the Institutional Animal Care and Use Committees at the California Institute of Technology.

Mouse digit dissection and skeletal staining. Mice were euthanized and digits 2, 3 and 4 were removed with a no. 10 scalpel (Sklar, 06-3110) through the first phalange. Excess skin and flesh were removed with spring scissors (Fine Science Tools, 91500-09) and fine dissecting forceps (Fine Science Tools, 11254-20). All digits analyzed by whole mount skeletal stains were prepared with a standard alizarin red and alcian blue staining protocol. Digits were dehydrated in 95% ethanol for 1d, and incubated 1d at 37 ° C. in staining solution (0.005% alizarin red, 0.015% alcian blue, 5% acetic acid, 60% ethanol). Tissue was cleared in 2% potassium hydroxide at room temperature for 1d, 1% potassium hydroxide for 1d, and then taken through an increasing glycerol series (25%, 50%, 75%, 100%) and imaged on a Zeiss AxioCam 503 color camera or a Zeiss Stemi 305 dissection microscope with an iPhone 6 camera.

### Example 2

A conserved strategy for inducing appendage regeneration

Can limb regeneration be induced? Few have pursued this question, and an evolutionarily conserved strategy has yet to emerge. This study reports a strategy for inducing regenerative response in appendages, which works across three species that span the animal phylogeny. In Cnidaria, the frequency of appendage regeneration in the moon jellyfish Aurelia was increased by feeding with the amino acid L-leucine and the growth hormone insulin. In insects, the same strategy induced tibia regeneration in adult Drosophila. Finally, in mammals, L-leucine and sucrose administration induced digit regeneration in adult mice, including dramatically from mid-phalangeal amputation. The conserved effect of L-leucine and insulin/sugar suggests a key role for energetic parameters in regeneration induction. The simplicity by which nutrient supplementation can induce appendage regeneration provides a testable hypothesis across animals.

### Leucine and Insulin Promote Appendage Regeneration in the Moon Jelly Aurelia

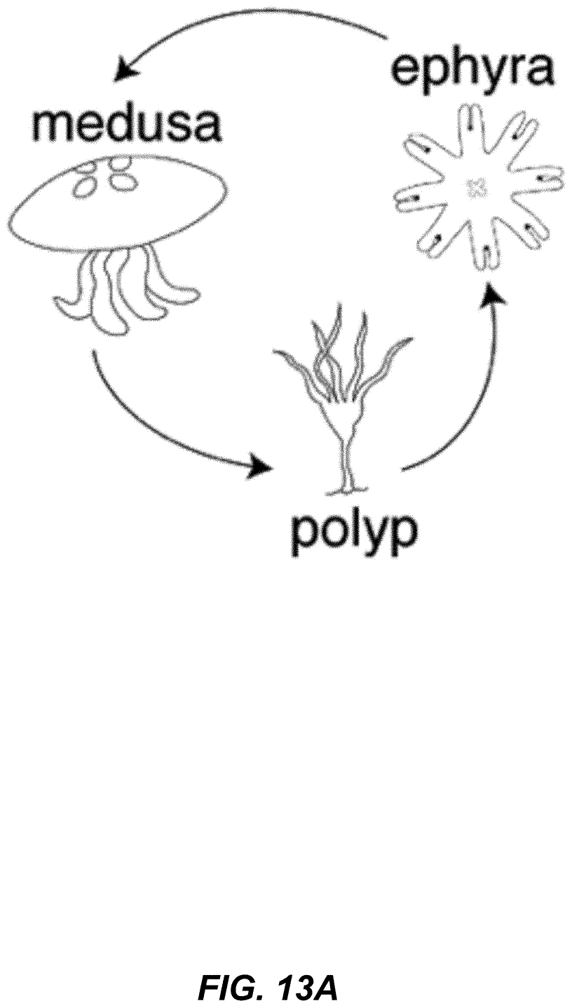

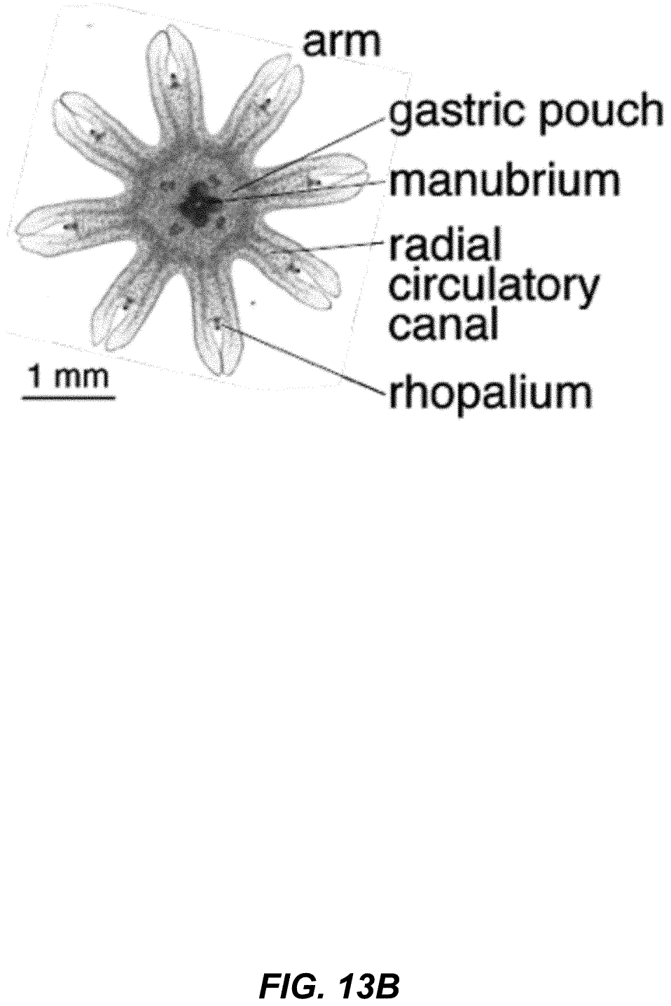

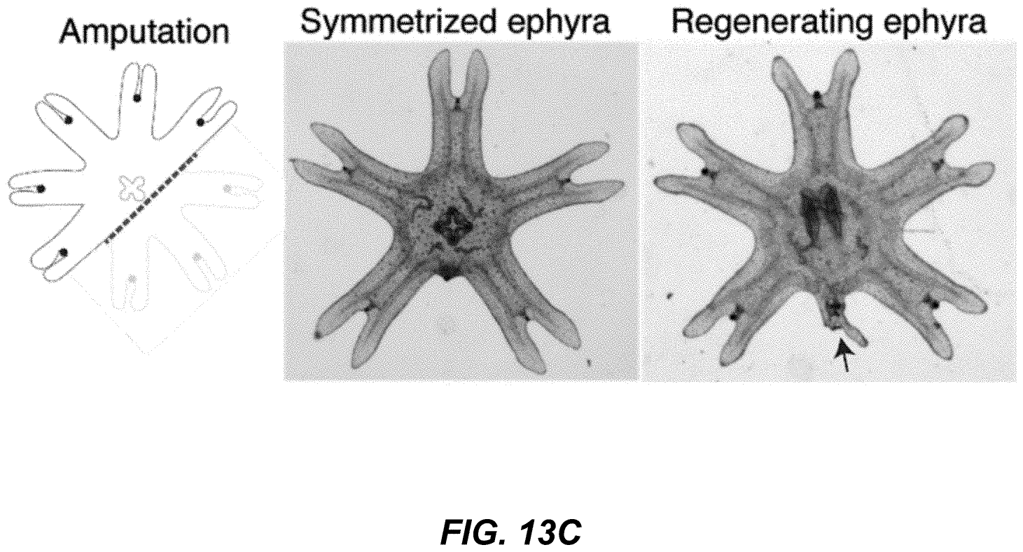

It was reasoned that if there was an ancestral mechanism to promote regeneration, it would more likely be intact in early-branching lineages. In Cnidaria, the ability to regenerate is established in polyps, e.g., hydras and sea anemones. Some cnidarians, notably jellyfish, not only exist as sessile polyps, but also as free-swimming ephyrae and medusae (FIG. 13A). In contrast to the polyps' ability to regenerate, regeneration in ephyrae and medusae appears more restricted. The moon jellyfish Aurelia coerulea (formally A. aurita sp. 1 strain) was focused on, specifically on the ephyra, whose eight arms facilitate morphological tracking (FIG. 13B). Aurelia ephyrae regenerate tips of arms and the distal sensory organ rhopalium, but upon more dramatic amputations such as removing a whole arm or halving the body, rapidly reorganize existing body parts and regain radial symmetry (FIG. 13C). Observed across four scyphozoan species, symmetrization occurs rapidly within 1-3 days and robustly across conditions. Ephyrae that symmetrized matured into medusae, whereas ephyrae that failed to symmetrize and simply healed the wound grew abnormally.

Intriguingly, in a few symmetrizing ephyrae, a small bud would appear at the amputation site. To follow this hunch, the experiment was repeated in the original habitat of the present polyp population, off the coast of Long Beach, Calif. (see Methods below). Two weeks after amputation, most ephyrae indeed symmetrized, but in 2 of 18 animals a small arm grew (FIG. 13C). This observation suggests that, despite symmetrization being the more robust response to injury, an inherent ability to regenerate arm is present and can be naturally manifest. The inherent arm regeneration presents an opportunity: Can arm regeneration be reproduced in the lab, as a way to identify factors that promote regenerative state?

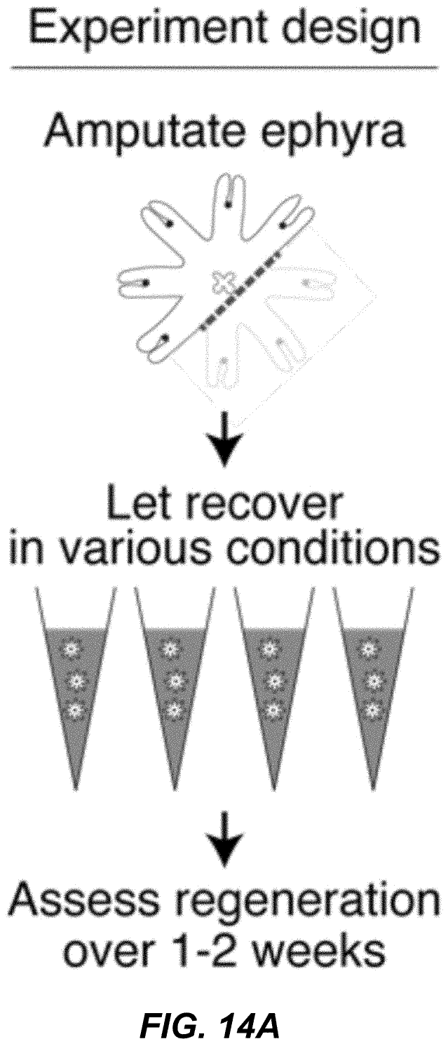

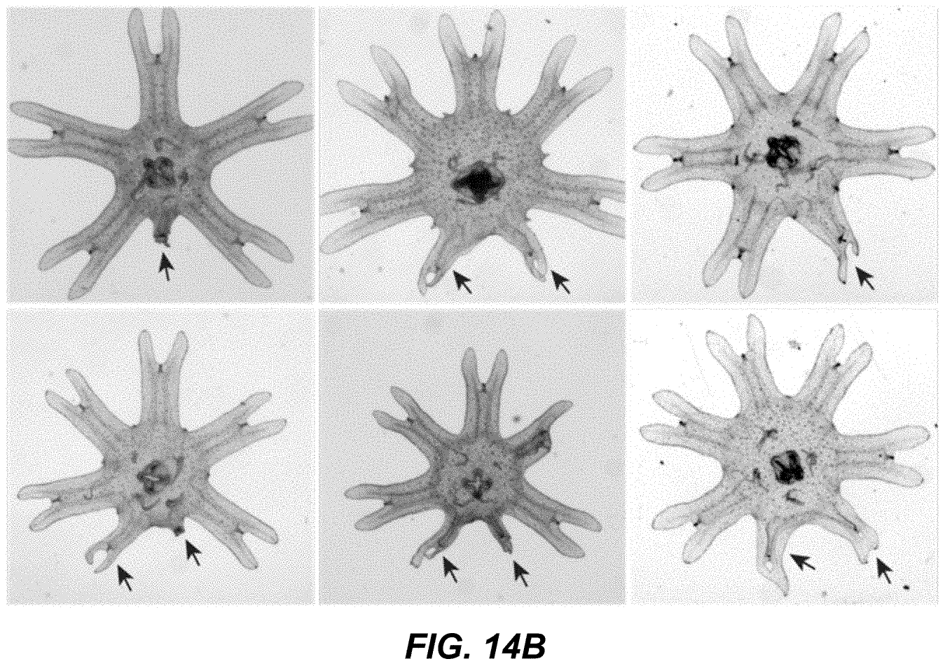

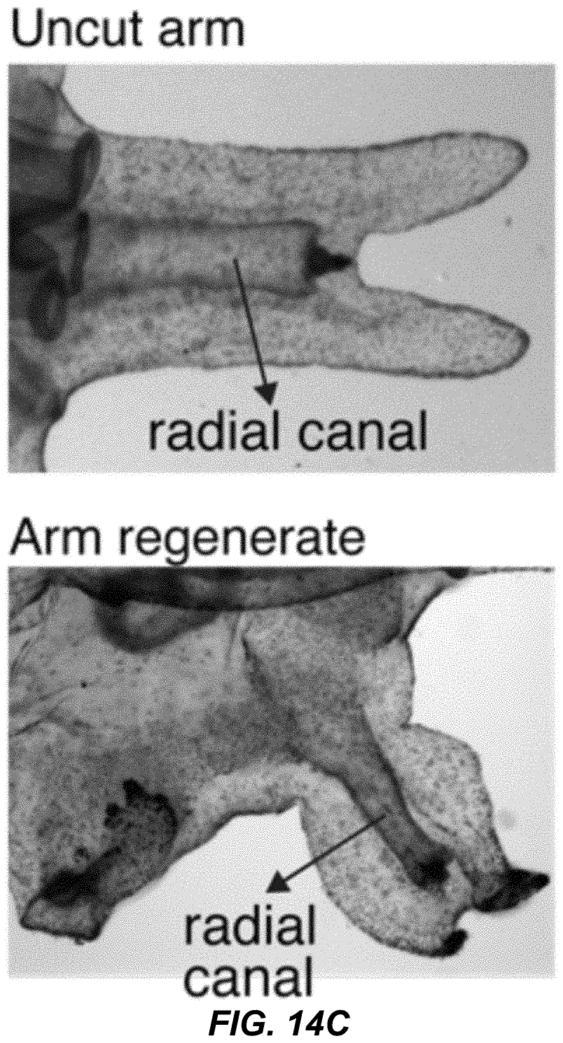

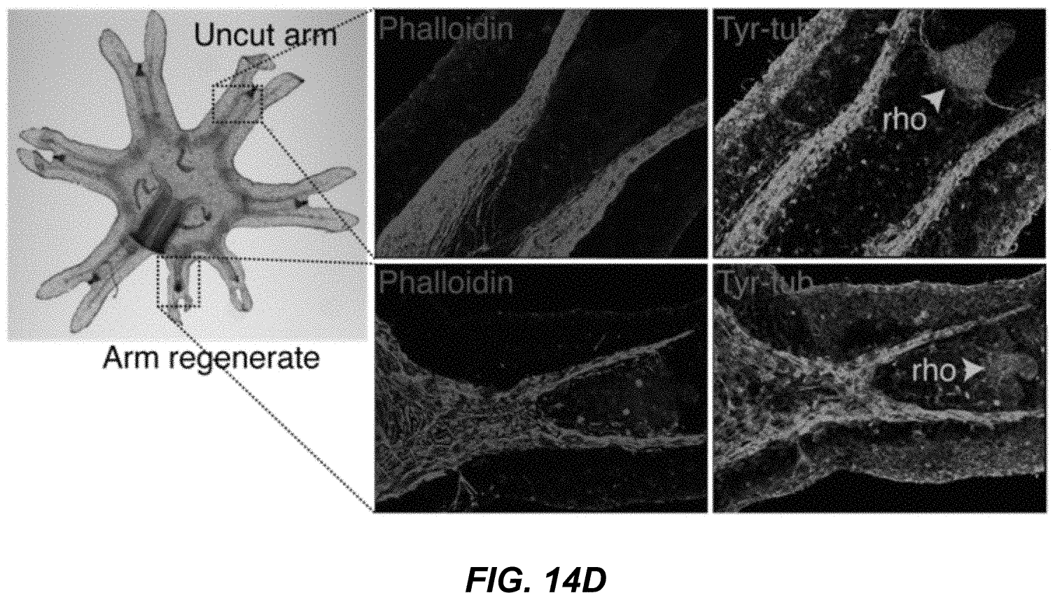

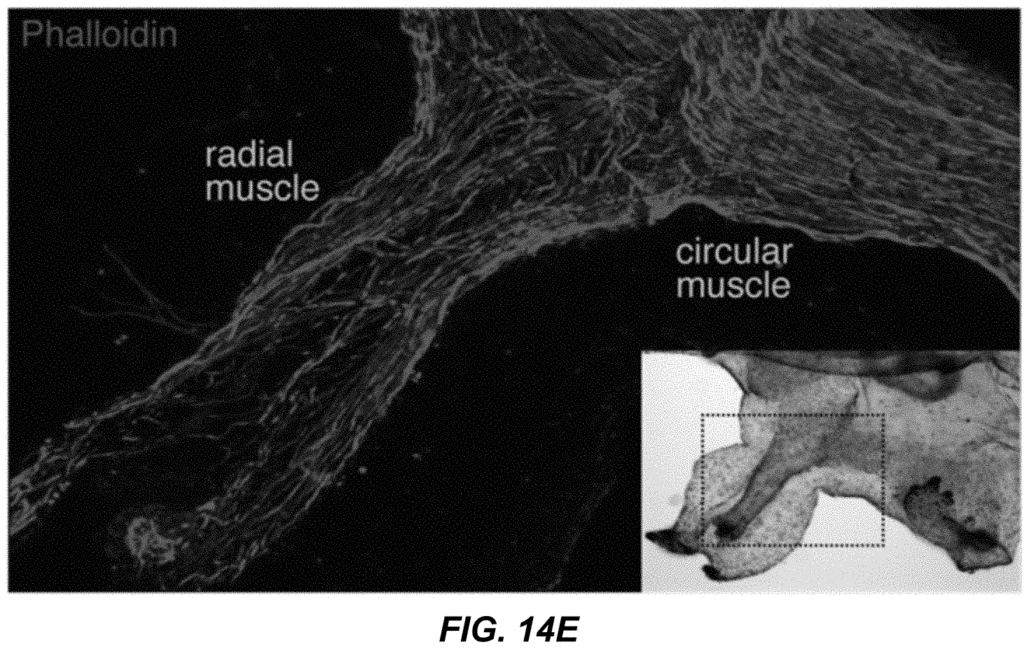

To answer this question, various molecular and physical factors were screened (FIG. 14A, Table 5A-Table 5B). Molecularly, modulators of developmental signaling pathways were tested as well as physiological pathways such as metabolism, stress response, immune and inflammatory response.

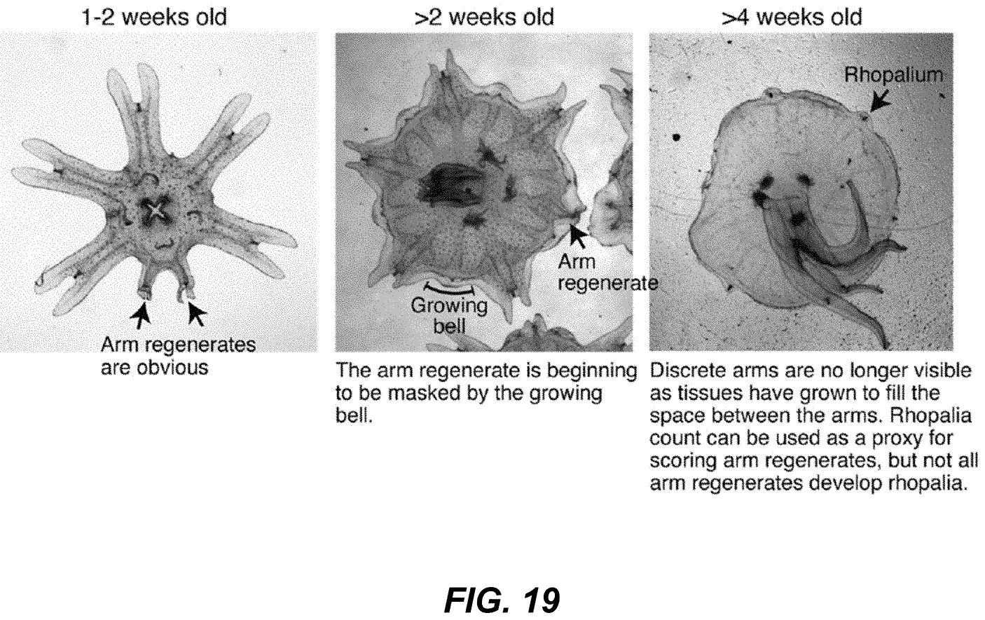

Physically, environmental parameters were explored, such as temperature, oxygen level, and water current. Amputation was performed across the central body removing 3 arms (FIG. 14A). Parameter changes were implemented or molecular modulators (e.g., peptides, small molecules) were introduced into the water immediately after amputation. Regenerative response was assessed for 1-2 weeks until the onset of bell growth, which hindered the scoring of arm regeneration (FIG. 19).

After 3 years of screening, only three factors emerged that strongly induced arm regeneration (FIG. 14B). The ephyrae persistently symmetrized in the majority of conditions tested. In the few conditions where regeneration occurred, arm regenerates show multiple tissues regrown in the right locations: circulatory canals, muscle, neurons, and rhopalium (FIG. 14C-FIG. 14E). The arm regenerates contracted synchronously with the original arms (FIG. 29), demonstrating a functional neuromuscular network. Thus, arm regeneration in Aurelia that was observed in the natural habitat can be recapitulated in the lab by administering specific exogenous factors.

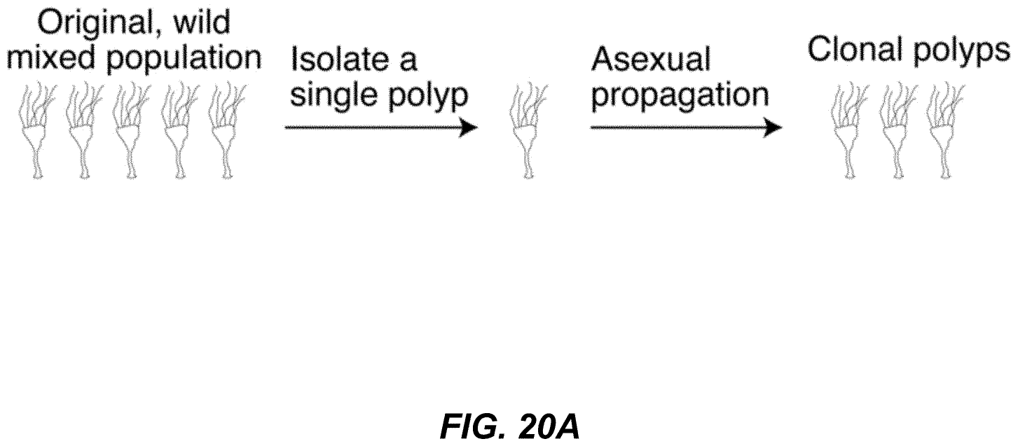

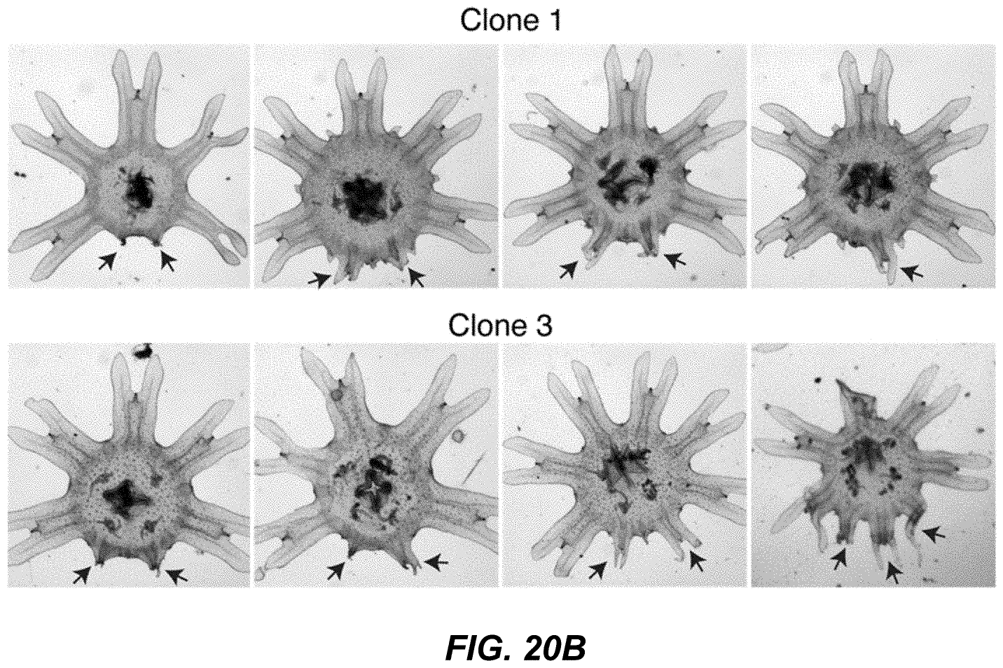

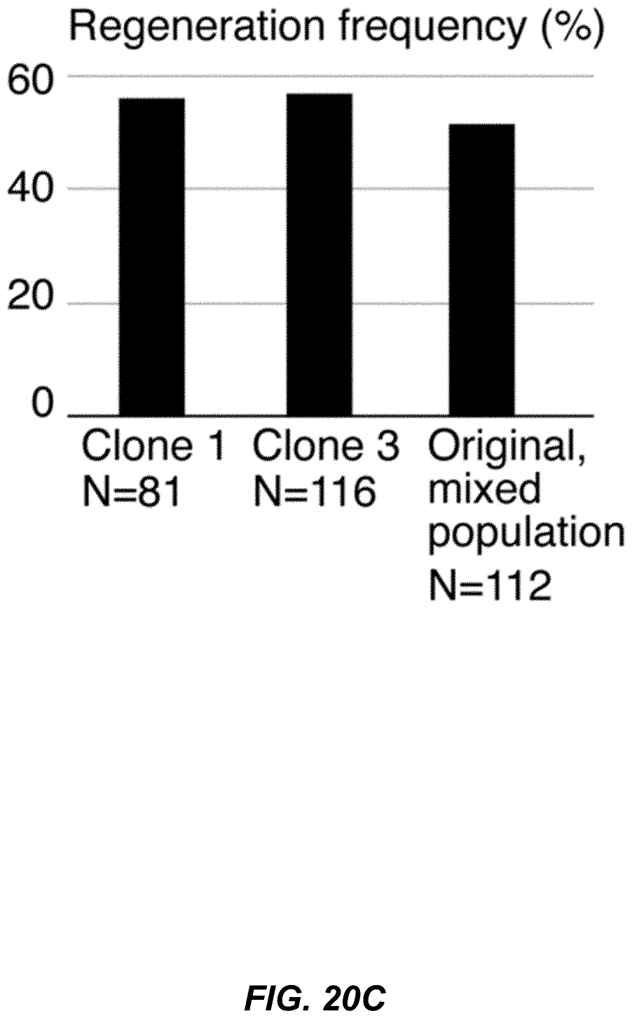

The extent of arm regeneration varied, from small to almost fully sized arms (FIG. 14B). The variation manifested even within individuals: a single ephyra could grow differently sized arms. Of the three arms removed, if regeneration occurred, generally one arm regenerated (67%), occasionally 2 arms (32%), and rarely 3 arms (1%, of the 4270 total ephyrae quantified in this study). Finally, the frequency of regeneration varied across clutches, i.e., strobilation cohorts. Some variability may be due to technical factors, e.g., varying feed culture conditions; however, variability persisted even with the same feed batch. It was verified that the variability was not entirely due to genetic differences, as it manifested across clonal populations (FIG. 20A-FIG. 20C). Thus, there appears to be stochasticity in the occurrence of arm regeneration in Aurelia and the extent to which regeneration proceeds.

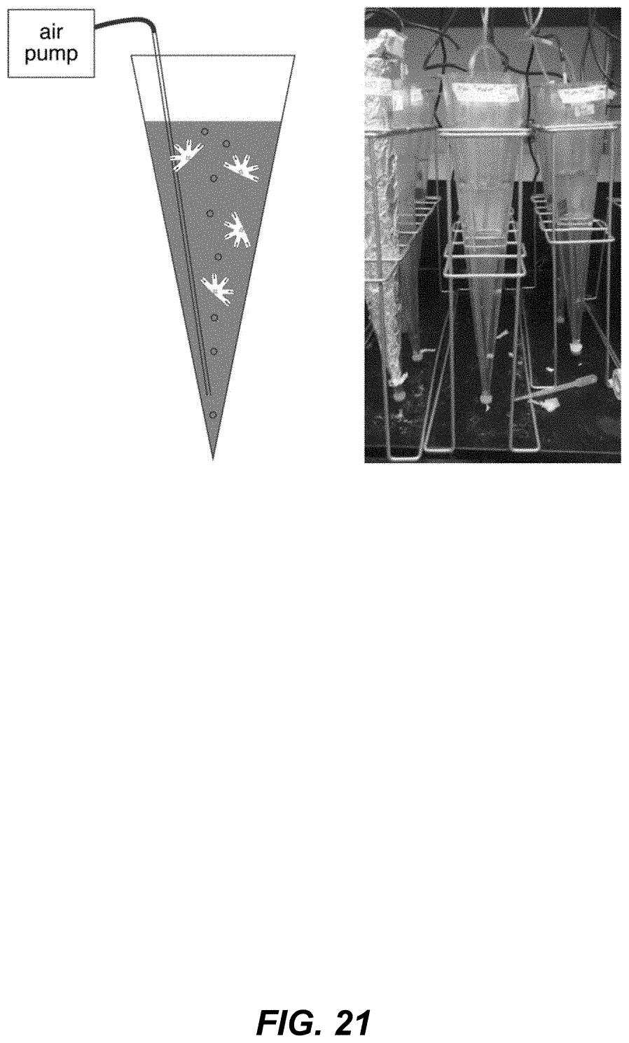

What are the factors that promote arm regeneration? Notably, modulation of developmental pathways often implicated in regeneration literature (e.g., Wnt, Bmp, Tgfβ) did not produce effect in the screen (Table 5A-Table 5B), although their involvement cannot be fully ruled out. A necessary condition was identified first: water current. Behaviorally, this condition promotes swimming, while in stagnant water ephyrae tend to rest at the bottom and pulse stationarily (FIG. 21 and FIG. 30 show the aquarium setup used to implement current). The requirement for water current is interesting because ephyrae and medusae in the lab are fine for several weeks in aquaria setups with no or less current. Thus, the requirement for constant activity may hint that a specific baseline physiological state is more permissive for regeneration.

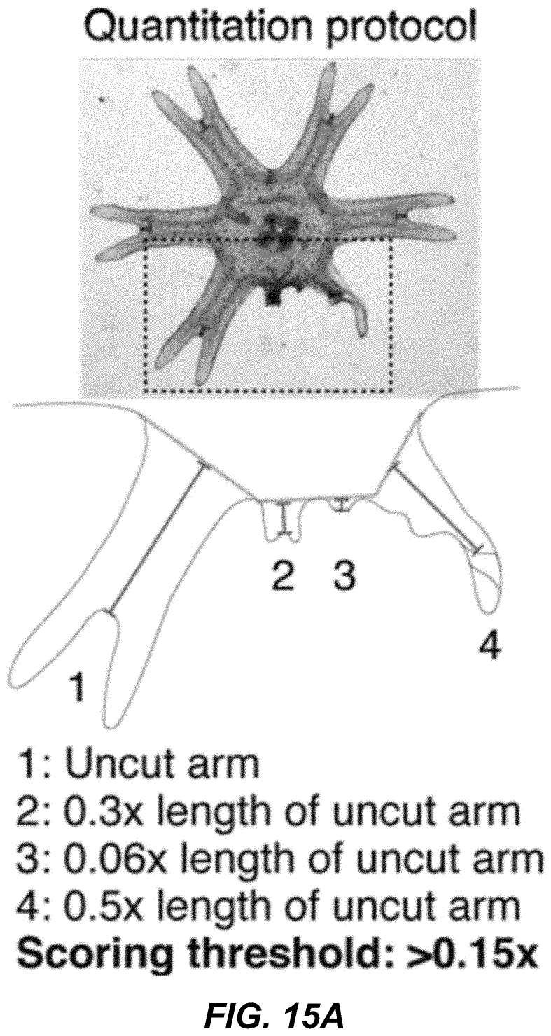

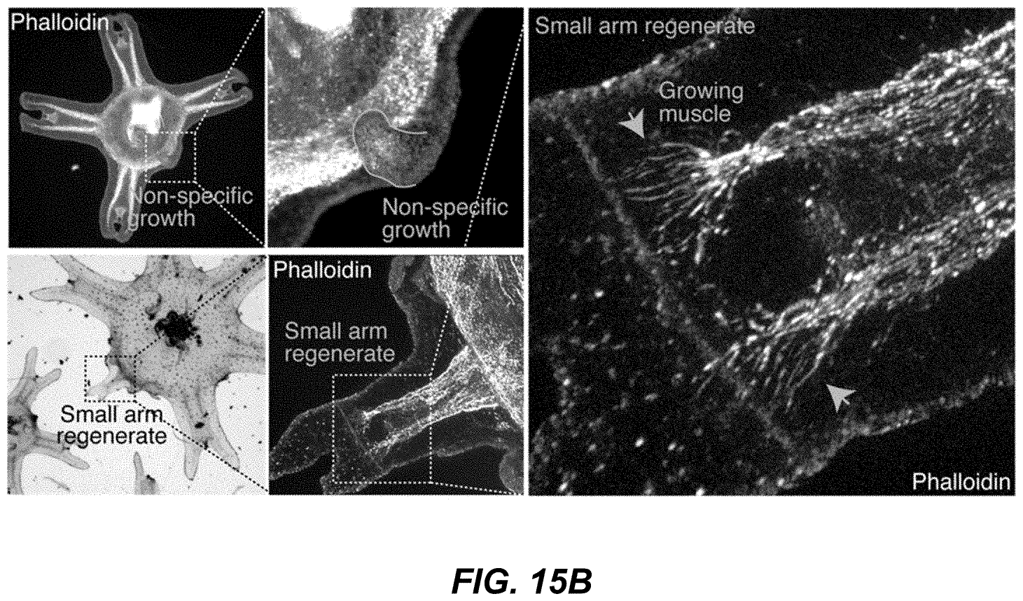

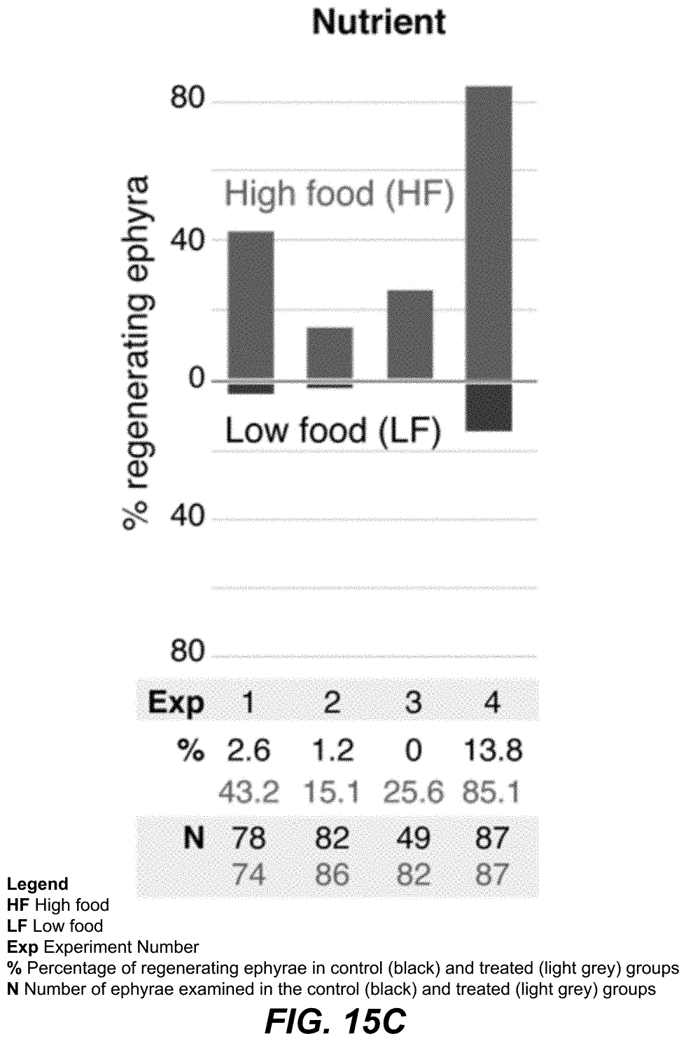

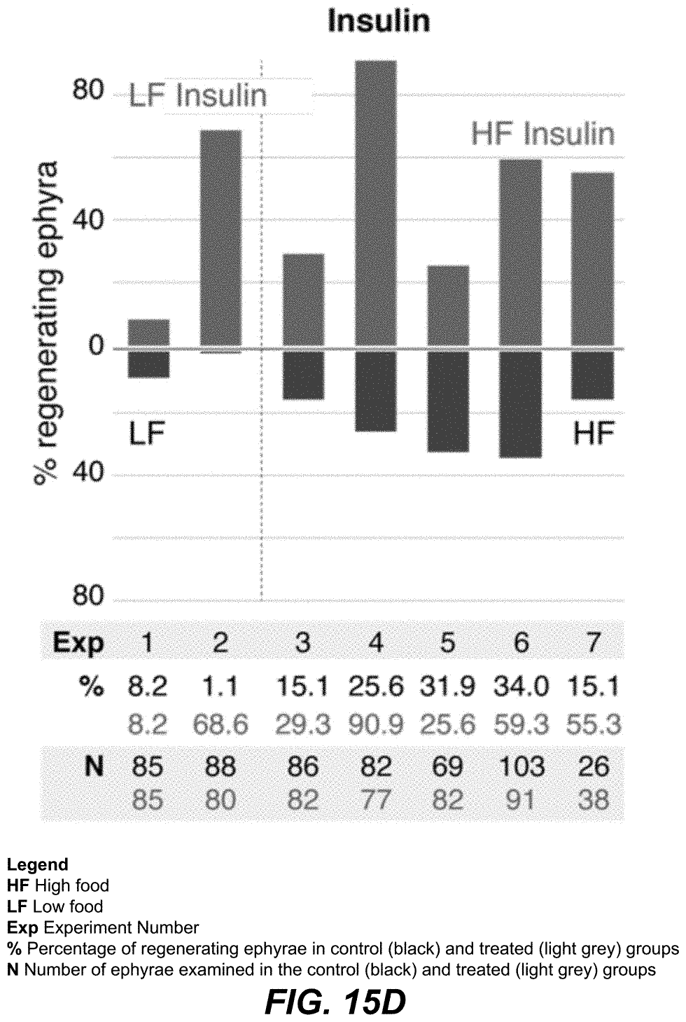

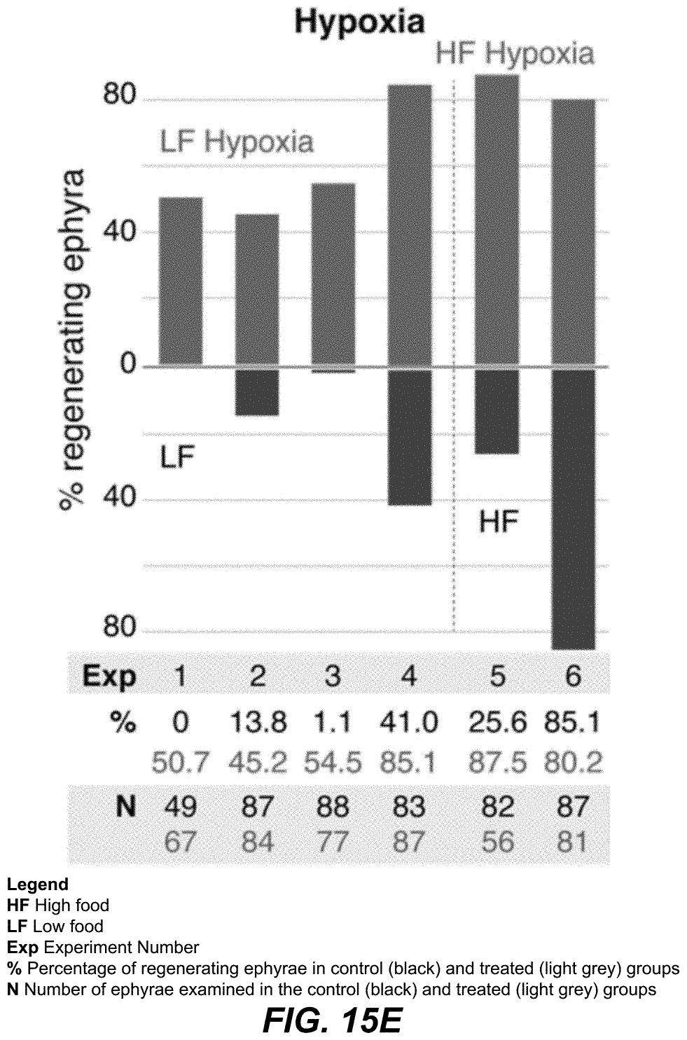

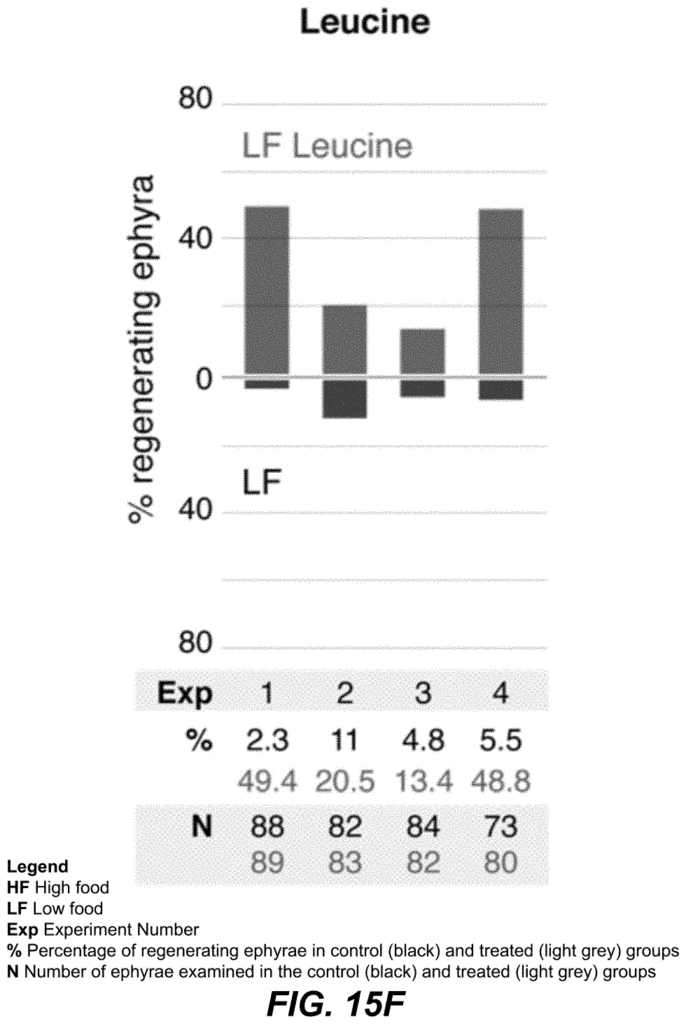

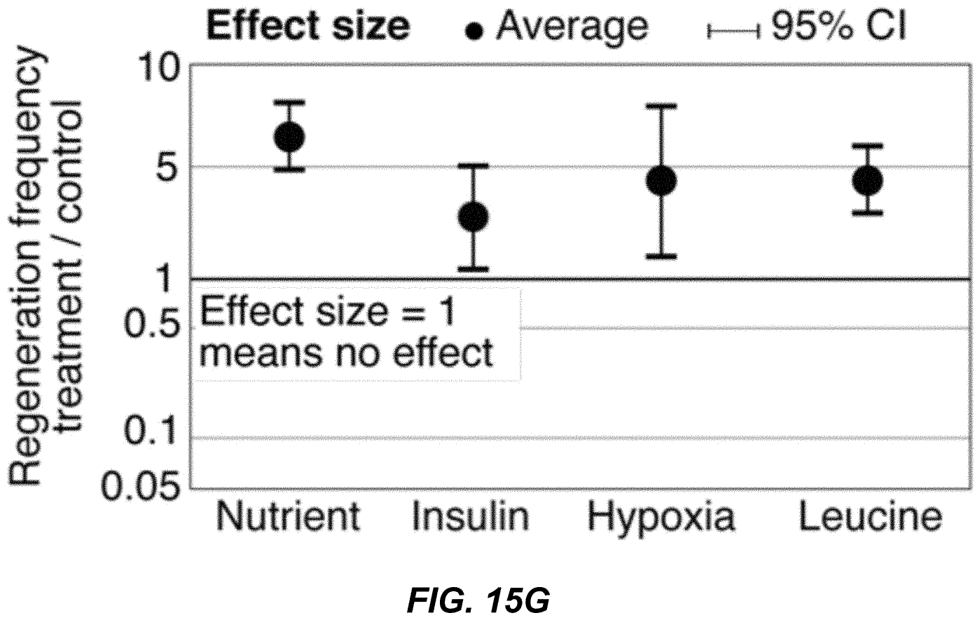

In this permissive condition, the first factor that induced regeneration is the nutrient level: increasing food amount increases the frequency of arm regeneration. To measure the regeneration frequency, any regenerates with lengths greater than 15% of that of an uncut arm were scored (FIG. 15A). This threshold was chosen to predominantly exclude non-specific growths or buds that show no morphological structures (FIG. 15B) while including small arm regenerates that show clear morphological features, i.e., lappets, radial canal, and radial muscle sometimes showing growing ends (FIG. 15B). Given the clutch-to-clutch variability, control and treatment were always performed side by side using ephyrae from the same clutch. The effect size of a treatment was assessed by computing the change in regeneration frequency relative to the internal control. Statistical significance of a treatment was assessed by evaluating the reproducibility of its effect size across independent experiments (see Methods below). With this measurement and statistical methodologies, it was found that although the baseline regeneration frequency varied across clutches, higher food amounts reproducibly increased regeneration frequency (FIG. 15C). The magnitude of the increase varied (FIG. 15G and Table 4, 95% CI [4.7, 12.1-fold]), but the increase was reproducible (95% CI excludes 1) and statistically significant (p-value<10⁻⁴).

TABLE 4

| Treatment | Average effect size | 95% CI | p-value |
|---|---|---|---|
| Nutrient | 7.5 | 4.7, 12.1 | <0.0001 *** |
| Insulin | 2.4 | 1.1, 5.0 | 0.023 * |
| Hypoxia | 4.1 | 1.4, 12.0 | 0.0099 ** |
| Leucine | 4.1 | 2.5, 6.6 | <0.0001 *** |

TABLE 5A
VARIOUS MOLECULAR AND PHYSICAL MODULATIONS WERE
SCREENED TO RECAPITULATE ARM REGENERATION.

| | Highest | | |
|---|---|---|---|
| Factor | dose | | Source |
| Modulators of signaling pathways | | | |
| Erbstatin | 5 | μM | Sigma D2667 |
| hEGF recombinant | 20 | ng/mL | Sigma E9644 |
| UO126 | 1 | μM | Millipore 6625 |
| Dorsomorphin | 1 | μM | Sigma P5499 |
| LiCl | 250 | mM | Sigma L4408 |
| CHIR99021 | 12.5 | μM | Sigma SML1046 |
| IWR-1 | 10 | μM | Sigma I0161 |
| XAV939 | 2 | μM | Sigma X3004 |
| Purmorphamine | 2 | μM | Sigma SML0868 |
| hTGF-β1 | 1.2 | ng/mL | Peprotech 100-21 |
| Modulations of metabolism, | | | |
| immune system, stress response | | | |
| Diosmetin | 10 | μM | Sigma D7321 |
| 17-DMAG | 1 | μM | TSZ Chemicals |
| | | | R1028 |
| Geranylgeranylacetone | 1 | μM | Sigma G5408 |
| KNK437 | 4 | nM | Sigma SML0964 |
| MKT-077 | 2.5 | μM | Sigma M5449 |
| Bromopyruvic acid | 125 | nM | Sigma 16490 |
| 6-Phosphogluconic acid | 20 | μM | Sigma P7877 |
| Antamycin A | 650 | nM | Sigma A8674 |
| 3PO | 10 | μM | Millipore 525330 |
| ATP | 5 | μM | Sigma A3377 |
| 3BDO | 3 | μM | Sigma SML1687 |
| D-Fructose 1.6-bisphosphate | 20 | μM | Sigma F6803 |
| DMOG | 50 | μM | Millipore 400091 |
| Rapamycin | 1 | μM | Sigma R8781 |
| L-Leucine methyl esther | 100 | μM | Sigma L 1002 |
| hydrochloride (cell permeable | | | |
| form) | | | |
| Resveratrol | 5 | μM | Sigma R5010 |
| Sapanisertib | 2 | nM | Selleck Chemicals |
| | | | S2811 |
| MHY1485 | 2 | μM | Sigma SML0810 |
| Insulin, human | 3 | μM | Sigma I0908 |
| AICAR | 25 | μM | Santa Cruz |
| | | | sc-200659A |
| A769662 | 5 | μM | Santa Cruz |
| | | | sc-203790 |
| D-Eryhtrose 4-phosphate | 20 | μM | Sigma E0377 |
| CoCl | 450 | nM | Sigma 60818 |
| Miscellaneous | | | |
| BSA | 500 | nM | Sigma A7906 |
| Ethanol | 20 | μL/L | VWR 89125-170 |
| CsCl | 5 | μL/L | Sigma C4036 |

Modulators were administered or physical parameters were implemented upon amputation. Some factors were dissolved in DMSO or ethanol; for these molecules, the control group was administered with an equal volume of the solvent.
Since few, if at all, of the molecular modulators had been tested in Aurelia, the maximum concentrations were tested to maximize the chance of seeing an effect.
Maximum concentration was determined by solubility in saltwater or onset of adverse effects (e.g., degrowth, paralysis, death) upon overnight incubation.
Where available, previously reported concentrations in cell culture or animal systems were included in the testing.
A negative result means no obvious effects were observed at the maximum concentration that warrant further investigation.
For factors that gave interesting effects (e.g, insulin), a range of lower concentrations were subsequently tested for optimization.

TABLE 5B
VARIOUS MOLECULAR AND PHYSICAL MODULATIONS WERE
SCREENED TO RECAPITULATE ARM REGENERATION.

| Factor | What was tested | Implementation |
|---|---|---|
| Nutrient | 1-50 rotifers/animal | Food was administered |
| | 0-5 brine shrimps/animal | daily |
| | Combination of both | |
| Water current | 0-60 bubbles per minute | Ambient air was pumped |
| | | to the cone |
| Aquarium | Beaker, plate, tube, cone | Amputated ephyrae were |
| geometry | | placed in different |
| | | aquaria |
| Water volume | 100 mL-1 L | |
| Animal density | 10-100 ephyrae/L | |
| Temperature | 18-25° C. | Cooler or heater |
| Heat shock | 30 sec at 42° C. | Water bath |
| | 30 min at 37° C. | |
| Dark | Ephyrae were kept in the | Aquaria were wrapped |
| | dark throughout the | with aluminum foil |
| | experiment | |
| Salinity | 18-55 ppt | Ephyrae were placed in |
| | | artificial seawater with |
| | | varying salinity |
| Means of | Shaking | Incubator, 60-120 rpM |
| Current | Rotating | Rotisserie, 5-8 rpM |
| Generation | Bubbling | Air tubing, 0-60 |
| | | bubbles/min |

Modulators were administered or physical parameters were implemented upon amputation.
Some factors were dissolved in DMSO or ethanol; for these molecules, the control group was administered with an equal volume of the solvent.
Since few, if at all, of the molecular modulators had been tested in Aurelia, the maximum concentrations were tested to maximize the chance of seeing an effect.
Maximum concentration was determined by solubility in saltwater or onset of adverse effects (e.g., degrowth, paralysis, death) upon overnight incubation.
Where available, previously reported concentrations in cell culture or animal systems were included in the testing.
A negative result means no obvious effects were observed at the maximum concentration that warrant further investigation.
For factors that gave interesting effects (e.g, insulin), a range of lower concentrations were subsequently tested for optimization.

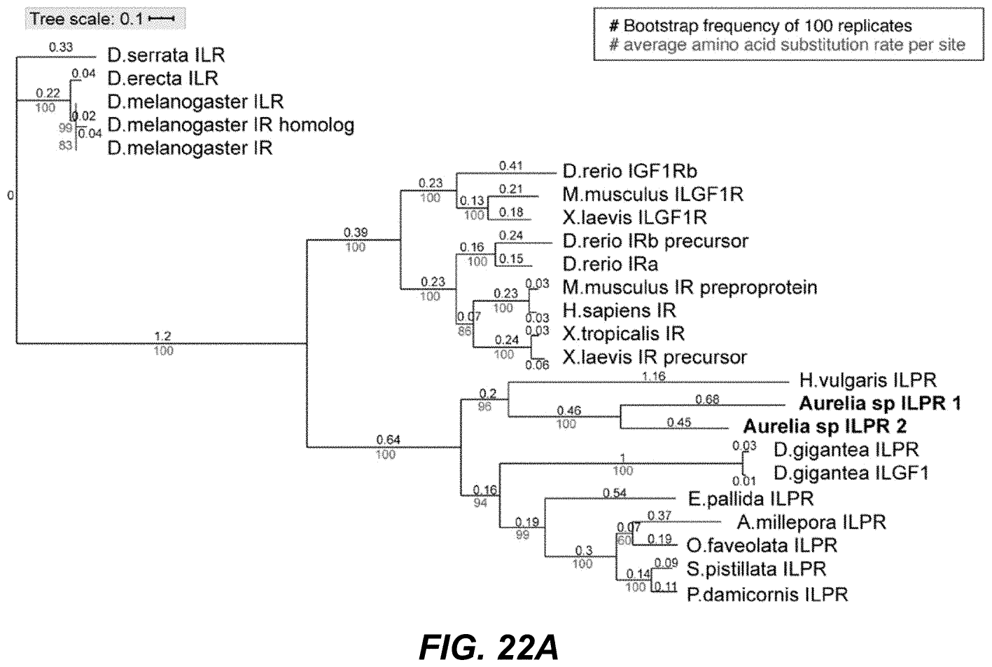

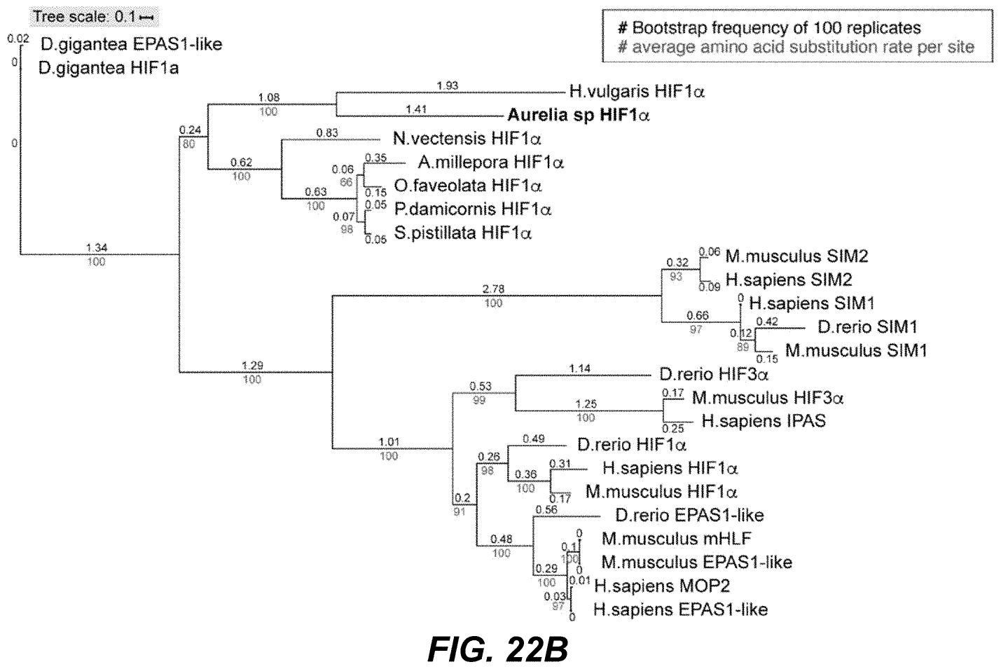

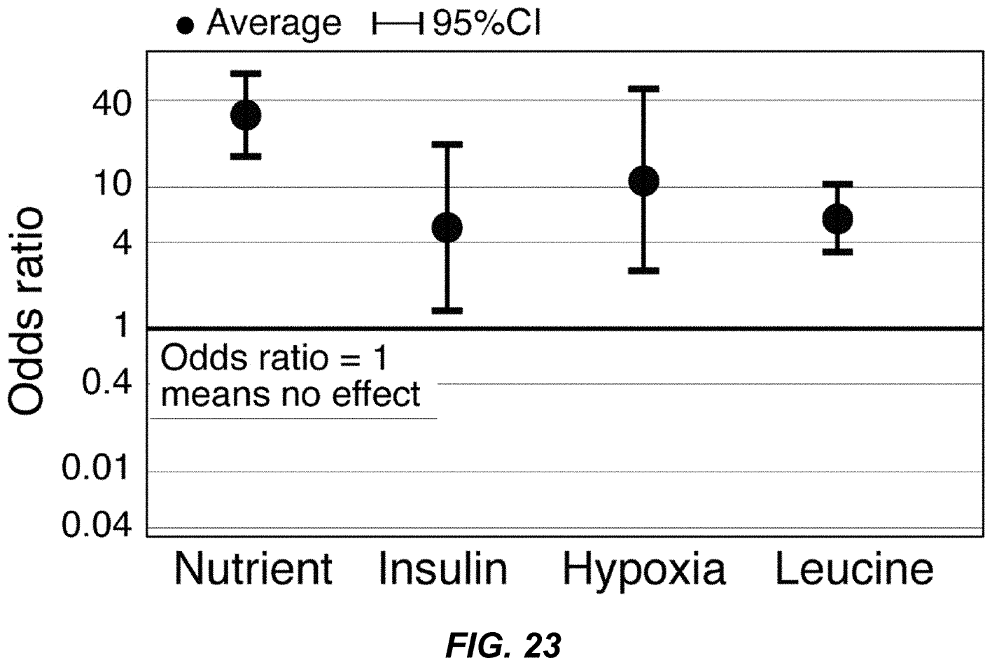

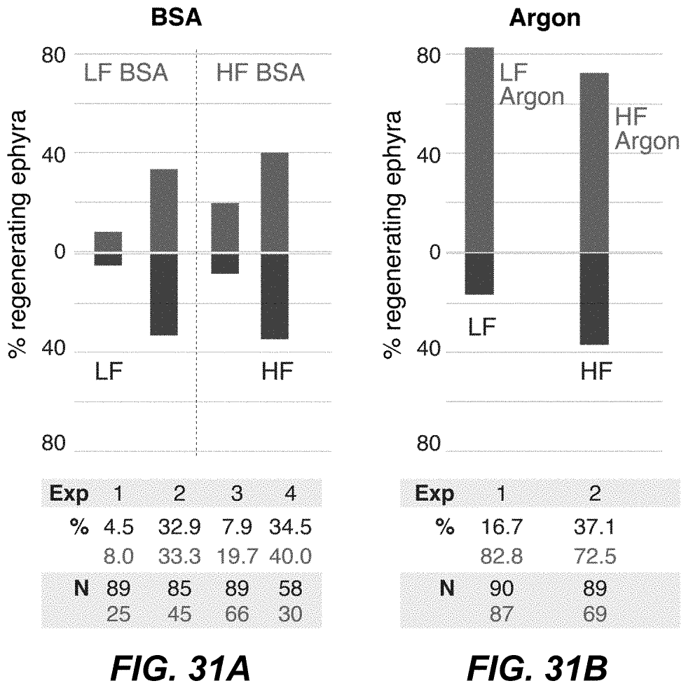

The second factor that promotes regeneration is insulin (FIG. 15D). It was verified that the insulin receptor is conserved in Aurelia (FIG. 22A-FIG. 22B). Administering insulin led to a reproducible (FIG. 15G and Table 4, 95% CI [1.1, 5.0-fold]) and statistically significant (p-value<0.05) increase in regeneration frequency. The insulin effect was unlikely to be due to non-specific addition of proteins, since bovine serum albumin at the same molarity showed no effect (FIG. 31A). Finally, the third promoter of regeneration is hypoxia (FIG. 15E). It was verified that the ancient oxygen sensor HIFα is present in Aurelia (FIG. 22A-FIG. 22B). Hypoxia led to a reproducible (FIG. 15G and Table 4, 95% CI [1.4, 12.0-fold]) and statistically significant (p-value<0.01) increase in regeneration frequency. To reduce oxygen, nitrogen was flown into the seawater, achieving ˜50% reduction in dissolved oxygen level (see Methods below). It was verified that the effect was due to reduced oxygen rather than increased nitrogen, since reducing oxygen using argon flow similarly increased regeneration frequency (FIG. 31B, 95% CI [1.99, 3.3-fold], N=2 experiments, 335 ephyrae, p-value<10⁻⁴). The factors can act synergistically (e.g., insulin and high nutrient level), but the effect appears to eventually saturate (e.g., hypoxia and high nutrient level).

TABLE 6
STATISTICAL SIGNIFICANCE OF REGENERATION INDUCTION
IN AURELIA ASSESSED USING ODDS RATIO.

| Treatment | Odds Ratio | 95% CI | p-value |
|---|---|---|---|
| Nutrient | 30.9 | 15.8, 60.6 | <0.0001 *** |
| Insulin | 5.0 | 1.3, 19.4 | 0.02 * |
| Hypoxia | 10.8 | 2.5, 46.6 | 0.001 ** |
| Leucine | 5.8 | 3.4, 10.1 | <0.0001 *** |

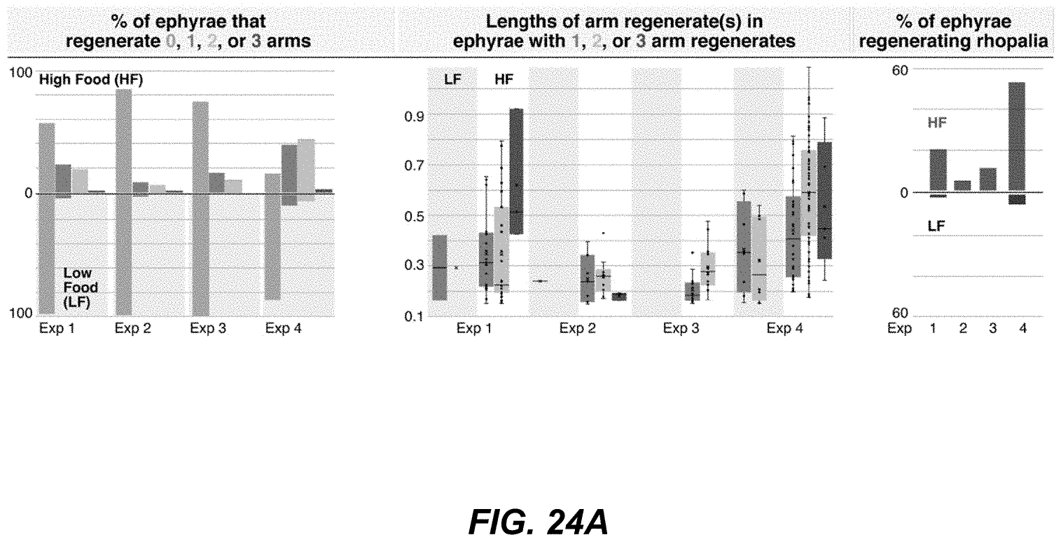

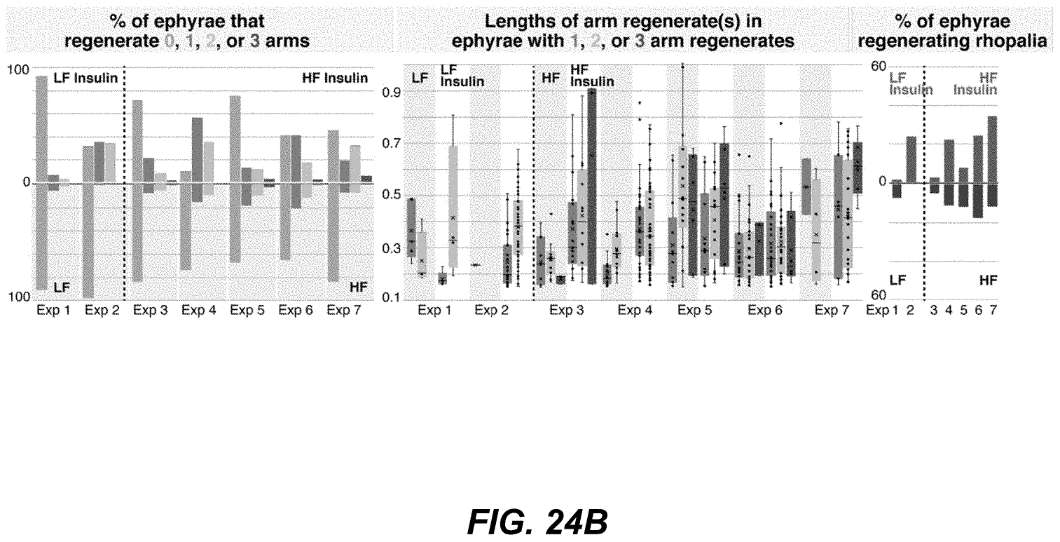

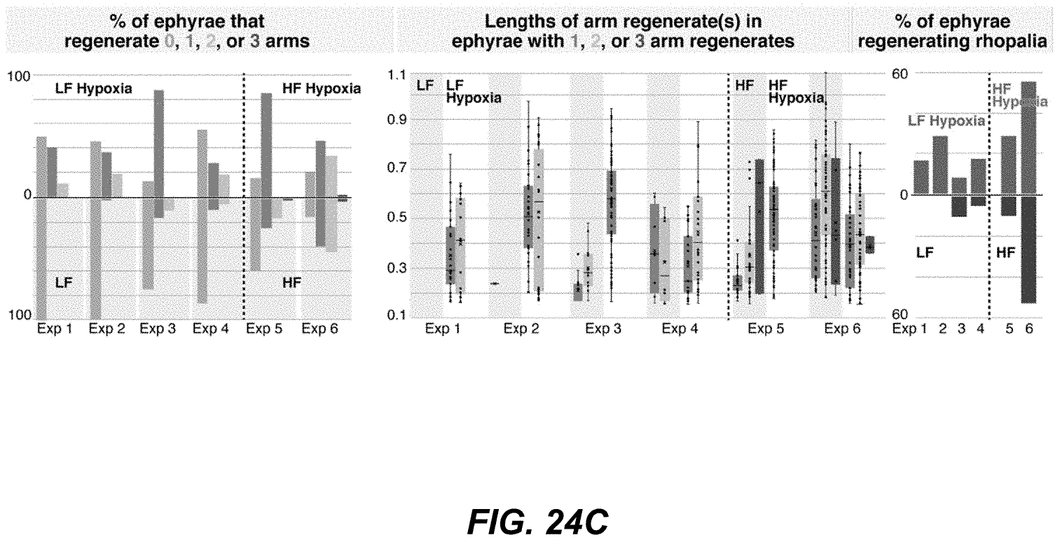

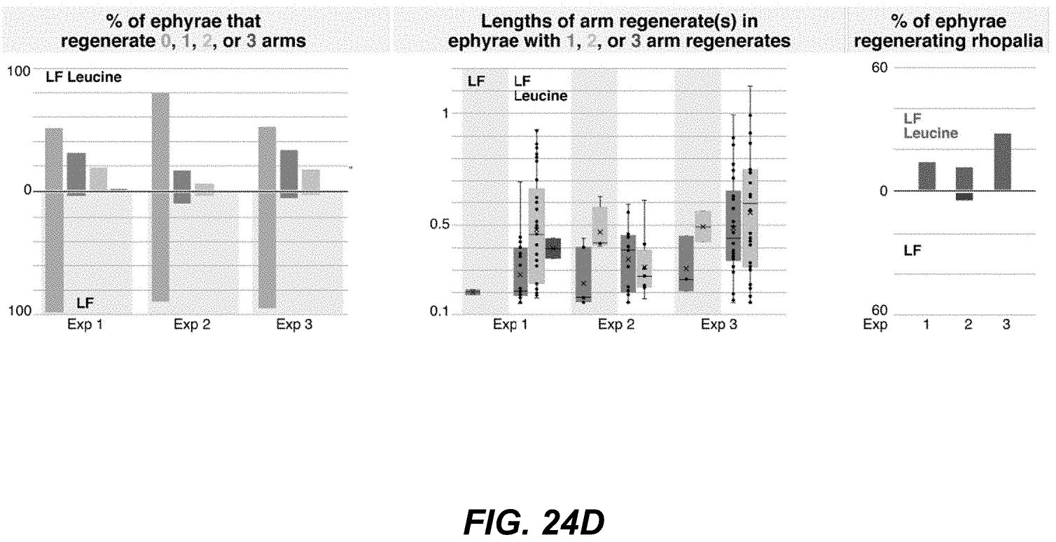

In addition to quantifying the number of ephyrae that regenerate, the regeneration phenotypes were further quantified in each ephyra, i.e., the number of arms regenerating, the length of arm regenerates, and the formation of rhopalia (FIG. 24A-FIG. 24D, Table 7). Nutrient level strikingly improved all phenotypic metrics: not only more ephyrae regenerated in higher nutrients, more ephyrae regenerated multiple arms, longer arms, and arms with rhopalia. Insulin and hypoxia, interestingly, show differential phenotypes. Most strikingly, while insulin induced more ephyrae to regenerate multiple arms, hypoxia induced largely single-arm regenerates, e.g., hypoxia experiments 3 and 5 in FIG. 24A-FIG. 24D. Thus, while all factors increased the probability to regenerate, they had differential effects on the regeneration phenotypes, suggesting a decoupling to a certain extent between the regulation of the decision to regenerate and the regulation of the subsequent morphogenesis.

TABLE 7
STATISTICAL ANALYSIS OF THE REGENERATION PHENOTYPES IN HIGH
AMOUNT OF NUTRIENTS, INSULIN, HYPOXIA, AND L-LEUCINE.

| | Effect size | 95% CI | p-value | Effect size | 95% CI | p-value |
|---|---|---|---|---|---|---|
| | High food | | | L-leucine | | |
| % ephyrae | 7.4 | 4.7, 12.1 | <0.0001 *** | 4.1 | 2.5, 6.6 | <0.0001 *** |
| regenerating | | | | | | |
| % ephyrae | 11.4 | 4.9, 5.3 | <0.0001 *** | 6.0 | 1.9, 19.1 | 0.003 ** |
| regenerating >1 | | | | | | |
| arm | | | | | | |
| Length of | 1.6 | 1.2, 2.0 | 0.0003 *** | 1.7 | 1.42, 1.9 | <0.0001 *** |
| arm | | | | | | |
| regenerates | | | | | | |
| % ephyrae | 11.8 | 5.3, 26.5 | <0.0001 *** | 6.1 | 2.1, 17.7 | 0.0009 *** |
| regenerating | | | | | | |
| rhopalia | | | | | | |
| | Insulin | | | Hypoxia | | |
| % ephyrae | 2.4 | 1.1, 5.0 | 0.023 * | 4.1 | 1.4, 12.0 | 0.0099 ** |
| regenerating | | | | | | |
| % ephyrae | 1.9 | 1.3, 2.8 | 0.0005 *** | 1.2 | 0.2, 9.0 | 0.833 n.s. |
| regenerating >1 | | | | | | |
| arm | | | | | | |
| Length of | 1.2 | 0.98, 1.5 | 0.080 n.s. | 1.3 | 0.8, 2.1 | 0.239 n.s. |
| arm | | | | | | |
| regenerates | | | | | | |
| % ephyrae | 1.3 | 0.7, 2.7 | 0.427 n.s. | 2.6 | 1.0, 6.7 | 0.047 * |
| regenerating | | | | | | |
| rhopalia | | | | | | |

For frequency measurements, the effect size of a treatment compares the probability of an outcome in treated vs. control group (i.e., Risk Ratio, see Methods below).
For length measurement, the effect size of a treatment compares the proportionate change that results from the treatment (i.e., Response Ratio, see Methods below).
Analysis of effect size across experiments was performed using the metafor package15 in R with statistical coefficients based on normal distribution (see Methods below).
A treatment is reproducible if the 95% confidence intervals (95% CI) exclude 1.
The p-value evaluates the null hypothesis that the estimate effect size is 1 (i.e., no effect).

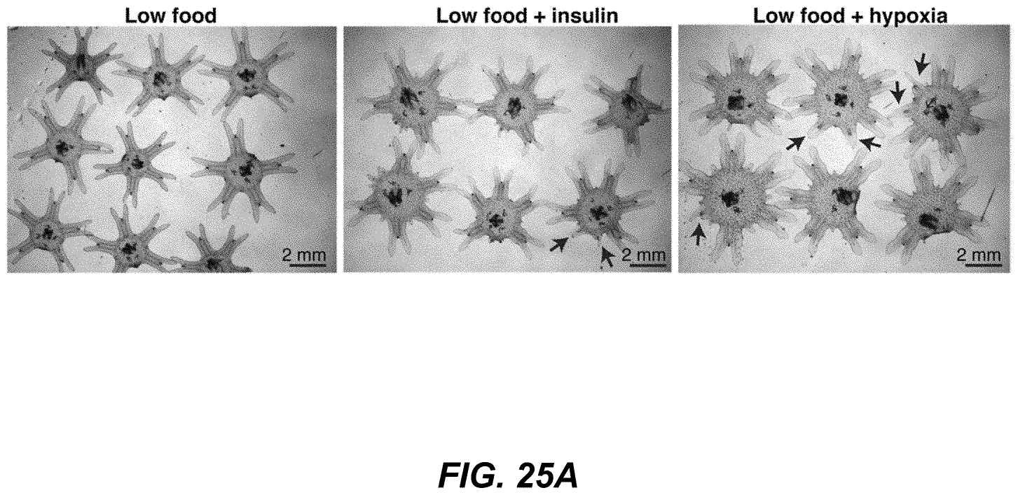

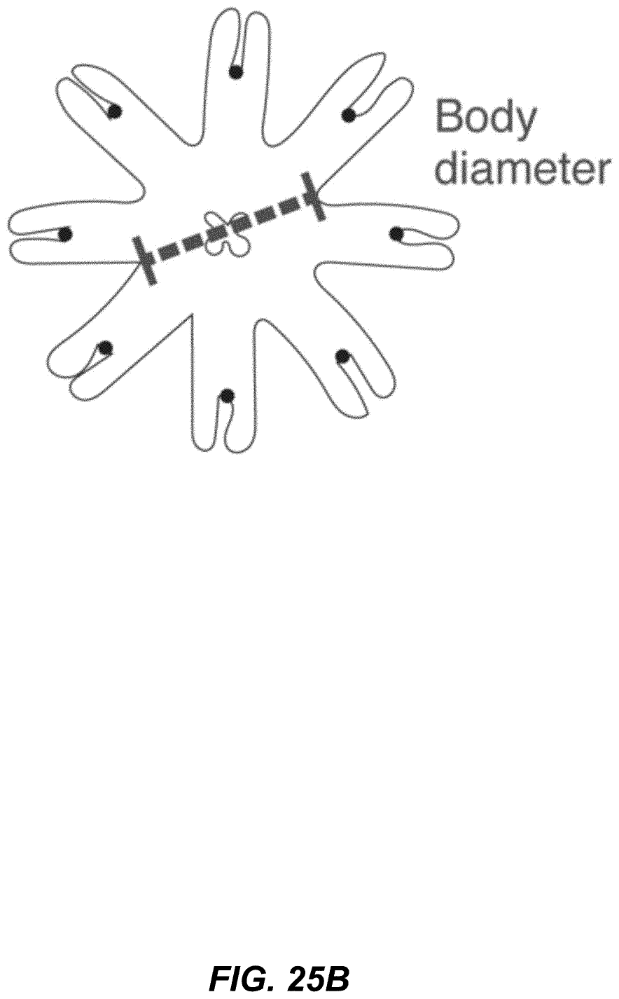

Of the three factors identified in the screen, nutrient input is the broadest, and prompted a search for a more specific nutritional component that could capture the effects of full nutrients in promoting regeneration. Jellyfish are carnivorous and eat protein-rich diets of zooplanktons and other smaller jellyfish. Notably, all three factors induced growth: treated ephyrae are larger than control ephyrae (FIG. 25 A-FIG. 25B, Table 8). The growth effect is interesting because of essential amino acids that must be obtained from food, branched amino acids supplementation correlates positively with protein synthesis and growth, and in particular, L-leucine appears to recapitulate most of the anabolic effects of high amino acid diet. Motivated by the correlation between growth and increased regeneration frequency, it was wondered if leucine administration could induce regeneration. Animals typically have a poor ability to metabolize leucine, such that the extracellular concentrations of leucine fluctuate with dietary consumption. As a consequence, dietary leucine directly influences cellular metabolism. Feeding amputated ephyrae with leucine indeed led to increased growth (FIG. 25A-FIG. 25B, Table 8). Assessing arm regeneration in the leucine-supplemented ephyrae, a significant increase in the regeneration frequency was observed (FIG. 15F-FIG. 15G, Table 4 95% CI [2.5, 6.6-fold], p-value<10⁻⁴). Furthermore, leucine treatment phenocopies the effect of high nutrients, improving all measured phenotypic metrics: increasing multi-arm regeneration, the length of arm regenerate, and the frequency of rhopalia formation (FIG. 24A-FIG. 24D and Table 7).

TABLE 8
EPHYRAE IN HIGH FOOD, INSULIN, OR HYPOXIA,
AND L-LEUCINE TEND TO BE BIGGER IN SIZE.

| | Ave. body diameter | | |
|---|---|---|---|
| Treatment | treatment/control | 95% CI | p-value |
| High food | 1.7 | 1.6, 1.8 | <0.0001 *** |
| Insulin | 1.4 | 1.1, 1.8 | 0.011 * |
| Hypoxia | 1.5 | 1.3, 1.9 | <0.0001 *** |
| Leucine | 1.1 | 1.04, 1.12 | <0.0001 *** |

Effect size analysis of the body size increase was performed using the metafor package in R (Methods).
A treatment effect is reproducible if the 95% CI exclude1.
The p-value evaluates the hypothesis that there is no effect.

These experiments demonstrate that abundant nutrients, the growth factor insulin, reduced oxygen level, and the amino acid L-leucine promote appendage regeneration in Aurelia ephyra. The identified factors are fundamental physiological factors across animals. Might the same factors promote appendage regeneration in other animal species?

### Leucine and Insulin Induce Regeneration in Drosophila Limb

To pursue this question, other poorly regenerating systems were searched for, which fortunately include most laboratory models. Drosophila, along with beetles and butterflies, belong to the holometabolans—a vast group of insects that undergo complete metamorphosis, and that as whole, do not regenerate limbs or other appendages as adults. Larval stages have imaginal disks, undifferentiated precursors of adult appendages such as the legs and antennae, and portions of imaginal disks have been shown to regenerate. Motivated by findings in Aurelia, it was asked if leucine and insulin administration can induce regenerative response in the limb of adult Drosophila. Testing leucine and insulin was focused on in this study because of considerations of specificity (i.e., nutrients are broad and composition of nutritional needs vary across species), pragmatism (i.e., administering hypoxia requires more complex setups), and in the case of Drosophila specifically, Drosophila being resistant to hypoxia.

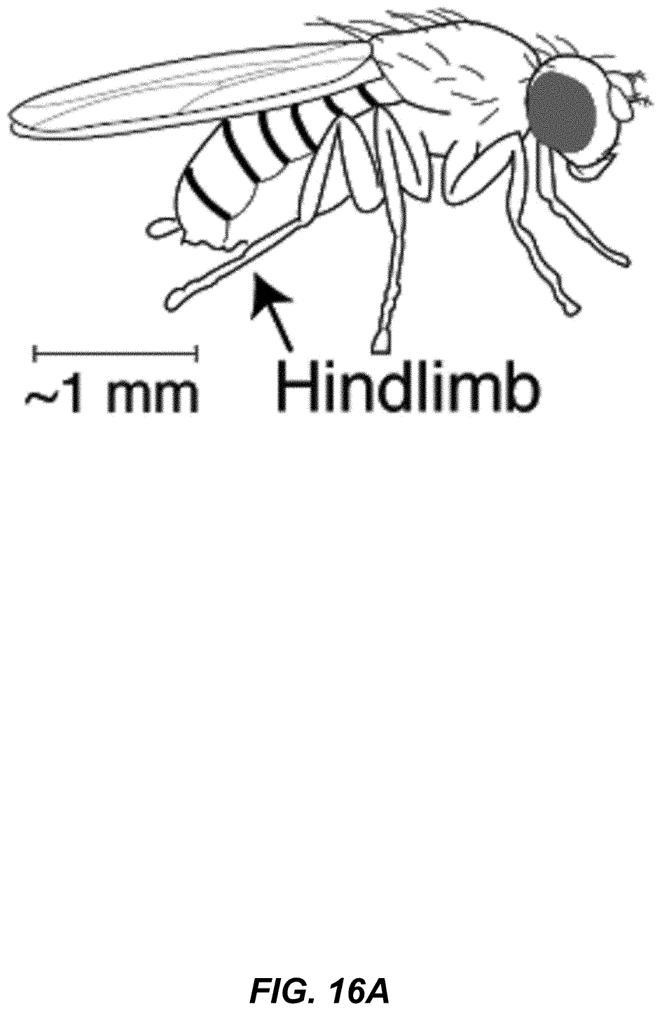

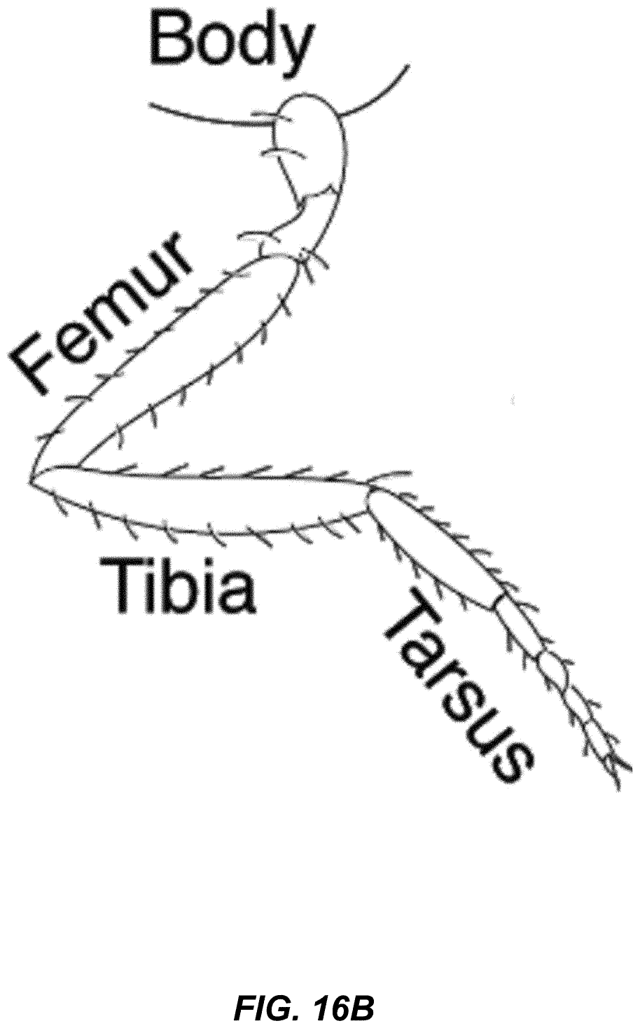

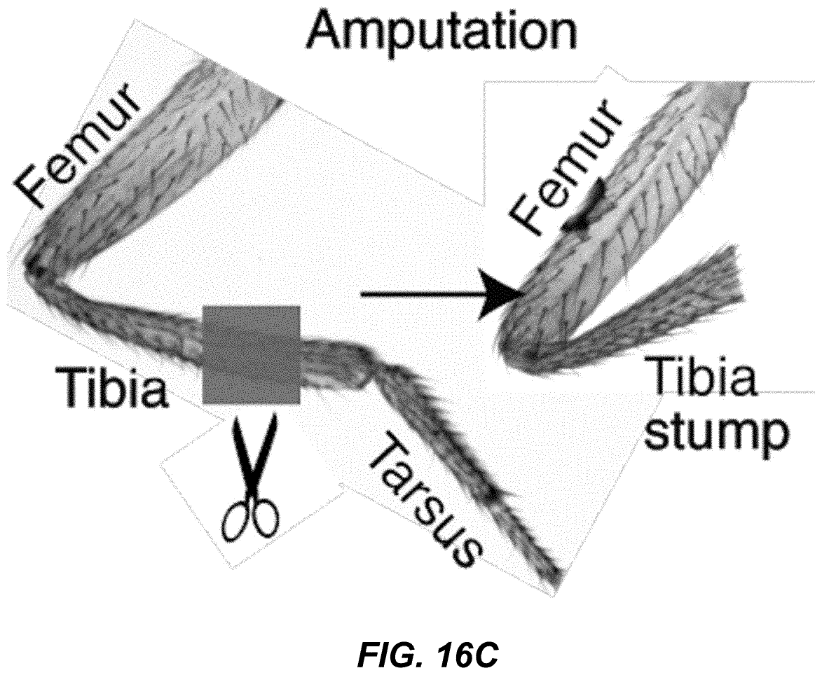

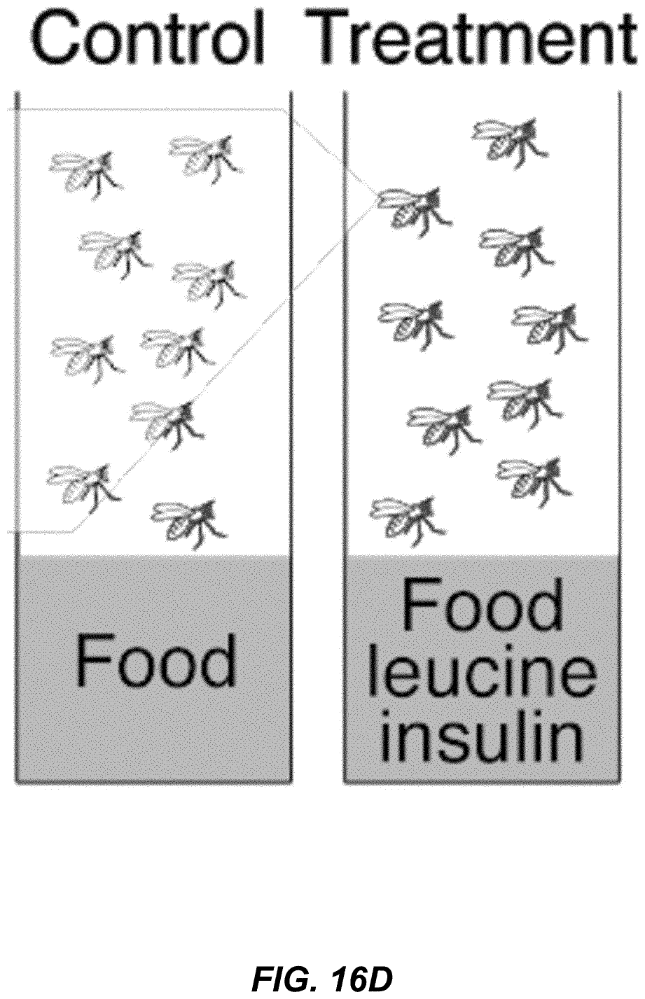

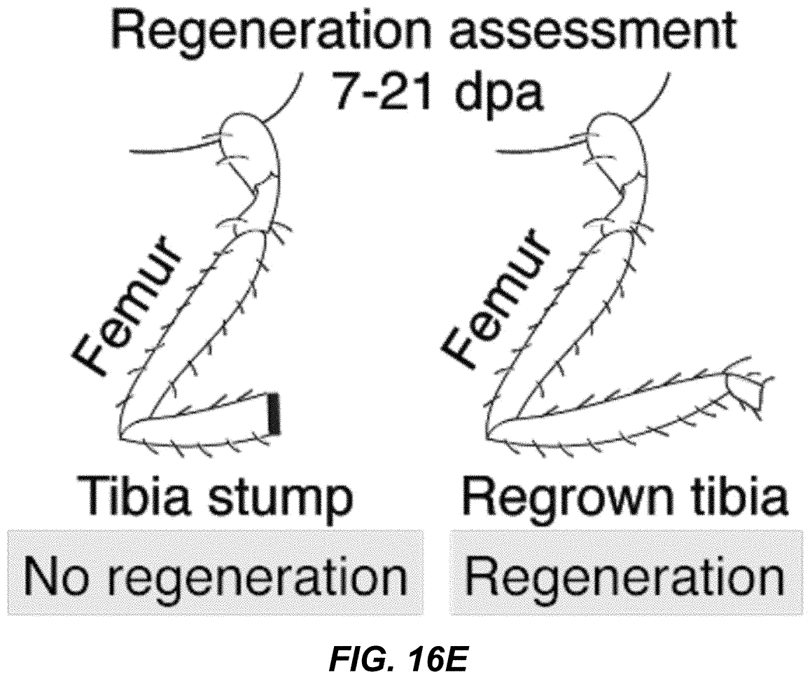

Drosophila were amputated on the hindlimb, across the fourth segment of the leg, the tibia (FIG. 16A-FIG. 16C). The amputation removed the distal half to third of the tibia and all tarsal segments. After amputation, flies were housed in vials with standard food (control) or standard food supplemented with leucine and insulin, with glutamine to promote leucine uptake (treated) (FIG. 16D). Each vial was examined multiple times, at 1, 3, 7, 14, and 21 days post amputation (dpa). Any contamination (e.g., flies with uncut tibias or wrong cuts), if any, was removed at 1 and 3 dpa. Regeneration was assessed between 7-21 dpa as the presence of a regrown tibia with a reformed distal joint (FIG. 16E).

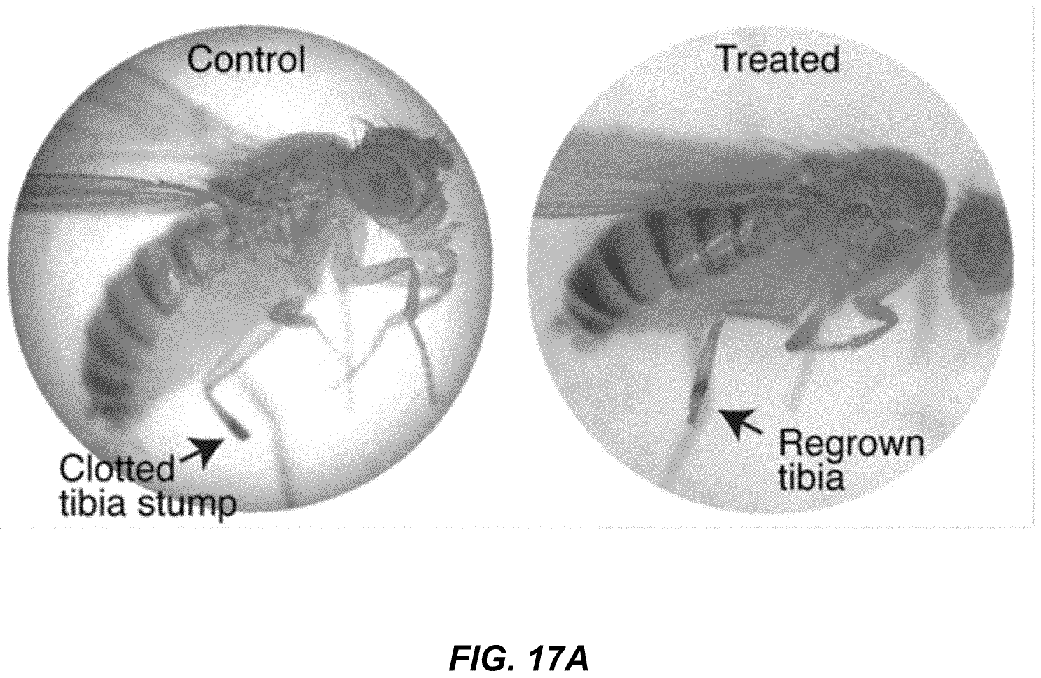

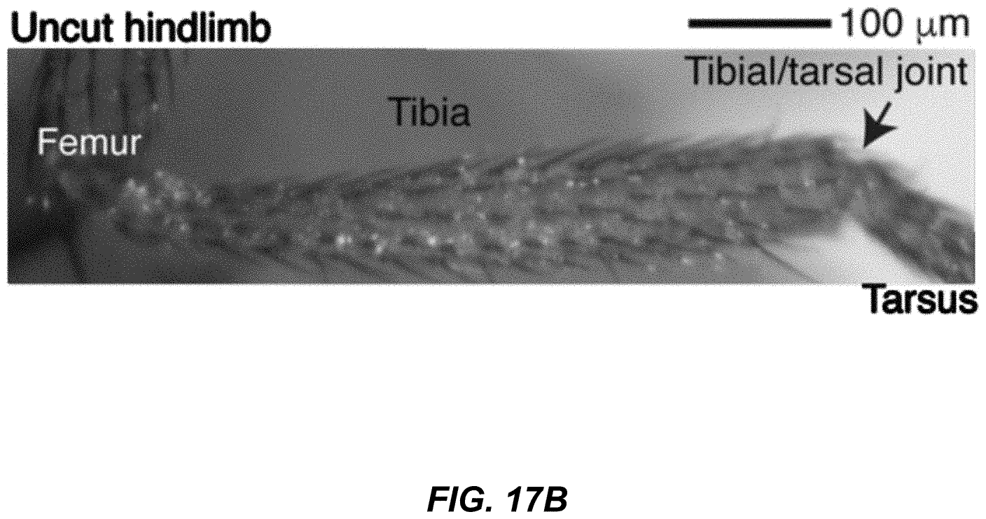

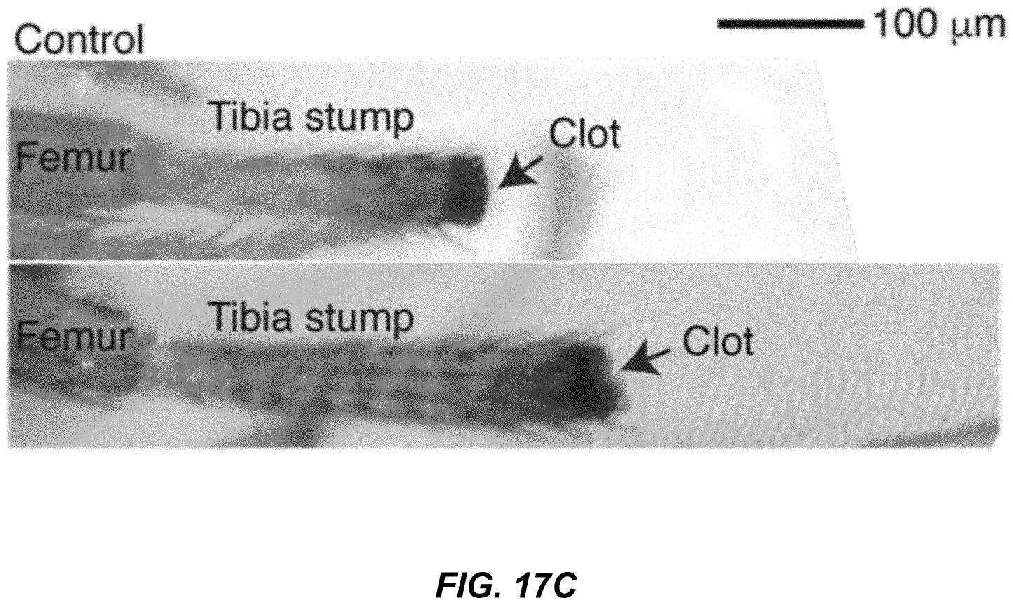

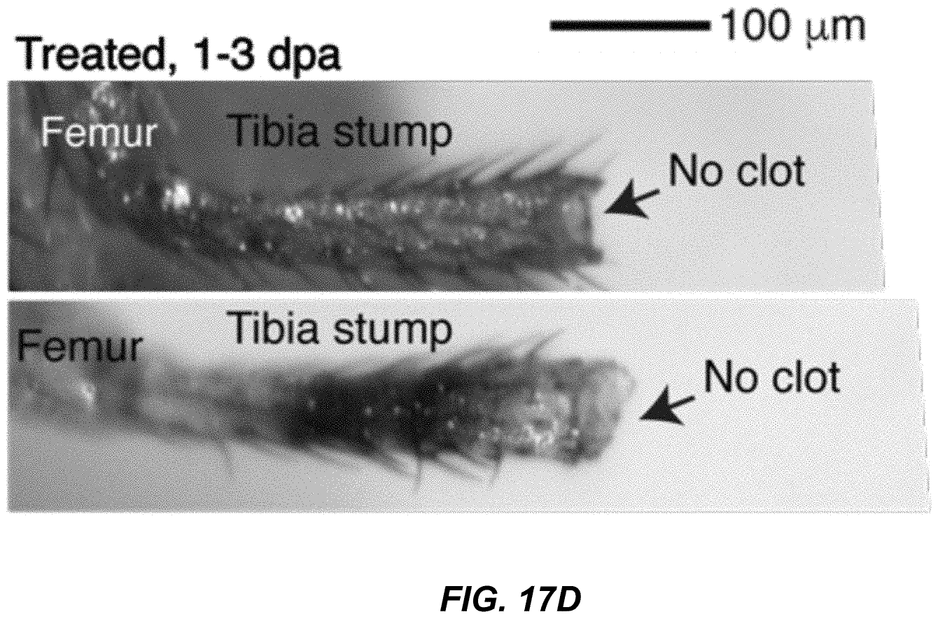

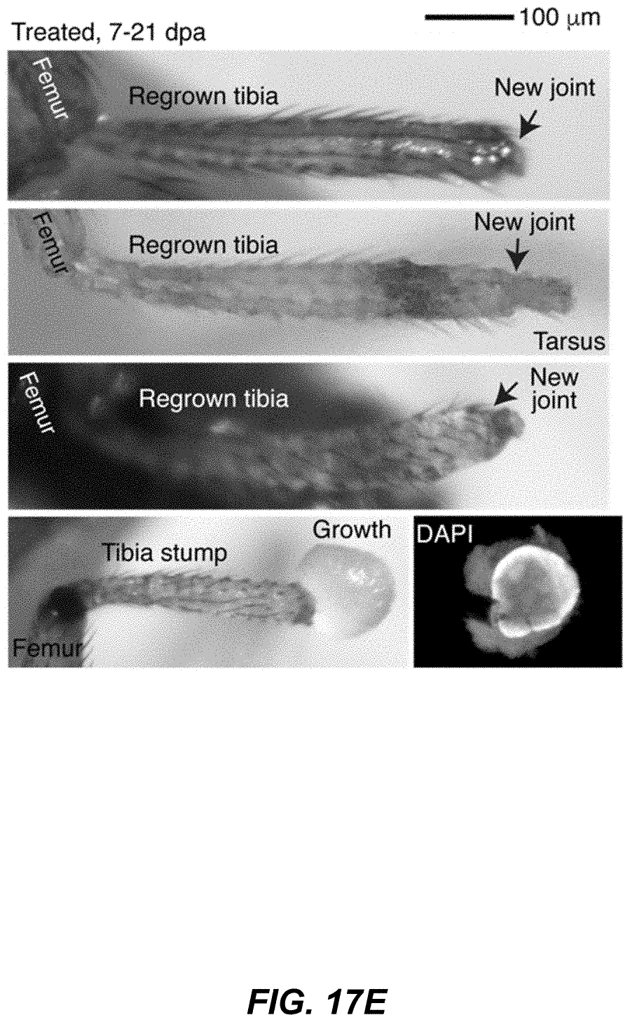

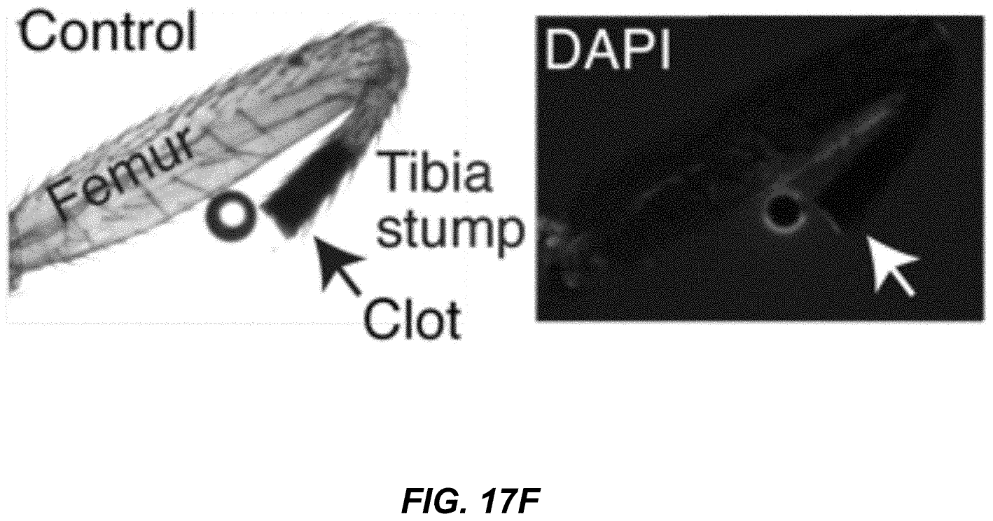

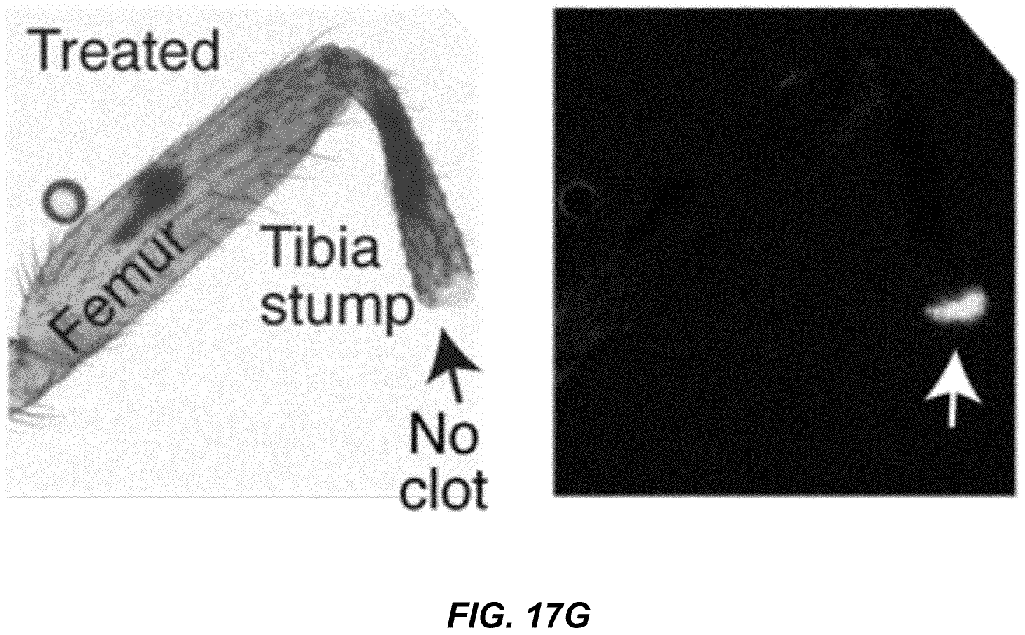

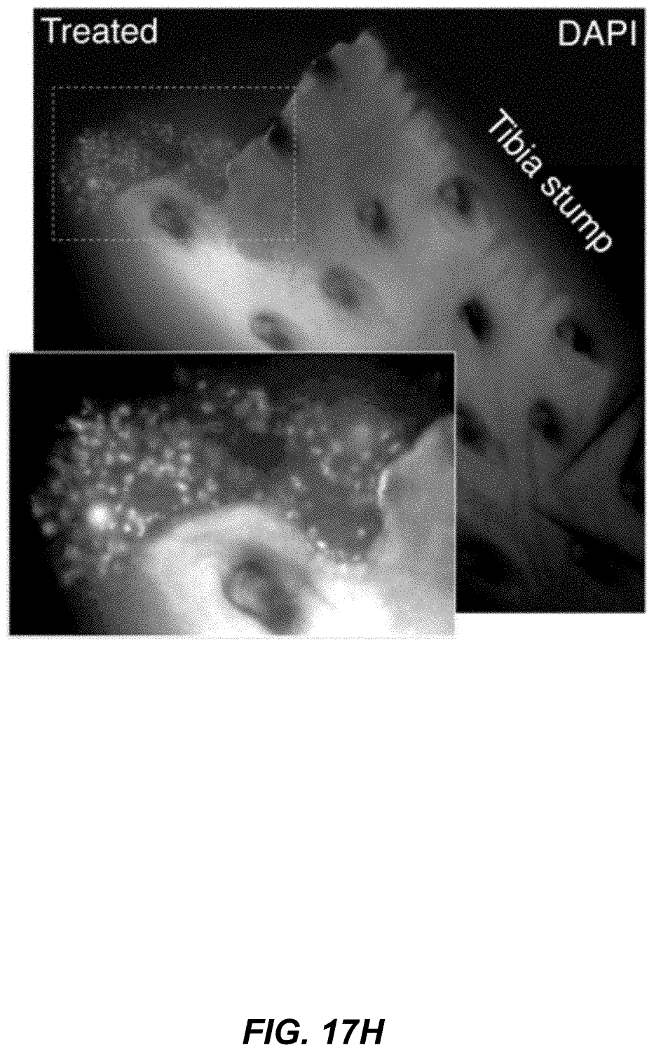

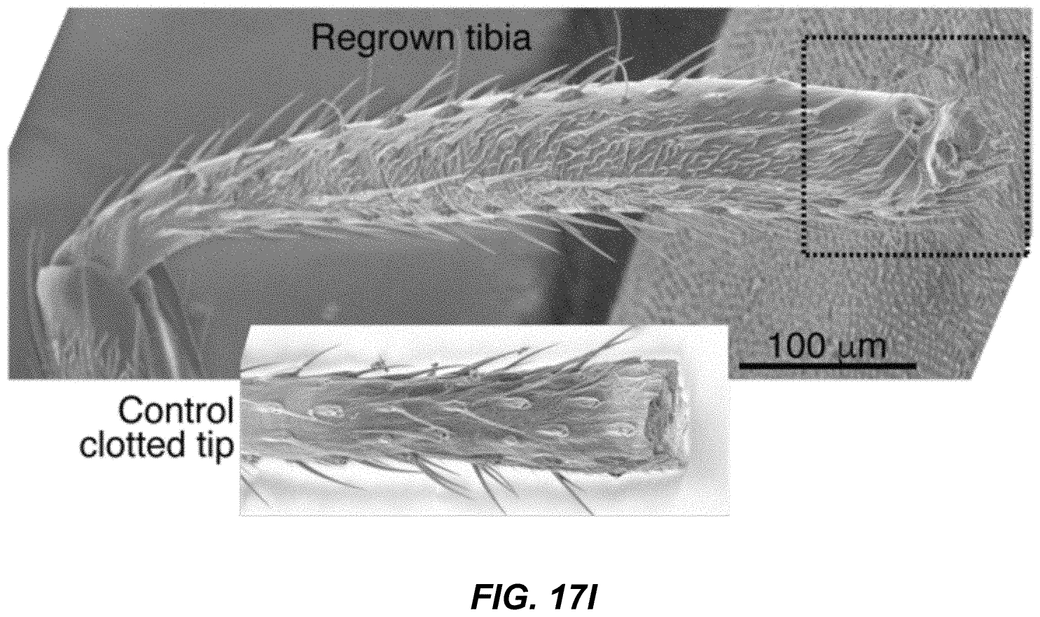

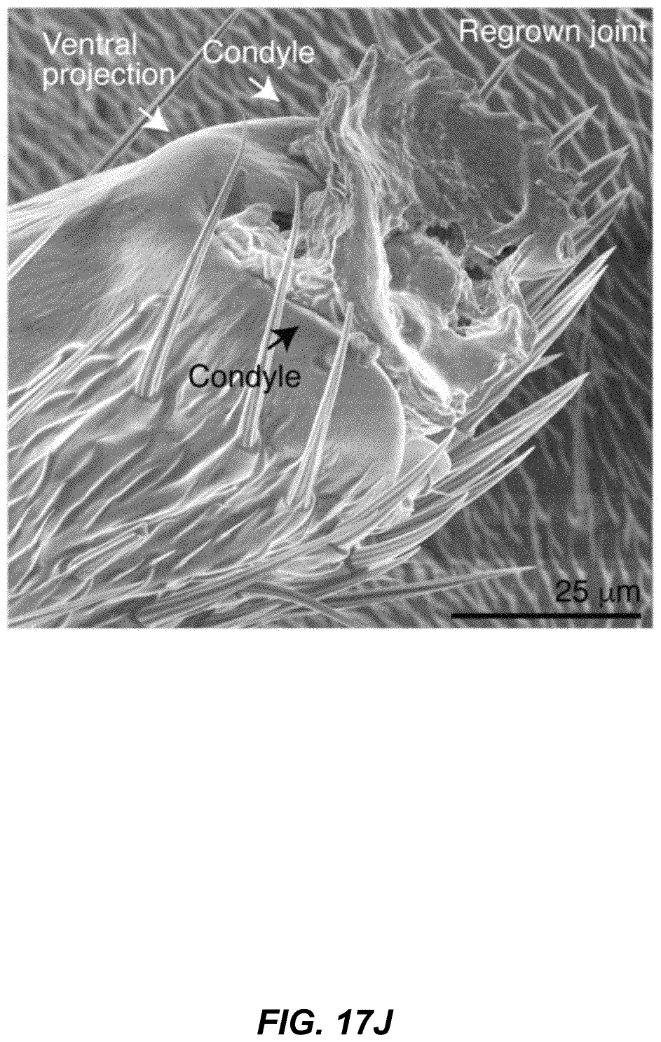

No regrown tibia was found in the 925 control flies examined (FIG. 17A). Tibia stumps in the control flies showed melanized clots within 1-3 dpa (FIG. 17C), as expected from normal wound healing process, and remained so at 7-21 dpa. In the treated flies, by contrast, some amputated tibias showed no clot at 3 dpa (FIG. 17D). The unclotted tips show white-colored tissues that stain positively with DAPI, indicating cellular materials, while clotted tips showed no DAPI signal (FIG. 17F-FIG. 17H). Flies with unclotted tibia stumps were moved into a separate housing. In this population, at 7-21 dpa, a few regrown tibias were observed (FIG. 17A, FIG. 17E). The regrown tibias culminate in reformed joints, articulating from which appears to be the beginning of a next segment. Induction of regenerative response in tibia was reproducible across genetic backgrounds, in Oregon R (12.1% white-tip tibia, 1.0% regrown tibia, N=387) and Canton S wild-type strains (29.9% white-tip tibia, 1.1% regrown tibia, N=284); the results are summarized in Table 9. Reminiscent of Aurelia. not all regenerative response was patterned, some flies showed non-specific outgrowth (FIG. 17E).

Scanning electron micrograph of a regrown tibia (the top tibia in FIG. 17E, taken one week later) morphologically confirms the regenerated joint as a tibial/tarsal joint. The completed tibia is enclosed in a sclerotized cuticle lined with longitudinal arrays of bristles, with no visible signs of the previous amputation (FIG. 17I). The joint-like structure shows the expected bilateral symmetry of a tibial/tarsal joint (as opposed to e.g., the radially symmetrical tarsal/tarsal joint) with rounded projections at the posterior and anterior end (arrows in FIG. 17J). These projections, called condyles, function as points of articulation between opposing leg segments. Indeed, articulating from the regrown condyles appears to be further growth. Finally, a unique feature of the tibial/tarsal joint of the hindlimb (but not of fore or midlimb) is an additional ventral projection between the side condyles, which serves to restrain bending of the leg upward. The ventral projection is indeed present in the regenerated joint (arrow in FIG. 17J).

TABLE 9
PHENOTYPES OBSERVED IN CONTROL AND LEUCINE/INSULIN-
TREATED FLIES, IN TWO WILD-TYPE STRAINS.

| | | | | Regrown |
|---|---|---|---|---|
| Strain | N | Clot | White tip | tibia |
| OregonR control | 860 | 100% | 0% | 0% |
| Oregon R + Leucine, Insulin | 387 | 86.9% | 12.1% | 1.0% |
| CantonS control | 65 | 100% | 0% | 0% |
| CantonS + Leucine, Insulin | 284 | 69% | 29.9% | 1.1% |

N: the number of flies examined.
Clot: clotted tibia stumps similar to those shown in FIG. 17D
White tip: non-clotted tibia stumps similar to those shown in FIG. 17E.
Regrown tibia: tibia stump that regrew a completed tibia segment, culminating in a joint.

### Leucine and Sucrose Induce Regeneration in Mouse Digit

The ability of leucine and insulin to induce regenerative response in Drosophila limb and Aurelia appendage motivated testing in vertebrates. One sign that limb regeneration may be feasible in humans is that fingertips regenerate. The mammalian model for studying limb regeneration is the house mouse, Mus musculus, which like humans regenerates digit tips. Although more proximal regions of digits do not regenerate, increasing evidence suggests that they have inherent regenerative capacity. In adult mice, implanting developmental signals in amputated digits led to specific tissue induction, i.e., bone growth with Bmp4 or joint-like structure with Bmp9. In neonates, reactivation of the embryonic gene 1in28 led to distal phalange regrowth. Thus, while patterned phalange regeneration can be induced in newborns, induction in adults so far involves a more fine-tuned stimulation, e.g., to elongate bone and then make joint, Bmp4 was first administered followed by Bmp9 in a timed manner. Motivated by the findings in Aurelia and Drosophila, it was tested if leucine and insulin administration could induce a more self-organized regeneration in adult mice.

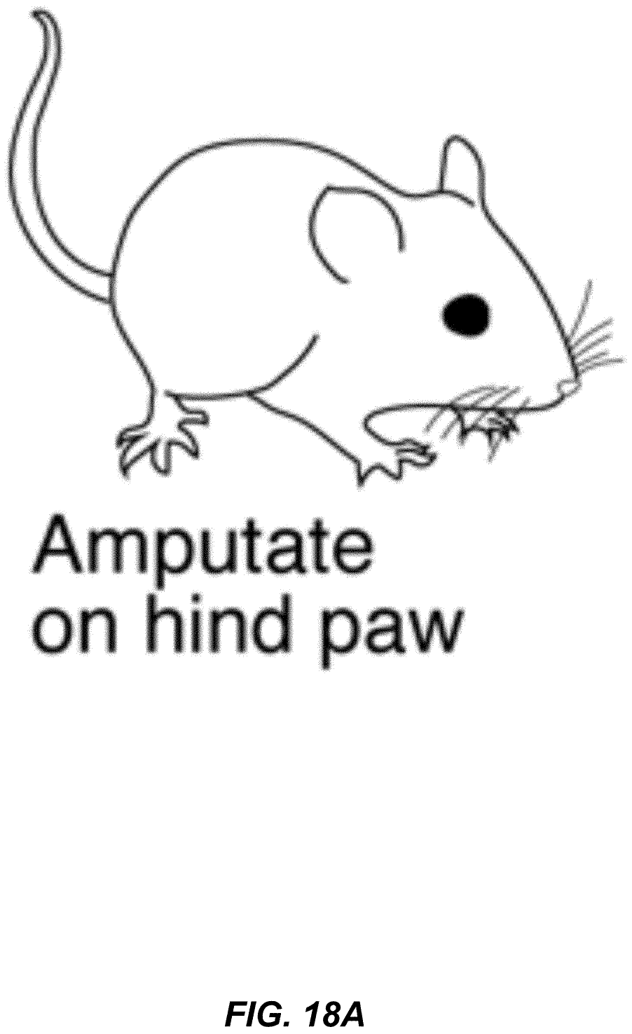

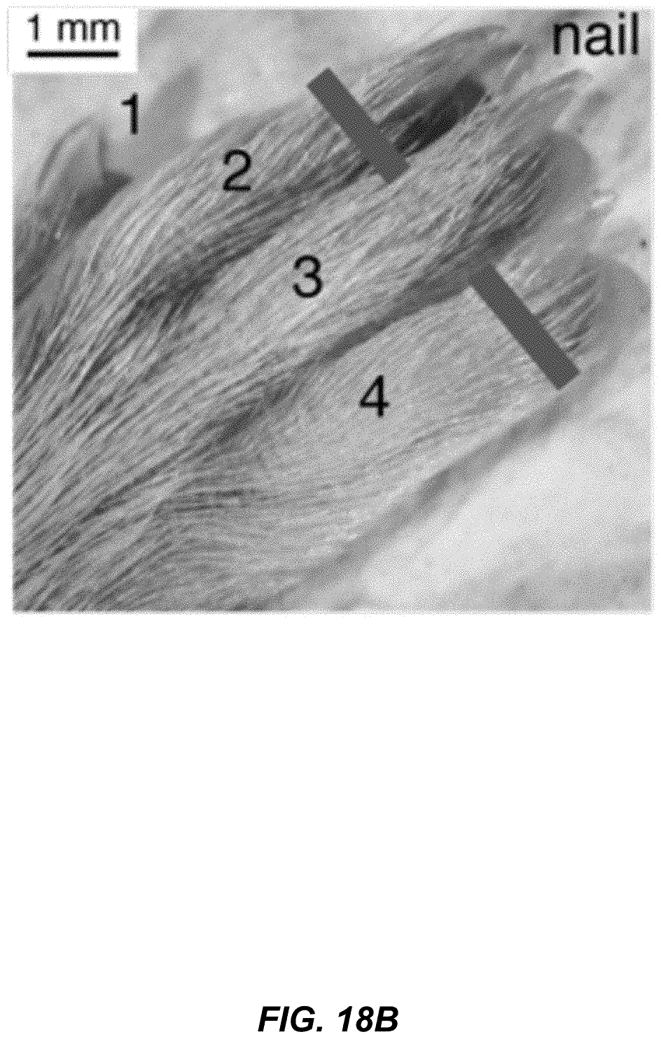

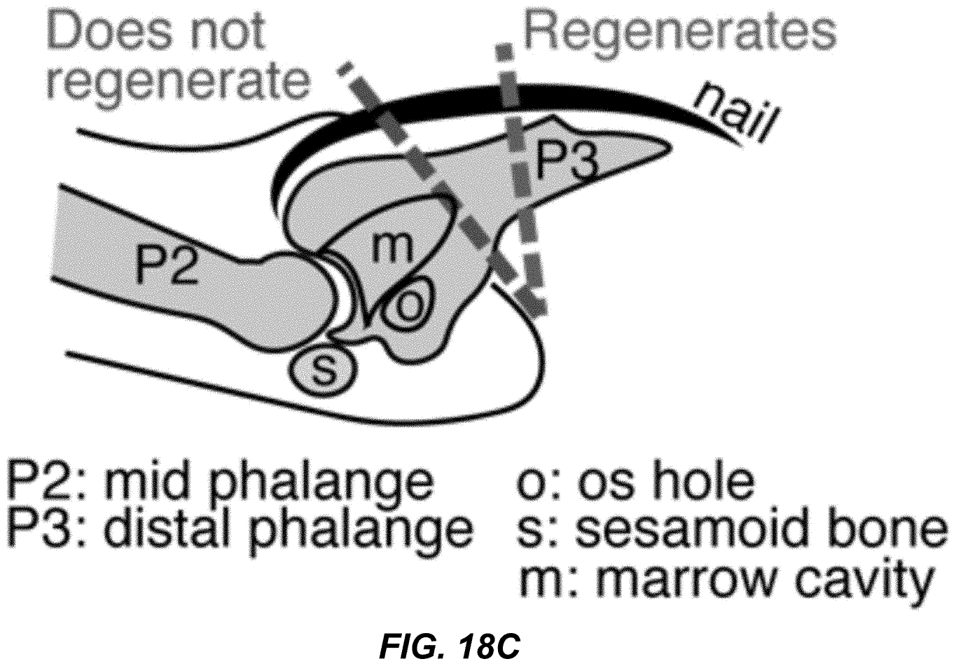

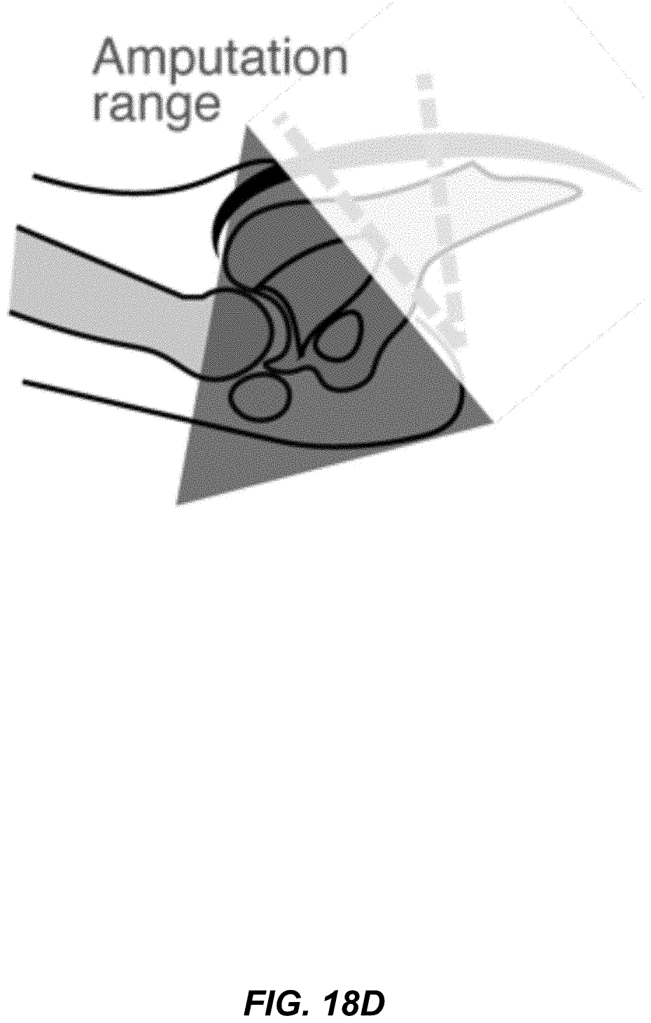

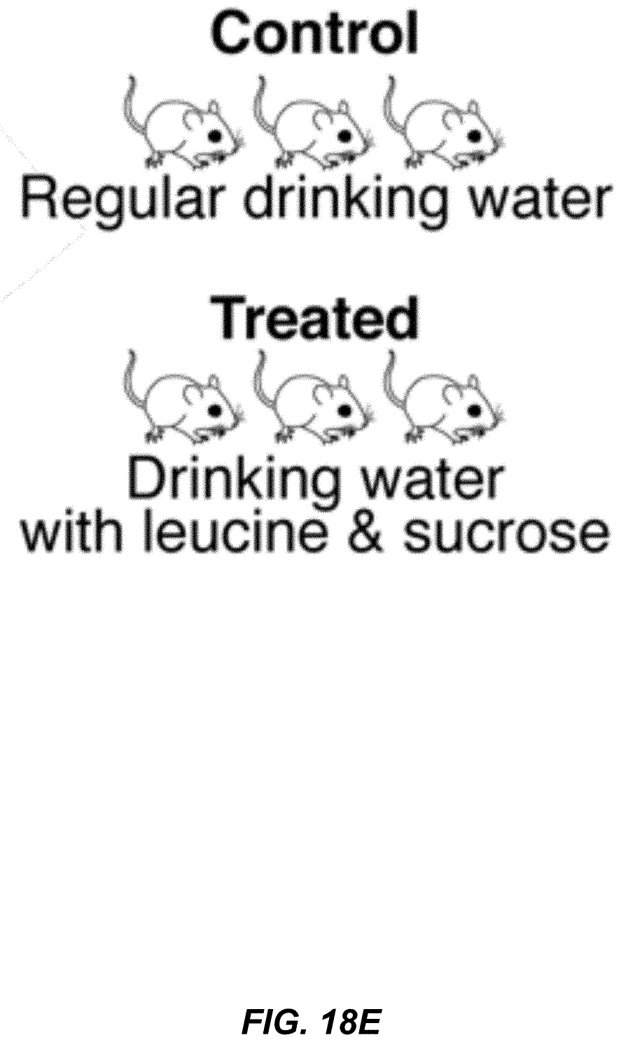

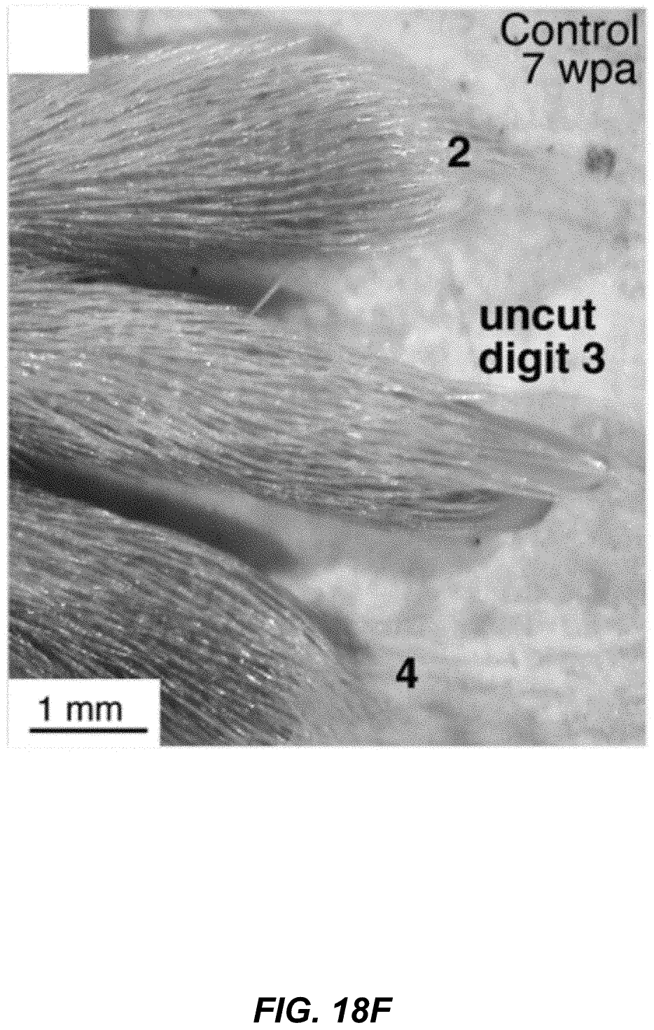

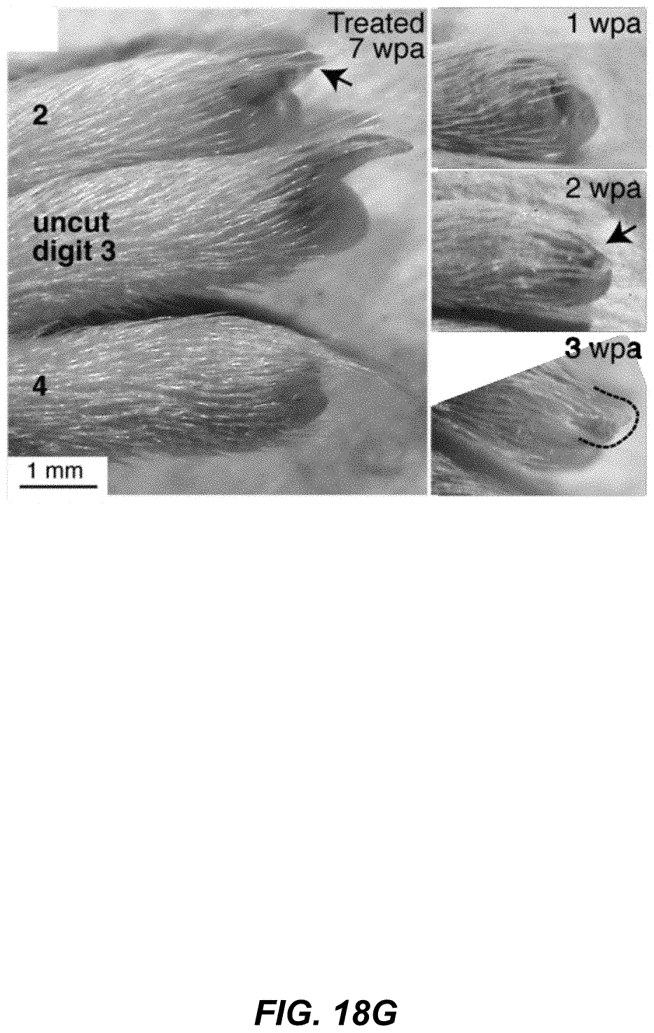

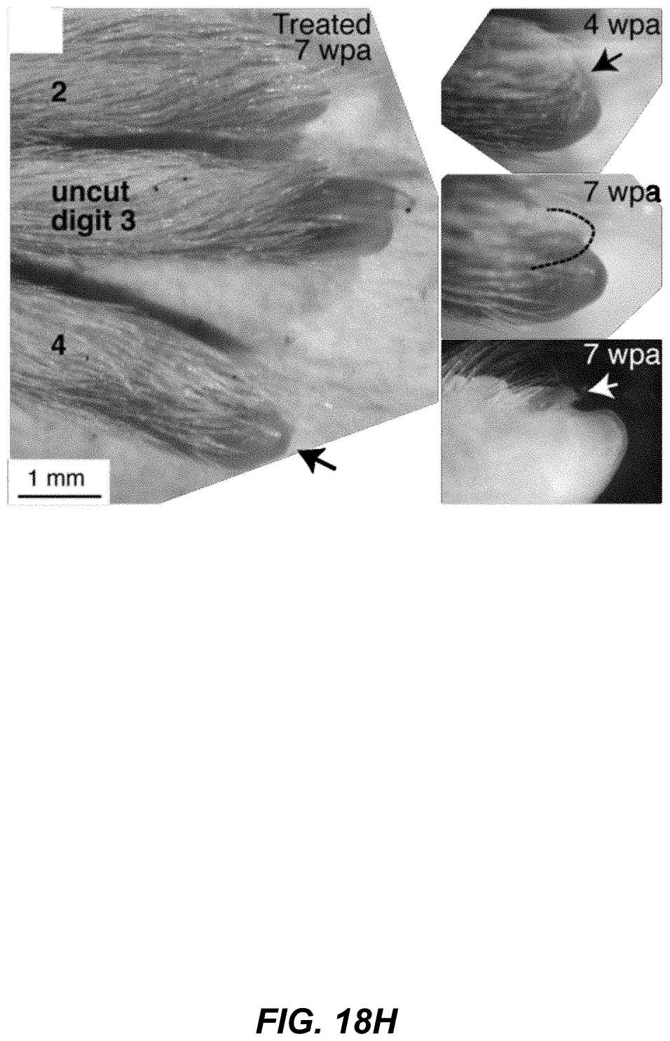

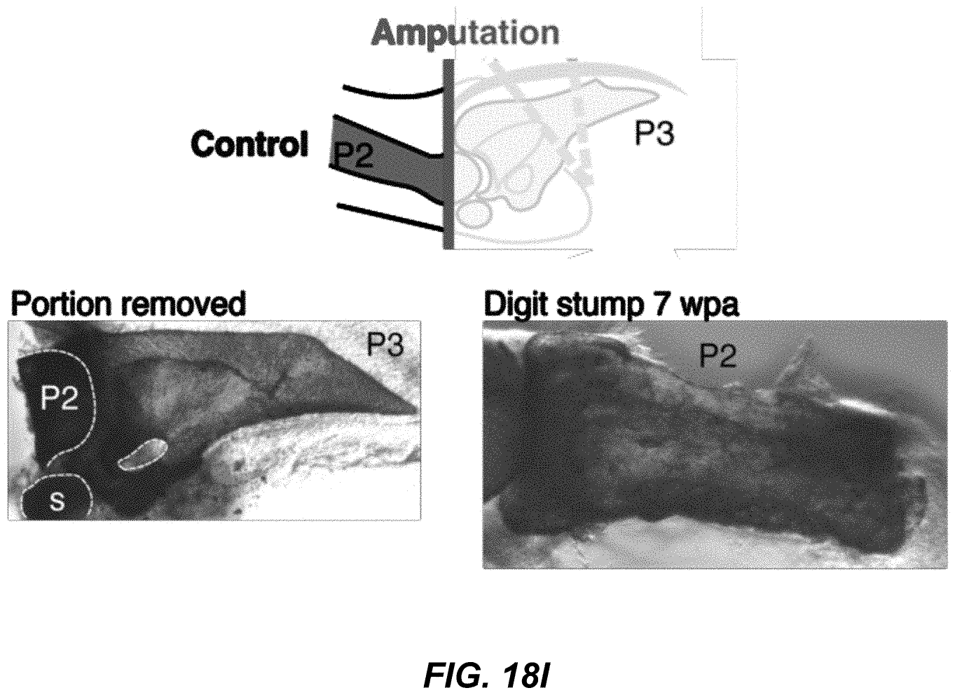

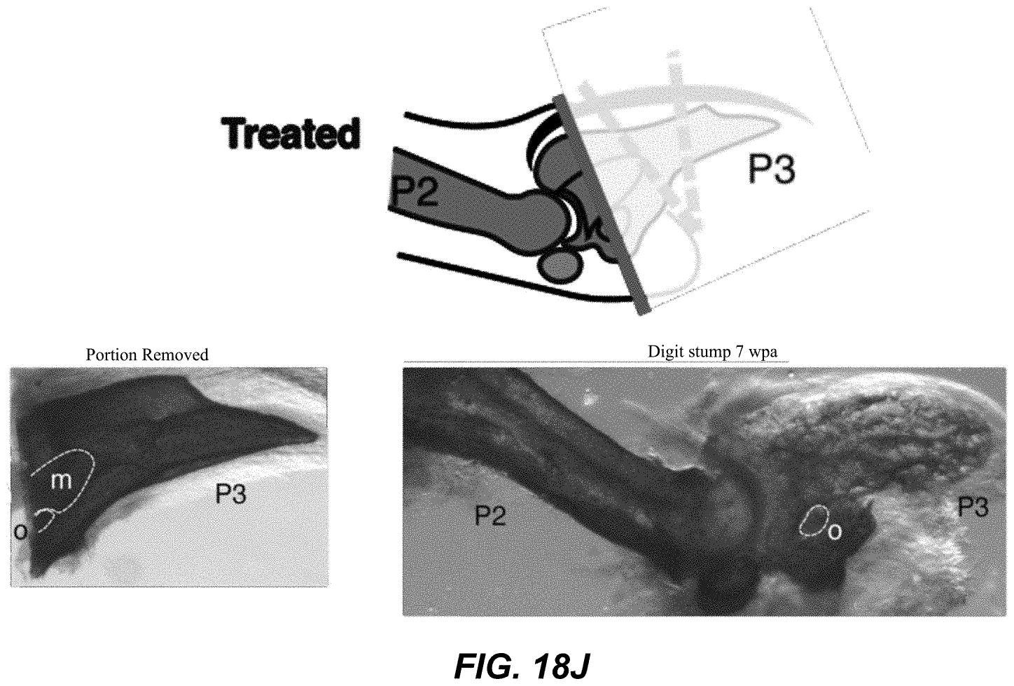

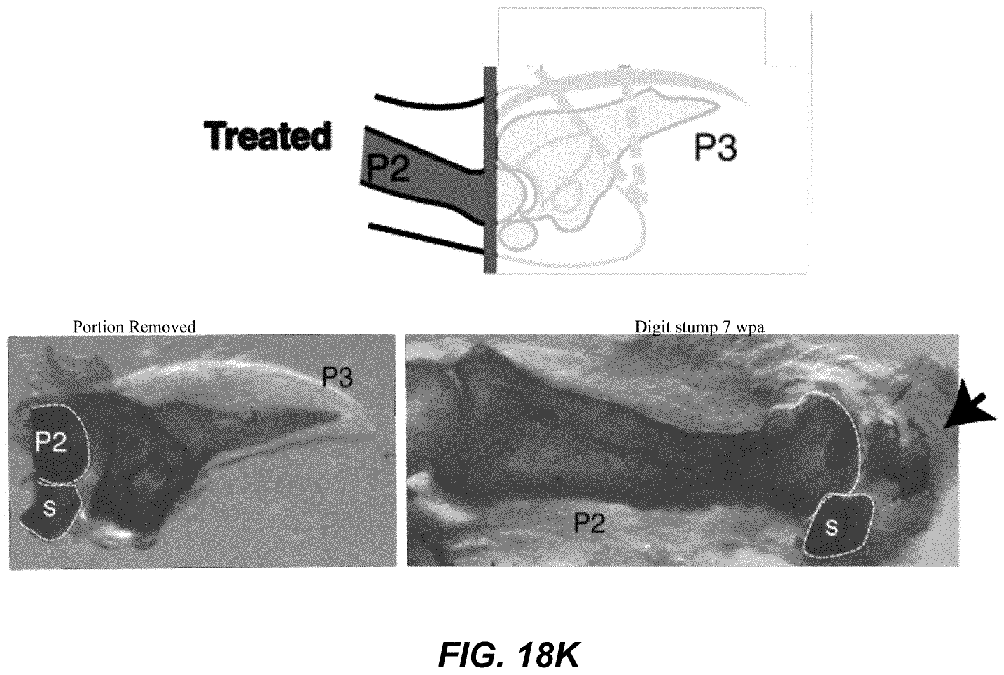

Amputation was performed on the hindpaw (FIG. 18A), on digit 2 and 4, leaving the middle digit 3 as an internal control (FIG. 18B). To perform non-regenerating amputation, a clear morphological marker is the nail, which is associated with the distal phalange (P3). Amputation that removes <30% of P3 length, that cuts within the nail, readily regenerates, whereas amputation that removes >60% of P3 length, corresponding to removing almost the entire visible nail, does not regenerate (FIG. 18C). Amputations were therefore performed entirely proximal to the visible nail—giving, within the precision of our amputation, a range of cut across somewhere between the proximal P3 and the distal middle phalange (P2) (FIG. 18D)—a range that is well below the regenerating tip region. Note additional morphological markers that lie within the non-regenerating region: the os hole (‘o’ in FIG. 18C), where vasculatures and nerves enter P3, the bone marrow cavity (‘m’ in FIG. 18C), and the sesamoid bone (‘s’ in FIG. 18C) adjacent to P2.

The digit portion removed was immediately fixed for control. The amputated mice were either provided with water as usual (control) or water supplemented with leucine and sucrose (treated) (FIG. 18E). Both groups were monitored for 7 weeks. Sucrose was used because insulin is proteolytically digested in the mammalian gut. The sucrose doses used are lower or the administration duration is shorter than those shown to induce insulin resistance. It was verified that control and treated mice had comparable initial weights (35.1±0.6 vs 34.1±1.1 grams, p-value=0.402, student's t-test), and that as expected from amino acid and sugar supplementation, treated mice gained more weight over the experimental duration (4.5±1.0 vs 7.8±1.0 grams, p-value=0.028, student's t-test).

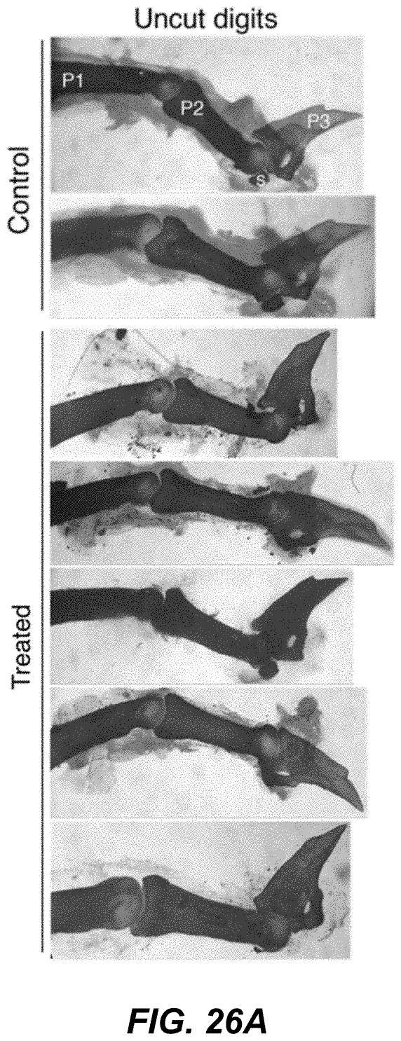

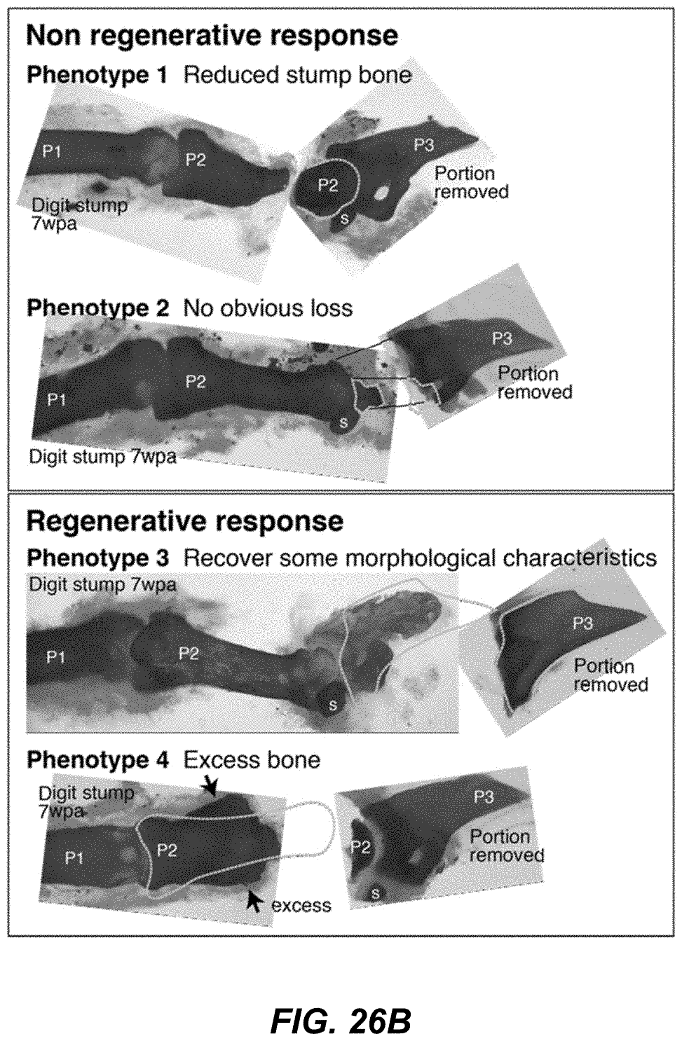

As expected for amputation proximal to the nail, no regeneration was observed in the control mice (N=20 digits, 10 mice). Amputated digits healed and re-epithelialized the wound as expected (FIG. 18F). Skeletal staining shows blunt-ended digit stumps (FIG. 18I) and in many instances, as expected, dramatic histolysis, a phenomenon where bone recedes further from the amputation plane (FIG. 26A-FIG. 26B, Table 10-Table 12). By contrast, 18.8% of the treated digits (N=48 digits, 24 mice) showed various extents of regenerative response (FIG. 26A-FIG. 26B, Table 10-Table 12).

TABLE 10
PHENOTYPE COUNTS IN ALL DIGITS

| | Control | | Treated | |
|---|---|---|---|---|
| Phenotype | # digits | Control % | # digits | Treated % |
| 1 | 16 | 80.0 | 26 | 54.1 |
| 2 | 4 | 20.0 | 13 | 27.1 |
| 3 | 0 | 0 | 6 | 12.5 |
| 4 | 0 | 0 | 3 | 6.3 |
| Total | 20 | | 48 | |

TABLE 11
P2 AMPUTATION, PHENOTYPE COUNTS
IN DIGITS AMPUTATED ACROSS P2

| | Control | | Treated | |
|---|---|---|---|---|
| Phenotype | # digits | Control % | # digits | Treated % |
| 1 | 11 | 91.7 | 23 | 65.7 |
| 2 | 1 | 8.3 | 6 | 17.1 |
| 3 | 0 | 0 | 3 | 8.6 |
| 4 | 0 | 0 | 3 | 8.6 |
| Total | 12 | | 35 | |

TABLE 12
P3+ JOINT AMPUTATION, PHENOTYPE COUNTS
IN DIGITS AMPUTATED ACROSS P3 OR JOINT

| | Control | | Treated | |
|---|---|---|---|---|
| Phenotype | # digits | Control % | # digits | Treated % |
| 1 | 5 | 62.5 | 3 | 23.1 |
| 2 | 3 | 37.5 | 7 | 53.8 |
| 3 | 0 | 0.0 | 3 | 23.1 |
| 4 | 0 | 0.0 | 0 | 0.0 |
| Total | 8 | | 13 | |

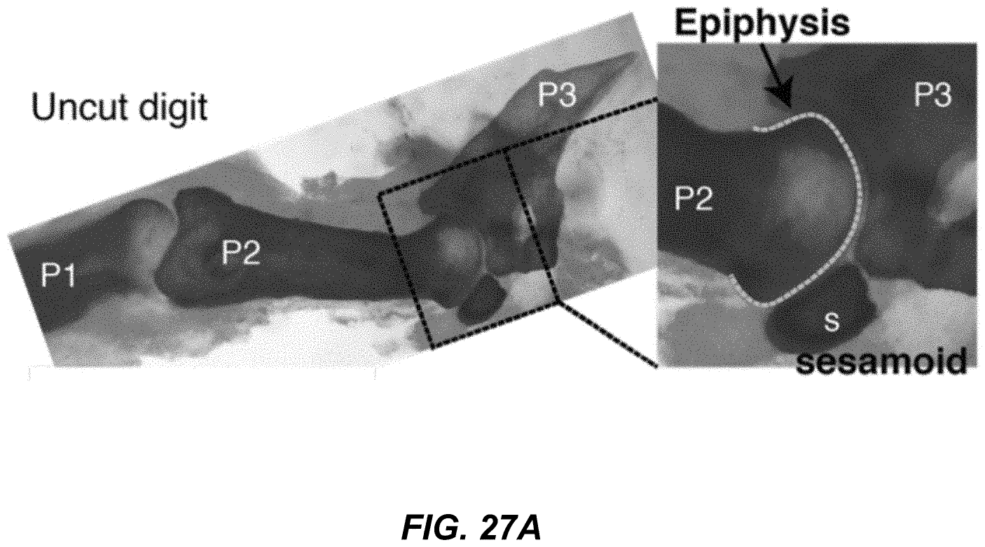

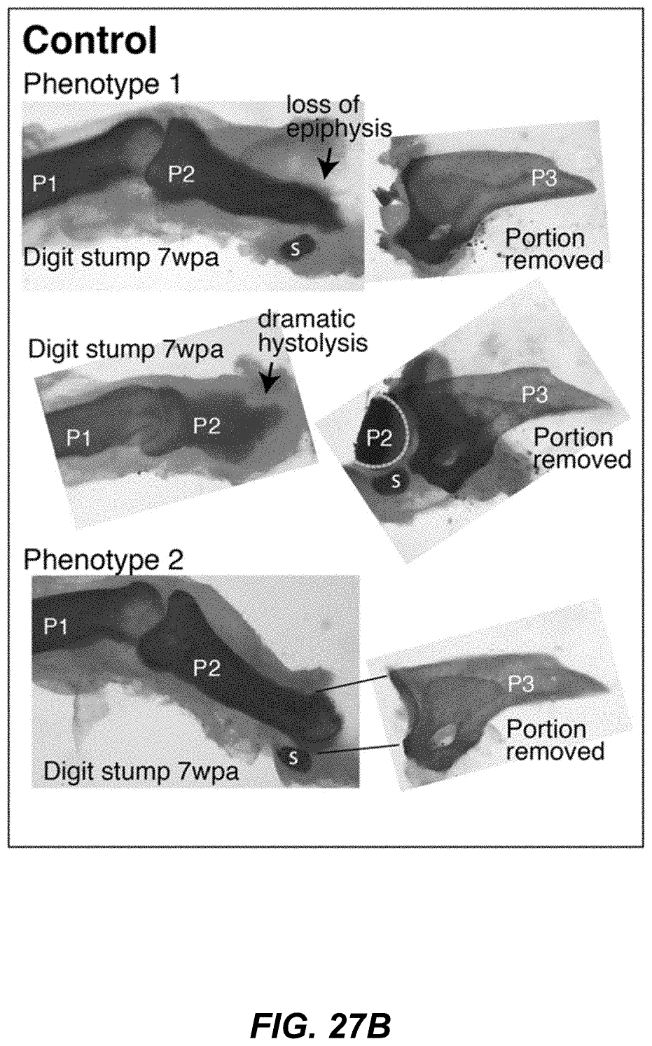

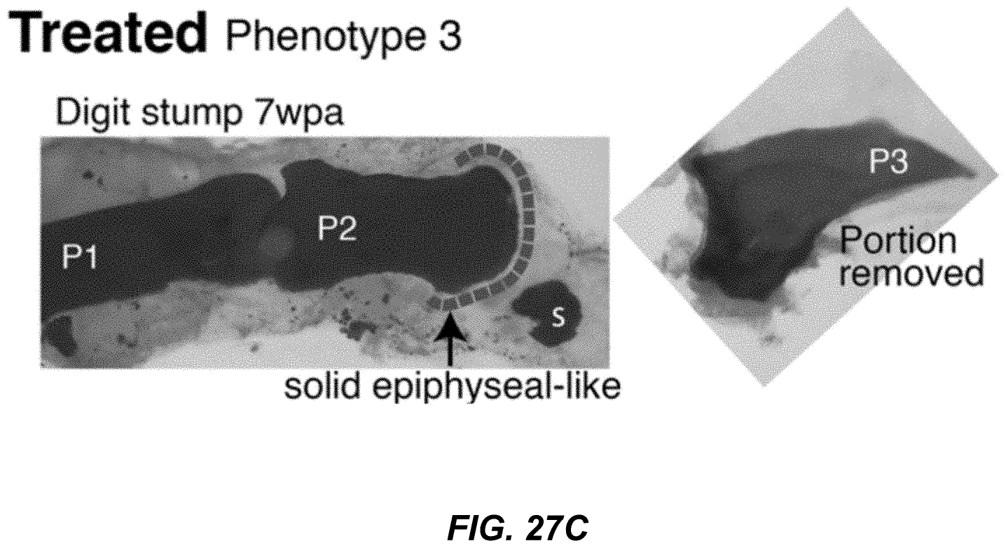

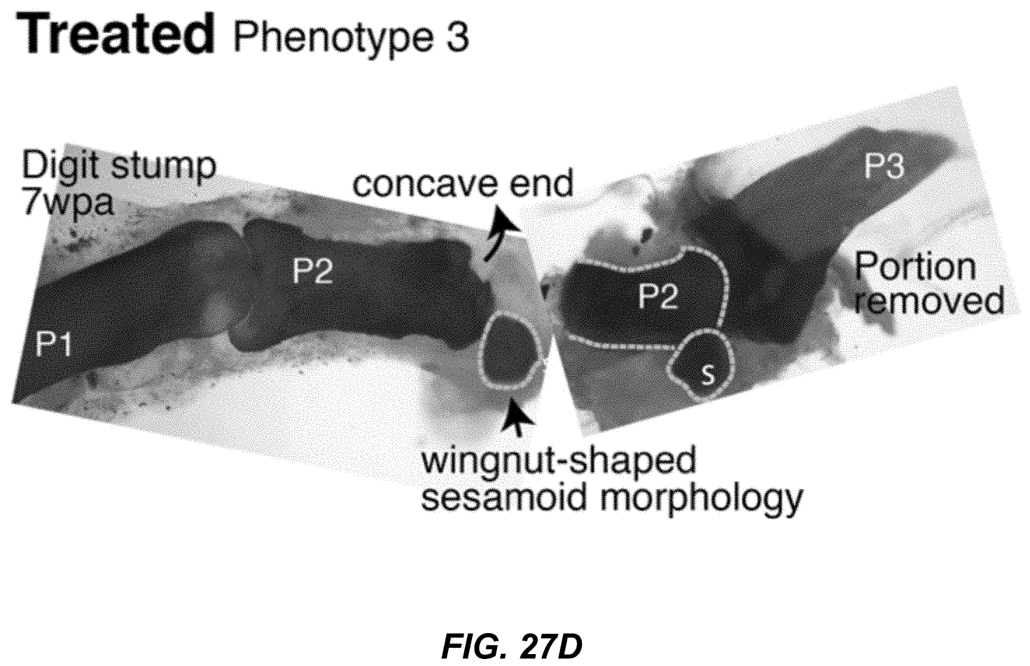

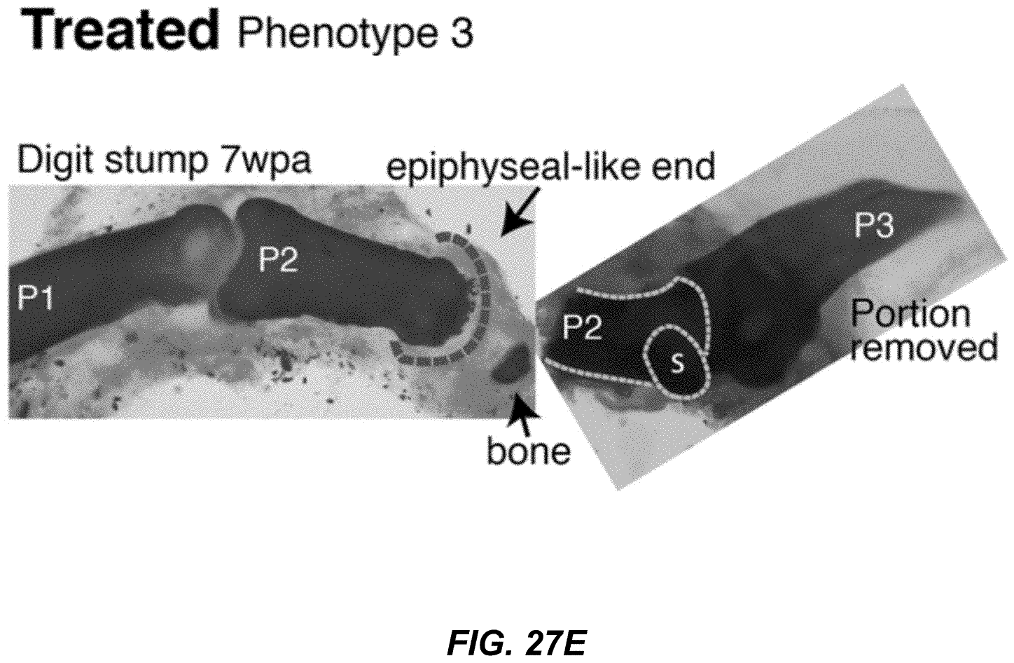

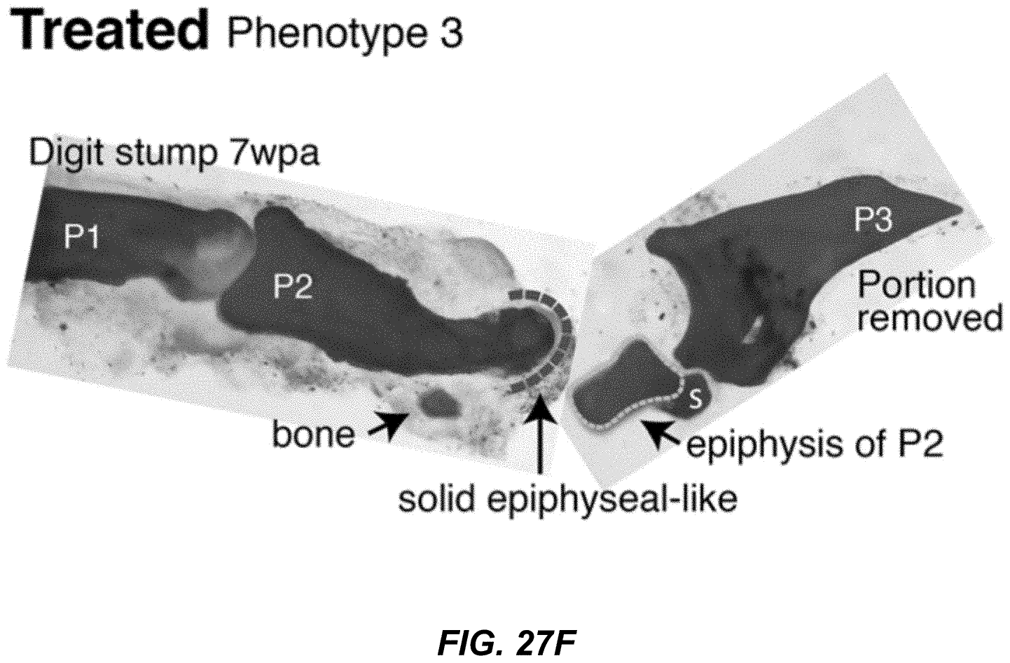

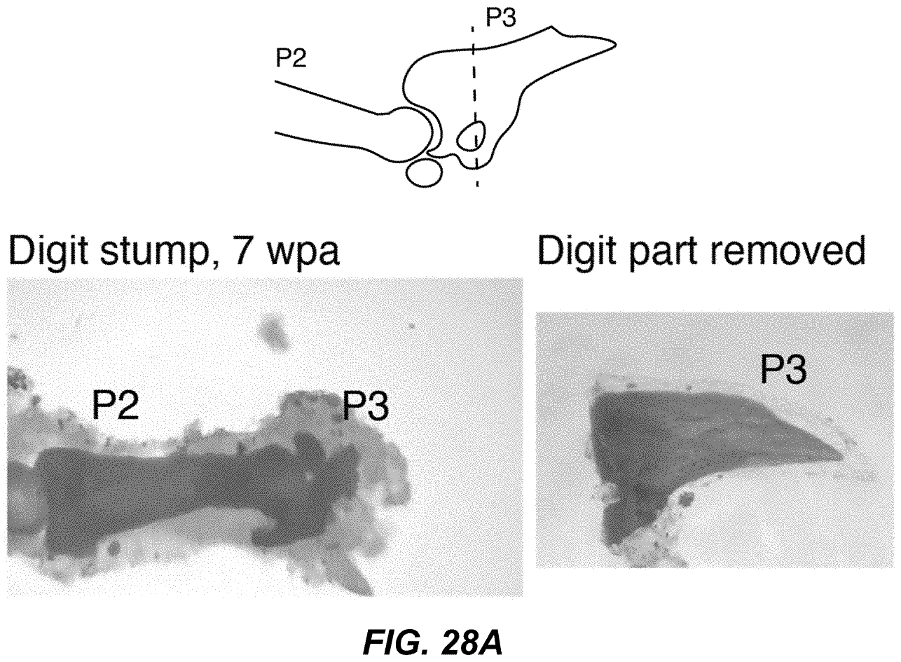

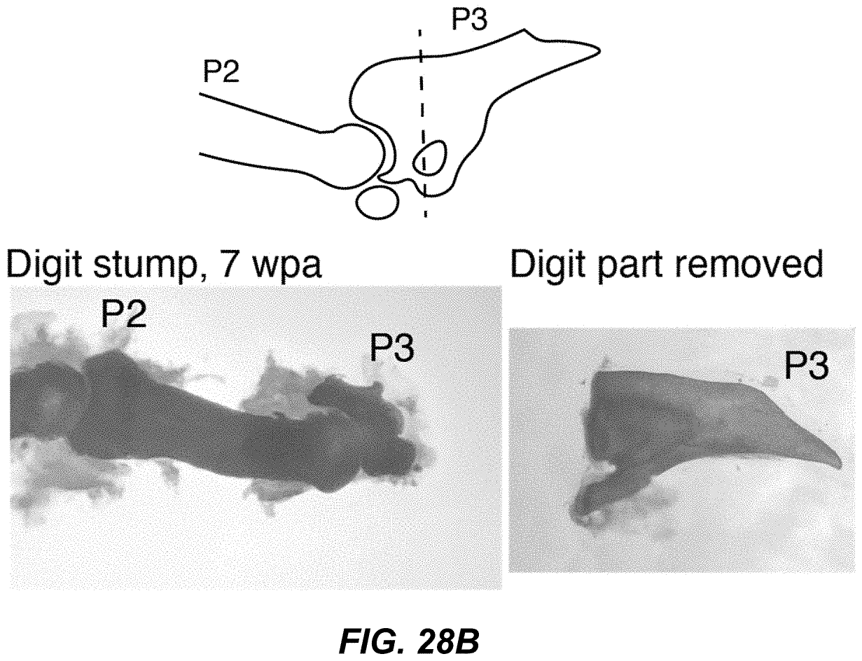

An unpatterned response was observed, as in Aurelia and Drosophila, (FIG. 26A-FIG. 26B, Table 10-Table 12), wherein skeletal staining reveals excessive bone mass around the digit stump, similarly to what was observed in some cases with BMP stimulation. However, patterned responses were also observed (FIG. 27A-FIG. 27F). The most dramatic regenerative response was observed in 2 digits (FIG. 18G-FIG. 18H). In one digit, an almost complete regrowth of the distal phalange and the nail was observed (FIG. 18G). Skeletal staining of the portion removed from this digit (FIG. 18J) shows that it was amputated at the proximal P3 transecting the os hole. By 7 weeks, skeletal staining of the regrown digit (FIG. 18J) shows that the P3 bone was almost completely regrown. The regrown P3 shows trabecular appearance that is similar in general structure but not identical to the original P3. Another dramatic response was observed from another digit, which began reforming the nail by 7 weeks (FIG. 18H). Skeletal staining of the portion removed from this digit shows that it was amputated across the P2 bone, removing the entire epiphyseal cap along with the sesamoid bone (FIG. 18K). Skeletal staining of the regenerating digit shows that the epiphyseal cap was regrown, along with its associated sesamoid bone. Moreover, articulating from the regenerated P2 appears to be the beginning of the next phalangeal bone (arrow, FIG. 18K). To our knowledge, the regenerative response observed in these digits represents the most dramatic extent of self-organized mammalian digit regeneration reported thus far. Distal phalange regeneration in adults has not been reported, while interphalangeal joint formation from a P2 amputation has been achieved only through sequential Bmp administration and there has been no documentation of the regrowth of the sesamoid bone.

### Conclusion

In this study, amputations were performed on Aurelia appendage, Drosophila limb, and mouse digit. None of these animals are known to regenerate robustly (Aurelia ) if at all (Drosophila and mouse) from these amputations. Upon administration of L-leucine and sugar/insulin, dramatic regenerative response was observed in all systems. The conserved effect of nutrient supplementation across three species that span 500 million years of evolutionary divergence suggests energetic parameters as ancestral regulators of regeneration activation in animals.

While the appendage regenerative effect of hypoxia beyond Aurelia was not tested, it is notable that in mice hypoxia coaxes cardiomyocytes to re-enter cell cycle and activating HIFα promotes healing of ear hole punch injury. The diverse physiologies of animals across phylogeny may seem difficult to reconcile with a conserved regulation of regeneration, especially in the view of regeneration as recapitulation of development. Growing a jellyfish appendage is different from building a fly leg or making a mouse digit. However, there is another way of looking at regeneration as a part of tissue plasticity. In this view of regeneration, before tissue-specific morphogenesis commences, a more upstream regulation is hypothesized that controls the broadly shared processes of growth, proliferation, and differentiation. In support of this idea, regeneration across species and organs relies one way or another on the presence of stem cells or differentiated cells re-entering cell cycle and re-differentiating. As disclosed herein, in animals that poorly regenerate, high nutrient input turns on growth and anabolic states that promote tissue rebuilding upon injury.

That regenerative response can be induced blurs the boundary between regenerating versus non-regenerating animals. The factors identified in the study are not exotic: variations in amino acids, carbohydrates, and oxygen levels are conditions that the animals can plausibly encounter in nature. These observations highlight two potential insights into regeneration. First, regeneration is environmentally dependent. An animal would stop at wound healing under low-energy conditions and regenerate in energy-replete conditions. In this view, for the animals examined in this study, the typical laboratory conditions may simply not be conducive to regeneration. Alternatively, what is observed is dormant regeneration, which can be activated with broad environmental factors. Without being bound by any particular theory, this interpretation is favored because the regenerative response was unusually variable. The variability stands in stark contrast to the robust regeneration in e.g., axolotl, planaria, or hydra. Just like mutations produce phenotypes with varying penetrance and expressivity, the variable regenerative response speaks to a fundamental consequence of activating a biological module that has been evolutionarily inactivated. The nature of the activators suggests ancestral regeneration as part of a response to broad environmental stimuli.

In particular, the conserved effects of nutrient supplementation suggest that regeneration might have originally been a part of growth response to abundant environments. No nutrient dependence has been observed in highly regenerating animal models such as planaria, hydra, and axolotl. Environment-dependent plasticity, however, is pervasive in development, physiology, behavior, and phenology. Without being bound by any particular theory, environment-dependent plasticity may have characterized the ancestral form of regeneration. Present regenerating lineages might have decoupled the linkage with environmental input and genetically assimilated regenerative response—because regeneration is adaptive or coupled to a strongly selected process, e.g., reproduction. Without being bound by any particular theory, non- or poorly regenerating animals might have also weakened the linkage with environmental input, but to silence the regenerative response. This predicts an ancient form of a robustly regenerative animal (like planaria, hydra, axolotl) that tunes its regeneration frequency to nutrient abundance. Such plasticity has been reported in the basal lineage Ctenophora.

In some embodiments, the present disclosure suggests that an inherent ability for appendage regeneration is retained in non-regenerating animals and can be unlocked with a conserved strategy. In some embodiments, the promoting factors disclosed herein may be combined with species- or tissue-specific morphogenetic regulators. Reiterating Spallanzani's hope, Marcus Singer supposed half a century ago that “. . . every organ has the power to regrow lying latent within it, needing only the appropriate ‘useful dispositions’ to bring it out” The surprising result disclosed herein is the simplicity by which the regenerative state can be promoted with ad libitum amino acid and sugar supplementation. This simplicity demonstrates a much broader possibility of organismal regeneration, and can help accelerate progress in regeneration induction across animals.

### Methods

Aurelia aurita. The experiments were performed in Aurelia aurita sp. 1 strain, also alternatively named Aurelia coerulea based on recent molecular classification. Polyps were reared at 68° F., in 32 ppt artificial seawater (ASW, Instant Ocean), and fed daily with brine shrimps (Artemia nauplii) enriched with Nannochloropsis algae (both from Brine Shrimp Direct). To induce strobilation, polyps were incubated in 25 M 5-methoxy-2-methyl-indole (Sigma M15451) at 68° F. for an hour. Ephyrae typically began to strobilate within a week.

Amputation. Strobilated ephyrae were fed daily with rotifers (Brachionus plicatilis, Reed Mariculture) until amputation time. 2-3 days old ephyrae were anesthetized in 400 μM menthol and amputated using a razor blade mounted on an x-acto knife handle. After amputation, ephyrae were let to recover in bubbler cones (FIG. 21). Regeneration was assessed at various times for 1-2 weeks after amputation, before onset of maturation to medusa. Experiment in the original habitat. The polyp population in the study arose from parental polyps collected off the coast of Long Beach, Calif. (33° 46′04.2″N 118° 07′44.2″W, GPS: 33.7678376,−118.1289559). Ephyrae were amputated in location and immediately after submersed in the ocean. For submerging the amputated ephyrae in the ocean, a two-layered aquarium was custom-built. Ephyrae were placed in plastic canisters with a 7 cm diameter hole cut in the lid and covered with a 250 μm plastic screen. The canisters were then placed in a thick plastic tank fitted with a 500 μm plastic screen on top. This design offers protection to the ephyrae against predators and strong waves, while at the same time allowing exchange of water, zooplanktons, and other particulates. Ephyrae were collected after two weeks.

Various Molecular and Physical Modulations were Screened to Recapitulate Arm Regeneration.

A. The choice of the factors screened was dictated by a combination of considerations: Evidence in literature for involvement in regeneration (in any systems), e.g., developmental pathways in various systems. Natural factors that have been shown, or are intuitively would be, relevant to Aurelia 's life history, e.g., nutrient level, oxygen level. Limitations such as commercial availability for drugs or feasible implementation for physical factors.

B. Experimental design and scoring methodologies were similar to those described in the Methods for the main experiment. Experiments were performed at 68° F., except for those testing the effects of different temperatures. Two-to-three-day-old ephyrae were anesthetized in 400 μM menthol and amputated using a razor blade. In all experiments, the amputation removed three arms, as illustrated in FIG. 13C. Except for experiments testing aquaria setups, amputated ephyrae were let to recover in 1 L sand settling cones (Nalgene Imhoff, FIG. 21). In each experiment, control and treated groups were set up side by side. Multiple nutrient levels were as controls, because a factor might be activating or inhibiting regeneration; Control and treated ephyrae were fed daily. Each experiment was repeated across 2-5 strobilation batches (biological replicates). Regeneration was assessed at 1-2 weeks after amputation.

C. Drug administration or implementation of physical parameter: Modulators were administered or physical parameters were implemented immediately upon amputation. For experiments testing small-molecule modulators, control and treated seawater was refreshed weekly. Some small molecules were dissolved in DMSO or ethanol; for these molecules, the control group was administered with an equal volume of the solvent.

D. To determine the range of parameters tested: When available, previously reported concentrations in cell culture or animal systems or order-of-magnitude range around these concentrations were tested. Absent of previous reports of physiologically relevant concentrations, the maximum possible concentration was tested. To determine the maximum concentration of a drug, ephyrae were incubated overnight in a wide range of concentrations, e.g., from 10 nM to 10 uM. Maximum concentration was determined by solubility in saltwater or onset of adverse effects (e.g., degrowth, paralysis, death). For factors that gave initial interesting effects (e.g., insulin), a range of lower concentrations were subsequently tested for optimization.

E. A negative result in the screen: Given the variability in the regenerative response, the goal of this screen was to look for strong effects. A “negative result” means that no obvious strong effects were observed that warrant further investigation. The negative result conclusion was limited to the specific drug or factor, concentration or parameter, and implementation method tested in the screen. For instance, it is possible that the optimal concentration or the optimal time period to deliver the drug was not found.

Regeneration experiments. All experiments were performed at 68° F. Amputated ephyrae were let to recover in 1 L sand settling cones (Nalgene Imhoff, FIG. 21). In each cone, an airline from a Tetra Whisper 100 pump was placed at the bottom to create a gentle upward current (-1 air bubble/sec, FIG. 30). In this “bubbler cone” setup, the ephyrae continually experienced water current, either the upward bubble-generated current or the downward gravity-generated current. The conical geometry helps avoid stagnant spots, where the ephyrae could get stuck. Each cone housed 30 ephyrae in 500 mL ASW to avoid crowding and fouling. ASW was changed weekly.

Two-to-three-day-old ephyrae were anesthetized in 400 μM menthol and amputated using a razor blade mounted on an x-acto knife handle. Amputated ephyrae were let to recover in 1 L sand settling cones (Nalgene Imhoff, FIG. 21). In each experiment, ˜90 animals were amputated for each condition (e.g., 90 animals for control and 90 animals for treated). Because of the varying baseline across strobilation batches, each experiment was repeated across 2-5 strobilation batches (biological replicates). These sample sizes were chosen to obtain a 95% confidence level on the treatment effect (statistical analysis described below). Hundreds of experimental animals were first amputated, mixed together in a beaker, and then randomly allocated to the control or treatment groups. Regeneration was assessed at various times for 1-2 weeks after amputation, before onset of maturation to medusa. All data were included in the analysis.

Rationale for the amputation scheme. Among the possible amputation schemes, 3-arm amputation was chosen because it could be performed fastest. Removing 1 arm requires carefully cutting across the base of the arm while avoiding injuring the surrounding body. Removing 2 arms is less hard but still requires awkward positioning of the knife. Removing 4 arms again takes more time because it requires cutting through the large protruding manubrium, which also affects the animal's feeding ability. The fast 3-arm amputation facilitates testing hundreds of ephyrae per experiment.

Nutrients. Amputated ephyrae were fed daily with rotifers. The number of rotifers was estimated using a 6-well plate fitted with STEMgridTM (the same principle as using a hemocytometer). In some embodiments, low food was ˜100-200 rotifers/ephyra and high food was 400 rotifers/ephyra. In some embodiments, “low” or “high” food amount may be relative to and easily vary across cultures (e.g., rotifer culture, differences across Aurelia strains, etc.). Most if not all rotifers were typically consumed within an hour (determined by measuring the rotifers in the water).

Insulin. Immediately after amputation, ephyrae were placed in ASW supplemented with 500 nM human recombinant insulin (Sigma 10908). Insulin was refreshed weekly. To determine the concentration used, a range of concentrations, 10 nM to 3 mM, were tested. The concentration 500 nM was chosen as it maximized regeneration frequency while avoiding solubility problems. To control that the effect of insulin was not due to non-specific additions of proteins, BSA at 500 nM and 3 mM were tested.

Hypoxia. Immediately after amputation, ephyrae were placed in hypoxic ASW. To create a hypoxic environment, nitrogen or argon, instead of ambient air, was pumped into the bubbler cone, beginning from the day before the experiment and maintained throughout the duration of the experiment. The bubbler cone was sealed with parafilm to maintain the lowered oxygen level. The nitrogen/argon flow was adjusted to achieve 50% reduction in the dissolved oxygen level. Dissolved oxygen level was measured using a Clark-type electrode Unisense OX-500 microsensor. The measurement was normalized to oxygen level in control ASW bubbled normally with ambient air. Oxygen measurement was performed prior to the experiment and subsequently every 3 days.

L-leucine. Immediately after amputation, ephyrae were placed in ASW supplemented with 100 mM L-leucine (Sigma L1002, the cell-permeable methyl ester hydrochloride form). L-leucine was refreshed weekly. To determine the concentration used, a range of concentrations from one to hundreds of mM was tested. The concentration of 100 mM was chosen as it maximized the regeneration frequency without non-specific, negative effects.

Statistical analysis. To assess the statistical significance of the treatments, meta-analysis of effect size was performed. For each experiment, the effect size of a treatment was computed relative to the internal control set up using ephyrae from the same clutch. The effect size metrics used are determined by the form of the dataset. For measurements of frequencies (e.g., regeneration frequency), the datasets are in the form of a 2×2 table of dichotomous variables,

TABLE 13

| | # ephyrae that | # ephyrae that do |
|---|---|---|
| | regenerate | not regenerate |
| ControL | a | b |
| Treatment | c | d |

For such 2×2 datasets, in situations where the baseline varies (e.g., varying baseline regeneration across clutches), the commonly used measures of effect size are the Risk Ratio (RR, EQUATION 1)

$$RR = \frac{\begin{matrix}( \frac{\# ephyrae that regenerate}{total \# ephyrae} ) \\ in treated group\end{matrix}}{\begin{matrix}( \frac{\# ephyrae that regenerate}{total \# ephyrae} ) \\ in control group\end{matrix}} = \frac{\frac{c}{( c + d )}}{\frac{a}{( a + b )}} \qquad EQUATION 1$$

and the Odds Ratio (OR, EQUATION 2),

$$OR = \frac{\begin{matrix}( \frac{\# ephyrae that regenerate}{\# ephyrae that do not regenerate} ) \\ in treated group\end{matrix}}{\begin{matrix}( \frac{\# ephyrae that regenerate}{\# ephyrae that do not regenerate} ) \\ in control group\end{matrix}} = \frac{\frac{c}{( c + d )}}{\frac{a}{( a + b )}} \qquad EQUATION 2$$

RR compares the probability of an outcome in treated vs control group, whereas OR compares the odds of an outcome in treated vs control group.

For measurements of arm length and body size, the datasets are in the form of continuous variables. For such data, the commonly used effect size is the Response Ratio (R, EQUATION 3),

$$R = \frac{mean arm length in treated group}{mean arm length in control group} \qquad EQUATION 3$$

R evaluates the proportionate change that results from a treatment, and is the meaningful effect size to use when the outcome of a treatment is measured on a physical scale, e.g., length or area (as opposed to arbitrary scale, e.g., happiness level). Experiments where regeneration in one of the groups occurred in 0 ephyra were necessarily excluded.

Having computed the effect size (RR, OR, or R) within each experiment, meta-analysis of the effect size across experiments was performed. The metafor package in R was used, with fixed-effect model (for nutrients and leucine) or random-effect restricted maximum likelihood model (for insulin and hypoxia, which had different control conditions across the experiments). Statistical coefficients were based on normal distribution.

Phalloidin and tyrosinated tubulin staining. All steps were performed at room temperature, unless indicated otherwise. Ephyrae were first anesthetized in 400 μM menthol, which minimizes curling during fixing. Next, ephyrae were fixed in 3.7% (v/v) formaldehyde (in PBS) for 15 minutes, permeabilized in 0.5% Triton X-100 (in PBS) for 5 minutes, and blocked in 3% (w/v) BSA for 2 minutes. For neuron staining, ephyrae were incubated in 1:200 mouse anti-tyrosinated alpha tubulin antibody (Sigma MAB1864-I) overnight at 4° C., and then in 1:200 goat-anti-mouse Alexa Fluor 488 (Sigma A11029) overnight in the dark at 4° C. Primary or secondary antibodies were diluted in 3% BSA. For actin staining, ephyrae were incubated in 1:20 Alexa Fluor 555 Phalloidin (Life Technologies A12379) overnight or for 2 hours in the dark at 4° C. For nuclei staining, ephyrae were incubated in 1:10 Hoechst 33342 (Sigma B2261) for 30 minutes in the dark.

Microscopy. Ephyrae were imaged anesthetized in menthol. Brightfield images, fluorescent images, and movies were taken with the Zeiss AxioZoom.V16 stereo zoom microscope and AxioCam HR 13-megapixel camera. Optical sectioning was performed with ApoTome.2.

Drosophila melanogaster. OregonR and CantonS wild type strains were reared under standard conditions at 23° C.

Amputation. Amputation was performed on adult flies 2-7 days after eclosion. Flies were anesthetized with CO2, placed under a dissection microscope, and tibia amputated using a spring scissors (Fine Science Tools, 91500-09) and superfine dissecting forceps (VWR, 82027-402). See FIG. 16A-FIG. 16E for detailed description of the amputation plane. Recovering Drosophila were fed with standard lab fly food (control) or standard lab fly food mixed with 5 mM L-Leucine (Sigma L8000), 5 mM L-Glutamine (Sigma G3126), and 0.1 mg/mL insulin (human recombinant, MP Biomedicals 0219390080). To introduce the molecular factors, the fly food was microwaved in short pulses, such that the topmost layer of the food was liquified. Molecular factors in aqueous medium were then pipetted into this liquified layer. Food was allowed to re-set at 4° C. for at least 20 minutes. New food was prepared fresh every 2 days, and flies were moved into freshly prepared treated food every 2 days, throughout the course of the 2- to 3-week experiment. The Drosophila data reported in this study were reproduced by 3 independent experimenters, with many experiments examined at multiple times by 2 experimenters.

Statistical analysis. The reported sample size was chosen to obtain >90% confidence level. As described for the Aurelia data, meta-analysis of the effect size across genetic was performed in R using the metafor package with random-effect restricted maximum likelihood model and statistical coefficients based on normal distribution. Since the baseline is 0% regeneration, effect size is computed as the difference in the regeneration frequency between treated and control groups(i.e., Risk Difference metric in metafor). All data were included in the analysis.

DAPI staining. Fly tibias were dissected and washed in 70% ethanol (<1min) to decrease the hydrophobicity of the cuticle and washed in PBS with 0.3% Triton-X for 10 minutes. The legs were fixed in 4% paraformaldehyde (in PBS) overnight at 4° C. and washed five times for 20 minutes each in PBS with 0.3% Triton-X. The legs were equilibrated in Vectashield mounting medium with DAPI (Vector H-1200) overnight at 4° C., and imaged using Zeiss AxioZoom.V16 stereo zoom microscope with AxioCam HR 13-megapixel camera. Confocal imaging was performed using X-Light V2 spinning disk mounted on the Olympus IX81 inverted microscope.

Live fly imaging. Flies anesthetized on a CO2 bed were imaged under a dissection scope equipped with the Zeiss AxioCam 503 color camera.

Electron microscopy. Environmental scanning electron microscopy (ESEM) was performed on a FEI Quanta 200F (FEI, Hillsboro, Oreg.). Whole live flies were mounted onto the SEM stub with copper tape. ESEM images were attained at a pressure of 0.1 mbar and 5 kV at a working distance of 9-12 mm, with water as the ionizing gas.

Mus musculus. All studies comply with relevant ethical regulations for animal testing and research, and received ethical approval by the Institutional Animal Care and Use Committees at the California Institute of Technology.

Strain. Adult female (3-6 months old) wild-type CD1 mice (Charles River Laboratories strain 022) were used for all regeneration studies.

Mouse digit amputation. Digit amputation was performed following the established protocol in the field. Mice were anesthetized with 1-5% isoflurane (in oxygen) in an induction chamber, followed by maintenance on a nosecone. The mouse was positioned on its belly with its hind paws outstretched and the ventral side of the paw facing upwards. Sustained-Release Buprenorphine was administered (Buprenorphine SR LAB®) at 0.5 mg/kg subcutaneously as an analgesic. Blood flow to the hindlimb was stemmed by tying a rubber band around the ankle and clamping it with a hemostat. All surgical procedures were carried out under a Zeiss Stemi 305 dissection microscope. An initial incision, parallel to the position of foot, was made through the ventral fat pad using Vannas spring scissors (World Precision Instruments, 14003). The length of this incision was determined by the amount of ventral skin needed to seal the digit amputation wound completely. The ventral skin freed in the initial incision was peeled back using surgical forceps, and a no. 10 scalpel (Sklar, 06-3110) was used to amputate and bisect the digit completely through the second or third phalange. Digits 2 and 4 on the right hind paw were operated on in this fashion, while digit 3 remained unamputated as a control. The amputation wound was immediately closed with the ventral skin flap and sealed with GLUture (Zoetis, Kalamazoo, MI). Amputated portions were immediately fixed as control for skeletal staining. Amputated digits were photographed weekly for 7 weeks, at which time the digits were dissected for skeletal staining.

Statistical analysis. The sample size in the experiment balanced the aim of achieving >90% confidence level with ethical consideration of minimizing the number of animals used. Animals were randomly allocated to the control or treatment group. No restricted randomization was applied. For weight measurement, the unit of analysis is a single animal. For regeneration phenotype, the unit of analysis is a single digit. Student's t-test was used to evaluate the null hypothesis that there is no difference between the control and treated groups. 95% confidence intervals were computed assuming normal distribution. All data were included in the analysis.

Mouse digit dissection and skeletal staining. Mice were euthanized and digits 2, 3 and 4 were removed with a no. 10 scalpel (Sklar, 06-3110) through the first phalange. Excess skin and flesh were removed with spring scissors (Fine Science Tools, 91500-09) and fine dissecting forceps (Fine Science Tools, 11254-20). All digits analyzed by whole-mount skeletal stains were prepared with a standard alizarin red and alcian blue staining protoco1.48. Digits were dehydrated in 95% ethanol for 1 day, and incubated in staining solution (0.005% alizarin red, 0.015% alcian blue, 5% acetic acid, 60% ethanol) for 1 day at 37° C. Tissue was cleared in 2% potassium hydroxide at room temperature for 1 day, 1% potassium hydroxide for 1 day, and then taken through an increasing glycerol series (25%, 50%, 75%, 100%). The stained samples were imaged on Zeiss AxioZoom.V16 stereo zoom microscope with a Zeiss AxioCam 503 color camera or a Zeiss Stemi 305 dissection microscope with an iPhone 6 camera.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g.,“ a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “ a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A method for inducing reparative regeneration or appendage regeneration, comprising administering to a subject in need thereof a therapeutically effective amount of one or more amino acids and a therapeutically effective amount of one or more sugars, and thereby inducing reparative regeneration or appendage regeneration in the subject.

2. A method for inducing reparative regeneration, comprising administering to a subject in need thereof:
a therapeutically effective amount of a first regenerative agent or a pharmaceutically acceptable salt, ester, solvate, stereoisomer, tautomer, or prodrug thereof, that stimulates mTOR signaling; and
a therapeutically effective amount of a second regenerative agent or a pharmaceutically acceptable salt, ester, solvate, stereoisomer, tautomer, or prodrug thereof, that stimulates insulin signaling, and thereby inducing reparative regeneration in the subject.

3. (canceled)

4. The method of claim 2, wherein the first regenerative agent comprises MHY1485, 3BDO, CL316,243, one or more amino acids, or any combination thereof.

5.-7. (canceled)

8. The method of claim 4, wherein the one or more amino acids comprises alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine, phosphoserine, phosphothreonine, phosphotyrosine, 4-hydroxyproline, hydroxylysine, demosine, isodemosine, gamma-carboxyglutamate, hippuric acid, octahydroindole-2-carboxylic acid, statine, 1,2,3,4-tetrahydroisoquinoline-3-carboxylic acid, penicillamine, ornithine, 3-methylhistidine, norvaline, beta-alanine, gamma-aminobutylic acid, cirtulline, homocysteine, homoserine, methyl-alanine, para-benzoylphenylalanine, phenylglycine, propargylglycine, sarcosine, methionine sulfone, tert-butylglycine, 3,5-dibromotyrosine, 3,5-diiodotyrosine, glycosylated threonine, glyclosylated serine, glycosylated asparagine, or any combination thereof.

9. (canceled)

10. (canceled)

11. The method of claim 4, wherein the one or more amino acids comprises L-leucine.

12. (canceled)

13. The method of any claim 2, wherein the second regenerative agent comprises an insulin receptor agonist.

14. (canceled)

15. The method of claim 2, wherein the second regenerative agent comprises insulin and/or one or more sugars.

16. The method of claim 15, wherein the one or more sugars comprise a monosaccharide, a disaccharide, a polysaccharide, or any combination thereof.

17. The method of claim 15, wherein the one or more sugars comprise sucrose, dextrose, maltose, dextrin, xylose, ribose, glucose, mannose, galactose, sucromalt, fructose (levulose), or any combination thereof.

18. The method of claim 2, wherein the second regenerative agent increases insulin secretion.

19. The method of claim 2, wherein the subject in need is a subject suffering from or at a risk to develop a disease or disorder, wherein the disease or disorder results in damage, injury, or loss of a limb, organ, tissue, cell, or any combination thereof.

20. The method of claim 2, wherein the subject in need is suffering from an acute injury.

21.-24. (canceled)

25. The method of claim 2, wherein the reparative regeneration comprises regeneration of one or more tissues.

26. (canceled)

27. (canceled)

28. The method of claim 2, wherein the reparative regeneration and/or appendage regeneration is patterned.

29.-34. (canceled)

35. The method of claim 2, wherein the second regenerative agent is administered before initiating administration of the first regenerative agent.

36. The method of claim 2, wherein the administration of the first regenerative agent continues after cessation of administration of the second regenerative agent, and/or wherein the administration of the second regenerative agent continues after cessation of administration of the first regenerative agent.

37. (canceled)

38. (canceled)

39. The method of claim 2, wherein the administration of one or both of the first and second regenerative agents is initiated within a therapeutically effective time window.

40.-57. (canceled)

58. The method of claim 2 any one of claims 1 57, comprising administering a third regenerative agent that activates mTOR signaling.

59. (canceled)

60. (canceled)

61. The method of claim 2, wherein the method does not induce insulin resistance.

62. The method of claim 2, wherein the method comprises contacting the subject in need with a scaffold,
wherein the scaffold comprises a bandage, beads, a hydrogel, a polymer, or other biomaterial, or any combination thereof; and
wherein the scaffold comprises a bone morphogenetic protein (BMP), a hormone, a growth factor, or other agent that induces reparative regeneration and/or appendage regeneration, or any combination thereof.

63. (canceled)


SEQUENCE LISTING (2 sequences)

SEQ ID NO: 1 - protein, 555 aa, Artificial Sequence
  Human mTOR kinase domain
LKNMCEHSNTLVQQAMMVSEELIRVAILWHEMWHEGLEEASRLYFGERNVKGMFEVLEPLHAMMERGPQTLKETSFNQAYGRDLMEAQEWCRKYMKSGNVKDLTQAWDLYYHVFRRISKQLPQLTSLELQYVSPKLLMCRDLELAVPGTYDPNQPIIRIQSIAPSLQVITSKQRPRKLTLMGSNGHEFVFLLKGHEDLRQDERVMQLFGLVNTLLANDPTSLRKNLSIQRYAVIPLSTNSGLIGWVPHCDTLHALIRDYREKKKILLNIEHRIMLRMAPDYDHLTLMQKVEVFEHAVNNTAGDDLAKLLWLKSPSSEVWFDRRTNYTRSLAVMSMVGYILGLGDRHPSNLMLDRLSGKILHIDFGDCFEVAMTREKFPEKIPFRLTRMLTNAMEVTGLDGNYRITCHTVMEVLREHKDSVMAVLEAFVYDPLLNWRLMDTNTKGNKRSRTRTDSYSAGQSVEILDGVELGEPAHKKTGTTVPESIHSFIGDGLVKPEALNKKAIQIINRVRDKLTGRDFSHDDTLDVPTQVELLIKQATSHENLCQCYIGWCPFW

SEQ ID NO: 2 - protein, 558 aa, Artificial Sequence
  Aurelia mTOR kinase domain
LKNMCEHSQTLVQQAMMVSEELIRVAILWHELWHEGLEEASRLYFGERNVKGMFTVLDPLHQMMERGPQTLNETSFQQAYGRELMEAQEWCRKYQRSANTKDLTQAWDLYYHVFRRISKQLPQLTSLELQYVSPKLLTCSDLELAVPGTYEPHNPIVQIKQVSTTLNVITSKQRPRKLSITGCNDQEYMFLLKGHEDLRQDERVMQLFGLVNTLLAADTETFKRHLSIQRYAVVPLSTNSGLIGWVPHCDTLHTLIRDYREKKKILLNIEHRIMLRMAPGYDHLTLMQKVEVFEHALGNTNGEDLAKVIWLKSPSSEVWFDRRTNYTRSLAVMSMVGYILGLGDRHPSNLMLDRMSGKILHIDFGDCFEVAMTREKFPEKIPFRLTRMLTNAMEVTGIDGNYRLTCQSVMQVLRENKDSVMAVLEAFVYDPLLNWRLMDAPKGKRSKGRSESYSSHSETNDMLESFEMTRDRPRKQQKEVSAEQPRASGNGDDEENAKPEALNKKALQIVKRVKDKLTGCDFNNDDSVDVNSQVDLLIKQATSHENLCQCYIGWCPFW